(19) United States
(12) Patent Application Publication
(10) Pub. No.: US 2010/0286152 A1
(43) Pub. Date: Nov. 11, 2010

(54) N-PHENYL HYDRAZIDES AS MODULATORS OF THE GHRELIN RECEPTOR

(75) Inventors: Giovanni Bernasconi, Verona (IT); Steven Mark Bromidge, Harlow, Essex (GB); Andrew James Carpenter, Research Triangle Park, NC (US); Lucilla D'Adamo, Verona (IT); Romano Di Fabio, Verona (IT); Sebastien Guery, Verona (IT); Francesca Pavone, Verona (IT); Alfonso Pozzan, Verona (IT); Marilisa Rinaldi, Verona (IT); Fabio Maria Sabbatini, Verona (IT); Yves St-Denis, Verona (IT)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited, Greenford (GB)

(21) Appl. No.: 12/602,829

(22) PCT Filed: Jun. 5, 2008
(86) PCT No.: PCT/EP2008/057018

§ 371 Date: Jul. 27, 2010

(30) Foreign Application Priority Data
Jun. 6, 2007 (GB) 0710865.7

(51) Int. Cl. A61K 31/495; C07D 409/06; C07D 405/06; C07D 295/13; C07D 401/06; C07D 403/06; C07D 211/28; A61K 31/496; A61K 31/4525; A61K 31/445; A61K 31/506; A61P 3/00

(52) U.S. Cl. 514/249; 544/375; 544/400; 544/376; 544/377; 544/360; 544/362; 546/197; 546/205; 544/295; 544/349; 514/252.13; 514/254.11; 514/252.12; 514/253.01; 514/253.04; 514/321; 514/319; 514/252.14

(57) ABSTRACT

The present invention relates to novel compounds of formula (I) or a pharmaceutically acceptable salt or solvate thereof,

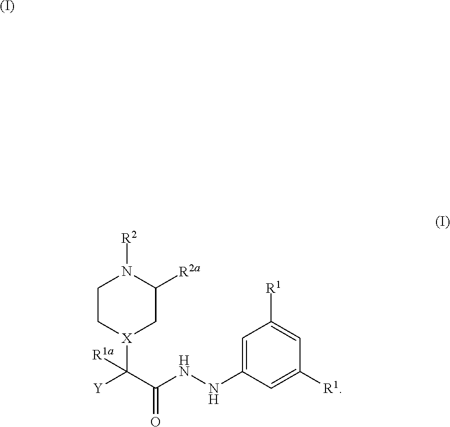

The present invention relates to novel acetohydrazide compounds, processes for their preparation, intermediates usable in these processes, and pharmaceutical compositions containing the compounds. The invention also relates to the use of the acetohydrazide compounds in therapy, for example as modulators of the growth hormone secretagogue receptor (also referred to as the ghrelin receptor or GHSR1a receptor) and/or for the treatment and/or prophylaxis of a disorder mediated by the ghrelin receptor.

Ghrelin is the endogenous ligand for the growth hormone (GH) secretagogue receptor. It was originally purified from stomach and is a 28 amino acid peptide hormone in which the serine at position 3 is n-octanoylated. It has potent GH releasing activity and thus is believed to play an important role in maintaining GH release and energy homeostasis. In particular, it appears to exert potent appetite-stimulating activities.

It is therefore desirable to find new compounds which modulate ghrelin receptor activity.

According to the invention there is provided a compound of formula (I) or a pharmaceutically acceptable salt or solvate thereof:

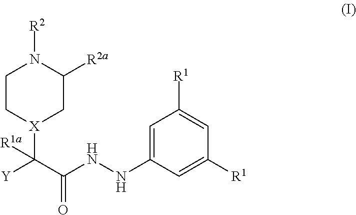

wherein:
each R¹ is independently selected from the group consisting of Cl, Br, CH₃ and CF₃;
X is carbon or nitrogen;
R^{1a} is H or a straight C₁₋₃ alkyl group;
R^{2a} is H or a methyl group
R² is selected from the group consisting of C₁₋₃alkyl, H and —(CH₂)ₙ—, wherein n is 3 or 4 and the terminal carbon of the chain is bonded to the carbon atom adjacent to the nitrogen bearing the R² group, such that a fused 6,5 or 6,6-bicyclic ring is formed.
Y is selected from the group consisting of: phenyl which may be unsubstituted or substituted by one or more substituents independently selected from the group consisting of C₁₋₃alkyl, C₁₋₃alkoxy, halogen, C₁₋₃alkyl substituted by 1 to 7 fluoro atoms and C₁₋₃alkoxy substituted by 1 to 7 fluoro atoms; pyridyl which may be unsubstituted or substituted by one or more substituents independently selected from the group consisting of C₁₋₃alkyl, OCH₃, CF₃, CN, and halogen; naphthyl which may be unsubstituted or substituted by one or more substituents independently selected from the group consisting of F and OCH₃; pyrimidinyl; imidazo[1,2-a]pyridine-6-yl; benzothiophen-2-yl; benzothiophen-5-yl; benzofuran-2-yl; dibenzo[b,d]furan-3-yl; dibenzo[b,d]thiophen-2-yl; dibenzo[b,d]thiophen-4-yl; 1,3-benzodioxol-5-yl; 2,3-dihydro-1,4-benzodioxin-5-yl; 2,3-dihydro-1,4-benzodioxin-6-yl; 2,3-dihydro-1-benzofuran-4-yl; 2,2-difluoro-1,3-benzodiox-4-yl; pyridazinyl; imidazolyl; oxazolyl; pyrazolyl; thiazolyl; and triazolyl;
with the proviso that when Y is 2,3-dihydro-1,4-benzodioxin-6-yl, R^{1a} and R^{2a} are both H, R¹ is not Cl.

As used herein, the term “alkyl” refers to straight or branched hydrocarbon chains containing the specified number of carbon atoms. For example, C₁₋₃ alkyl means a straight or branched alkyl chain containing at least 1, and at most 3, carbon atoms. Examples of “alkyl” as used herein include, but are not limited to, methyl, ethyl, n-propyl and iso-propyl.

As used herein, the term “halogen” refers to F, Cl, Br or I.

In one embodiment each R¹ is independently CF₃ or Cl.

In one embodiment R^{1a} is a methyl group

In another embodiment R² is hydrogen, methyl, or —(CH₂)₃— wherein the terminal carbon atom of this chain is bonded to the carbon atom adjacent to the nitrogen bearing the R² group such that a fused 6,5-bicyclic ring is formed.

In another embodiment Y is selected from the group consisting of: phenyl which may be unsubstituted or substituted by one or more substituents independently selected from the group consisting of CH₃, OCH₃, OCF₃, F and Cl; pyridyl which may be unsubstituted or substituted by one or more substituents independently selected from the group consisting of CH₃ and Cl; naphthyl which may be unsubstituted or substituted by one or more substituents independently selected from the group consisting of F and OCH₃; pyrimidinyl; imidazo[1,2-a]pyridine-6-yl; benzothiophen-2-yl; benzothiophen-5-yl; benzofuran-2-yl; dibenzo[b,d]furan-3-yl; dibenzo[b,d]thiophen-2-yl; dibenzo[b,d]thiophen-4-yl; 1,3-benzodioxol-5-yl; 2,3-dihydro-1,4-benzodioxin-5-yl; 2,3-dihydro-1,4-benzodioxin-6-yl; 2,3-dihydro-1-benzofuran-4-yl; and 2,2-difluoro-1,3-benzodiox-4-yl.

In a further embodiment Y is selected from the group consisting of: 1,3-benzodioxol-5-yl, 2,3-dihydro-1,4-benzodioxin-6-yl, 2-chlorophenyl, 2-pyridyl, 3-pyridyl, 2-fluorophenyl, 2-methoxy-6-fluorophenyl, 3-methylphenyl, 2-methylpyridin-5-yl, 2-methylpyridin-3-yl, naphth-1-yl and 2,6-dimethylpyridin-3-yl.

In a further embodiment there is provided a group of compounds of formula (I) (Group A) wherein: each R¹ is independently CF₃ or Cl, X is nitrogen, R² is hydrogen or methyl, and Y is selected from the group consisting of: 1,3-benzodioxol-5-yl, 2,3-dihydro-1,4-benzodioxin-6-yl, 2-chlorophenyl, 2-pyridyl, 3-pyridyl, 2-fluorophenyl, 2-methoxy-6-fluorophenyl, 3-methylphenyl, 2-methylpyridin-5-yl, and 2-methylpyridin-3-yl.

In a further embodiment there is provided another group of compounds of formula (I) (Group B) wherein: each R¹ is independently CF₃ or Cl, X is nitrogen, R² is —(CH₂)₃— wherein the terminal carbon atom of this chain is bonded to the carbon atom adjacent to the nitrogen bearing the R² group such that a fused 6,5-bicyclic ring is formed, and Y is selected from the group consisting of 2,6-dimethylpyridin-3-yl, 2-chlorophenyl, pyridin-3-yl and naphth-1-yl.

It is to be understood that the present invention covers all combinations of substituent groups referred to herein above.

It will be appreciated by the person skilled in the art that the compounds of formula (I) may exist either in racemic form or homochiral form. All such forms are included within the scope of the present invention. * indicates a stereocentre of fixed but unknown stereochemistry i.e. either R or S stereochemistry. Diastereoisomer 1 or Diastereoisomer 2 means a compound of the invention or an intermediate thereof in homochiral form as a single diastereoisomer whose absolute configuration at one stereocentre was not determined. Enantiomer 1 or Enantiomer 2 means a compound of the invention or an intermediate thereof as a single enantiomer of unknown absolute stereochemistry.

In a further embodiment there is provided a compound of formula (I)′ or a pharmaceutically acceptable salt or solvate thereof:

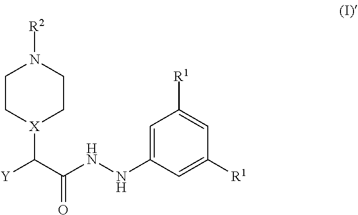

wherein
each R¹ is independently selected from the group consisting of Cl, Br, CH₃ and CF₃;
X is carbon or nitrogen;
R² is selected from the group consisting of C₁₋₃alkyl, H and —(CH₂)ₙ—, wherein n is 3 or 4 and the terminal carbon of the chain is bonded to the carbon atom adjacent to the nitrogen bearing the R² group, such that a fused 6, 5 or 6,6-bicyclic ring is formed.

Y is selected from the group consisting of: phenyl which may be unsubstituted or substituted by one or more substituents independently selected from the group consisting of C₁₋₃alkyl, C₁₋₃alkoxy, halogen, C₁₋₃alkyl substituted by 1 to 7 fluoro atoms and C₁₋₃alkoxy substituted by 1 to 7 fluoro atoms; pyridyl which may be unsubstituted or substituted by one or more substituents independently selected from the group consisting of C₁₋₃alkyl, OCH₃, CF₃, CN, and halogen; naphthyl which may be unsubstituted or substituted by one or more substituents independently selected from the group consisting of F and OCH₃; pyrimidinyl; imidazo[1,2-a]pyridine-6-yl; benzothiophen-2-yl; benzothiophen-5-yl; benzofuran-2-yl; dibenzo[b,d]furan-3-yl; dibenzo[b,d]thiophen-2-yl; dibenzo[b,d]thiophen-4-yl; 1,3-benzodioxol-5-yl; 2,3-dihydro-1,4-benzodioxin-5-yl; 2,3-dihydro-1,4-benzodioxin-6-yl; 2,3-dihydro-1-benzofuran-4-yl; 2,2-difluoro-1,3-benzodiox-4-yl; pyridazinyl; imidazolyl; oxazolyl; pyrazolyl; and triazolyl;

with the proviso that when Y is 2,3-dihydro-1,4-benzodioxin-6-yl R¹ is not Cl.

In a further embodiment there is provided a compound of formula (IA) or a pharmaceutically acceptable salt or solvate thereof:

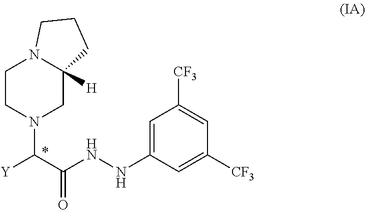

wherein:
Y is selected from the group consisting of pyridyl which may be unsubstituted or substituted once or twice by C₁₋₃alkyl; and phenyl substituted once by halogen.

In a further embodiment there is provided a compound of formula (IB) or a pharmaceutically acceptable salt or solvate thereof:

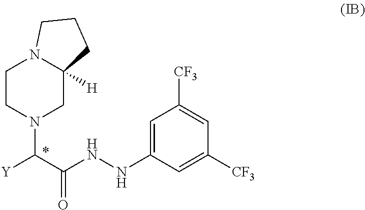

wherein:
Y is selected from the group consisting of pyridyl; phenyl substituted once by halogen; and naphthyl.

Particular compounds according to the invention include those mentioned in the examples and their pharmaceutically acceptable salts and solvates. Specific examples which may be mentioned include:
formic acid-(±)-2-dibenzo[b,d]thien-4-yl-N′-(3,5-dichlorophenyl)-2-(4-methyl-1-piperazinyl)acetohydrazide, mixture of enantiomers;
formic acid-(±)-2-dibenzo[b,d]furan-4-yl-N′-(3,5-dichlorophenyl)-2-(4-methyl-1-piperazinyl)acetohydrazide, mixture of enantiomers;
formic acid-(±)-N′-(3,5-dichlorophenyl)-2-(4-methyl-1-piperazinyl)-2-(2-naphthalenyl)-acetohydrazide, mixture of enantiomers;
formic acid-(±)-2-(1-benzofuran-2-yl)-N′-(3,5-dichlorophenyl)-2-(4-methyl-1-piperazinyl)acetohydrazide, mixture of enantiomers;
(±)-N′-(3,5-dichlorophenyl)-2-(4-methyl-1-piperazinyl)-2-{4-[(trifluoromethyl)-oxy]phenyl}acetohydrazide, mixture of enantiomers;
formic acid-(±)-2-(1-benzothien-2-yl)-N′-(3,5-dichlorophenyl)-2-(4-methyl-1-piperazinyl)acetohydrazide, mixture of enantiomers;
formic acid-(±)-2-(1-benzothien-5-yl)-N′-(3,5-dichlorophenyl)-2-(4-methyl-1-piperazinyl)acetohydrazide, mixture of enantiomers;
formic acid-(±)-2-dibenzo[b,d]thien-2-yl-N′-(3,5-dichlorophenyl)-2-(4-methyl-1-piperazinyl)acetohydrazide, mixture of enantiomers;
N′-[3,5-bis(trifluoromethyl)phenyl]-2-(2-chlorophenyl)-2-(4-methyl-1-piperazinyl)-acetohydrazide, enantiomer 1;
N′-[3,5-bis(trifluoromethyl)phenyl]-2-(2-chlorophenyl)-2-(4-methyl-1-piperazinyl)-acetohydrazide, enantiomer 2;
(±)-N′-[3,5-bis(trifluoromethyl)phenyl]-2-(4-methyl-1-piperazinyl)-2-{2-[(trifluoromethyl)-oxy]phenyl}acetohydrazide, mixture of enantiomers;
N′-[3,5-bis(trifluoromethyl)phenyl]-2-(3-methylphenyl)-2-(4-methyl-1-piperazinyl)-acetohydrazide, enantiomer 1;
N′-[3,5-bis(trifluoromethyl)phenyl]-2-(3-methylphenyl)-2-(4-methyl-1-piperazinyl)acetohydrazide, enantiomer 2;
N′-[3,5-bis(trifluoromethyl)phenyl]-2-(2-methylphenyl)-2-(4-methyl-1-piperazinyl)-acetohydrazide, enantiomer 1;
N′-[3,5-bis(trifluoromethyl)phenyl]-2-(2-methylphenyl)-2-(4-methyl-1-piperazinyl)-acetohydrazide, enantiomer 2;
N′-[3,5-bis(trifluoromethyl)phenyl]-2-(2-fluorophenyl)-2-(4-methyl-1-piperazinyl)-acetohydrazide, enantiomer 1;
N′-[3,5-bis(trifluoromethyl)phenyl]-2-(2-fluorophenyl)-2-(4-methyl-1-piperazinyl)-acetohydrazide, enantiomer 2;
N′-(3,5-dichlorophenyl)-2-(2,3-dihydro-1-benzofuran-5-yl)-2-(4-methyl-1-piperazinyl)-acetohydrazide enantiomer 1
N′-(3,5-dichlorophenyl)-2-(2,3-dihydro-1-benzofuran-5-yl)-2-(4-methyl-1-piperazinyl)-acetohydrazide, enantiomer 2;
N′-[3,5-bis(trifluoromethyl)phenyl]-2-(2,3-dihydro-1-benzofuran-5-yl)-2-(4-methyl-1-piperazinyl)acetohydrazide, enantiomer 1;
N′-[3,5-bis(trifluoromethyl)phenyl]-2-(2,3-dihydro-1-benzofuran-5-yl)-2-(4-methyl-1-piperazinyl)acetohydrazide, enantiomer 2;
N′-[3,5-bis(trifluoromethyl)phenyl]-2-(2,3-difluorophenyl)-2-(4-methyl-1-piperazinyl)-acetohydrazide, enantiomer 1;
N′-[3,5-bis(trifluoromethyl)phenyl]-2-(2,3-difluorophenyl)-2-(4-methyl-1-piperazinyl)-acetohydrazide, enantiomer 2;
N′-[3,5-bis(trifluoromethyl)phenyl]-2-(2,6-difluorophenyl)-2-(4-methyl-1-piperazinyl)-acetohydrazide, enantiomer 1;
N′-[3,5-bis(trifluoromethyl)phenyl]-2-(2,6-difluorophenyl)-2-(4-methyl-1-piperazinyl)-acetohydrazide, enantiomer 2;
N′-[3,5-bis(trifluoromethyl)phenyl]-2-[2-(methyloxy)phenyl]-2-(4-methyl-1-piperazinyl)-acetohydrazide, enantiomer 1;
N′-[3,5-bis(trifluoromethyl)phenyl]-2-[2-(methyloxy)phenyl]-2-(4-methyl-1-piperazinyl)-acetohydrazide, enantiomer 2;
N′-[3,5-bis(trifluoromethyl)phenyl]-2-[2-fluoro-6-(methyloxy)phenyl]-2-(4-methyl-1-piperazinyl)acetohydrazide enantiomer 1
N′-[3,5-bis(trifluoromethyl)phenyl]-2-[2-fluoro-6-(methyloxy)phenyl]-2-(4-methyl-1-piperazinyl)acetohydrazide, enantiomer 2;
N′-[3,5-bis(trifluoromethyl)phenyl]-2-(2,3-dihydro-1,4-benzodioxin-5-yl)-2-(4-methyl-1-piperazinyl)acetohydrazide, enantiomer 1;
N′-[3,5-bis(trifluoromethyl)phenyl]-2-(2,3-dihydro-1,4-benzodioxin-5-yl)-2-(4-methyl-1-piperazinyl)acetohydrazide enantiomer 2
(±)-N′-(3,5-dichlorophenyl)-2-(4-fluoro-1-naphthalenyl)-2-(4-methyl-1-piperazinyl)-ethanohydrazide, mixture of enantiomers;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 1;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 2;
(±)-N′-[3,5-bis(trifluoromethyl)phenyl]-2-(4-methyl-1-piperazinyl)-2-{3-[(trifluoromethyl)-oxy]phenyl}acetohydrazide, mixture of enantiomers;
(±)-N′-(3,5-dichlorophenyl)-2-[6-(methyloxy)-2-naphthalenyl]-2-(4-methyl-1-piperazinyl)-acetohydrazide, mixture of enantiomers;
(±)-N′-(3,5-Dimethylphenyl)-2-(4-methyl-1-piperazinyl)-2-(1-naphthalenyl)-acetohydrazide, mixture of enantiomers;
(±)-N′-[3,5-Bis(trifluoromethyl)phenyl]-2-(4-methyl-1-piperazinyl)-2-(1-naphthalenyl)-acetohydrazide hydrochloride, mixture of enantiomers;
2-(1,3-Benzodioxol-5-yl)-N′-[3,5-bis(trifluoromethyl)phenyl]-2-(4-methyl-1-piperazinyl)-acetohydrazide hydrochloride, mixture of enantiomers;
(±)-2-(2-chlorophenyl)-N′-(3,5-dichlorophenyl)-2-(4-methyl-1-piperazinyl)acetohydrazide, mixture of enantiomers;
2-(3-chlorophenyl)-N′-(3,5-dichlorophenyl)-2-(4-methyl-1-piperazinyl)acetohydrazide, enantiomer 1;
2-(3-chlorophenyl)-N′-(3,5-dichlorophenyl)-2-(4-methyl-1-piperazinyl)acetohydrazide, enantiomer 2;
formic acid-(±)-N′-[3,5-bis(trifluoromethyl)phenyl]-2-(3-chlorophenyl)-2-(4-methyl-1-piperazinyl)acetohydrazide (1:1), mixture of enantiomers;
(±)-N′-[3,5-bis(trifluoromethyl)phenyl]-2-(2,2-difluoro-1,3-benzodioxol-4-yl)-2-(4-methyl-1-piperazinyl)acetohydrazide, mixture of enantiomers;
2-(1,3-benzodioxol-5-yl)-N′-(3,5-dichlorophenyl)-2-(4-methyl-1-piperazinyl)ethano-hydrazide hydrochloride, enantiomer 2;
(±)-N′-(3,5-dichlorophenyl)-2-(4-methyl-1-piperazinyl)-2-(1-naphthalenyl)acetohydrazide formate, mixture of enantiomers;
N′-[3,5-bis(trifluoromethyl)phenyl]-2-(4-methyl-1-piperazinyl)-2-(3-pyridinyl)aceto-hydrazide, enantiomer 1
N′-[3,5-bis(trifluoromethyl)phenyl]-2-(4-methyl-1-piperazinyl)-2-(3-pyridinyl)aceto-hydrazide, enantiomer 2;
N′-[3,5-bis(trifluoromethyl)phenyl]-2-imidazo[1,2-a]pyridin-6-yl-2-(4-methyl-1-piperazinyl)-acetohydrazide, enantiomer 1;
N′-[3,5-bis(trifluoromethyl)phenyl]-2-imidazo[1,2-a]pyridin-6-yl-2-(4-methyl-1-piperazinyl)-acetohydrazide, enantiomer 2;
N′-[3,5-bis(trifluoromethyl)phenyl]-2-(4-methyl-1-piperazinyl)-2-(2-methyl-3-pyridinyl)-acetohydrazide, enantiomer 2;
N′-[3,5-bis(trifluoromethyl)phenyl]-2-(4-methyl-1-piperazinyl)-2-(4-methyl-1-piperazinyl)-acetohydrazide, enantiomer 1;
N′-[3,5-bis(trifluoromethyl)phenyl]-2-(4-methyl-1-piperazinyl)-2-(4-methyl-1-piperazinyl)-acetohydrazide, enantiomer 2;
N′-[3,5-bis(trifluoromethyl)phenyl]-2-(4-methyl-1-piperazinyl)-2-(6-methyl-3-pyridinyl)-acetohydrazide, enantiomer 2;
N′-[3,5-bis(trifluoromethyl)phenyl]-2-(3-chloro-2-pyridinyl)-2-(4-methyl-1-piperazinyl)-acetohydrazide, enantiomer 1;
N′-[3,5-bis(trifluoromethyl)phenyl]-2-(3-chloro-2-pyridinyl)-2-(4-methyl-1-piperazinyl)-acetohydrazide, enantiomer 2;
N′-[3,5-bis(trifluoromethyl)phenyl]-2-(1-piperazinyl)-2-(2-pyridinyl)acetohydrazide, enantiomer 1;
N′-[3,5-bis(trifluoromethyl)phenyl]-2-(1-piperazinyl)-2-(2-pyridinyl)acetohydrazide, enantiomer 2;
N′-[3,5-bis(trifluoromethyl)phenyl]-2-(4-methyl-1-piperazinyl)-2-(4-methyl-2-pyridinyl)-acetohydrazide, enantiomer 1;
N′-[3,5-bis(trifluoromethyl)phenyl]-2-(4-methyl-1-piperazinyl)-2-(4-methyl-2-pyridinyl)-acetohydrazide, enantiomer 2;
N′-[3,5-bis(trifluoromethyl)phenyl]-2-(4-methyl-1-piperazinyl)-2-(6-methyl-2-pyridinyl)-acetohydrazide, enantiomer 1;
N′-[3,5-bis(trifluoromethyl)phenyl]-2-(4-methyl-1-piperazinyl)-2-(6-methyl-2-pyridinyl)-acetohydrazide, enantiomer 2;
N′-[3,5-bis(trifluoromethyl)phenyl]-2-(4-methyl-1-piperazinyl)-2-(2-pyridinyl)-acetohydrazide, enantiomer 1;
N′-[3,5-bis(trifluoromethyl)phenyl]-2-(4-methyl-1-piperazinyl)-2-(2-pyridinyl)-acetohydrazide, enantiomer 2;
(±)-2-(1,3-benzodioxol-5-yl)-N′-(3,5-dichlorophenyl)-2-(1-methyl-4-piperidinyl)-acetohydrazide, mixture of enantiomers;
2-(1,3-benzodioxol-5-yl)-N′-(3,5-dichlorophenyl)-2-(1-methyl-4-piperidinyl)acetohydrazide enantiomer 1;
2-(1,3-benzodioxol-5-yl)-N′-(3,5-dichlorophenyl)-2-(1-methyl-4-piperidinyl)acetohydrazide enantiomer 2;
(±)-N′-(3,5-dichlorophenyl)-2-(1-methyl-4-piperidinyl)-2-(1-naphthalenyl)acetohydrazide mixture of enantiomers;
N′-(3,5-dichlorophenyl)-2-(1-methyl-4-piperidinyl)-2-(1-naphthalenyl)acetohydrazide enantiomer 1;
N′-(3,5-dichlorophenyl)-2-(1-methyl-4-piperidinyl)-2-(1-naphthalenyl)acetohydrazide enantiomer 2;
N′-[3,5-bis(trifluoromethyl)phenyl]-2-(2-chlorophenyl)-2-(1-piperazinyl)acetohydrazide hydrochloride enantiomer 2;
N′-[3,5-bis(trifluoromethyl)phenyl]-2-(2-chlorophenyl)-2-(4-methyl-1-piperazinyl)aceto-hydrazide hydrochloride, enantiomer 1;
N′-[3,5-bis(trifluoromethyl)phenyl]-2-(2,6-dimethyl-3-pyridinyl)-2-(4-methyl-1-piperazinyl)-acetohydrazide, enantiomer 2
N′-[3,5-bis(trifluoromethyl)phenyl]-2-(4-methyl-1-piperazinyl)-2-(5-pyrimidinyl)aceto-hydrazide, enantiomer 1;
N′-[3,5-bis(trifluoromethyl)phenyl]-2-(4-methyl-1-piperazinyl)-2-(5-pyrimidinyl)aceto-hydrazide, enantiomer 2;
N′-[3,5-bis(trifluoromethyl)phenyl]-2-(2,6-dimethyl-3-pyridinyl)-2-[(8aR)-hexahydropyrrolo-[1,2-a]pyrazin-2(1H)-yl]acetohydrazide, diastereoisomer 1;
N′-[3,5-bis(trifluoromethyl)phenyl]-2-(2,6-dimethyl-3-pyridinyl)-2-[(8aR)-hexahydropyrrolo-[1,2-a]pyrazin-2(1H)-yl]acetohydrazide, diastereoisomer 2;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 1;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 2;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 1;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 2;
(2S)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide hydrochloride;
N′-[3,5-bis(trifluoromethyl)phenyl]-2-[(8aR)-hexahydropyrrolo[1,2-a]pyrazin-2(1H)-yl]-2-(1,3-thiazol-2-yl)acetohydrazide, diastereomeric mixture;
N′-[3,5-bis(trifluoromethyl)phenyl]-2-(8a-methylhexahydropyrrolo[1,2-a]pyrazin-2(1H)-yl)-2-(3-methylphenyl)acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[2-(ethyloxy)phenyl]-2-[(8aR)-hexahydropyrrolo[1,2-a]-pyrazin-2(1H)-yl]acetohydrazide, mixture of diastereoisomers;
N′-[3,5-bis(trifluoromethyl)phenyl]-2-(2-chloro-3-pyridinyl)-2-[(8aR)-hexahydropyrrolo[1,2-a]pyrazin-2(1H)-yl]acetohydrazide, diastereomeric mixture;
N′-[3,5-bis(trifluoromethyl)phenyl]-2-(4-fluoro-2-methylphenyl)-2-[(8aR)-hexahydropyrrolo-[1,2-a]pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(6-methyl-2-pyridinyl)propanohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereoisomer 2;
N′[3,5-bis(trifluoromethyl)phenyl]-2-[(8aR)-hexahydropyrrolo[1,2-a]pyrazin-2(1H)-yl]-2-(3-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[6-(trifluoromethyl)-3-pyridinyl]acetohydrazide diastereomeric mixture
N′-[3,5-bis(trifluoromethyl)phenyl]-2-[(8aR)-hexahydropyrrolo[1,2-a]pyrazin-2(1H)-yl]-2-{2-[(trifluoromethyl)oxy]phenyl}acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-(methyloxy)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide diastereomeric mixture;
N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, mixture of diastereoisomers;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, diastereoisomers 2;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereomeric mixture;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, mixture of diastereoisomers;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, mixture of diastereoisomer;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, diastereoisomer 2;
N′-(3,5-dichlorophenyl)-2-(2,3-dihydro-1-benzofuran-5-yl)-2-(4-methylpiperazin-1-yl)acetohydrazide-enantiomer 1;
N′-(3,5-dichlorophenyl)-2-(2,3-dihydro-1-benzofuran-5-yl)-2-(4-methylpiperazin-1-yl)acetohydrazide-enantiomer 2;
N′-[3,5-bis(trifluoromethyl)phenyl]-2-(2,3-dihydro-1-benzofuran-5-yl)-2-(4-methyl-1-piperazinyl)acetohydrazide-enantiomer 1;
N′-[3,5-bis(trifluoromethyl)phenyl]-2-(2,3-dihydro-1-benzofuran-5-yl)-2-(4-methyl-1-piperazinyl)acetohydrazide-enantiomer 2;
N′-[3,5-bis(trifluoromethyl)phenyl]-2-(2-chlorophenyl)-2-(1-piperazinyl)acetohydrazide enantiomer 1;
N′-[3,5-bis(trifluoromethyl)phenyl]-2-(2-chlorophenyl)-2-(1-piperazinyl)acetohydrazide-enantiomer 2;
(±)-2-(1,3-Benzodioxol-5-yl)-N′-[3,5-bis(trifluoromethyl)phenyl]-2-(4-methyl-1-piperazinyl)acetohydrazide hydrochloride;
2-(1,3-benzodioxol-5-yl)-N′-(3,5-dichlorophenyl)-2-(4-methyl-1-piperazinyl)acetohydrazide hydrochloride-enantiomer 2;
N′-[3,5-bis(trifluoromethyl)phenyl]-2-(4-methyl-1-piperazinyl)-2-(6-methyl-3-pyridinyl)-acetohydrazide, enantiomer 1;
(±)-N′-(3,5-dichlorophenyl)-2-(4-methylpiperazin-1-yl)-2-(1-naphthyl)aceto-hydrazide diformate;
2-(4-methylpiperazin-1-yl)-N′-[3,5-bis(trifluoromethyl)phenyl]-2-pyridin-3-ylacetohydrazide-enantiomer 1;
2-(4-methylpiperazin-1-yl)-N′-[3,5-bis(trifluoromethyl)phenyl]-2-pyridin-3-ylacetohydrazide-enantiomer 2;
N′-[3,5-bis(trifluoromethyl)phenyl]-2-(4-methylpiperazin-1-yl)-2-(2-methylpyridin-3-yl)acetohydrazide enantiomer 1;
N′-[3,5-bis(trifluoromethyl)phenyl]-2-(4-methylpiperazin-1-yl)-2-(2-methylpyridin-3-yl)acetohydrazide enantiomer 2;
N′-[3,5-bis(trifluoromethyl)phenyl]-2-(4-methyl-1-piperazinyl)-2-(3-methyl-2-pyridinyl)acetohydrazide-enantiomer 1;
N′-[3,5-bis(trifluoromethyl)phenyl]-2-(4-methyl-1-piperazinyl)-2-(3-methyl-2-pyridinyl)acetohydrazide-enantiomer 2;
N′-[3,5-bis(trifluoromethyl)phenyl]-2-(4-methyl-1-piperazinyl)-2-(2-methyl-3-pyridinyl)-acetohydrazide, enantiomer 1;
2-(1,3-benzodioxol-5-yl)-N′-[3,5-bis(trifluoromethyl)phenyl]-2-(4-methyl-1-piperazinyl)propanohydrazide-enantiomer 1;
2-(1,3-benzodioxol-5-yl)-N′-[3,5-bis(trifluoromethyl)phenyl]-2-(4-methyl-1-piperazinyl)propanohydrazide-enantiomer 2;
N′-[3,5-bis(trifluoromethyl)phenyl]-2-(4-methyl-1-piperazinyl)-2-(2-pyridinyl)propanohydrazide-enantiomer 1;
N′-[3,5-bis(trifluoromethyl)phenyl]-2-(4-methyl-1-piperazinyl)-2-(2-pyridinyl)propanohydrazide-enantiomer 2;
   and pharmaceutically acceptable salts and solvates thereof, wherein “enantiomer 1” or “enantiomer 2” indicates a single enantiomer of unknown absolute stereochemistry, prepared according to that described in the Experimental hereinbelow and “diastereoisomer 1” or “diastereoisomer 2” indicates a single diastereoisomer in homochiral form whose absolute configuration at one stereocentre was not determined, prepared according to that described in the Experimental hereinbelow.

In another embodiment the invention provides:
formic acid-(±)-2-dibenzo[b,d]thien-4-yl-N′-(3,5-dichlorophenyl)-2-(4-methyl-1-piperazinyl)acetohydrazide, mixture of enantiomers;
formic acid-(±)-2-dibenzo[b,d]furan-4-yl-N′-(3,5-dichlorophenyl)-2-(4-methyl-1-piperazinyl)acetohydrazide, mixture of enantiomers;
formic acid-(±)-N′-(3,5-dichlorophenyl)-2-(4-methyl-1-piperazinyl)-2-(2-naphthalenyl)-acetohydrazide, mixture of enantiomers;
formic acid-(±)-2-(1-benzofuran-2-yl)-N′-(3,5-dichlorophenyl)-2-(4-methyl-1-piperazinyl)acetohydrazide, mixture of enantiomers;
(±)-N′-(3,5-dichlorophenyl)-2-(4-methyl-1-piperazinyl)-2-{4-[(trifluoromethyl)-oxy]phenyl}acetohydrazide, mixture of enantiomers;
formic acid-(±)-2-(1-benzothien-2-yl)-N′-(3,5-dichlorophenyl)-2-(4-methyl-1-piperazinyl)acetohydrazide, mixture of enantiomers;
formic acid-(±)-2-(1-benzothien-5-yl)-N′-(3,5-dichlorophenyl)-2-(4-methyl-1-piperazinyl)acetohydrazide, mixture of enantiomers;
formic acid-(±)-2-dibenzo[b,d]thien-2-yl-N′-(3,5-dichlorophenyl)-2-(4-methyl-1-piperazinyl)acetohydrazide, mixture of enantiomers;
N′-[3,5-bis(trifluoromethyl)phenyl]-2-(2-chlorophenyl)-2-(4-methyl-1-piperazinyl)-acetohydrazide, enantiomer 1;
N′-[3,5-bis(trifluoromethyl)phenyl]-2-(2-chlorophenyl)-2-(4-methyl-1-piperazinyl)-acetohydrazide, enantiomer 2;
N′-(3,5-dichlorophenyl)-2-(2,3-dihydro-1-benzofuran-5-yl)-2-(4-methylpiperazin-1-yl)acetohydrazide-enantiomer 1;
N′-(3,5-dichlorophenyl)-2-(2,3-dihydro-1-benzofuran-5-yl)-2-(4-methylpiperazin-1-yl)acetohydrazide-enantiomer 2;
N′-[3,5-bis(trifluoromethyl)phenyl]-2-(2,3-dihydro-1-benzofuran-5-yl)-2-(4-methyl-1-piperazinyl)acetohydrazide-enantiomer 1;
N′-[3,5-bis(trifluoromethyl)phenyl]-2-(2,3-dihydro-1-benzofuran-5-yl)-2-(4-methyl-1-piperazinyl)acetohydrazide-enantiomer 2;
N′-[3,5-bis(trifluoromethyl)phenyl]-2-(2-chlorophenyl)-2-(1-piperazinyl)acetohydrazide enantiomer 1;
N′-[3,5-bis(trifluoromethyl)phenyl]-2-(2-chlorophenyl)-2-(1-piperazinyl)acetohydrazide enantiomer 2;
(±)-2-(1,3-Benzodioxol-5-yl)-N′-[3,5-bis(trifluoromethyl)phenyl]-2-(4-methyl-1-piperazinyl)acetohydrazide hydrochloride;
2-(1,3-benzodioxol-5-yl)-N′-(3,5-dichlorophenyl)-2-(4-methyl-1-piperazinyl)acetohydrazide hydrochloride-enantiomer 2;
(±)-N′-(3,5-dichlorophenyl)-2-(4-methylpiperazin-1-yl)-2-(1-naphthyl)aceto-hydrazide diformate;
2-(4-methylpiperazin-1-yl)-N′-[3,5-bis(trifluoromethyl)phenyl]-2-pyridin-3-ylacetohydrazide-enantiomer 1;
2-(4-methylpiperazin-1-yl)-N′-[3,5-bis(trifluoromethyl)phenyl]-2-pyridin-3-ylacetohydrazide-enantiomer 2;
N′-[3,5-bis(trifluoromethyl)phenyl]-2-(4-methylpiperazin-1-yl)-2-(2-methylpyridin-3-yl)acetohydrazide enantiomer 1;
N′-[3,5-bis(trifluoromethyl)phenyl]-2-(4-methylpiperazin-1-yl)-2-(2-methylpyridin-3-yl)acetohydrazide enantiomer 2;
N′-[3,5-bis(trifluoromethyl)phenyl]-2-(4-methyl-1-piperazinyl)-2-(3-methyl-2-pyridinyl)acetohydrazide-enantiomer 1;
N′-[3,5-bis(trifluoromethyl)phenyl]-2-(4-methyl-1-piperazinyl)-2-(3-methyl-2-pyridinyl)acetohydrazide-enantiomer 2;
(±)-N′-[3,5-bis(trifluoromethyl)phenyl]-2-(4-methyl-1-piperazinyl)-2-{2-[(trifluoromethyl)-oxy]phenyl}acetohydrazide, mixture of enantiomers;
N′-[3,5-bis(trifluoromethyl)phenyl]-2-(3-methylphenyl)-2-(4-methyl-1-piperazinyl)-acetohydrazide, enantiomer 1;
N′-[3,5-bis(trifluoromethyl)phenyl]-2-(3-methylphenyl)-2-(4-methyl-1-piperazinyl)acetohydrazide, enantiomer 2;
N′-[3,5-bis(trifluoromethyl)phenyl]-2-(2-methylphenyl)-2-(4-methyl-1-piperazinyl)-acetohydrazide, enantiomer 1;
N′-[3,5-bis(trifluoromethyl)phenyl]-2-(2-methylphenyl)-2-(4-methyl-1-piperazinyl)-acetohydrazide, enantiomer 2;
N′-[3,5-bis(trifluoromethyl)phenyl]-2-(2-fluorophenyl)-2-(4-methyl-1-piperazinyl)-acetohydrazide, enantiomer 1;
N′-[3,5-bis(trifluoromethyl)phenyl]-2-(2-fluorophenyl)-2-(4-methyl-1-piperazinyl)-acetohydrazide, enantiomer 2;
N′-(3,5-dichlorophenyl)-2-(2,3-dihydro-1-benzofuran-5-yl)-2-(4-methyl-1-piperazinyl)-acetohydrazide enantiomer 1
N′-(3,5-dichlorophenyl)-2-(2,3-dihydro-1-benzofuran-5-yl)-2-(4-methyl-1-piperazinyl)-acetohydrazide, enantiomer 2;
N′-[3,5-bis(trifluoromethyl)phenyl]-2-(2,3-dihydro-1-benzofuran-5-yl)-2-(4-methyl-1-piperazinyl)acetohydrazide, enantiomer 1;
N′-[3,5-bis(trifluoromethyl)phenyl]-2-(2,3-dihydro-1-benzofuran-5-yl)-2-(4-methyl-1-piperazinyl)acetohydrazide, enantiomer 2;
N′-[3,5-bis(trifluoromethyl)phenyl]-2-(2,3-difluorophenyl)-2-(4-methyl-1-piperazinyl)-acetohydrazide, enantiomer 1;
N′-[3,5-bis(trifluoromethyl)phenyl]-2-(2,3-difluorophenyl)-2-(4-methyl-1-piperazinyl)-acetohydrazide, enantiomer 2;
N′-[3,5-bis(trifluoromethyl)phenyl]-2-(2,6-difluorophenyl)-2-(4-methyl-1-piperazinyl)-acetohydrazide, enantiomer 1;
N′-[3,5-bis(trifluoromethyl)phenyl]-2-(2,6-difluorophenyl)-2-(4-methyl-1-piperazinyl)-acetohydrazide, enantiomer 2;
N′-[3,5-bis(trifluoromethyl)phenyl]-2-(2-chlorophenyl)-2-(1-piperazinyl)acetohydrazide, enantiomer 1;
N′-[3,5-bis(trifluoromethyl)phenyl]-2-(2-chlorophenyl)-2-(1-piperazinyl)acetohydrazide, enantiomer 2;
N′-[3,5-bis(trifluoromethyl)phenyl]-2-[2-(methyloxy)phenyl]-2-(4-methyl-1-piperazinyl)-acetohydrazide, enantiomer 1;
N′-[3,5-bis(trifluoromethyl)phenyl]-2-[2-(methyloxy)phenyl]-2-(4-methyl-1-piperazinyl)-acetohydrazide, enantiomer 2;
N′-[3,5-bis(trifluoromethyl)phenyl]-2-[2-fluoro-6-(methyloxy)phenyl]-2-(4-methyl-1-piperazinyl)acetohydrazide enantiomer 1
N′-[3,5-bis(trifluoromethyl)phenyl]-2-[2-fluoro-6-(methyloxy)phenyl]-2-(4-methyl-1-piperazinyl)acetohydrazide, enantiomer 2;
N′-[3,5-bis(trifluoromethyl)phenyl]-2-(2,3-dihydro-1,4-benzodioxin-5-yl)-2-(4-methyl-1-piperazinyl)acetohydrazide, enantiomer 1;
N′-[3,5-bis(trifluoromethyl)phenyl]-2-(2,3-dihydro-1,4-benzodioxin-5-yl)-2-(4-methyl-1-piperazinyl)acetohydrazide enantiomer 2
(±)-N′-(3,5-dichlorophenyl)-2-(4-fluoro-1-naphthalenyl)-2-(4-methyl-1-piperazinyl)-ethanohydrazide, mixture of enantiomers;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 1;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 2;
(±)-N′-[3,5-bis(trifluoromethyl)phenyl]-2-(4-methyl-1-piperazinyl)-2-{3-[(trifluoromethyl)-oxy]phenyl}acetohydrazide, mixture of enantiomers;
(±)-N′-(3,5-dichlorophenyl)-2-[6-(methyloxy)-2-naphthalenyl]-2-(4-methyl-1-piperazinyl)-acetohydrazide, mixture of enantiomers;
(±)-N′-(3,5-Dimethylphenyl)-2-(4-methyl-1-piperazinyl)-2-(1-naphthalenyl)-acetohydrazide, mixture of enantiomers;
(±)-N′-[3,5-Bis(trifluoromethyl)phenyl]-2-(4-methyl-1-piperazinyl)-2-(1-naphthalenyl)-acetohydrazide hydrochloride, mixture of enantiomers;
2-(1,3-Benzodioxol-5-yl)-N′-[3,5-bis(trifluoromethyl)phenyl]-2-(4-methyl-1-piperazinyl)-acetohydrazide hydrochloride, mixture of enantiomers;
(±)-2-(2-chlorophenyl)-N′-(3,5-dichlorophenyl)-2-(4-methyl-1-piperazinyl)acetohydrazide, mixture of enantiomers;
2-(3-chlorophenyl)-N′-(3,5-dichlorophenyl)-2-(4-methyl-1-piperazinyl)acetohydrazide, enantiomer 1;
2-(3-chlorophenyl)-N′-(3,5-dichlorophenyl)-2-(4-methyl-1-piperazinyl)acetohydrazide, enantiomer 2;
formic acid-(±)-N′-[3,5-bis(trifluoromethyl)phenyl]-2-(3-chlorophenyl)-2-(4-methyl-1-piperazinyl)acetohydrazide (1:1), mixture of enantiomers;
(±)-N′-[3,5-bis(trifluoromethyl)phenyl]-2-(2,2-difluoro-1,3-benzodioxol-4-yl)-2-(4-methyl-1-piperazinyl)acetohydrazide, mixture of enantiomers;
2-(1,3-benzodioxol-5-yl)-N′-(3,5-dichlorophenyl)-2-(4-methyl-1-piperazinyl)ethano-hydrazide hydrochloride, enantiomer 2;
(±)-N′-(3,5-dichlorophenyl)-2-(4-methyl-1-piperazinyl)-2-(1-naphthalenyl)acetohydrazide formate, mixture of enantiomers;
N′-[3,5-bis(trifluoromethyl)phenyl]-2-(4-methyl-1-piperazinyl)-2-(3-pyridinyl)aceto-hydrazide, enantiomer 1
N′-[3,5-bis(trifluoromethyl)phenyl]-2-(4-methyl-1-piperazinyl)-2-(3-pyridinyl)aceto-hydrazide, enantiomer 2;
N′-[3,5-bis(trifluoromethyl)phenyl]-2-imidazo[1,2-a]pyridin-6-yl-2-(4-methyl-1-piperazinyl)-acetohydrazide, enantiomer 1;
N′-[3,5-bis(trifluoromethyl)phenyl]-2-imidazo[1,2-a]pyridin-6-yl-2-(4-methyl-1-piperazinyl)-acetohydrazide, enantiomer 2;
N′-[3,5-bis(trifluoromethyl)phenyl]-2-(4-methyl-1-piperazinyl)-2-(2-methyl-3-pyridinyl)-acetohydrazide, enantiomer 1;
N′-[3,5-bis(trifluoromethyl)phenyl]-2-(4-methyl-1-piperazinyl)-2-(2-methyl-3-pyridinyl)-acetohydrazide, enantiomer 2;
N′-[3,5-bis(trifluoromethyl)phenyl]-2-(4-methyl-1-piperazinyl)-2-(4-methyl-1-piperazinyl)-acetohydrazide, enantiomer 1;
N′-[3,5-bis(trifluoromethyl)phenyl]-2-(4-methyl-1-piperazinyl)-2-(4-methyl-1-piperazinyl)-acetohydrazide, enantiomer 2;
N′-[3,5-bis(trifluoromethyl)phenyl]-2-(4-methyl-1-piperazinyl)-2-(6-methyl-3-pyridinyl)-acetohydrazide, enantiomer 1;
N′-[3,5-bis(trifluoromethyl)phenyl]-2-(4-methyl-1-piperazinyl)-2-(6-methyl-3-pyridinyl)-acetohydrazide, enantiomer 2;
N′-[3,5-bis(trifluoromethyl)phenyl]-2-(3-chloro-2-pyridinyl)-2-(4-methyl-1-piperazinyl)-acetohydrazide, enantiomer 1;
N′-[3,5-bis(trifluoromethyl)phenyl]-2-(3-chloro-2-pyridinyl)-2-(4-methyl-1-piperazinyl)-acetohydrazide, enantiomer 2;
N′-[3,5-bis(trifluoromethyl)phenyl]-2-(1-piperazinyl)-2-(2-pyridinyl)acetohydrazide, enantiomer 1;
N′-[3,5-bis(trifluoromethyl)phenyl]-2-(1-piperazinyl)-2-(2-pyridinyl)acetohydrazide, enantiomer 2;
N′-[3,5-bis(trifluoromethyl)phenyl]-2-(4-methyl-1-piperazinyl)-2-(4-methyl-2-pyridinyl)-acetohydrazide, enantiomer 1;
N′-[3,5-bis(trifluoromethyl)phenyl]-2-(4-methyl-1-piperazinyl)-2-(4-methyl-2-pyridinyl)-acetohydrazide, enantiomer 2;
N′-[3,5-bis(trifluoromethyl)phenyl]-2-(4-methyl-1-piperazinyl)-2-(6-methyl-2-pyridinyl)-acetohydrazide, enantiomer 1;
N′-[3,5-bis(trifluoromethyl)phenyl]-2-(4-methyl-1-piperazinyl)-2-(6-methyl-2-pyridinyl)-acetohydrazide, enantiomer 2;
N′-[3,5-bis(trifluoromethyl)phenyl]-2-(4-methyl-1-piperazinyl)-2-(2-pyridinyl)-acetohydrazide, enantiomer 1;
N′-[3,5-bis(trifluoromethyl)phenyl]-2-(4-methyl-1-piperazinyl)-2-(2-pyridinyl)-acetohydrazide, enantiomer 2;
(±)-2-(1,3-benzodioxol-5-yl)-N′-(3,5-dichlorophenyl)-2-(1-methyl-4-piperidinyl)-acetohydrazide, mixture of enantiomers;
2-(1,3-benzodioxol-5-yl)-N′-(3,5-dichlorophenyl)-2-(1-methyl-4-piperidinyl)acetohydrazide enantiomer 1;
2-(1,3-benzodioxol-5-yl)-N′-(3,5-dichlorophenyl)-2-(1-methyl-4-piperidinyl)acetohydrazide enantiomer 2;
(±)-N′-(3,5-dichlorophenyl)-2-(1-methyl-4-piperidinyl)-2-(1-naphthalenyl)acetohydrazide mixture of enantiomers;
N′-(3,5-dichlorophenyl)-2-(1-methyl-4-piperidinyl)-2-(1-naphthalenyl)acetohydrazide enantiomer 1;
N′-(3,5-dichlorophenyl)-2-(1-methyl-4-piperidinyl)-2-(1-naphthalenyl)acetohydrazide enantiomer 2;
N′-[3,5-bis(trifluoromethyl)phenyl]-2-(2-chlorophenyl)-2-(1-piperazinyl)acetohydrazide hydrochloride enantiomer 2;
N′-[3,5-bis(trifluoromethyl)phenyl]-2-(2-chlorophenyl)-2-(4-methyl-1-piperazinyl)aceto-hydrazide hydrochloride, enantiomer 1;
N′-[3,5-bis(trifluoromethyl)phenyl]-2-(2,6-dimethyl-3-pyridinyl)-2-(4-methyl-1-piperazinyl)-acetohydrazide, enantiomer 2;
N′-[3,5-bis(trifluoromethyl)phenyl]-2-(4-methyl-1-piperazinyl)-2-(5-pyrimidinyl)aceto-hydrazide, enantiomer 1;
N′-[3,5-bis(trifluoromethyl)phenyl]-2-(4-methyl-1-piperazinyl)-2-(5-pyrimidinyl)aceto-hydrazide, enantiomer 2;
2-(1,3-benzodioxol-5-yl)-N′-[3,5-bis(trifluoromethyl)phenyl]-2-(4-methyl-1-piperazinyl)propanohydrazide-enantiomer 1;
2-(1,3-benzodioxol-5-yl)-N′-[3,5-bis(trifluoromethyl)phenyl]-2-(4-methyl-1-piperazinyl)propanohydrazide-enantiomer 2;
N′-[3,5-bis(trifluoromethyl)phenyl]-2-(4-methyl-1-piperazinyl)-2-(2-pyridinyl)propanohydrazide-enantiomer 1
N′-[3,5-bis(trifluoromethyl)phenyl]-2-(4-methyl-1-piperazinyl)-2-(2-pyridinyl)propanohydrazide-enantiomer 2;
   and pharmaceutically acceptable salts and solvates thereof, wherein “enantiomer 1” or “enantiomer 2” indicates a single enantiomer of unknown absolute stereochemistry prepared according to that described in the Experimental hereinbelow.

In another embodiment the invention provides:
(2S)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide hydrochloride;
   and pharmaceutically acceptable salts and solvates thereof, prepared according to that described in the Experimental hereinbelow.

In another embodiment the invention provides:
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
   and pharmaceutically acceptable salts and solvates thereof.

In another embodiment the invention provides:
N′-[3,5-bis(trifluoromethyl)phenyl]-2-(2,6-dibromo-3-pyridinyl)-2-[(8aR)-hexahydropyrrolo[1,2-a]pyrazin-2(1H)-yl]acetohydrazide-diastereoisomer 1;
N′-[3,5-bis(trifluoromethyl)phenyl]-2-(2,6-dimethyl-3-pyridinyl)-2-[(8aR)-hexahydropyrrolo-[1,2-a]pyrazin-2(1H)-yl]acetohydrazide, diastereoisomer 1;
N′-[3,5-bis(trifluoromethyl)phenyl]-2-(2,6-dimethyl-3-pyridinyl)-2-[(8aR)-hexahydropyrrolo-[1,2-a]pyrazin-2(1H)-yl]acetohydrazide, diastereoisomer 2;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 1;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 2;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 1;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 2;
(2S)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide hydrochloride;
N′-[3,5-bis(trifluoromethyl)phenyl]-2-[(8aR)-hexahydropyrrolo[1,2-a]pyrazin-2(1H)-yl]-2-(1,3-thiazol-2-yl)acetohydrazide, diastereomeric mixture;
N′-[3,5-bis(trifluoromethyl)phenyl]-2-(8a-methylhexahydropyrrolo[1,2-a]pyrazin-2(1H)-yl)-2-(3-methylphenyl)acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[2-(ethyloxy)phenyl]-2-[(8aR)-hexahydropyrrolo[1,2-a]-pyrazin-2(1H)-yl]acetohydrazide, mixture of diastereoisomers;
N′-[3,5-bis(trifluoromethyl)phenyl]-2-(2-chloro-3-pyridinyl)-2-[(8aR)-hexahydropyrrolo[1,2-a]pyrazin-2(1H)-yl]acetohydrazide, diastereomeric mixture;
N′-[3,5-bis(trifluoromethyl)phenyl]-2-(4-fluoro-2-methylphenyl)-2-[(8aR)-hexahydropyrrolo-[1,2-a]pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(6-methyl-2-pyridinyl)propanohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(3-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[6-(trifluoromethyl)-3-pyridinyl]acetohydrazide diastereomeric mixture N′-[3,5-bis(trifluoromethyl)phenyl]-2-[(8aR)-hexahydropyrrolo[1,2-a]pyrazin-2(1H)-yl]-2-{2-[(trifluoromethyl)oxy]phenyl}acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-(methyloxy)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide diastereomeric mixture N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, mixture of diastereoisomers;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, diastereoisomers 2;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereomeric mixture;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, mixture of diastereoisomers;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, mixture of diastereoisomer;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, diastereoisomer 2;
   and pharmaceutically acceptable salts and solvates thereof, wherein “diastereoisomer 1” and “diastereoisomer 2” indicates a single diastereoisomer in homochiral form whose absolute configuration at one stereocentre was not determined, prepared according to that described in the Experimental hereinbelow.

Salts of the compounds of the present invention are also encompassed within the scope of the invention. Because of their potential use in medicine, the salts of the compounds of formula (I) are preferably pharmaceutically acceptable. Suitable pharmaceutically acceptable salts can include acid addition salts. A pharmaceutically acceptable acid addition salt can be formed by reaction of a compound of formula (I) with a suitable inorganic or organic acid (such as hydrobromic, hydrochloric, hydroiodic, sulfuric, nitric, phosphoric, p-toluenesulfonic, methanesulfonic or naphthalenesulfonic acid), optionally in a suitable solvent such as an organic solvent, to give the salt which is usually isolated for example by crystallisation and filtration. Examples of pharmaceutically acceptable acid addition salts of a compound of formula (I) include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497. Other salts, which are not pharmaceutically acceptable, for example the trifluoroacetate salt, may be useful in the preparation of compounds of this invention and these form a further aspect of the invention. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹F, ³²F, ³⁵S, ¹⁸F and ³⁶Cl, respectively. Certain isotopic variations of the invention, for example, those in which a radioactive isotope such as or ¹⁴C is incorporated, are useful in drug and/or substrate tissue distribution studies. Tritiated, i.e., ³H, and carbon-14, i.e., ¹⁴C isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples hereafter using appropriate isotopic variations of suitable reagents.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

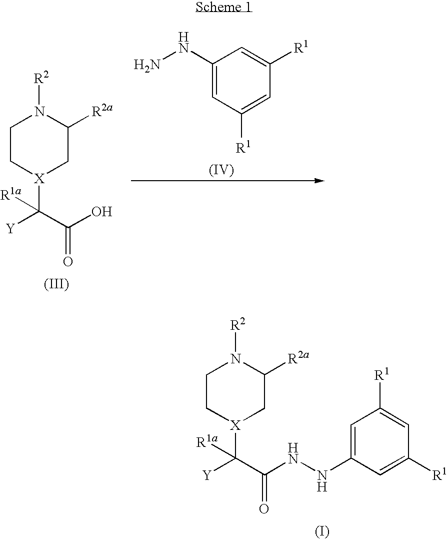

Compounds of formula (I) may be prepared by reacting (III) and (IV) together in the presence of an amide coupling reagent such as TBTU, HATU, DCC/HOBt, DCE/HOBt, BOP or pyBOP in presence of a base e.g. TEA, diisopropylethylamine or N-methylmorpholine. The reaction may be conveniently carried out in an aprotic solvent e.g. DMF, DCM or THF at room temperature for between 15 hours and one day.

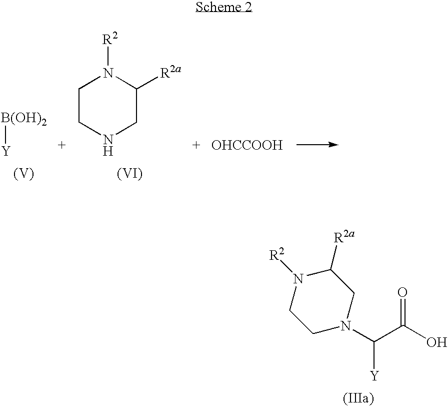

Compounds of formula (IIIa), corresponding of compounds of formula (III) in which X═N may conveniently be prepared as shown in Scheme 2 above by reacting compounds of formulae (V), (VI) and (VII) together under microwave irradiation in the presence of a suitable solvent such as EtOH or acetonitrile at a suitable temperature such as 120° C. for a suitable time such as 20 minutes. Compounds of formulae (V), (VI) and (VII) are generally commercially available.

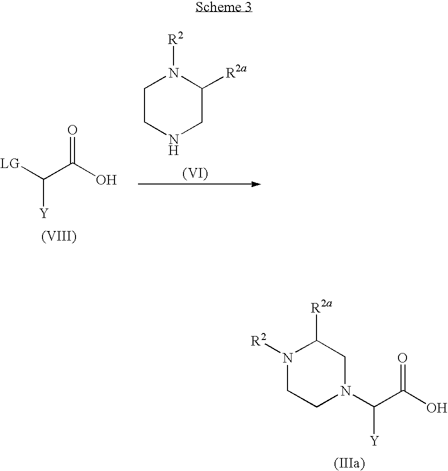

Alternatively, compounds of formula (IIIa) may be prepared according to Scheme 3 above via reaction of compounds (VIII), where LG means a leaving group selected from the group consistently of halogen, or a reactive residue of sulphonic acid (e.g. mesylate, tosylate), and (VI) in the presence of a base (e.g. K₂CO₃ or triethylamine) in an aprotic solvent such as THF at a suitable temperature, such as room temperature, for a suitable time, such as 16 hours,

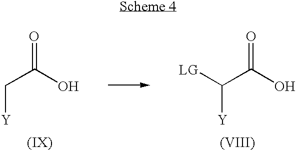

Compounds of formula (VIII) may be prepared via reaction of (IX) with an appropriate reagent (e.g. NBS in the presence of a radical initiator such as benzoyl peroxide in an aprotic solvent, e.g. CCl₄ or benzene) at a suitable temperature such as 80° C. for a suitable time such as 5 hours. Compounds of formula (IX) are commercially available (scheme 4).

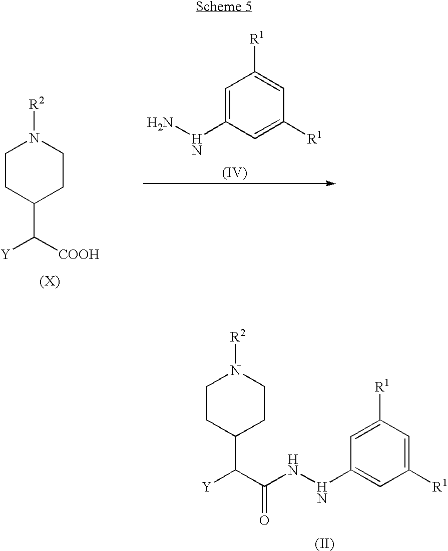

Compounds of formula (II), corresponding to compounds of formula (I) in which X═C, R^{1a}═H, R^{2a}═H may be prepared according to Scheme 5. Conveniently, compounds of formula (X) may be reacted with compounds of formula (IV) in the presence of a suitable amide coupling reagent (e.g. TBTU, HATU, DCC/HOBt.H₂O, DCE/HOBt.H₂O, BOP, pyBOP) in presence of a base such as TEA, diisopropylethylamine or N-methylmorpholine, in a suitable aprotic solvent such as DMF, DCM or THF at room temperature, for a suitable time such as from 15 hours to one day, to afford a compound of formula (II).

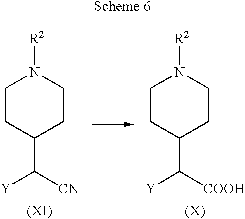

Compounds of formula (X) may conveniently be prepared from compounds of formula (XI) via basic hydrolysis employing a suitable aqueous base (e.g. KOH in water) in a protic solvent such as ethylene glycol, at a suitable temperature such as 130° C., for a suitable length of time such as 5 days (Scheme 6 above).

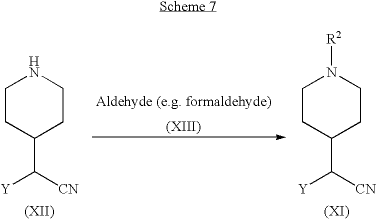

Compounds of formula (XI), wherein R² is not hydrogen, may conveniently be prepared via reaction of a compound of formula (XII) with the required aldehyde in the presence of a suitable hydride donor agent such as NaBH(OAc)₃ in a suitable aprotic solvent (e.g. acetonitrile) at room temperature, for a suitable time such as 3 hours (Scheme 7 above).

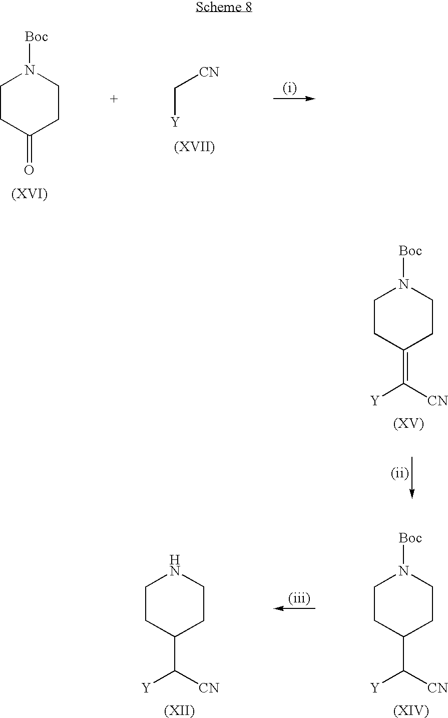

Compounds of formula (XII) may be prepared from commercially available starting materials (XVI) and (XVII) according to Scheme 8 above. Conveniently, step (i) may be performed in the presence of a base (e.g. MeONa) in a suitable solvent such as MeOH, at a suitable temperature such as 75° C., for a suitable time such as 16 hours. Conveniently, the reduction in step (ii) may be prepared using a suitable hydride donor agent (e.g. NaBH₄) in a suitable solvent such as isopropanol at an elevated temperature such as 85° C., for a suitable time such as 16 hours. The deprotection step (iii) may be performed under standard conditions at room temperature, employing a suitable acid (e.g. TFA), in a solvent such as DCM, for a suitable time such as 2 hours to afford (XIII).

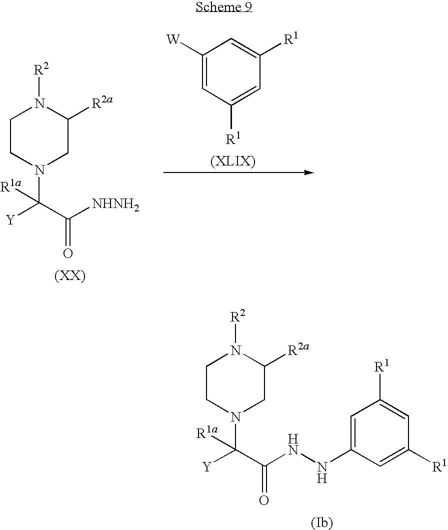

Compounds of formula (Ib) may be prepared according to Scheme 9. Conveniently compounds of formula (XX) may be reacted with compounds of formula (XLIX) (where W is Iodine or Bromine) in the presence of a suitable ligand (e.g. diaminocyclohexane), catalyst (e.g. CuI) and base (e.g. K₂CO₃), in a suitable solvent such as dioxane at an elevated temperature such as 100° C., for a suitable time such as 3 hours, to afford a compound of formula (Ib).

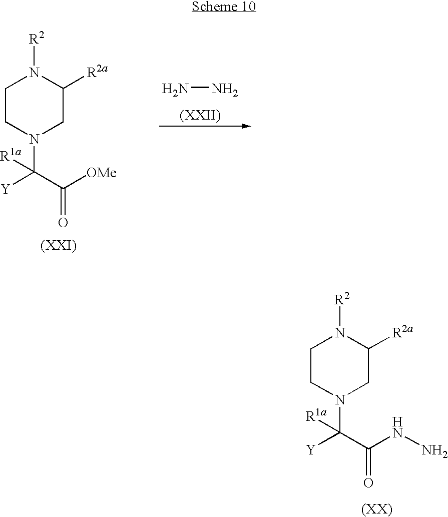

Compounds of formula (XX) may be prepared by reaction of (XXI) with hydrazine (XXII) in the absence of solvent at room temperature for a suitable time such as 3 hours.

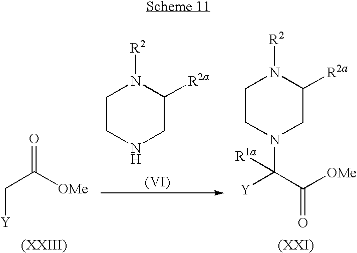

Compounds of formula (XXI) may conveniently be prepared by reaction of (XXIII) with a brominating agent (e.g. N-bromosuccinimide) in presence of a radical initiator (e.g. benzoyl peroxide), followed by treatment with (VI) in the presence of a base (e.g. K₂CO₃), in a suitable solvent such as DCM at room temperature, for a suitable time such as 3 to 4 hours.

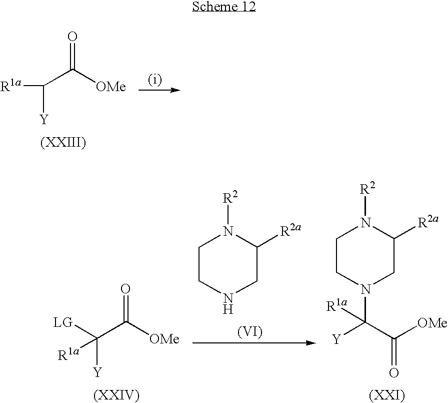

Alternatively, compounds of formula (XXI) may be prepared in a two-step process according to Scheme 12 above. Conveniently, step (i) may be performed by reaction of (XXIII) with a brominating agent (e.g. N-bromosuccinimide) in the presence of a radical initiator (e.g. benzoyl peroxide), in a suitable aprotic solvent such as CCl₄ at room temperature for a suitable time, such as 5 hours, to give a compound of formula (XXIV). Compounds of formula (XXIII) are commercially available. Step (ii) may be performed via reaction of (XXIV) and (VI) in the presence of a suitable base such as K₂CO₃ in an aprotic solvent (e.g. THF) at elevated temperature, such as 85° C., for a suitable time such as 1 hour.

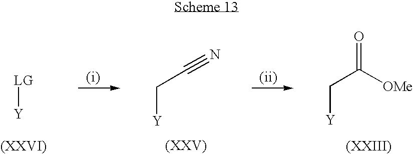

Compounds of formula (XXIII) may conveniently be prepared according to Scheme 13 above. In step (i) (XXVI) may be reacted with acetonitrile in the presence of a base (e.g. nBuLi) in an aprotic solvent (e.g. THF) at a suitable temperature, such as −78° C., for a suitable time, such as 2 hours, to afford compound (XXV). In step (ii) (XXV) may be reacted with an aqueous solution of an inorganic acid (e.g. HCl 37% in water) followed by treatment with a suitable methylating agent (e.g. TMS-CH₂N₂), in a suitable solvent such as water or ethanol, at a suitable temperature, such as from 90° C. to room temperature, for a suitable reaction period, such as 1 hour.

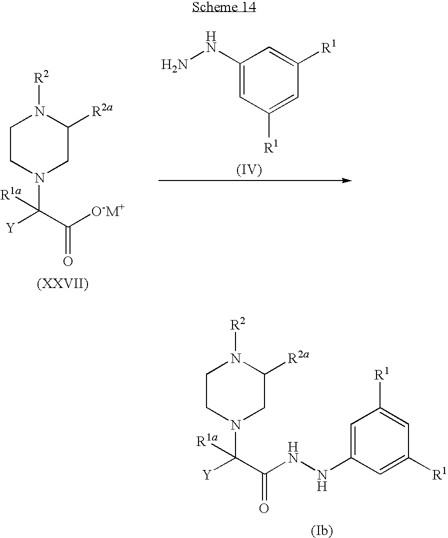

Compounds of formula (Ib) wherein X is N, may be prepared according to Scheme 14. Conveniently, compounds of formula (XXVII) in which M⁺ is a suitable cation (such as K⁺, Na⁺) may be reacted with compounds of formula (IV) in the presence of a suitable amide coupling reagent (e.g. TBTU, TOTU, HATU, DCC/HOBt.H₂O, DCE/HOBt.H₂O, BOP, pyBOP) in the presence of a base such as TEA, diisopropylethylamine or N-methylmorpholine, or alternatively in the absence of a base, in a suitable aprotic solvent such as DMF, DCM or THF at room temperature, for a suitable time such as from 30 minutes to one day, to afford a compound of formula (Ib).

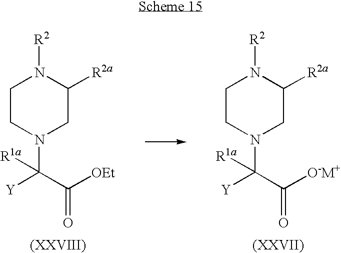

Compounds of formula (XXVII) may be prepared from compounds of formula (XXVIII) via basic ester hydrolysis employing an aqueous solution of an inorganic base (e.g. KOH in water) in a protic solvent such as MeOH at room temperature for a suitable time period, such as from one to five days (Scheme 15 above).

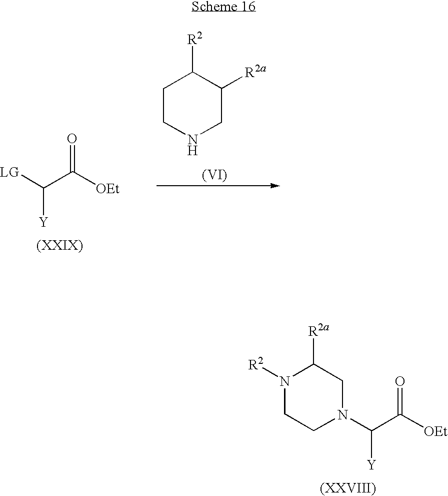

Compounds of formula (XXVIII), corresponding to compounds of formula (XXVII) in which R^{1a} is H, may be prepared by reacting compounds of formula (XXIX) with compounds of formula (VI) in the presence of a suitable base (e.g. triethylamine) and an aprotic solvent (e.g. THF) at room temperature for a suitable time period, such as 2.5 days (Scheme 16 above).

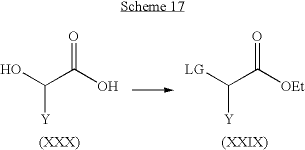

Compounds of formula (XXIX) may be prepared from compounds of formula (XXX) via treatment with a sulfonylating agent (e.g. mesyl chloride) in the presence of a suitable base (e.g. triethylamine) and an aprotic solvent (e.g. THF) at a suitable temperature, such as 0° C., for a suitable period of time, such as 1 hour (Scheme 17 above).

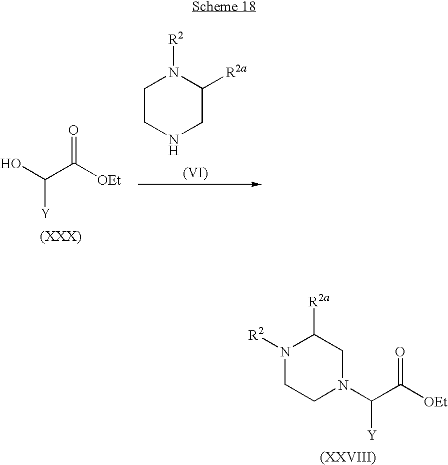

Alternatively, compounds of formula (XXVIII) may be prepared directly from compounds of formula (XXX) via treatment with a sulfonylating agent (e.g. mesyl chloride) in the presence of a suitable base (e.g. triethylamine), followed by reaction with (VI) in an aprotic solvent (e.g. DCM), at a suitable temperature, such as from 0° C. to room temperature, for a suitable time, such as from 1 hour to 2 days (Scheme 18 above).

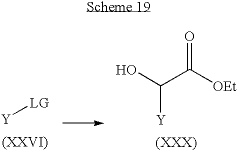

Compounds of formula (XXX) may be prepared from compounds of formula (XXVI) via reaction with ethyl glyoxylate in the presence of a Grignard reagent (e.g. iPrMgCl.LiCl) and an aprotic solvent (e.g. THF), at a suitable temperature, such as from 0° C. to room temperature, for a suitable time, such as 1 hour (Scheme 19 above).

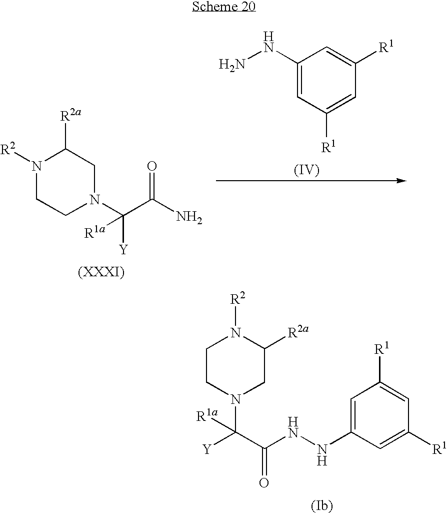

Alternatively, compounds of formula (Ib) wherein X is N, may be prepared according to Scheme 20. Conveniently, compounds of formula (XXXI) may be reacted with compounds of formula (IV) in the presence of a carbamoylating agent (e.g. di-tertbutylcarbonate) and an organic catalyst (e.g. DMAP). The reaction may be carried out in a suitable aprotic solvent such as DCM at a suitable temperature such as room temperature, for a suitable time such as 4.5 days.

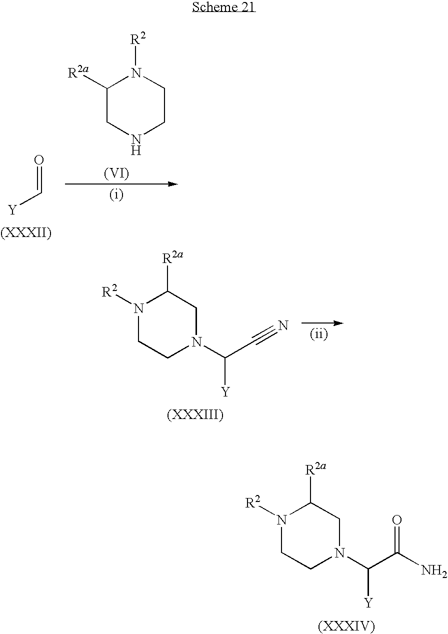

Compounds of formula (XXXIV) corresponding to compounds of formula (XXXI) where R^{1a} is H, may be prepared from compounds of formula (XXXII) in a two-step process according to Scheme 21 above. Conveniently, step (i) may be performed by reacting (XXXII) with (VI) in the presence of a Lewis acid (e.g. Ti(iPrO)₄) and a cyanide donor (e.g. Et₂AlCN). The reaction may be performed in a suitable aprotic solvent (e.g. 1,2-dichloroethane), at a suitable temperature such as room temperature, for a suitable time such as 1 day. Alternatively step (i) may be performed by reacting (XXXII) with (VI) in the presence of an inorganic catalyst (e.g. ZnI₂) with cyanides (e.g. TMSCN) in a protic solvent (e.g. MeOH) at a suitable temperature such as 80° C. for a suitable time such as 3 hours. In step (ii) (XXXII) may be hydrolysed in the presence of an acid (e.g. H₂SO₄) in suitable solvent such as hexane, at a suitable temperature, such as from −20° C. to room temperature, for a suitable time, such as 42 hours.

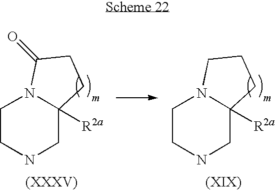

Compounds of formula (XIX), corresponding to compounds of formula (VI) where R² is bonded to the carbon adjacent to the nitrogen bearing the R² group such that a fused 6,5 or 6,6 bicyclic ring system is formed (m=1 or 2), may be prepared from compounds of formula (XXXV), according to Scheme 22 above. Conveniently, (XXXV) may be reduced using a suitable reducing agent such as BH₃/THF in an aprotic solvent, such as THF. The reaction may be performed at a suitable temperature, such as 0° C. to 55° C. for a suitable period of time, such as overnight. Compounds of formula (XXXV) may be prepared according to the procedure described in described in WO03066635A1 (Glaxo Group Limited).

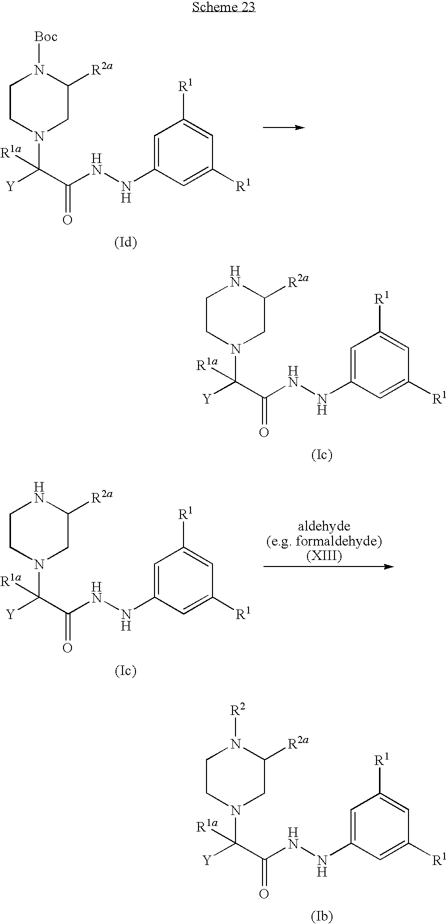

Compounds of formula (Ib) may also be prepared from compounds of formula (Ic) where R² is H, via reaction with the required aldehyde in the presence of a suitable hydride donor agent such as NaBH(OAc)₃ in a suitable aprotic solvent (e.g. acetonitrile) at room temperature, for a suitable time such as 3 hours (Scheme 23). Compounds of formula (Ic) may in turn be prepared by a process of deprotection of protected derivatives such as compounds of formula (Id). Examples of suitable protecting groups and the means for their removal can be found in T. W. Greene and P. G. M. Wuts ‘Protective Groups in Organic Synthesis’ (3^{rd} Ed., J. Wiley and Sons, 1999). In particular, compounds of formula (Ic) wherein R² is H may be prepared from the corresponding N-Boc-protected derivative via removal of the Boc protecting group under standard acidic conditions.

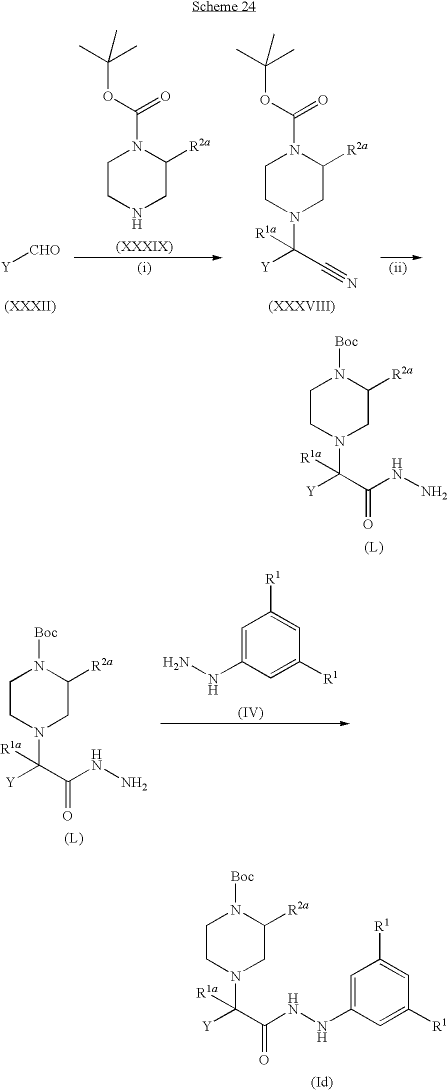

Compounds of formula (L), may be prepared from compounds of formula (XXXII) in a two-step process. Conveniently, step (i) may be performed by reacting (XXXII) with (XXXIX) in the presence of a Lewis acid (e.g. Ti(iPrO)₄) and a cyanide donor (e.g. Et₂AlCN) to form compounds of formula (XXXVIII). The reaction may be performed in a suitable aprotic solvent (e.g. 1,2-dichloroethane), at a suitable temperature such as room temperature, for a suitable time such as 1 day. Alternatively step (i) may be performed by reacting (XXXII) with (XXXIX) in the presence of an inorganic catalyst (e.g. ZnI₂) with cyanides (e.g. TMSCN) in a protic solvent (e.g. MeOH) at a suitable temperature such as 80° C. for a suitable time such as 3 hours to form compounds of formula (XXXVIII). In step (ii) (XXXVIII) may be hydrolysed in the presence of an acid (e.g. H₂SO₄) in suitable solvent such as hexane, at a suitable temperature, such as from −20° C. to room temperature, for a suitable time, such as 42 hours to form compounds of formula (L). Compounds of formula (Id), may be conveniently prepared by reacting compounds of formula (L) with compounds of formula (IV) in the presence of a carbamoylating agent (e.g. di-tertbutylcarbonate) and an organic catalyst (e.g. DMAP). The reaction may be carried out in a suitable aprotic solvent such as DCM at a suitable temperature such as room temperature, for a suitable time such as 4.5 days (Scheme 24)

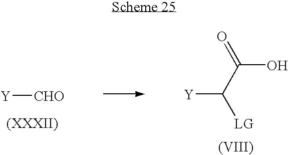

Compounds of formula (VIII) can also be prepared from compounds of formula (XXXII) by reacting (XXXII) in the presence of bromoform and KOH in an aprotic solvent such as 1,4-dioxane. The reaction may be performed at a suitable temperature, such as 0° C. to RT for a suitable period of time, such as overnight (scheme 25).

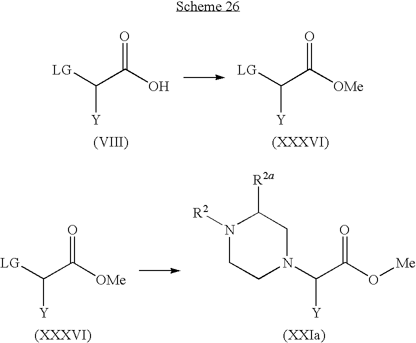

Compounds of formula (XXIa) in which R^{1a} is H, can be prepared from compounds of formula (VIII) by reacting (VIII) in the presence of methanol and sulphuric acid and heat the reaction at suitable temperature such as 85° C. for a suitable time period such as 5 hours, to form (XXXVI), which may be in turn reacted with compounds of formula (VI) in the presence of a base such as triethylamine or sodium bicarbonate, in a suitable solvent such as MeOH, at suitable temperature such as 80° C. for a suitable time period such as 3 hours to form (XXIa).

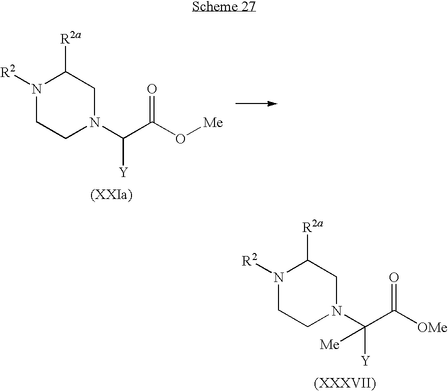

Compounds of formula (XXXVII) can be prepared from compounds of formula (XXIa) by reacting (XXIa) in the presence of a suitable base such as lithium bis(trimethylsilyl)amide, at a suitable temperature such as −78° C., followed by addition of a suitable alkylating agent such as iodomethane in a suitable aprotic solvent such as THF, at a suitable temperature such as −78° C., for a suitable time period such as 3 hours, to form (XXXVII).

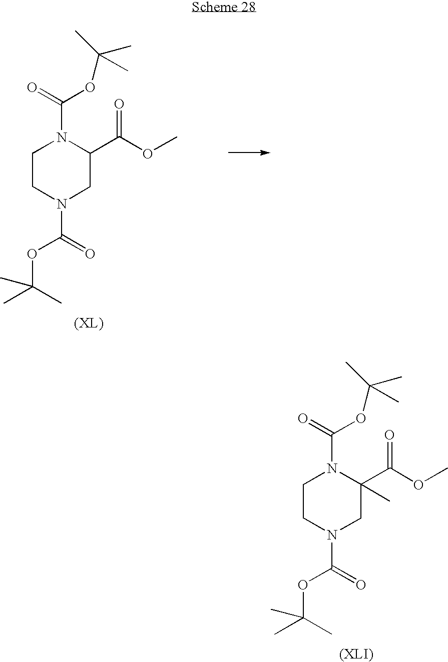

Compounds of formula (XLI) can be prepared from compounds of formula (XL) by reacting (XL) in the presence of a suitable base such as lithium bis(trimethylsilyl)amide, at a suitable temperature such as −78° C., followed by addition of a suitable alkylating agent such as iodomethane in a suitable aprotic solvent such as THF, at a suitable temperature such as −20° C. for a suitable time period such as 3 hours, to form (XLI).

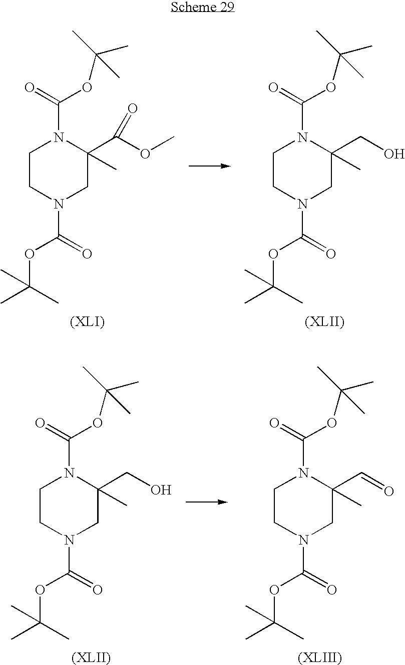

Compounds of formula (XLIII) can be prepared from compounds of formula (XLI) in a two steps procedure by reacting (XLI) in an aprotic solvent, such as THF, in the presence of a suitable reducing agent such as lithium aluminium hydride, at a suitable temperature, such as 0° C., for a suitable time period such as 3 hours, to form (XLII), and then reacting (XLII) in an aprotic solvent such as DCM in the presence of a suitable base such as sodium carbonate and an oxidizing agent such as Dess-Martin periodinane at a suitable temperature such as room temperature and for a suitable time period such as 3 hours, to form (XLIII).

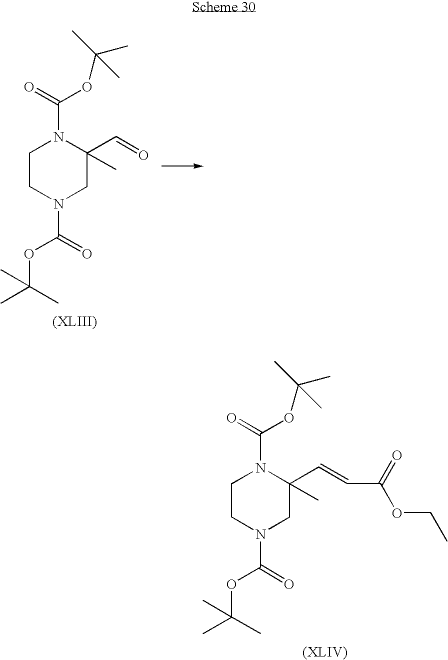

Compounds of formula (XLIV) can be prepared from compounds of formula (XLIII) by reacting (XLIII) in an aprotic solvent such as toluene, in the presence of a suitable alkenylating agent such as ethyl(triphenyl-5-phosphanylidene)acetate, at a suitable temperature such as 80° C., for a suitable time period such as 7 hours, to form (XLIV).

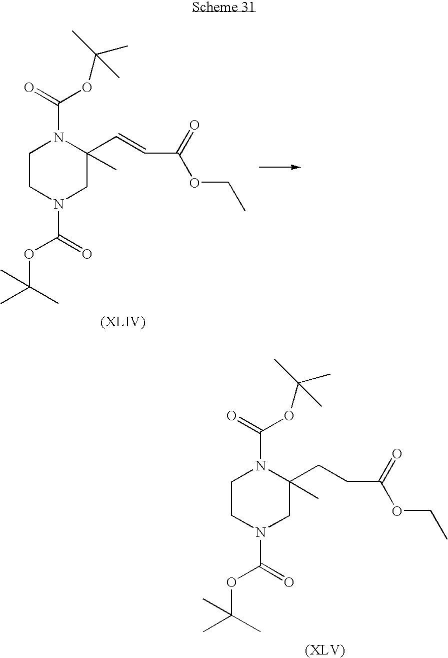

Compounds of formula (XLV) can be prepared by compounds of formula (XLIV) by reacting (XLIV) in a protic solvent such as ethanol, in the presence of a suitable catalyst such as Pd on carbon and under a hydrogen atmosphere, at a suitable temperature such as room temperature and for a suitable time period such as 20 hours, to form (XLV).

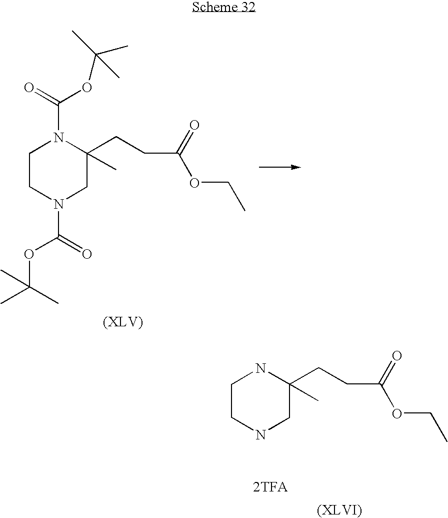

Compounds of formula (XLVI) can be prepared by compounds of formula (XLV) by reacting (XLV) in a suitable solvent such as DCM, at a suitable temperature such as from 0° C. to room temperature, with a suitable broensted acid such as trifluoroacetic acid, and for a suitable time period such as 2 hours, to form (XLVI).

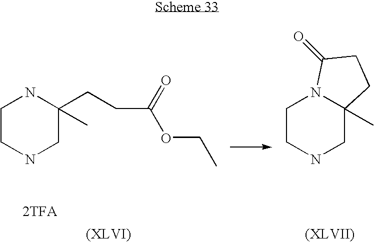

Compounds of formula (XLVII) can be prepared by reacting compounds of formula (XLVI) in a suitable solvent such as DCM, at a suitable temperature such as from 0° C. to room temperature, with a suitable Broensted acid such as trifluoroacetic acid, and for a suitable time period such as 2 hours, to form (XLVII).

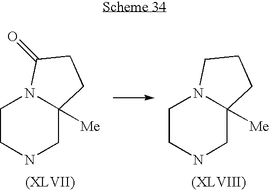

Compounds of formula (XLVIII), can be prepared by reacting compounds of formula (XLVII) corresponding to compounds of formula (XXXV) wherein R^{2a}═CH₃ in a suitable solvent such as THF, at a suitable temperature such as from 0° C. to 50° C., in the presence of a suitable reducing agent such as borane tetrahydrofurane complex, and for a suitable time period such as 24 hours, followed by treatment, in a protic solvent such as methanol with a Broensted acid such as hydrochloric acid, to form (XLVIII).

In one aspect the present invention provides a process for preparing compounds of formula (XLIX) as depicted in Scheme 35, which comprises:

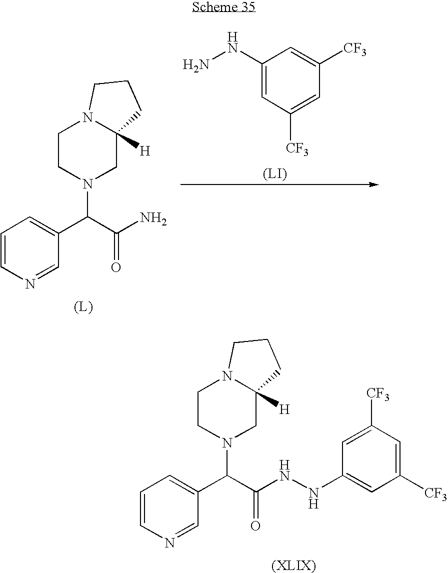

(A) reacting a compound of formula (L) with [3,5-bis(trifluoromethyl)phenyl]hydrazine of formula (LI), together in the presence of a carbamoylating reagent and an organic catalyst in an aprotic solvent; followed by
(B) chiral separation, if required;
   or in the alternative:
(C) reacting a compound of formula (LII) with [3,5-bis(trifluoromethyl)phenyl]hydrazine of formula (LI)

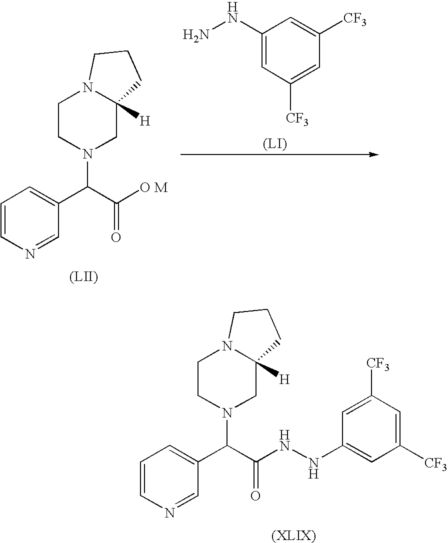

together in the presence of an amide coupling reagent and a suitable base in an aprotic solvent; followed by
(D) chiral separation if required.

In step (A) a carbamoylating reagent may be for example tert-Butyl dicarbonate (Boc₂O). In step (C) M means an appropriate metal cation selected from a group consisting of alkaline—earth-metal or alkaline metal, for example potassium.

In another embodiment the present invention provides a method for increasing the yields of (2R)—N′-[3,5-bis(trifluoromethyl)phenyl]-2-[(8aR)-hexahydropyrrolo[1,2-a]pyrazin-2(1H)-yl]-2-(3-pyridinyl)ethanohydrazide (diastereoisomer 2), as illustrated in the alternative preparation, by epimerisation of the reaction mixture enriched in diastereoisomer 1 (i.e. mother liquor) obtained after removal of the diastereoisomer 2 by filtration and convenient purification through a silica pad, in presence of a suitable base, such as 1,4-diazabicyclo[2.2.2]octane (DABCO) or 1,5,7-triazabiciclo[4.4.0]dec-5-ene (TBD), 2-tert-butylimino-2-diethylamino-1,3-dimethyl-perhydro-1,3,2-diazaphosphorine (BEMP), 1,8-diazabicyclo[5.4.0]undec-7-ene (DBU); in a solvent where the diastereoisomer 2 is unsoluble particularly selected from a group consisting of C₂-C₄ dialkyl ethers such as Et₂O or iPr₂O or THF at an appropriate temperature such as at solvent reflux.

In a further embodiment the present invention thus also provides compounds of formula (I) and pharmaceutically acceptable salts or solvates thereof, for use in medical therapy, and particularly in the treatment of disorders mediated by the ghrelin receptor.

In a further embodiment the present invention is directed to methods of modulating ghrelin receptor activity for the prevention and/or treatment of disorders mediated by the ghrelin receptor.

In a further embodiment the present invention provides a method of treatment of a mammal suffering from a disorder mediated by the ghrelin receptor, which comprises administering to said subject an effective amount of a compound of formula (I) or a pharmaceutically acceptable salt or solvate thereof. Such treatment comprises the step of administering a therapeutically effective amount of the compound of formula (I), including a pharmaceutically acceptable salt or solvate thereof. Such treatment can also comprise the step of administering a therapeutically effective amount of a pharmaceutical composition containing a compound of formula (I), including a pharmaceutically acceptable salt or solvate thereof. As used herein, the term “treatment” refers to alleviating the specified condition, eliminating or reducing the symptoms of the condition, slowing or eliminating the progression of the condition, and preventing or delaying the reoccurrence of the condition in a previously afflicted patient or subject.

A further embodiment of the present invention provides the use of a compound of formula (I), or a pharmaceutically acceptable salt or solvate thereof, in the preparation of a medicament for the treatment of a disorder mediated by the ghrelin receptor.

A further embodiment of the present invention provides the use of a compound of formula (I), or a pharmaceutically acceptable salt or solvate thereof, for the treatment of a disorder mediated by the ghrelin receptor.

The action of the endogenous ligand ghrelin at the ghrelin receptor has been shown to result in potent growth-hormone releasing activity, appetite stimulation, stimulation of gastric motility and acid secretion, positive cardiovascular effects and direct action on bone formation. Thus, a ghrelin receptor modulator may achieve a beneficial effect in the treatment of growth-hormone deficiencies, eating disorders, gastrointestinal disease, cardiovascular diseases, osteoporosis, aging and catabolic states or chronic wasting syndromes (Kojima and Kangawa, Nature Clinical Practice, February 2006, Vol 2, No. 2, 80-88). A ghrelin receptor modulator may also achieve a beneficial effect in the treatment of sleep disorders (Brain Research, 1088 (2006) 131-140).

Particular disorders which are associated with the ghrelin receptor and thus may be mediated by the ghrelin receptor such that a ghrelin receptor modulator may achieve a beneficial effect include obesity and risk factors associated with obesity, including but not limited to diabetes, complications associated with diabetes, metabolic syndrome, cardiovascular disorders (including atherosclerosis and dyslipidemia).

Other diseases and/or conditions mediated by the ghrelin receptor wherein a ghrelin include the following, treating a growth hormone deficient state, increasing muscle mass, increasing bone density, treating sexual disfunction in males and females, facilitating a weight gain, facilitating weight maintenance, facilitating appetite increase (for example facilitating weight gain, maintenance or appetite increase is useful in a patient having a disorder, or under going a treatment, accompanied by weight loss). Examples of diseases or disorders accompanied by weight loss include anorexia, bulimia, cancer cachexia, AIDS, wasting, cachexia and wasting in frail elderly. Examples of treatments accompanied by weight loss include chemiotherapy, radiation therapy, temporary or permanent immobilisation, and dialysis.

Further diseases or conditions include sleep disorders, congestive heart failure, metabolic disorder, improvements in memory function, breast cancer, thyroid cancer, ameliorating ischemic nerve or muscle damage.

The compounds of the invention function by modulating the activity of the ghrelin receptor. They may activate/inactivate the receptor by acting as an agonist, partial agonist, inverse agonist, antagonist or partial antagonist.

Eating disorders include Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50). [the numbers in brackets after the listed diseases above refer to the classification code in Diagnostic and Statistical Manual of Mental Disorders, 4th Edition, published by the American Psychiatric Association (DSM-IV) and/or the International Classification of Diseases, 10th Edition (ICD-10)].

In a further embodiment the invention provides the use of a compound of formula (I), or a pharmaceutically acceptable salt or solvate thereof, in the preparation of a medicament for the treatment of an eating disorder

In a further embodiment the present invention provides a method of treatment of a mammal suffering from an eating disorder which comprises administering to said subject an effective amount of a compound of formula (I) or a pharmaceutically acceptable salt or solvate thereof.

Gastrointestinal diseases include gastric ileus, gastric ulcer and inflammatory bowel diseases such as Crohn's disease and ulcerative colitis. The compounds of the invention may also be useful for treatments to alleviate symptoms associated with gastro-esophageal reflux (GER) and/or with dyspepsia, with or without appetite-/metabolic-related cachexia, and in the treatment of paralytic ileus or pseudo obstruction, and of conditions associated with constipation, such as constipation-predominant irritable bowel syndrome.

Cardiovascular diseases include heart failure and dilated cardiomyopathy.

Catabolic states or chronic wasting syndromes may be seen in post-operative patients and also include AIDS-associated and cancer-associated wasting syndromes, such as cancer cachexia.

While it is possible that, for use in therapy a therapeutically effective amount of a compound of formula (I), or a pharmaceutically acceptable salt or solvate thereof, may be administered as the raw chemical, it is possible to present the active ingredient as a pharmaceutical composition. Thus, in a further embodiment the invention provides a pharmaceutical composition comprising a compound of formula (I), or a pharmaceutically acceptable salt or solvate thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of (I), or a pharmaceutically acceptable salt or solvate thereof, with one or more pharmaceutically acceptable carriers, diluents or excipients.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

A therapeutically effective amount of a compound of formula (I), or a pharmaceutically acceptable salt or solvate thereof will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the ghrelin receptor will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt or solvate thereof, may be determined as a proportion of the effective amount of the compound of formula (I) per se.

A compound of formula (I), or a pharmaceutically acceptable salt or solvate thereof for use in the instant invention may be used in combination with one or more other therapeutic agents. The invention thus provides in a further embodiment a combination comprising a compound of formula (I), or a pharmaceutically acceptable salt or solvate thereof together with a further therapeutic agent, which may be for example an additional anti-obesity agent. In a yet further embodiment the invention also provides the use of a combination comprising a compound of formula (I), or a pharmaceutically acceptable salt or solvate thereof with a further therapeutic agent in the treatment of disorders mediated by the ghrelin receptor.

When a compound of formula (I), or a pharmaceutically acceptable salt or solvate thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

All publications, including but not limited to patents and patent applications, cited in this specification are herein incorporated by reference as if each individual publication were specifically and individually indicated to be incorporated by reference herein as though fully set forth.

### EXPERIMENTAL

The invention is illustrated by the Compounds described below.

Compounds are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 300, 400 or 500 MHz, or on a Bruker instrument at 300 or 400 MHz. Chemical shifts are reported in ppm (δ) using the residual solvent line as internal standard. Splitting patterns are designed as s, singlet; d, doublet; t, triplet; q, quartet; m, multiplet; br, broad. The NMR spectra were recorded at a temperature ranging from 25 to 90° C.

Mass spectra (MS) were taken on an Agilent MSD 1100 Mass Spectrometer, operating in ES (+) and ES (−) ionization mode or on an Agilent LC/MSD 1100 Mass Spectrometer, operating in ES (+) and ES (−) ionization mode coupled with HPLC instrument Agilent 1100 Series [LC/MS-ES (+/−): analysis performed using a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [MeCN] in 5 min, finally under these conditions for 2 min; T=40° C.; flux=1 mL/min; LC/MS-ES (+/−): analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [MeCN] in 5 min, finally under these conditions for 2 min; T=40° C.; flux=1 mL/min]. In the mass spectra only one peak in the molecular ion cluster is reported. Total ion current (TIC) and DAD UV chromatographic traces together with MS and UV spectra associated with the peaks were taken also on a UPLC/MS Acquity™ system equipped with 2996 PDA detector and coupled to a Waters Micromass ZQ™ mass spectrometer operating in positive or ne.g.ative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)].

For the chiral separation and the chiral quality control two different techniques were used: 1) Supercritical Fluid Choromatography (SFC): analytical chromatography was performed on a Berger SFC Analytix, while for the preparative SFC, a Gilson SFC series SF3 was used 2) High Performance Liquid Chromatography (HPLC): chiral Preparative HPLC was performed using a Waters 600 HPLC system and Agilent series 1100 instrument, while for analytical chromatography an Agilent series 1100 HPLC was used.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Me.g.a Be—Si pre-packed cartridges or over pre-packed Biotage silica cartridges.

SPE-SCX cartridges are ion exchange solid phase extraction columns by supplied by Varian. The eluent used with SPE-SCX cartridges is MeOH followed by ammonia solution in MeOH.

Oasis HLB-LP extraction cartridges are ion exchange solid phase extraction columns supplied by Waters. The eluent used with HLB cartridges is water followed by MeOH.

In a number of preparations purification was performed using either Biotage manual flash chromatography (Flash+) or automatic flash chromatography on Horizon or ISCO-Companion systems. All these instruments work with Silica cartridge.

SPE-SI cartridges are silica solid phase extraction columns supplied by Varian or IST Isolute.

In a number of preparations purification was performed on a Mass directed autopurification (MDAP) system FractionlynxTM™ equipped with Waters 2996 PDA detector and coupled with ZQ™ mass spectrometer (Waters) operating in positive and ne.g.ative electrospray ionisation mode ES+, ES− (mass range 100-1000).

A set of acidic as well as basic semipreparative gradients have been used:

### METHOD A: Chromatographic Acidic Conditions for 30 mg of Crude:

Column: 100×21.2 mm Supelcosil™ ABZ+Plus (5 μm particle size)
Mobile phase: A: [water+0.1% HCOOH]/B: [MeCN+0.1% HCOOH]
Flow rate: 20 mL/min
Gradient: 5% B for 1 min, 95% B in 9 min, 100% B in 3.5 min

### METHOD B: Chromatographic Acidic Conditions for 100 mg of Crude:

Column: 150×30 mm XTerra Prep MS C18 (10 μm particle size)
Mobile phase: A: [water+0.1% HCOOH]/B: [MeCN+0.1% HCOOH]
Flow rate: 40 mL/min
Gradient: 1% B to 100% B in 7 min lasting for 7.5 min.

### METHOD C: Chromatographic Basic Conditions for 100 mg of Crude

Column: 150×30 mm XTerra Prep MS C18 (10 μm particle size)
Mobile phase: A: [water+10 mM NH₄HCO₃ (adjusted to pH 10 with ammonia)]/B: [MeCN]
Flow rate: 40 mL/min
Gradient: 10% B for 0.5 min, 95% B in 12.5 min

### METHOD D: Chromatographic Acidic Conditions for 30 mg of Crude

Column: 150×30 mm XTerra Prep MS C18 (10 μm particle size)
Mobile phase: A: [water+0.1% Formic acid]/B: [MeCN+0.1% Formic acid]
Run time: 25 min
Flow rate: 20 mL/min
UV: 210:400 nm
Gradient: 10% B for 2 min, 20% B in 18 min, 99% B in 4 min
MS (ES+/ES−): mass range 150:900 a.m.u.
   For compounds 39, 40 and 41:

¹H-NMR spectra were recorded on a Varian VXR-300, a Varian Unity-300, a Varian Unity-400 instrument, or a General Electric QE-300. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Microwave experiments were carried out on a Microsynth microwave from Milestone, Conn., USA, using a wattage range from 0→900 Watts to achieve the appropriate internal temperatures.

Where a percentage yield has been quoted for a single enantiomer isolated from a racemic mixture this has been calculated on the basis that the maximum possible yield of material (i.e. 100%) is half of the total mass of the racemate.

Optical rotations where given were recorded using a JASCO digital polarimeter DIP-360 employing the following conditions:

### Wavelength: 589 nm

Cell length: 100 mm
Inte.g.ration time: 20 sec
T=20° C. (thermometer: HAAKE K10)
Solvent: MeOH

Experimental Part valid for intermediates 141, 142, 160, 161, 162, 172, 173, 174, 175, 176, 177, 178, 185, 186, 187, 188 only.

Proton Magnetic Resonance (NMR) spectra were recorded on Bruker Avance II instruments at 300 or 400 MHz or Varian 400 MHz, chemical shifts are reported in ppm (δ) using the residual solvent line as internal standard. Splitting patterns are designed as s, singlet; d, doublet; t, triplet; q, quartet; m, multiplet; br, broad. The NMR spectra were recorded at temperature ranging from 25 to 90° C.; when more than one conformer was detected the chemical shifts for the most abundant one is reported. Mass spectra (MS) were taken on an ion-trap Finnigan MS LCQ, operating in ES (+) and ES (−) ionization mode. LCMS were taken on a quadrupole Mass Spectrometer on a Shimadzu Icms 2010 or Agilent LC/MSD 1100 Series, operating in ES (+) and ES (−) ionization mode. (GC-MS) were taken on a Shimadzu 2010 GCMS with El ion source (Column: DB-5 Carrier gas: He). LC/MS-ES (+): analysis performed on YMC ODS (50×2.0 mm, 5 μm) (mobile phase: from 90% [water+0.1% TFA] and 10% [CH₃CN+0.1% TFA] to 20% [water+0.1% TFA] and 80% [CH₃CN+0.1% TFA] in 2.5 min, finally under these conditions for 0.5 min; T=50° C.; flux=1.0 mL/min; LC/MS-ES (−): analysis performed on YMC ODS (50×2.0 mm, 5 μm) (mobile phase: from 90% [water+0.1% TFA] and 10% [CH₃CN+0.1% TFA] to 20% [water+0.1% TFA] and 80% [CH₃CN+0.1% TFA] in 3 min, finally under these conditions for 2 min; T=50° C.; flux=1.0 mL/min; HPLC (walk-up) were taken on Shimadzu 20AB HPLC with PDA detector (column: YMC ODS 50×4.6 mm, 5 cm). Mobile phase: 90% [water+0.1% TFA] and 10% [CH₃CN+0.1% TFA] to 20% [water+0.1% TFA] and 80% [CH₃CN+0.1% TFA] in 6 min, finally under these conditions for 2 min; T=40° C.; flux=3.0 mL/min. T.L.C. refers to thin layer chromatography on 0.25 mm silica gel plates (60F-254. China National Medicines) and visualized with UV light. For phase separations performed by using microfiltration Devices: phase separation cartridge with polypropylene frit by Whatman or Alltech. SCX means: SCX-cartridges (loading 0.75 mmol/g) by Varian. Solutions were dried over anhydrous sodium sulphate. Methylene chloride and DMF were redistilled over calcium hydride and THF was redistilled over sodium.

### ABBREVIATIONS

AcOEt: ethyl acetate
BOP: (benzotriazol-1-yloxy)tris(dimethyl-amino)phosphonium-hexafluorophosphate
CCl₄: carbon tetrachloride
CH: cyclohexane
DCM: dichloromethane
DIPEA: diisopropylethylamine
DMAP: 2,6-dimethylaminopyridine

### DMF: N,N-dimethylformamide

EDCI.HCl: N-(3-Dimethylaminopropyl)-N′-ethylcarbodiimide hydrochloride
ee: enantiomeric excess
Et₂O: diethyl ether
EtOH: ethanol
HATU: O-(7-azabenzotriazol-1-yl)-N,N,N′N′-tetramethyluronium hexafluorophosphate
HOBT.H₂O: hydroxybenzotriazol monohydrate

### HPLC: High Pressure Liquid Chromatography

IPA: isopropanol

### LAH: Lithium Aluminium Hydride

### MDAP: Mass Directed AutoPurification

MeCN: acetonitrile
MeOH: methanol

### NBS: N-bromosuccinimide

n-BuLi: n-Butyllithium
n-Hex: n-hexane
NMM: 4-methylmorpholine

### NMP: N-methylpyrrolidinone

### PE: Petroleum Ether

PL-DCC: polymer supported cyclohexyl carbodiimide
PL-DIPAM: Polymer supported Diisopropylamine
PS-DIPEA: polymer supported diisopropylethylamine
PS-isocyanate: polymer supported isocyanate
PS-trisamine: polymer supported trisamine
PyBOP: benzotriazol-1-yloxytripyrrolidinophosphonium hexafluorophosphate
RT: room temperature
TBTU: O-(Benzotriazol-1-yl)-N,N,N′,N′-tetramethyluronium tetrafluoroborate
TEA: triethylamine
TFA: trifluoroacetic acid
THF: tetrahydrofuran

### TLC Thin Layer Chromatography

TMEDA: N,N,N′,N′-tetramethylethylendiamine
TMSCHN₂: (trimethylsilyl)diazomethane
TMSCN: trimethylsilylcyanide
TOTU: O-[(Ethoxycarbonyl)cyanomethylenamino]-N,N,N′,N′-tetramethyluronium tetrafluoroborate

In the procedures that follow, after each starting material, reference to a Description or Example by number is typically provided. This is provided merely for assistance to the skilled chemist. The starting material may not necessarily have been prepared from the batch referred to.

### Intermediate 1: (±)-dibenzo[b,d]thien-4-yl(4-methyl-1-piperazinyl)acetic acid hydrochloride

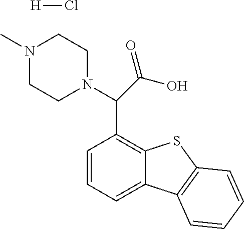

To a solution of 300 mg of dibenzo[b,d]thien-4-ylboronic acid (1.5 mmole, Frontier Scientific Catalog) in 5 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in HCl aq. (aqueous hydrochloric acid, 1N solution, 1 mL) and purified on HLB-LP extraction cartridges (from water to water/MeOH 7:3 as an eluent), the eluent was evaporated under reduced pressure to give the title compound as a yellow foam (270 mg, 53%); ¹H-NMR (400 MHz, DMSO-d₆): δ 2.63 (8H, s), 2.68 (3H, s), 4.35 (1H, s), 7.50 (3H, m), 7.65 (1H, d), 8.02 (1H, d), 8.15 (1H, s), 8.36 (2H, d), 8.42 (1H, s); m/z (ES): 341.1 [M+H]⁺.

### Compound 1: (±)-2-dibenzo[b,d]thien-4-yl-N′-(3,5-dichlorophenyl)-2-(4-methyl-1-pipera-zinyl)acetohydrazide formate

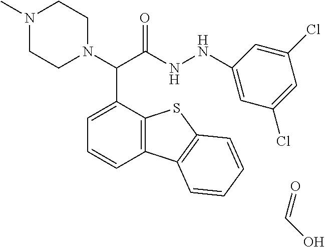

To a solution of 140 mg of dibenzo[b,d]thien-4-yl(4-methyl-1-piperazinyl)acetic acid hydrochloride (intermediate 1, 0.41 mmole) in 5 mL of anhydrous DMF, at RT and under N₂, were added 158 mg of TBTU (0.48 mmole, Aldrich), 380 mg of PL-DIPAM (3.24 mmole/g, 1.23 mmole, Polymer Lab), and 84 mg of 3,5-dichlorophenylhydrazine (0.48 mmole, Lancaster). The reaction was stirred at RT for 24 h. The reaction mixture was then filtered and the solvent was evaporated under reduced pressure. The residue was purified by mass directed autopurification (MDAP) system FractionlynxTM™ (method B), the eluent was evaporated under reduced pressure to give the title compound as a yellow solid (64 mg, 31%); ¹H-NMR (400 MHz, DMSO-d₆): δ 2.22 (3H, s), 2.49 (8H, m), 4.28 (1H, s), 6.45 (2H, s), 6.75 (1H, s), 7.50 (3H, m), 7.65 (1H, d), 8.02 (1H, d), 8.15 (1H, s), 8.36 (2H, d), 8.42 (1H, s); m/z (ES): 499 [M+H]⁺.

### Intermediate 2: (±)-dibenzo[b,d]furan-4-yl(4-methyl-1-piperazinyl)acetic acid hydrochloride

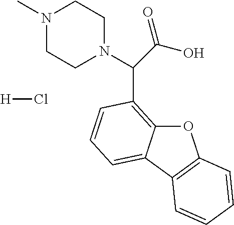

To a solution of 273 mg of dibenzo[b,d]furan-4-ylboronic acid (1.5 mmole, Aldrich) in 5 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in HCl aq. 1 N (1 mL) and purified on HLB-LP extraction cartridges (from water to water/MeOH 7:3 as an eluent), the eluent was evaporated under reduced pressure to give the title compound as a yellow foam (250 mg, 51.5%); ¹H-NMR (400 MHz, DMSO-d₆): δ 2.41 (1H, s), 2.73 (8H, d), 4.77 (1H, s), 7.43 (2H, t), 7.54 (1H, d), 7.58 (1H, d), 7.74 (1H, d), 8.13 (1H, d), 8.17 (3H, m); m/z (ES): 325.2 [M+H]⁺.

### Compound 2: (±)-formic acid-2-dibenzo[b,d]furan-4-yl-N′-(3,5-dichlorophenyl)-2-(4-methyl-1-piperazinyl)acetohydrazide (1:1)

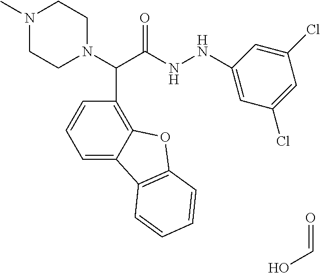

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B), the eluent was evaporated under reduced pressure to give the title compound as a yellow solid (75 mg, 38%); ¹H-NMR (400 MHz, DMSO-d₆): δ 2.28 (3H, s), 2.58 (8H, m), 4.78 (1H, s), 6.48 (2H, s), 6.75 (1H, s), 7.45 (2H, m), 7.56 (1H, t), 7.72 (2H, m), 8.17 (3H, m), 8.40 (1H, s); m/z (ES): 483 [M+H]⁺.

### Intermediate 3: (±)-(4-methyl-1-piperazinyl)(2-naphthalenyl)acetic acid hydrochloride

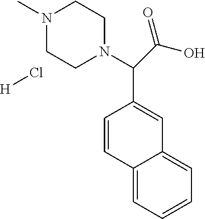

To a solution of 213 mg of 2-naphthalenylboronic acid (1.5 mmole, Aldrich) in 5 mL of dry MeCN at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in 1 mL of HCl aq. 1 N and purified on HLB-LP extraction cartridges (from water to water/MeOH 7:3 as an eluent), the eluent was evaporated under reduced pressure to give the title compound as a yellow foam (160 mg, 37.5%); ¹H-NMR (400 MHz, DMSO-d₆): δ 2.36 (3H, s), 2.51-2.73 (8H, d), 4.19 (1H, s), 7.53 (2H, m), 7.58 (1H, d), 7.93 (4H, m); m/z (ES): 285.2 [M+H]⁺.

### Compound 3: (±)-N′-(3,5-dichlorophenyl)-2-(4-methyl-1-piperazinyl)-2-(2-naphthalenyl)acetohydrazide formate

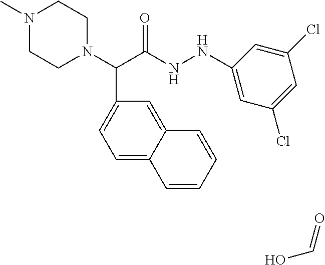

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B), the eluent was evaporated under reduced pressure to give the title compound as a yellow solid (90 mg, 50%); ¹H-NMR (400 MHz, DMSO-d₆): δ 2.28 (3H, s), 2.50 (8H, m), 4.09 (1H, s), 6.38 (2H, s), 6.70 (1H, s), 7.51 (2H, d), 7.69 (1H, d), 7.93 (4H, m), 8.13 (1H, s), 8.39 (1H, s); m/z (ES): 444.2 [M+H]⁺.

### Intermediate 4: (±)-1-benzofuran-2-yl(4-methyl-1-piperazinyl)acetic acid hydrochloride

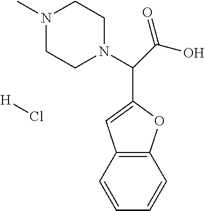

To a solution of 198 mg of 1-benzofuran-2-ylboronic acid (1.5 mmole, Aldrich) in 5 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in 1 mL of HCl aq. 1N and purified on HLB-LP extraction cartridges (from water to water/MeOH 7:3 as an eluent), the eluent was evaporated under reduced pressure to give the title compound as a yellow foam (190 mg, 58.3%); ¹H-NMR (400 MHz, DMSO-d₆): δ 2.54 (3H, s), 2.91 (8H, s), 4.68 (1H, s), 6.82 (1H, s), 7.53-7.65 (2H, m), 7.21-7.31 (2H, m); m/z (ES): 275.2 [M+H]⁺.

### Compound 4: (±)-2-(1-benzofuran-2-yl)-N′-(3,5-dichlorophenyl)-2-(4-methyl-1-piperazinyl)-acetohydrazide formate

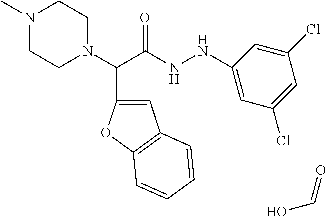

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B), the eluent was evaporated under reduced pressure to give the title compound as a yellow solid (38 mg, 21%); ¹H-NMR (400 MHz, DMSO-d₆): δ 2.24 (3H, s), 2.60 (8H, m), 4.51 (1H, s), 6.64 (2H, s), 6.82 (1H, s), 6.93 (1H, s), 7.29 (2H, m), 7.60 (2H, dd), 8.15 (1H, s), 8.49 (1H, s); m/z (ES): 433 [M+H]⁺.

### Intermediate 5: (±)-(4-methyl-1-piperazinyl){4-[(trifluoromethyl)oxy]phenyl}acetic acid hydrochloride

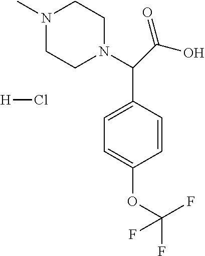

To a solution of 260 mg of {4-[(trifluoromethyl)oxy]phenyl}boronic acid (1.5 mmole, Lancaster) in 5 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled to RT. The solvent was evaporated and the residue dissolved in 1 mL of HCl aq. 1N and purified on HLB-LP extraction cartridges (from water to water/MeOH 7:3 as an eluent), the eluent was evaporated under reduced pressure to give the title compound as a yellow foam (320 mg, 67%); ¹H-NMR (400 MHz, DMSO-d₆): δ 2.73 (3H, s), 2.81 (4H, s), 3.15 (4H, s), 4.60 (1H, s), 7.44 (2H, d), 7.61 (2H, d); m/z (ES): 319.1 [M+H]⁺.

### Compound 5: (±)-N′-(3,5-dichlorophenyl)-2-(4-methyl-1-piperazinyl)-2-{4-[(trifluoromethyl)-oxy]phenyl}acetohydrazide formate

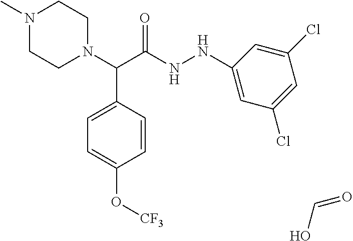

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B), the eluent was evaporated under reduced pressure to give the title compound as a yellow solid (36 mg, 17%); ¹H-NMR (400 MHz, DMSO-d₆): δ 2.18 (3H, s), 2.40 (8H, m), 3.91 (1H, s), 6.35 (2H, s), 6.76 (1H, s), 7.40 (2H, d), 7.62 (2H, d), 8.18 (1H, s), 8.39 (1H, s); m/z (ES): 478.1 [M+H]⁺.

### Intermediate 6: (±)-1-benzothien-2-yl(4-methyl-1-piperazinyl)acetic acid hydrochloride

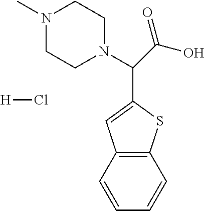

To a solution of 222 mg of 1-benzothien-2-ylboronic acid (1.5 mmole, Aldrich) in 5 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in 1 mL of HCl aq. 1N and purified on HLB-LP extraction cartridges (from water to water/MeOH 7:3 as an eluent), the eluent was evaporated under reduced pressure to give the title compound as a yellow foam (260 mg, 60%); ¹H-NMR (400 MHz, DMSO-d₆): δ 2.47 (3H, s), 2.81 (8H, s), 4.52 (1H, s), 7.32 (2H, m), 7.41 (1H, s), 7.80 (1H, d), 7.90 (1H, d); m/z (ES): 291.1 [M+H]⁺.

### Compound 6: (±)-2-(1-benzothien-2-yl)-N′-(3,5-dichlorophenyl)-2-(4-methyl-1-piperazinyl)-acetohydrazide formate

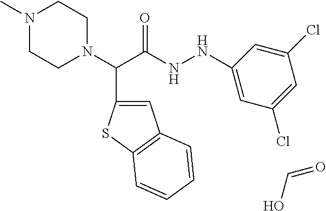

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B), the eluent was evaporated under reduced pressure to give the title compound as a yellow solid (120 mg, 59%); ¹H-NMR (400 MHz, DMSO-d₆): δ 2.28 (3H, s), 2.60 (8H, m), 4.48 (1H, s), 6.55 (2H, s), 6.78 (1H, s), 7.35 (2H, m), 7.45 (1H, s), 7.89 (2H, dd), 8.15 (1H, s), 8.49 (1H, s); m/z (ES): 449 [M+H]⁺.

### Intermediate 7: (±)-1-benzothien-5-yl(4-methyl-1-piperazinyl)acetic acid hydrochloride

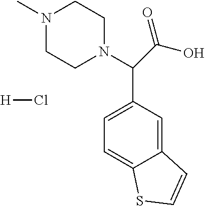

To a solution of 390 mg of 2-(1-benzothien-5-yl)-4,4,5,5-tetramethyl-1,3,2-dioxaborolane (1.5 mmole, Maybridge) in 5.0 mL of dry MeCN, at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue dissolved in 1 mL of HCl aq. 1N and purified on HLB-LP extraction cartridges (from water to water/MeOH 7:3 as an eluent). The eluent was evaporated under reduced pressure to give the title compound as a yellow foam (55 mg, 13%); ¹H-NMR (400 MHz, DMSO-d₆): δ 2.35 (3H, s), 2.81 (4H, s), 3.33 (4H, s), 4.16 (1H, s), 7.42 (1H, d), 7.49 (1H, d), 7.80 (1H, d), 7.91 (1H, s), 8.00 (1H, d); m/z (ES): 291.1 [M+H]⁺.

### Compound 7: (±)-2-(1-benzothien-5-yl)-N′-(3,5-dichlorophenyl)-2-(4-methyl-1-piperazinyl)-acetohydrazide formate

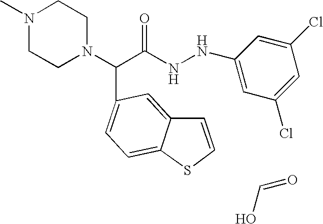

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B). The eluent was evaporated under reduced pressure to give the title compound as a yellow solid (8 mg, 4%); ¹H-NMR (400 MHz, DMSO-d₆): δ 2.14 (3H, s), 2.40 (8H, m), 3.98 (1H, s), 6.32 (1H, s), 6.54 (1H, m), 6.70 (1H, m), 7.35 (1H, m), 7.50 (2H, m), 7.78 (1H, d), 7.99 (1H, m), 8.26 (1H, s), 8.35 (1H, s); m/z (ES): 449 [M+H]⁺.

### Intermediate 8: (±)-dibenzo[b,d]thien-2-yl(4-methyl-1-piperazinyl)acetic acid hydrochloride

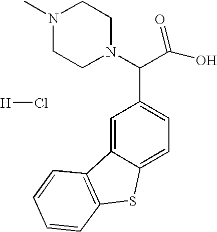

To a solution of 300 mg of dibenzo[b,d]thien-2-ylboronic acid (1.5 mmole, Maybridge) in 5 mL of dry MeCN (5.0 mL), at RT, were added 138 mg of glyoxylic acid hydrate (1.5 mmole, Aldrich) and 166 μL of 1-methylpiperazine (1.5 mmole, Aldrich). The mixture was subjected to microwave irradiation (120° C., 20 minutes) and was cooled down to RT. The solvent was evaporated and the residue was dissolved in 1 mL of HCl aq. 1N and the residue was purified on HLB-LP extraction cartridges (from water to water/MeOH 7:3 as an eluent), the eluent was evaporated under reduced pressure to give the title compound as a yellow foam (280 mg, 55%); ¹H-NMR (400 MHz, DMSO-d₆): δ 2.33 (3H, s), 2.55-2.67 (8H, d), 4.20 (1H, s), 7.53 (2H, m), 7.61 (1H, d), 8.03 (1H, d), 8.05 (1H, d), 8.34 (1H, d), 8.36 (1H, s); m/z (ES): 341.0 [M+H]⁺.

### Compound 8: (±)-2-dibenzo[b,d]thien-2-yl-N′-(3,5-dichlorophenyl)-2-(4-methyl-1-pipera-zinyl)acetohydrazide formate

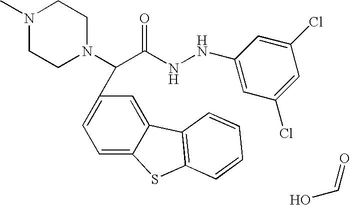

This compound was prepared by a similar procedure to that described for compound 1 from 3,5-dichlorophenylhydrazine and the appropriate carboxylic acid derivative. The residue was purified by mass directed autopurification (MDAP) system Fractionlynx™ (method B). The eluent was evaporated under reduced pressure to give the title compound as a yellow solid (25 mg, 11%); ¹H-NMR (400 MHz, DMSO-d₆): δ 2.17 (3H, s), 2.45 (8H, m), 4.06 (1H, s), 6.33 (2H, s), 6.68 (1H, s), 7.53 (2H, m), 7.68 (1H, dd), 8.03 (2H, m), 8.18 (1H, s), 8.31 (1H, m), 8.35 (1H, dd), 8.42 (1H, dd); m/z (ES): 499 [M+H]⁺.

### Intermediate 9: (±)-bromo(2-chlorophenyl)acetic acid

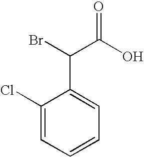

To a solution of 2.0 g of (2-chlorophenyl)acetic acid (11.76 mmole, Aldrich) and 2.1 g of NBS (11.76 mmole, Aldrich) in 60 mL of CCl₄ (carbon tetrachloride) in a dry flask under nitrogen were added 569 mg of benzoyl peroxide (2.35 mmole, Fluka). The solution was stirred at 80° C. for 5 h. The reaction mixture was filtered and the mother liquors were evaporated under reduced pressure. The resulting crude was purified by flash chromatography on 50 g silica gel cartridge using a gradient of DCM/AcOEt (DCM/Ethyl acetate) 10/0 to 5/5 as an eluent. Solvents were removed under reduced pressure to give the title compound (1.7 g, 48%); ¹H-NMR (400 MHz, CDCl₃): δ 5.95 (1H, s), 7.35 (3H, m), 7.80 (1H, d).

### Intermediate 10: (±)-(2-chlorophenyl)(4-methyl-1-piperazinyl)acetic acid hydrochloride

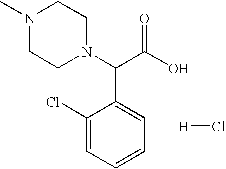

To a solution of 1.7 g of bromo(2-chlorophenyl)acetic acid (Intermediate 9, 6.83 mmole) in 60 mL of THF in a dry flask under nitrogen were added 1.88 g of K₂CO₃ (13.65 mmole) and 836 μL of N-methylpiperazine (7.51 mmole, Aldrich). The solution was then stirred at RT overnight. The reaction mixture was filtered. Then solvents were removed under reduced pressure and the resulting crude was treated with HCl 1N in water. The aqueous phase was passed through HLB-LP extraction cartridges using a gradient of water/MeOH 10/0 to 7/3 as an eluent. Solvents were removed under reduced pressure and the crude was triturated with Et₂O to give the title compound as an orange solid (540 mg, 36%); ¹H-NMR (400 MHz, CDCl₃): δ2.88 (3H, s), 3.45 (8H, m), 5.04 (1H, s), 7.32 (2H, m), 7.48 (1H, dd), 7.66 (1H, dd); m/z (ES): 269 [M+H]⁺.

### Intermediate 11: (±)-N′-[3,5-bis(trifluoromethyl)phenyl]-2-(2-chlorophenyl)-2-(4-methyl-1-piperazinyl)acetohydrazide

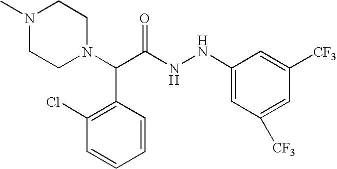

To a suspension of 250 mg of (2-chlorophenyl)(4-methyl-1-piperazinyl)acetic acid hydrochloride (Intermediate 10, 0.93 mmole) and 243 mg of HOBT.H₂O (1.57 mmole) in 8 mL of DCM were added 1 mL of NMP (N-methylpyrrolidininone), 782 mg of PL-DCC (loading: 1.24 mmole, polymer lab), 720 mg of PL-DIPAM (1.86 mmole, polymer lab) and 151 mg of 3,5-bis(trifluoromethyl)phenylhydrazine (0.62 mmole, Fluka). The reaction mixture was stirred at RT overnight. Then PS-isocyanate (loading 1.7 mmole/g, 260 mg, 0.44 mmole) and PS-trisamine (loading 3.5 mmole/g, 864 mg, 3.0 mmole) were added as scavengers. The mixture was stirred overnight at RT. The resins were filtered off and the filtrate was concentrated in vacuo. The crude was dissolved in DCM and the solution was passed through a SCX cartridge. The cartridge was washed with DCM, MeOH and NH₃ 2M/MeOH. Solvents were removed under reduced pressure to give the title compound (170 mg, 42%); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% B to 70% B in 0.5 min, 70% B to 99% B in 0.5 min., 99% B to 3% B in 0.35 min, flow rate: 1 mL/min]: Rₜ=0.63 min; m/z (ES): 495 [M+H]⁺.

### Compound 9: N′-[3,5-bis(trifluoromethyl)phenyl]-2-(2-chlorophenyl)-2-(4-methyl-1-piperazinyl)acetohydrazide-enantiomer 1

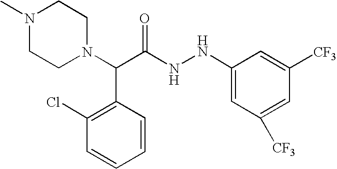

170 mg of racemic of (±)-N′-[3,5-bis(trifluoromethyl)phenyl]-2-(2-chlorophenyl)-2-(4-methyl-1-piperazinyl)acetohydrazide (Intermediate 11) were purified by semipreparative chiral HPLC [Chiralpak AD-H. Eluent: n-Hex/IPA+0.1% isopropylamine 95/5. Flow rate: 14 mL/min. UV detection: 245 nm. Injection: 21 mg/mL in EtOH]. Solvents were removed under reduced pressure to give the title compound as a white solid (39 mg, 46%). ¹H-NMR (400 MHz, DMSO-d₆): δ 2.13 (3H, s), 2.40 (8H, m), 4.40 (1H, s), 7.09 (2H, s), 7.28 (1H, s), 7.35 (2H, m), 7.45 (1H, d), 7.78 (1H, s), 8.80 (1H, s), 10.46 (1H, s); LC-MS [Supelcosil ABZ+Plus, 33×4.6 mm, 3 μm, gradient: A: H₂O+0.1% HCOOH/B: MeCN: 0% to 95% B in 5 min., 95% B for 1.5 min., 95% B to 0% B in 0.1 min., flow rate: 1 mL/min]: R=4.17 min (100%) m/z (ES): 495 [M+H]⁺; Chiral HPLC [Chiralpak AD-H, 250×4.6 mm. Flow rate: 1.0 mL/min. UV detection: 200-400 nm. Mobile phase: A: n-Hex/B: EtOH+0.1% isopropylamine. Gradient: isocratic 5% B]: Rₜ=8.60 min (89.8% ee).

### Compound 10: N′-[3,5-bis(trifluoromethyl)phenyl]-2-(2-chlorophenyl)-2-(4-methyl-1-piperazinyl)acetohydrazide-enantiomer 2

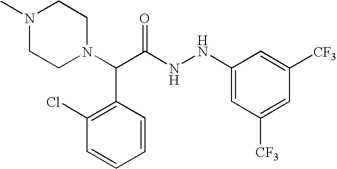

170 mg of (±)-N′-[3,5-bis(trifluoromethyl)phenyl]-2-(2-chlorophenyl)-2-(4-methyl-1-piperazinyl)acetohydrazide (Intermediate 11) were purified by semipreparative chiral HPLC [Chiralpak AD-H. Eluent: n-Hex/IPA+0.1% isopropylamine 95/5. Flow rate: 14 mL/min. UV detection: 245 nm. Injection: 21 mg/mL in EtOH]. Solvents were removed under reduced pressure to give the title compound as a white solid (22 mg, 26%). ¹H NMR (400 MHz, DMSO-d₆): δ 2.45 (11H, m), 4.49 (1H, s), 7.10 (2H, s), 7.27 (1H, s), 7.35 (2H, m), 7.49 (1H, d), 7.75 (1H, d), 8.80 (1H, s), 10.49 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% B to 70% B in 0.5 min, 70% B to 99% B in 0.5 min., 99% B to 3% in 0.35 min, flow rate: 1 mL/min]: Rₜ=0.65 min, m/z (ES): 495 [M+H]⁺; Chiral HPLC [Chiralpak AD-H, 250×4.6 mm. Flow rate: 1.0 mL/min. UV detection: 200-400 nm. Mobile phase: A: n-Hex/B: EtOH+0.1% isopropylamine. Gradient: isocratic 5% B]: Rₜ=11.8 min (>99.9% ee).

### Alternative Preparation of Intermediate 11: (±)-N′-[3,5-bis(trifluoromethyl)phenyl]-2-(2-chlorophenyl)-2-(4-methyl-1-piperazinyl)acetohydrazide

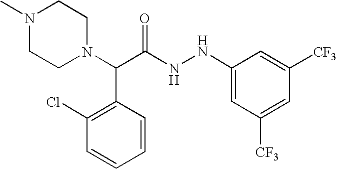

20 g of bromo (2-chlorophenyl)acetic acid (80 mmole, Apollo) were suspended in 120 mL of dry toluene under a N₂ atmosphere. 9 mL of SOCl₂ (113 mmole, Riedel-Haen) was added and the mixture was refluxed for 2 h. The resulting solution was cooled to RT. The solvent and residual SOCl₂ were removed under reduced pressure to obtain 23 g of the crude intermediate which was dissolved in 100 mL of dry THF and added dropwise to a 0° C. mixture of 21.47 g of 3,5-bis(trifluoromethyl)phenylhydrazine (87.9 mmole, Lancaster) and 33 g of K₂CO₃ (240 mmole) in 300 mL of dry THF. The suspension was allowed to stir for 15 minutes at room temperature then 22.3 mL of 1-Methyl piperazine (Sigma-Aldrich), dissolved in 100 mL of dry THF were added at RT. After stirring for 2 h, 800 mL of water was added to the reaction mixture and the solution extracted 3 times with 800 mL of AcOEt. The organic phase was dried on Na₂SO₄, filtered and the residual solvent removed under reduced pressure. The crude material was then purified by Flash Chromatography (9:1=DCM:MeOH) to give 22 g of the title compound as a pale yellow solid. (71%); ¹H-NMR (400 MHz, DMSO-d₆) δ 2.11 (3H, s), 2.21-2.36 (4H, m), 2.34-2.45 (4H, m), 4.40 (1H, s), 7.04-7.08 (2H, m), 7.21-7.24 (1H, m), 7.24-7.35 (2H, m), 7.41 (1H, dd), 7.72 (1H, dd), 8.73 (1H, d), 10.41 (1H, d).

### Alternative Preparation of Compound 9: N′-[3,5-bis(trifluoromethyl)phenyl]-2-(2-chlorophenol)-2-(4-methyl-1-piperazinyl)acetohydrazide-enantiomer 1

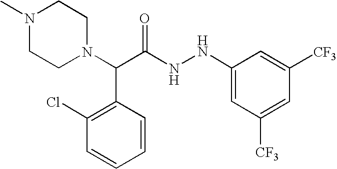

Intermediate 11 (±)-N′-[3,5-bis(trifluoromethyl)phenyl]-2-(2-chlorophenyl)-2-(4-methyl-1-piperazinyl)acetohydrazide was purified by chiral preparative HPLC; This separation was achieved using a Varian SD-2 800G Prep HPLC system set-up with a 75 mm i.d.×250 mm preparative HPLC column packed with Chiralpak AD 20 micron chiral stationary phase. The Intermediate 11 (racemate) was dissolved in EtOH (0.5 g to 0.6 g in 1 mL) and diluted with n-heptane (9 mL) then injected onto the column. The column was eluted with 97:3:0.1 n-heptane:EtOH:iso-propylamine at a flow rate of 200 mL/min. The first eluting enantiomer (Compound 9) was collected in a fraction eluting typically between 15.4 min and 19.5 min. Fractions containing the first eluting enantiomer were analysed by HPLC using a Chiralpak AD analytical column (250×4.6 mm) eluting with 95:5:0.1 n-heptane:EtOH:iso-propylamine at 1 mL/min and a column temperature of 25° C. 22.9 g of racemate was processed in this way. Fractions containing material of the required purity were evaporated, dissolved in EtOH, filtered to remove particulates, evaporated, reslurried in n-heptane and evaporated to give Compound 9 (9.63 g, 84%) as a yellow solid which contained 0.7% of the other enantiomer.

### Intermediate 12: (±)-(4-methyl-1-piperazinyl){2-[(trifluoromethyl)oxy]phenyl}acetic acid hydrochloride

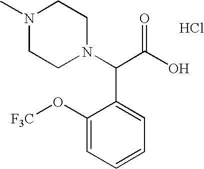

A solution of 500 mg of 2-(trifluoromethoxy)phenylboronic acid (2.4 mmole, Lancaster), 270 μL of N-methylpiperazine (2.4 mmole, Aldrich) and 222 mg of glyoxylic acid monohydrate (2.4 mmole, Aldrich) in 10 mL of MeCN were refluxed for 24 hours. Then it was allowed to cool to RT and concentrated in vacuo HCl 2N was added and the suspension was passed through HLB cartridge (from H₂O to H₂O/MeOH 6:4 as an eluent) to give the title compound as a white solid (100 mg, 12%); ¹H-NMR (400 MHz, DMSO-d₆): 2.2 (3H, s), 2.30-2.75 (8H, m), 3.5 (1H, br s), 4.3 (1H, s), 7.3-7.6 (3H, m), 7.7 (1H, m).

### Compound 11: (±)-N′-[3,5-bis(trifluoromethyl)phenyl]-2-(4-methyl-1-piperazinyl)-2-{2-[(trifluoromethyl)oxy]phenyl}acetohydrazide

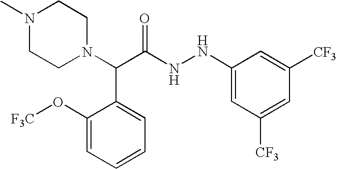

To a suspension of 47.5 mg of (±)-(4-methyl-1-piperazinyl){2-[(trifluoromethyl)oxy]phenyl}acetic acid (Intermediate 12, 0.15 mmole) and 26 mg of HOBT.H₂O (0.25 mmole) in 4 mL of DCM were added 1 mL of NMP, 153 mg of PS-DCC (0.20 mmole) and 92 mg of PL-DIPAM (0.30 mmole). The mixture reaction was stirred at RT overnight. Then 44 mg of PS-isocyanate (loading: 1.58 mmole/g, 0.07 mmole, Argonaut Technologies) and 130 mg of PS-Trisamine (loading 3.5 mmole/g, 0.46 mmole, Argonaut Technologies) were added as scavengers. The mixture was stirred overnight at RT. The crude was filtered and purified first using a SCX cartridge, then with Fractionlynx™ (method B), to give the title compound (50 mg, 62%); ¹H-NMR (400 MHz, DMSO-d₆): δ 2.18 (3H, s), 2.44 (8H, m), 4.37 (1H, s), 7.12 (2H, s), 7.30 (1H, s), 7.42 (2H, m), 7.85 (1H, d), 8.18 (1H, s), 8.80 (1H, s), 10.50 (1H, s); LC-MS [Supelcosil ABZ+Plus, 33×4.6 mm, 3 μm, gradient: A: H₂O+0.1% HCOOH/B: MeCN: 0% to 95% B in 5 min., 95% B for 1.5 min., 95% B to 0% B in 0.1 min., flow rate: 1 mL/min]. Rₜ=4.18 min (100%); m/z (ES): 545 [M+H]⁺.

### Intermediate 13: (±)-(3-methylphenyl)(4-methyl-1-piperazinyl)acetic acid hydrochloride

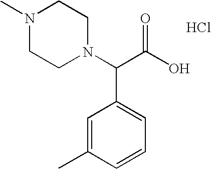

To a suspension of 400 mg of m-tolylboronic acid (2.94 mmole) in 3 mL of anhydrous MeCN at RT and under nitrogen atmosphere, were added 270 mg of glyoxylic acid hydrate (2.94 mmole) and 326 μL of 1-methylpiperazine (2.94 mmole, Aldrich). The reaction mixture was heated under microwave irradiation (120° C., 20 minutes) and then was cooled to RT. The solvent was evaporated and the crude was purified using a HLB-LP cartridge. The title compound was obtained as a brown oil (275 mg, 23%); ¹H-NMR (400 MHz, CDCl₃): δ 2.32 (3H, s), 2.65 (3H, s), 3.04 (8H, m), 4.16 (1H, s), 7.10 (1H, d), 7.24 (3H, m); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% B to 70% B in 0.5 min, 70% B to 99% B in 0.5 min., 99% B to 3% B in 0.35 min, flow rate: 1 mL/min]: Rₜ=0.38 min, m/z (ES): 249 [M+H]⁺.

### Intermediate 14: (±)-N′-[3,5-bis(trifluoromethyl)phenyl]-2-(3-methylphenyl)-2-(4-methyl-1-piperazinyl)acetohydrazide

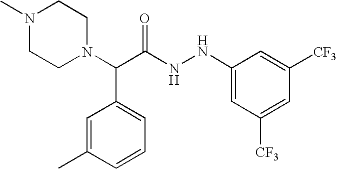

To a solution of 143 mg of (±)-(3-methylphenyl)(4-methyl-1-piperazinyl)acetic acid hydrochloride (intermediate 13, 0.49 mmole) in 1.3 mL of DMF were added successively 130 mg of 3,5-bis(trifluoromethyl)phenylhydrazine (0.54 mmole, Alfa Aesar), 60 μL of NMM (0.54 mmole, Aldrich) and 239 mg of BOP (0.54 mmole, Fluka). The mixture was stirred at RT for 7 h. Solvents were removed under reduced pressure and the residue was dissolved in AcOEt. The organic phase was washed with a solution of NaOH 1N, a saturated solution of NaHCO₃, brine, dried over Na₂SO₄, filtered and evaporated to dryness. The crude was purified by chromatography (5 g silica gel column) using a gradient of DCM/MeOH 10/0 to 8/2 to give the title compound as a white solid (157 mg, 67%); ¹H-NMR (400 MHz, CDCl₃): δ 2.34 (3H, s), 2.38 (3H, s), 2.62 (8H, m), 4.00 (1H, s), 6.31 (1H, s), 7.08 (2H, s), 7.25 (5H, m), 8.60 (1H, s); LC-MS [Supelcosil ABZ+Plus, 33×4.6 mm, 3 μm, gradient: A: H₂O+0.1% HCOOH, B: MeCN: 0% B for 1 min, 0% to 95% B in 5 min., 95% B for 1.5 min., 95% B to 0% B in 0.1 min., flow rate: 1 mL/min]: Rₜ=4.14 min. (100%); m/z (ES): 475 [M+H]⁺.

### Compound 12: N′-[3,5-bis(trifluoromethyl)phenyl]-2-(3-methylphenyl)-2-(4-methyl-1-piperazinyl)acetohydrazide-enantiomer 1

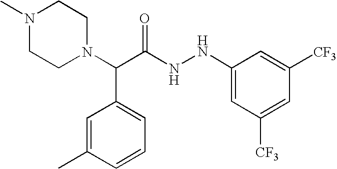

157 mg of (±)-N′-[3,5-bis(trifluoromethyl)phenyl]-2-(3-methylphenyl)-2-(4-methyl-1-piperazinyl)acetohydrazide (Intermediate 14, 0.33 mmole) were purified by semipreparative chiral HPLC [Chiralpak AD-H. Eluent: n-Hex/IPA 92/8. Flow rate: 14 mL/min. UV detection: 245 nm. Injection: 7 mg/mL in EtOH]. Solvents were removed under reduced pressure and the crude was triturated with Et₂O to give the title compound as a white solid (55.7 mg, 46%); ¹H-NMR (400 MHz, CDCl₃): δ 2.33 (3H, s), 2.39 (3H, s), 2.60 (8H, m), 3.99 (1H, s), 6.27 (1H, s), 7.06 (2H, s), 7.26 (4H, m), 8.60 (s, 1H); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% B to 0% B in 0.1 min., flow rate: 2 mL/min]. R=1.75 min (100%); m/z (ES): 475 [M+H]⁺; Chiral HPLC [Chiralpak AD-H, 250×4.6 mm. Flow rate: 1.0 mL/min. UV detection: 200-400 nm. Mobile phase: A: n-Hex/B: IPA. Gradient: 10% B]: Rₜ=4.80 min (>99.5% ee).

### Compound 13: N′-[3,5-bis(trifluoromethyl)phenyl]-2-(3-methylphenyl)-2-(4-methyl-1-piperazinyl)acetohydrazide-enantiomer 2

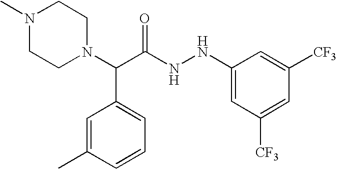

57 mg of (±)-N′-[3,5-bis(trifluoromethyl)phenyl]-2-(3-methylphenyl)-2-(4-methyl-1-piperazinyl)acetohydrazide (Intermediate 14, 0.33 mmole) were purified by semipreparative chiral HPLC [Chiralpak AD-H. Eluent: n-Hex/IPA 92/8. Flow rate: 14 mL/min. UV detection: 245 nm. Injection: 7 mg/mL in EtOH.] Solvents were removed under reduced pressure and the crude was triturated with Et₂O to give the title compound as a white solid (51.5 mg, 44%); ¹H-NMR (400 MHz, CDCl₃): δ 2.33 (3H, s), 2.38 (3H, s), 2.54 (8H, m), 3.98 (1H, s), 6.28 (1H, s), 7.06 (2H, s), 7.45 (4H, m), 8.60 (s, 1H); LC-MS [Supelcosil ABZ+Plus, 33×4.6 mm, 3 μm, gradient: A: H₂O+0.1% HCOOH/B: MeCN: 0% to 95% B in 5 min., 95% B for 1.5 min., 95% to 0% B in 0.1 min., flow rate: 1 mL/min]. R=4.69 min (100%); m/z (ES): 475 [M+H]⁺; Chiral HPLC [Chiralpak AD-H, 250×4.6 mm. Flow rate: 1.0 mL/min. UV detection: 200-400 nm. Mobile phase: A: n-Hex/B: IPA. Gradient: 10% B]: Rₜ=4.88 min (>99.5% ee).

### Intermediate 15: (±)-(2-methylphenyl)(4-methyl-1-piperazinyl)acetic acid hydrochloride

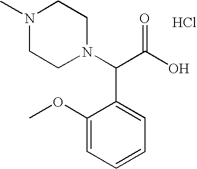

This intermediate was prepared following a similar procedure to that described for Intermediate 13, starting from 400 mg of o-tolylboronic acid (2.94 mmole, Aldrich) to give the title compound (389 mg, 46%); ¹H-NMR (400 MHz, DMSO-d₆): δ 2.37 (3H, s), 2.51 (3H, s), 2.63 (8H, m), 4.28 (1H, s), 7.19 (3H, s), 7.39 (1H, d); UPLC-MS [Acquity™ UPLC BEH C18, 50×21 mm, 1.7 μm, gradient: A: H₂O+0.1% HCOOH/B: MeCN+0.075% HCOOH: 3% B to 6% B in 0.1 min., 6% B to 70% B in 0.5 min, 70% B to 99% B in 0.5 min., 99% B to 3% B in 0.35 min, flow rate: 1 mL/min]: Rₜ=0.40 min; m/z (ES): 249 [M+H]⁺.

### Intermediate 16: (±)-N′-[3,5-bis(trifluoromethyl)phenyl]-2-(2-methylphenyl)-2-(4-methyl-1-piperazinyl)acetohydrazide

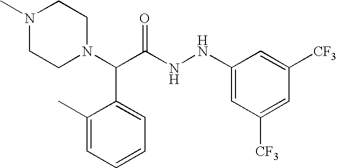

To a solution of 150 mg of (±)-(2-methylphenyl)(4-methyl-1-piperazinyl)acetic acid hydrochloride (Intermediate 15, 0.52 mmole) in 1.25 mL of DMF, were added successively 133 mg of 3,5-bis(trifluoromethyl)phenylhydrazine (0.55 mmole, Alfa Aesar), 60 μL of NMM (0.55 mmole, Aldrich) and 245 mg of BOP (0.55 mmole, Fluka). The mixture was stirred at RT for 3 h. Then AcOEt was added and the organic phase was washed with a solution of NaOH 1N, a saturated solution of NaHCO₃, brine, dried over Na₂SO₄, filtered and evaporated to dryness, to give the title compound as a brown solid (260 mg, 90%); ¹H-NMR (400 MHz, DMSO-d₆): δ2.15 (3H, s), 2.39 (11H, m), 4.18 (1H, s), 7.01 (2H, s), 7.19 (3H, s), 7.27 (1H, d), 7.62 (1H, m), 8.73 (1H, s), 10.23 (1H, s). LC-MS [Supelcosil ABZ+Plus, 33×4.6 mm, 3 μm, gradient: A: H₂O+0.1% HCOOH, B: MeCN: 0% B for 1 min, 0% to 95% B in 5 min., 95% B for 1.5 min., 95% to 0% B in 0.1 min., flow rate: 1 mL/min]: Rₜ=4.14 min. (100%) m/z (ES): 475 [M+H]⁺.

### Compound 14: N′-[3,5-bis(trifluoromethyl)pheny]-2-(2-methylphenyl)-2-(4-methyl-1-piperazinyl)acetohydrazide-enantiomer 1

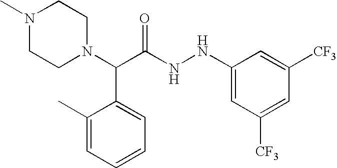

260 mg of racemic of (±)-(N′-[3,5-bis(trifluoromethyl)phenyl]-2-(2-methylphenyl)-2-(4-methyl-1-piperazinyl)acetohydrazide (Intermediate 16, 0.55 mmole) were purified by semipreparative chiral HPLC [Chiralpak AD-H. Eluent: n-Hex/IPA+0.1% isopropylamine 92/8. Flow rate: 14 mL/min. UV detection: 245 nm. Injection: 13 mg/mL in EtOH]. Solvents were removed under reduced pressure and the crude was triturated with Et₂O to give the title compound as a white solid (93 mg, 72%); ¹H-NMR (400 MHz, DMSO-d₆): δ 2.19 (3H, s), 2.40 (11H, m), 4.17 (1H, s), 6.99 (2H, s), 7.15 (2H, s), 7.24 (1H, s), 7.26 (1H, s), 7.63 (1H, m), 8.70 (1H, s), 10.25 (1H, s); LC-MS [Supelcosil ABZ+Plus, 33×4.6 mm, 3 μm, gradient: A: H₂O+0.1% HCOOH/B: MeCN: 0% to 95% B in 5 min., 95% B for 1.5 min., 95% to 0% B in 0.1 min., flow rate: 1 mL/min]. Rₜ=4.12 min (100%) m/z (ES): 475 [M+H]⁺; Chiral HPLC [Chiralpak AD-H, 250×4.6 mm. Flow rate: 1.0 mL/min. UV detection: 200-400 nm. Mobile phase: A: n-Hex/B: IPA. Gradient: 10% B]: Rₜ=5.50 min (91.4% ee).

### Compound 15: N′-[3,5-bis(trifluoromethyl)phenyl]-2-(2-methylphenyl)-2-(4-methyl-1-piperazinyl)acetohydrazide-enantiomer 2

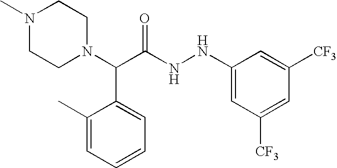

260 mg of racemic of (±)-(N′-[3,5-bis(trifluoromethyl)phenyl]-2-(2-methylphenyl)-2-(4-methyl-1-piperazinyl)acetohydrazide (Intermediate 16, 0.55 mmole) were purified by semipreparative chiral HPLC [Chiralpak AD-H. Eluent: n-Hex/IPA+0.1% isopropylamine 92/8. Flow rate: 14 mL/min. UV detection: 245 nm. Injection: 13 mg/mL in EtOH]. Solvents were removed under reduced pressure and the crude was triturated with Et₂O to give the title compound as a white solid (70 mg, 54%); ¹H-NMR (400 MHz, DMSO-d₆): δ 2.15 (3H, s), 2.20 (11H, m), 4.17 (1H, s), 7.01 (2H, s), 7.19 (3H, s), 7.21 (1H, s), 7.61 (1H, m), 8.71 (1H, s), 10.25 (1H, s); LC-MS [Supelcosil ABZ+Plus, 33×4.6 mm, 3 μm, gradient: A: H₂O+0.1% HCOOH/B: MeCN: 0% to 95% B in 5 min., 95% B for 1.5 min., 95% to 0% B in 0.1 min., flow rate: 1 mL/min]. Rₜ=4.12 min (100%) m/z (ES): 475 [M+H]⁺; Chiral HPLC [Chiralpak AD-H, 250×4.6 mm. Flow rate: 1.0 mL/min. UV detection: 200-400 nm. Mobile phase: A: n-Hex/B: IPA. Gradient: 10% B]: Rₜ=7.95 min (>99.9% ee).

### Intermediate 17: (±)-bromo(2-fluorophenyl)acetic acid

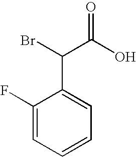

To a solution of 50 mg of (2-fluorophenyl)acetic acid (0.32 mmole, Aldrich) and of 57 mg of N-bromosuccinimide (0.32 mmole, Aldrich) in 2 mL of CCl₄ in a dry flask under nitrogen were added 15.5 mg of benzoyl peroxide (0.06 mmole, Fluka). The solution was then stirred at 80° C. for 5 h. The reaction was repeated with the same procedure on 500 mg of (2-fluorophenyl)acetic acid. The two reaction mixtures were mixed together and filtered. Mother liquors were evaporated under reduced pressure and the resulting crude was purified on flash chromatography on 20 g silica gel cartridge using a gradient of DCM/AcOEt 10/0 to 8/2 as eluent. Solvents were removed under reduced pressure to give the title compound as a yellow oil (690 mg, 83%); ¹H-NMR (400 MHz, DMSO-d₆): 6.00 (1H, s), 7.25 (2H, m), 7.45 (1H, m), 7.55 (1H, m), 13.6 (1H, s).

### Intermediate 18: (±)-(2-fluorophenyl)(4-methyl-1-piperazinyl)acetic acid hydrochloride

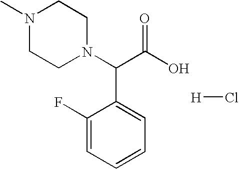

This compound was prepared with the same procedure than intermediate 10 starting from 690 mg of bromo(2-fluorophenyl)acetic acid (2.96 mmole, Intermediate 17) and using acetonitrile as a solvent, to give the title compound (110 mg; 13%); ¹H-NMR (400 MHz, DMSO-d₆): δ 2.50 (4H, m), 2.72 (3H, s), 3.01 (4H, m), 4.59 (1H, s), 7.25 (2H, m), 7.40 (2H, m), 9.73 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.34 min, m/z (ES): 251 [M−H]⁺.

### Intermediate 19: (±)-N′-[3,5-bis(trifluoromethyl)phenyl]-2-(2-fluorophenyl)-2-(4-methyl-1-piperazinyl)acetohydrazide

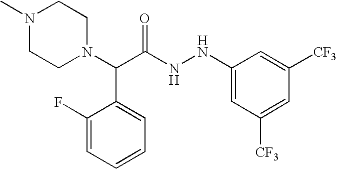

To a solution of 110 mg of (±)-(2-fluorophenyl)(4-methyl-1-piperazinyl)acetic acid hydrochloride (Intermediate 18, 0.38 mmole) in 1.3 mL of DMF, were added successively 98 mg of 3,5-bis(trifluoromethyl)phenylhydrazine (0.42 mmole, Fluka), 44 μL of NMM (0.42 mmole, Aldrich) and 177 mg of BOP (0.42 mmole, Fluka). The mixture was stirred at RT under nitrogen atmosphere, overnight. AcOEt was added and the organic phase was washed with a solution of NaOH 1N, a saturated solution of NaHCO₃, brine, dried over Na₂SO₄, filtered and evaporated to dryness. The crude was dissolved in DCM and the solution was passed through a SCX cartridge. The cartridge was washed with DCM, MeOH and NH₃ 2M/MeOH. Solvents were removed to give the title compound as yellow oil (169 mg, 92%); LC-MS [Supelcosil ABZ+Plus, 33×4.6 mm, 3 μm, gradient: A: H₂O+0.1% HCOOH, B: MeCN: 0% B for 1 min, 0% to 95% B in 5 min., 95% B for 1.5 min., 95% to 0% B in 0.1 min., flow rate: 1 mL/min]: Rₜ=4.02 min. (100%) m/z (ES): 479 [M+H]⁺.

### Compound 16: N′-[3,5-bis(trifluoromethyl)phenyl]-2-(2-fluorophenyl)-2-(4-methyl-1-piperazinyl)acetohydrazide-enantiomer 1

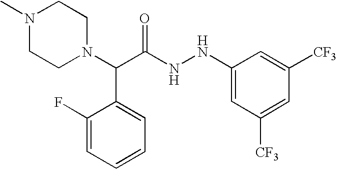

210 mg of (±)-N′-[3,5-bis(trifluoromethyl)phenyl]-2-(2-fluorophenyl)-2-(4-methyl-1-piperazinyl)acetohydrazide (Intermediate 19, 0.45 mmole) were purified by semipreparative chiral SFC. Solvents were removed under reduced pressure and the crude was triturated with Et₂O to give the title compound as a yellow solid (53 mg, 50%); ¹H-NMR (400 MHz, DMSO-d₆): δ 2.15 (3H, s), 2.41 (8H, m), 4.36 (1H, s), 7.08 (2H, s), 7.22 (2H, d), 7.29 (1H, s), 7.37 (1H, m), 7.64 (1H, t), 8.76 (1H, s), 10.39 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]. R=1.70 min (100%) m/z (ES): 479 [M+H]⁺; Chiral SFC [Chiralpak AS-H, 25×0.46 cm. Pressure: 100 bar. Flow rate: 2.0 mL/min. UV detection: 240 nm. Modifier: EtOH+0.1% isopropylamine 12%]: Rₜ=2.90 min (100% ee).

### Compound 17: N′-[3,5-bis(trifluoromethyl)phenyl]-2-(2-fluorophenyl)-2-(4-methyl-1-piperazinyl)acetohydrazide-enantiomer 2

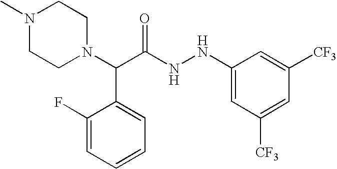

210 mg of (±)-N′-[3,5-bis(trifluoromethyl)phenyl]-2-(2-fluorophenyl)-2-(4-methyl-1-piperazinyl)acetohydrazide (Intermediate 19, 0.45 mmole) were purified by semipreparative chiral SFC. Solvents were removed under reduced pressure and the crude was triturated with Et₂O to give the title compound as a yellow solid (52 mg, 50%); ¹H-NMR (400 MHz, DMSO-d₆): δ 2.15 (3H, s), 2.40 (8H, m), 4.36 (1H, s), 7.08 (2H, s), 7.22 (2H, d), 7.29 (1H, s), 7.37 (1H, m), 7.65 (t, 1H), 8.76 (1H, s), 10.39 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]. R=1.69 min (100%) m/z (ES): 479 [M+H]⁺; Chiral SFC [Chiralpak AS-H, 25×0.46 cm. Pressure: 100 bar. Flow rate: 2.0 mL/min. UV detection: 240 nm. Modifier: EtOH+0.1% isopropylamine 12%]: Rₜ=3.85 min (100% ee).

### Intermediate 20: (±)-2,3-dihydro-1-benzofuran-5-yl(4-methylpiperazin-1-yl)acetic acid

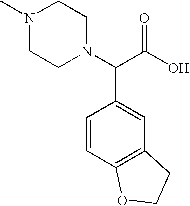

To a suspension of 502 mg of 2,3-dihydro-1-benzofuran-5-ylboronic acid (3.06 mmole, ABCR) in 3 mL of anhydrous MeCN, at RT, under N₂, were added 282 mg of glyoxylic acid hydrate (3.06 mmole) and 340 μL of 1-methylpiperazine (3.06 mmole). The mixture was subjected to microwave irradiation (120° C., 20 minutes) and was cooled down to RT. The solvent was evaporated and the dark residue triturated with Et₂O until a light brown solid formed. The solid was filtered, rinsed with Et₂O and dried under high vacuum. The title compound was obtained as a brown solid (434 mg, 51%); ¹H-NMR (DMSO-d₆): δ 2.13 (3H, s), 2.20-2.60 (8H, m), 3.18 (2H, t), 3.80 (1H, s), 4.50 (2H, t), 6.67 (1H, d), 7.10 (1H, d), 7.25 (1H, s); m/z (ES): 277.3 [M+H]⁺.

### Intermediate 21: (±)-N′-(3,5-dichlorophenyl)-2-(2,3-dihydro-1-benzofuran-5-yl)-2-(4-methylpiperazin-1-yl)acetohydrazide

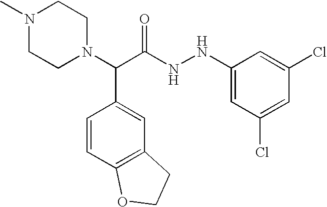

To a suspension of 80 mg of Intermediate 20 (0.29 mmole) in 3 mL of anhydrous DCM, at RT, under N₂, were added 290 mg of PS-DIPEA (3.24 mmole/g: 0.87 mmole) and 93 mg of TBTU (0.44 mmole). The reaction mixture was stirred at RT for 15 minutes and 51 mg of (3,5-dichlorophenyl)hydrazine (0.29 mmole, Aldrich) were added. The reaction mixture was stirred 18 h at RT, the solids were filtered off and the solvent was evaporated. The residue was purified by flash chromatography on silica cartridges (100% DCM to 10% MeOH/DCM) to give the title compound (78 mg, 62%). ¹H-NMR (DMSO-d₆): δ 2.13 (3H, s), 2.35 (8H, m), 3.16 (2H, t), 3.71 (1H, s), 4.50 (2H, t), 6.35 (2H, s), 6.72 (1H, d), 6.74 (1H, t), 7.17 (1H, d), 7.33 (1H, s), 8.33 (1H, s), 10.05 (1H, s); m/z (ES): 435 [M]⁺.

### Compound 18: N′-(3,5-dichlorophenyl)-2-(2,3-dihydro-1-benzofuran-5-yl)-2-(4-methylpiperazin-1-yl)acetohydrazide-enantiomer 1

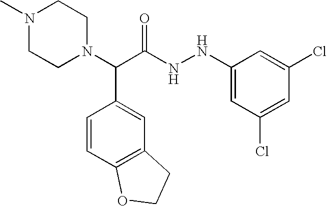

The enantiomers of intermediate 21 (75 mg, 0.17 mmole) were separated by semipreparative chiral HPLC [Chiralpak AD-H. Mobile phase: n-Hex/(2-propanol+0.1% isopropylamine) 75/25% v/v. Flow rate: 14 mL/min. UV detection: 225 nm. Solvents were removed under reduced pressure to give the title compound as a yellow solid (22 mg, 60%); m/z (ES): 435 [M]⁺; Chiral HPLC [Chiralpak AD-H, 250×4.6 mm. Flow rate: 0.8 mL/min. UV detection: 200-400 nm. Mobile phase: n-Hex/(2-propanol+0.1% isopropylamine) 75/25% v/v]. Rₜ=10.6 min (100% ee).

### Compound 19: N′-(3,5-dichlorophenyl)-2-(2,3-dihydro-1-benzofuran-5-yl)-2-(4-methylpiperazin-1-yl)acetohydrazide-enantiomer 2

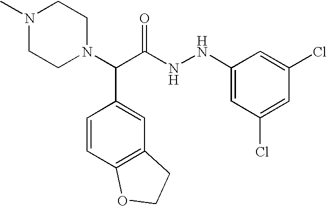

The enantiomers of intermediate 21 (racemate, 75 mg) were separated by semipreparative chiral HPLC [Chiralpak AD-H. Mobile phase: n-Hex/(2-propanol+0.1% isopropylamine) 75/25% v/v. Flow rate: 14 mL/min. UV detection: 225 nm. Solvents were removed under reduced pressure to give the title compound as a yellow solid (24 mg, 64%); m/z (ES): 435 [M]⁺; Chiral HPLC [Chiralpak AD-H, 250×4.6 mm. Flow rate: 0.8 mL/min. UV detection: 200-400 nm. Mobile phase: n-Hex/(2-propanol+0.1% isopropylamine) 75/25% v/v]. Rₜ=17.3 min (100% ee).

### Intermediate 22: (±)-N′-[3,5-bis(trifluoromethyl)phenyl]-2-(2,3-dihydro-1-benzofuran-5-yl)-2-(4-methyl-1-piperazinyl)acetohydrazide

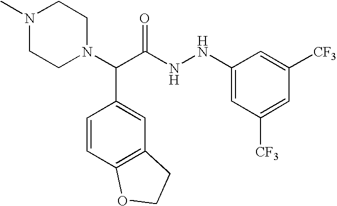

To a suspension of 80 mg of intermediate 20 (0.29 mmole) in 3 mL of anhydrous DCM, at RT, under N₂, were added 290 mg of polymer supported DIPEA (3.24 mmole/g, 0.87 mmole) and 93 mg of TBTU (0.44 mmole). The reaction mixture was stirred at RT for 15 min and 71 mg of [3,5-bis(trifluoromethyl)phenyl]hydrazine (0.29 mmole, Fluka) were added. The reaction mixture was stirred 18 h at RT, the solids were filtered off and the solvent evaporated. The residue was purified by flash chromatography on silica cartridges (100% DCM to 10% MeOH/DCM) and repurified by Fractionlynx™ (method C) to give the title compound (60 mg, 0.12 mmole, 41%); ¹H-NMR (DMSO-d₆): δ 2.15 (3H, s), 2.39 (8H, m), 3.14 (2H, m), 3.75 (1H, s), 4.50 (2H, t), 6.72 (1H, d), 6.96 (2H, br s), 7.18 (1H, d), 7.26 (1H, s), 7.34 (1H, s), 8.71 (1H, s), 10.25 (1H, s); m/z (ES): 503 [M+H]⁺

### Compound 20: N′-[3,5-bis(trifluoromethyl)phenyl]-2-(2,3-dihydro-1-benzofuran-5-yl)-2-(4-methyl-1-piperazinyl)acetohydrazide-enantiomer 1

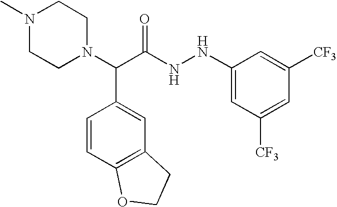

The enantiomers of 56 mg of intermediate 22 (racemate, 0.11 mmole) were separated by semipreparative chiral HPLC [Chiralpak AS-H. Mobile phase: n-Hex/EtOH 89/11% v/v). Flow rate: 14 mL/min. UV detection: 245 nm]. Solvents were removed under reduced pressure to give the title compound as a yellow solid (21 mg, 76%, enantiomer 1); m/z (ES): 503 [M+H]⁺; Chiral HPLC [Chiralpak AS-H, 250×4.6 mm. Flow rate: 0.8 mL/min. UV detection: 200-400 nm. Mobile phase: n-Hex/EtOH 88/12% v/v]. Rₜ=4.8 min (99.5% ee).

### Compound 21: N′-[3,5-bis(trifluoromethyl)phenyl]-2-(2,3-dihydro-1-benzofuran-5-yl)-2-(4-methyl-1-piperazinyl)acetohydrazide-enantiomer 2

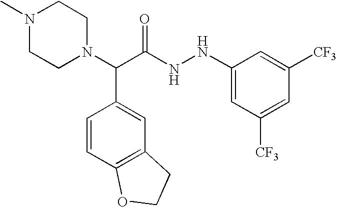

The enantiomers of Intermediate 22 (racemate, 56 mg) were separated by semipreparative chiral HPLC [Chiralpak AS-H. Mobile phase: n-Hex/EtOH 89/11% v/v). Flow rate: 14 mL/min. UV detection: 245 nm]. Solvents were removed under reduced pressure to give the title compound as a yellow solid (20 mg, 62%); m/z (ES): 503 [M+H]⁺; Chiral HPLC [Chiralpak AS-H, 250×4.6 mm. Flow rate: 0.8 mL/min. UV detection: 200-400 nm. Mobile phase: n-Hex/EtOH 88/12% v/v]. Rₜ=7.3 min (99.5% ee).

### Intermediate 23: (±)-bromo(2,3-difluorophenyl)acetic acid

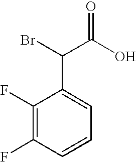

To a solution of 738 mg of (2,3-difluorophenyl)acetic acid (4.3 mmole, Aldrich) and of 761 mg of NBS in 15 mL of CCl₄ in a dry flask under nitrogen were added 208 mg of benzoyl peroxide (0.86 mmole, Fluka) The solution was then stirred at 80° C. for 5 h. The solids were filtered off and the solvent was removed under reduced pressure and the resulting crude was purified on flash chromatography on 25 g silica gel cartridge using a gradient of AcOEt/DCM 0:10 to 8:2 as an eluent. Solvents were removed under reduced pressure to give the title compound as a yellow solid (460 mg, 42%). ¹H-NMR (400 MHz, DMSO-d₆): δ 6.09 (1H, s), 7.15-7.18 (1H, m), 7.23-7.41 (1H, m), 7.47-7.52 (1H, m), 14.76 (1H, br s); UPLC/MS [Acquity™ UPLC BEH C18, 50×21 mm, 1.7 μm, Gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.63 min. (65%) m/z (ES): 249 [M−H]⁻.

### Intermediate 24: (±)-(2,3-difluorophenyl)(4-methyl-1-piperazinyl)acetic acid hydrochloride

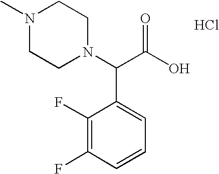

To a solution of 460 mg of (±)-bromo(2,3-difluorophenyl)acetic acid (intermediate 23, 1.8 mmole) in 30 mL of MeCN in a dry flask under nitrogen were added 497 mg of K₂CO₃ (3.6 mmole) and 223 μL of N-methylpiperazine (2 mmole, Aldrich). The solution was then stirred at RT overnight. Solvents were removed under reduced pressure and the resulting crude was treated with HCl 1N in water and washed with DCM. The aqueous phase was separated and passed through HLB-LP cartridge (eluent: MeOH 5% water solution to MeOH). Solvent was removed under reduced pressure to give the title compound as a white solid (110 mg, 20%); ¹H-NMR (DMSO-d₆, 400 MHz): δ 2.27 (3H, s), 2.57-2.68 (8H, m), 4.48 (1H, s), 7.27 (2H, m), 7.42 (1H, m); m/z (ES): 271 [M+H]⁺.

### Intermediate 25: (±)-N′-[3,5-bis(trifluoromethyl)phenyl]-2-(2,3-difluorophenyl)-2-(4-methyl-1-piperazinyl)acetohydrazide

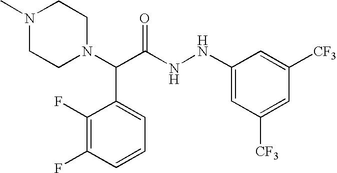

To a solution of 111 mg of (±)-(2,3-difluorophenyl)(4-methyl-1-piperazinyl)acetic acid hydrochloride (intermediate 24, 0.41 mmole) dissolved in 10 mL of dry THF were added 214 mg of PyBOP (0.41 mmole, Aldrich), 169 mg of 3,5-bis(trifluoromethyl)phenylhydrazine (0.69 mmole, 1.1 eq., Fluka), 197.5 μL of DIPEA (1.1 mmole, Aldrich) and the mixture was then stirred at RT overnight. The reaction mixture was washed with NaOH 1N and saturated NaHCO₃, the aqueous phase was extracted with DCM. The combined organic layers were evaporated under reduced pressure. The resulting crude compound was purified by 10 g silica cartridge using MeOH/DCM 9:1, then by Fractionlynx™ method C. Solvents were removed under reduced pressure to give the title compound as a sand colored solid (72 mg, 35%); LC-MS [Supelcosil ABZ+Plus, 33×4.6 mm, 3 μm, gradient: C: NH₄HCO₃ 5 mM, pH=10 (NH₄OH) B: MeCN: 0% to 50% B in 0.4 min., 50% to 95% B for 3.6 min., 95% B for 1 min, 95% to 0% B in 0.1 min., flow rate: 1.5 mL/min]: Rₜ=2.11 min. (100%) m/z (ES): 497.1 [M+H]⁺.

### Compound 22: N′-[3,5-bis(trifluoromethyl)phenyl]-2-(2,3-difluorophenyl)-2-(4-methyl-1-piperazinyl)acetohydrazide-enantiomer 1

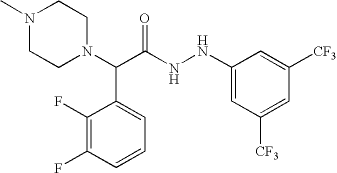

72 mg of (±)-N′-[3,5-bis(trifluoromethyl)phenyl]-2-(2,3-difluorophenyl)-2-(4-methyl-1-piperazinyl)acetohydrazide (intermediate 25, 0.14 mmole) was purified by semipreparative chiral SFC [Chiralpak AS-H, 25×0.46 cm, pressure: 100 bar, Flow rate: 2.0 mL/min, T: 35 C, UV detection: 210-340 nm, modifier: EtOH+0.1% isopropylamine 10%, run time: 25 min]: Rₜ=2.88 min. Solvents were removed under reduced pressure to give the title compound as a white solid (19 mg, 54%). ¹H-NMR (400 MHz, CDCl₃): δ 2.33 (3H, s), 2.55-2.68 (8H, m), 4.60 (1H, s), 6.43 (2H, s), 7.07-7.19 (3H, m), 7.20 (1H, s), 7.4 (1H, s), 8.98 (1H, s); LC-MS [Supelcosil ABZ+Plus, 33×4.6 mm, 3 μm, gradient: A: H₂O+0.1% HCOOH, B: MeCN: 0% to 95% B in 3 min., 95% B for 1 min., 95% to 0% B in 0.1 min; run time: 4.5 min; flow rate: 2 mL/min]: Rₜ=1.74 min. (100%) m/z (ES): 497.1 [M+H]⁺; Chiral SFC [Chiralpak AS-H, 25×0.46 cm, pressure: 100 bar, Flow rate: 2.0 mL/min, UV detection: 240 nm, 10% modifier: EtOH+0.1% isopropylamine, run time 10 min]: Rₜ=2.97 min. (100% ee).

### Compound 23: N′-[3,5-bis(trifluoromethyl)phenyl]-2-(2,3-difluorophenyl)-2-(4-methyl-1-piperazinyl)acetohydrazide-enantiomer 2

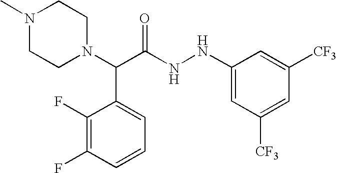

72 mg of (±)-N′-[3,5-bis(trifluoromethyl)phenyl]-2-(2,3-difluorophenyl)-2-(4-methyl-1-piperazinyl)acetohydrazide (Intermediate 25, 0.14 mmole) were purified by semipreparative chiral SFC [Chiralpak AS-H, 25×0.46 cm, pressure: 100 bar, Flow rate: 2.0 mL/min, T: 35 C, UV detection: 210-340 nm, modifier: EtOH+0.1% isopropylamine 10%, run time: 25 min]: Rₜ=4.33 min. Solvents were removed under reduced pressure to give the title compound as a white solid (19 mg, 54%). ¹H-NMR (400 MHz, CDCl₃): δ 2.33 (3H, s), 2.55-2.68 (8H, m), 4.60 (1H, s), 6.43 (2H, s), 7.07-7.19 (3H, m), 7.20 (1H, s), 7.4 (1H, s), 8.98 (1H, s); LC-MS [Supelcosil ABZ+Plus, 33×4.6 mm, 3 μm, gradient: A: H₂O+0.1% HCOOH, B: MeCN: 0% to 95% B in 3 min., 95% B for 1 min., 95% to 0% B in 0.1 min; run time: 4.5 min; flow rate: 2 mL/min]: Rₜ=1.74 min. (100%) m/z (ES): 497.1 [M+H]⁺; Chiral SFC [Chiralpak AS-H, 25×0.46 cm, pressure: 100 bar, Flow rate: 2.0 mL/min, UV detection: 240 nm, 10% modifier: EtOH+0.1% isopropylamine, run time 10 min]: Rₜ=4.55 min. (100% ee).

### Intermediate 26: (±)-Bromo(2,6-difluorophenyl)acetic acid

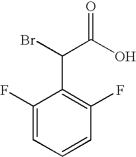

This Intermediate was prepared by a similar procedure to that described for intermediate 9 starting from 500 mg of (2,6-difluorophenyl)acetic acid (2.9 mmole, Aldrich) to give the title compound as a yellow solid (632 mg, 86%); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.63 min (19.73%) m/z (ES): 249 [M−H]⁻,

### Intermediate 27: (±)-(2,6-difluorophenyl)(4-methyl-1-piperazinyl)acetic acid hydrochloride

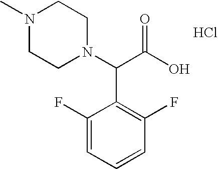

This compound was prepared by a similar procedure to that used for intermediate 10 starting from 632 mg of (±)-bromo(2,6-difluorophenyl)acetic acid (intermediate 26, 2.5 mmole) to give the title compound as a white solid (350 mg, 45%); ¹H-NMR (400 MHz, DMSO-d₆): δ 2.27 (3H, s), 2.49-2.57 (8H, m), 4.28 (1H, s), 7.19-7.32 (3H, m); ES-MS m/z (ES): 271 [M+H]⁺.

### Intermediate 28: (±)-N′-[3,5-bis(trifluoromethyl)phenyl]-2-(2,6-difluorophenyl)-2-(4-methyl-1-piperazinyl)acetohydrazide

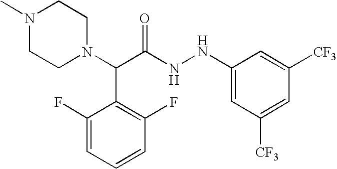

This compound was prepared by a similar procedure to that used for intermediate 11 starting from 350 mg of (±)-2,6-difluorophenyl)(4-methyl-1-piperazinyl)acetic acid hydrochloride (intermediate 27, 1.3 mmole) to give a crude as a white solid (100 mg); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.65 min (8.44%) m/z (ES): 497 [M+H]⁺. The peak at Rₜ=0.36 min (19%) m/z (ES): 270 [M]⁺ was interpreted as presence of 2,6-difluorophenyl)(4-methyl-1-piperazinyl)acetic acid. Therefore the crude (100 mg) was processed for a second time with the same procedure, (Fractionlynx™ method C) to obtain the title compound as a white solid (22 mg, 3.4%); LC-MS [Supelcosil ABZ+Plus, 33×4.6 mm, 3 μm, gradient: A: H₂O+0.1% HCOOH, B: MeCN: 0% to 95% B in 3 min., 95% B for 1 min., 95% to 0% B in 0.1 min; run time: 4.5 min; flow rate: 2 mL/min]: Rₜ=1.73 min. (100%) m/z (ES): 497.1 [M+H]⁺.

### Compound 24: N′-[3,5-bis(trifluoromethyl)phenyl]-2-(2,6-difluorophenyl)-2-(4-methyl-1-piperazinyl)acetohydrazide-enantiomer 1

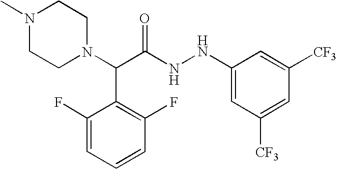

22 mg of (±)-N′-[3,5-bis(trifluoromethyl)phenyl]-2-(2,6-difluorophenyl)-2-(4-methyl-1-piperazinyl)acetohydrazide (intermediate 28, 0.044 mmole) was purified by semipreparative chiral SFC [Chiralpak AS-H, 25×2.1 cm, pressure: 100 bar, Flow rate: 22 mL/min, T: 35° C., UV detection: 240 nm, 17% modifier: 2-Propanol+0.1% isopropylamine, run time: 25 min]: Rₜ=1.24 min. Solvents were removed under reduced pressure to give the title compound as a white solid (4.7 mg, 43%); ¹H-NMR (400 MHz, CDCl₃): δ 2.35 (3H, s), 2.60-2.66 (8H, m), 4.52 (1H, s), 6.40 (1H, s), 7.01-7.07 (3H, m), 7.18 (2H, s), 7.38 (1H, s), 8.88 (1H, s); Chiral SFC [Chiralpak AS-H, 25×0.46 cm, pressure: 100 bar, Flow rate: 2.0 mL/min, UV detection: 240 nm, 17% modifier: 2-Propanol+0.1% isopropylamine, run time 10 min]: Rₜ=2.31 min. (100% ee).

### Compound 25: N′-[3,5-bis(trifluoromethyl)phenyl]-2-(2,6-difluorophenyl)-2-(4-methyl-1-piperazinyl)acetohydrazide-enantiomer 2

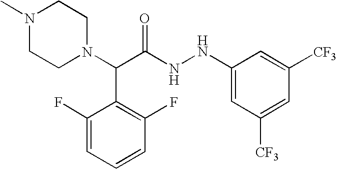

22 mg of racemic (±)-N′-[3,5-bis(trifluoromethyl)phenyl]-2-(2,6-difluorophenyl)-2-(4-methyl-1-piperazinyl)acetohydrazide (intermediate 28, 0.044 mmole) were purified by semipreparative chiral SFC [Chiralpak AS-H, 25×2.1 cm, pressure: 100 bar, Flow rate: 22 mL/min, T: 35 C, UV detection: 240 nm, modifier: 2-Propanol+0.1% isopropylamine 17%, run time: 25 min]: Rₜ=2.30 min. Solvents were removed under reduced pressure to give the title compound as a white solid (4.1 mg, 36%); ¹H-NMR (400 MHz, CDCl₃): δ 2.35 (3H, s), 2.60-2.66 (8H, m), 4.52 (1H, s), 6.53 (1H, s), 7.01-7.07 (3H, m), 7.18 (2H, s), 7.37 (1H, s), 8.97 (1H, s); Chiral SFC [Chiralpak AS-H, 25×0.46 cm, pressure: 100 bar, Flow rate: 2.0 mL/min, UV detection: 240 nm, 15% modifier: 2-Propanol+0.1% isopropylamine, run time 10 min]: Rₜ=8.65 min. (100% ee).

### Intermediate 29: (±)-1-methylethyl 4-[2-{2-[3,5-bis(trifluoromethyl)phenyl]hydrazino}-1-(2-chlorophenyl)-2-oxoethyl]-1-piperazinecarboxylate

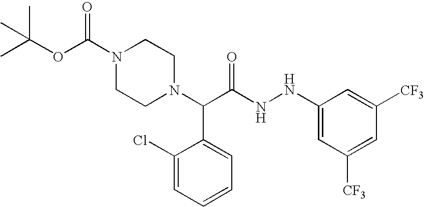

To a suspension of 9 g of (±)-bromo(2-chlorophenyl)acetic acid (Intermediate 9, 36 mmole) in 15 mL of dry toluene was added 3.9 mL of SOCl₂ (54 mmole, Aldrich), and the mixture was heated to reflux for 1 h. The reaction mixture was cooled to room temperature. The solvent and SOCl₂ were removed under reduced pressure to give the acid chloride as a yellow oil. The acid chloride dissolved in 10 mL of dry THF was added over 10 minutes to a solution, cooled at 0° C., of 20 g of 3,5-bis(trifluoromethyl)phenylhydrazine (108 mmole, Fluka) and 15 g of K₂CO₃ (108 mmole) in dry THF. The mixture was then allowed to reach room temperature and stirred for 30 min. Then 20 g of 1,1-dimethylethyl-1-piperazinecarboxylate (108 mmole, Fluka) dissolved in 30 mL of dry THF were added and the reaction mixture was stirred for 1 h. The salts were filtered and washed with THF, the filtrate was evaporated under reduced pressure, the water soluble residue was extracted with DCM. The organic phase was separated from the aqueous phase and the organic solvent was removed under reduced pressure to give the crude which was purified by silica cartridge 75 M using a gradient CH/AcOEt 8:2 to CH/AcOEt 0:10 to AcOEt/MeOH 9:1. Solvents were removed under reduced pressure to give the title compound (4 g, 19%); ¹H-NMR (400 MHz, DMSO-d₆): δ 1.39 (9H, s), 2.42 (4H, m), 3.35 (4H, m), 4.52 (1H, s), 7.00-9.00 (9H, m).

### Intermediate 30: (±)-N′-[3,5-bis(trifluoromethyl)phenyl]-2-(2-chlorophenyl)-2-(1-piperazinyl)acetohydrazide

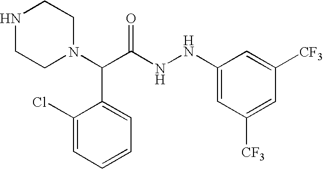

To a solution of 4 g (±)-1-methylethyl-4-[2-{2-[3,5-bis(trifluoromethyl)phenyl]hydrazino}-1-(2-chlorophenyl)-2-oxoethyl]-1-piperazinecarboxylate (intermediate 29, 6.8 mmole) in 100 mL of DCM, 10 mL of TFA were added and the solution was then stirred at RT for 2 hours. The solvents were removed under reduced pressure. The crude was purified by SCX (eluent DCM, MeOH then NH₃ 0.5 M in MeOH) and then by chromatography on a 10 g silica gel cartridge with a gradient of CH/AcOEt 8:2 to CH/AcOEt 0:10 to AcOEt/MeOH 9:1 and then MeOH to obtain 1.64 g of crude which was purified by preparative HPLC [Gemini, C18 AXIA, 50×21 mm, 5 μm, gradient: C: NH₄HCO₃ 10 mM, pH=10/B: MeCN: 40% B for 0.5 min., 40% to 75% B for 5 min., 75% to 100% B in 1.5 min, 100% B in 1.5 min, flow rate: 17 mL/min]: Rₜ=2.63 min. (100%) m/z (ES): 481 [M+H]⁺ to obtain 806 mg of the title compound (24%). HPLC-MS [Gemini, C18 AXIA, 50×21 mm, 5 μm, gradient: C: NH₄HCO₃ 10 mM, pH=10/B: MeCN: 35% B for 0.5 min., 35% to 95% B for 4.5 min., 95% B in 1.5 min, flow rate: 2 mL/min]: Rₜ=2.63 min.

### Compound 26: N′-[3,5-bis(trifluoromethyl)phenyl]-2-(2-chlorophenyl)-2-(1-piperazinyl)acetohydrazide-enantiomer 1

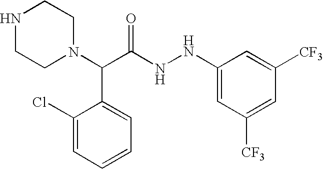

806 mg of (±)-N′-[3,5-bis(trifluoromethyl)phenyl]-2-(2-chlorophenyl)-2-(1-piperazinyl)acetohydrazide (intermediate 30, 1.67 mmole) were purified by Chiral HPLC [Chiralpak AS-H, 25×2.1 cm, Flow rate: 14 mL/min, UV detection: 245 nm, 15% modifier: n-Hex 85%, EtOH+0.1% isopropylamine, 20 mg/mL injection]: Rₜ=6.49 min. Solvents were removed under reduced pressure to give the title compound (320 mg, 84%); ¹H-NMR (400 MHz, CDCl₃): δ 2.57 (4H, s), 2.97 (4H, m), 4.78 (1H, s), 6.45 (1H, br s), 7.13 (2H, br s), 7.31 (2H, m), 7.35 (1H, br s), 7.45 (1H, dd), 7.52 (1H, dd), 8.69 (1H, br s).

### Compound 27: N′-[3,5-bis(trifluoromethyl)phenyl]-2-(2-chlorophenyl)-2-(1-piperazinyl)-acetohydrazide-enantiomer 2

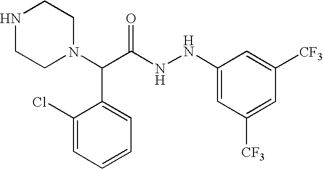

806 mg of (±)-N′-[3,5-bis(trifluoromethyl)phenyl]-2-(2-chlorophenyl)-2-(1-piperazinyl)-acetohydrazide (Intermediate 30, 1.67 mmole) were purified by Chiral HPLC [Chiralpak AS-H, 25×2.1 cm, Flow rate: 14 mL/min, UV detection: 245 nm, 15% modifier: n-Hex 85%, EtOH+0.1% isopropylamine, 20 mg/mL injection]: Rₜ=10.6 min. Solvents were removed under reduced pressure to give the title compound as a white solid (343 mg, 84%); ¹H-NMR (400 MHz, CDCl₃): δ 2.57 (4H, s), 2.97 (4H, m), 4.78 (1H, s), 6.45 (1H, br s), 7.13 (2H, br s), 7.31 (2H, m), 7.35 (1H, br s), 7.45 (1H, dd), 7.52 (1H, dd), 8.69 (1H, br s). Chiral HPLC [Chiralpak AS-H, 25×4.6 cm, Flow rate: 1 mL/min, CD 245 nm, DAD 225 nm, 15% modifier: n-Hex 85%, EtOH+0.1% isopropylamine, 20 mg/mL injection]: Rₜ=8.65 min. (96.44% ee).

### Intermediate 31: (±)-[2-(methyloxy)phenyl](4-methyl-1-piperazinyl)acetic acid

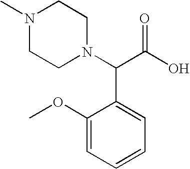

200 mg of [6-(methyloxy)phenyl]boronic acid (1.32 mmol, Aldrich) were added to a suspension of 126 mg of glyoxylic acid hydrate (1.33 mmol, Aldrich) and 147 μL of 1-methylpiperazine (1.33 mmol, Aldrich) in 3 mL of MeCN in a microwave vial. The mixture was irradiated at 120° C. for 20 minutes to give a thick brown crude which was evaporated to dryness. The final compound was purified on a SPE HLB cartridge (H₂O 100% to H₂O/MeOH 90/1) to give 300 mg (87%) of the title compound. ¹H-NMR (400 MHz, CDCl₃): δ 2.50 (3H, s), 3.79 (3H, s), 2.90-3.96 (8H, m), 4.74 (1H, br s), 7.00 (1H, t), 7.10 (1H, d), 7.34-7.38 (2H, t), 10.42 (1H, br s); m/z (ES): 265.3 [M+H]⁺.

### Intermediate 32: (±)-N′-[3,5-bis(trifluoromethyl)phenyl]-2-[2-(methyloxy)phenyl]-2-(4-methyl-1-piperazinyl)acetohydrazide

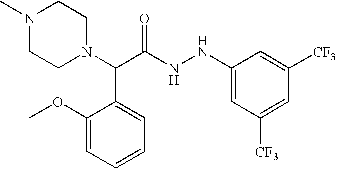

To a solution of 146 mg of (±)-[6-(methyloxy)phenyl](4-methyl-1-piperazinyl)acetic acid (intermediate 31, 0.55 mmole) and 168 mg of [3,5-bis(trifluoromethyl)phenyl]hydrazine (0.69 mmol, Lancaster) in 1.5 mL of dry DMF, were added 63 μL of NMM (0.57 mmol, Aldrich) and 256 mg of BOP (0.57 mmol, Fluka) at RT. The solution was stirred at RT for 3 h. Then 2 mL of NaOH 1 N were added and the aqueous layer was extracted with AcOEt (3×3 mL). The final compound was isolated by elution on a SPE cartridge (stationary phase Si, DCM 100% to DCM/MeOH 9/1) to give 85 mg (31%) of the title compound. ¹H-NMR (400 MHz, CDCl₃): δ 2.32 (3H, m), 2.59 (8H, m), 3.85 (3H, m), 4.61 (1H, m), 6.52 (1H, br s), 6.95 (1H, dd), 6.99 (1H, t), 7.13 (2H, m), 7.33 (3H, m), 8.81 (1H, br s); m/z (ES): 491 [M+H]⁺.

### Compound 28: N′-[3,5-bis(trifluoromethyl)phenyl]-2-[2-(methyloxy)phenyl]-2-(4-methyl-1-piperazinyl)acetohydrazide-enantiomer 1

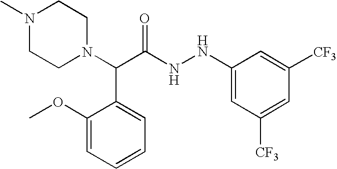

The enantiomeric mixture of (±)-N′-[3,5-bis(trifluoromethyl)phenyl]-2-[2-(methyloxy)phenyl]-2-(4-methyl-1-piperazinyl)acetohydrazide (intermediate 32, 85 mg, 0.17 mmole) was separated by chiral SFC (Column: CHIRALCEL OD-H 250×46 mm, 10% modifier: EtOH+0.1% isopropylamine, Flow rate 2 mL/min, Rₜ=7.61 min) to give the title compound (26 mg, 61%); ¹H-NMR (500 MHz, CDCl₃): δ 2.32 (3H, m), 2.59 (8H, m), 3.85 (3H, m), 4.61 (1H, m), 6.52 (1H, br s), 6.95 (1H, dd), 6.99 (1H, t), 7.13 (2H, m), 7.33 (3H, m), 8.81 (1H, br s); m/z (ES): 491 [M+H]⁺.

### Compound 29: N′-[3,5-bis(trifluoromethyl)phenyl]-2-[2-(methyloxy)phenyl]-2-(4-methyl-1-piperazinyl)acetohydrazide-enantiomer 2

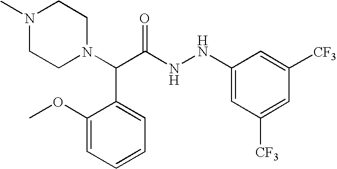

The enantiomeric mixture of (±)-N′-[3,5-bis(trifluoromethyl)phenyl]-2-[2-(methyloxy)phenyl]-2-(4-methyl-1-piperazinyl)acetohydrazide (intermediate 32, 85 mg, 0.17 mmole) was separated by chiral SFC (Column: CHIRALCEL OD-H 250×46 mm, 10% modifier: EtOH+0.1% isopropylamine, Flow rate 2 mL/min, Rₜ=9.93 min) to give the title compound (30 mg, 72%). ¹H-NMR (500 MHz, CDCl₃): δ 2.32 (3H, m), 2.59 (8H, m), 3.85 (3H, m), 4.61 (1H, m), 6.52 (1H, br s), 6.95 (1H, dd), 6.99 (1H, t), 7.13 (2H, m), 7.33 (3H, m), 8.81 (1H, br s); m/z (ES): 491 [M+H]⁺.

### Intermediate 33: (±)-[2-fluoro-6-(methyloxy)phenyl](4-methyl-1-piperazinyl)acetic acid

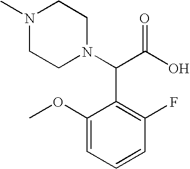

200 mg of (±)-[2-fluoro-6-(methyloxy)phenyl]boronic acid (0.71 mmole, Aldrich) were added to a suspension of 114 mg of glyoxylic acid hydrate (0.71 mmole, Aldrich) and 130 μL of 1-methylpiperazine (0.71 mmole) in 3 mL of MeCN in a microwave vial. The mixture was irradiated at 80° C. for 20 min to give a thick brown crude which was evaporated to dryness. The final compound was purified on a SPE HLB cartridge (H₂O 100% to H₂O/MeOH 90/1), to give 125 mg (58%) of the title compound; ¹H-NMR (500 MHz, CDCl₃): δ 2.5 (3H, s), 3.0-3.3 (8H, m), 3.8 (3H, s), 4.9 (1H, s), 6.8 (1H, t), 6.9 (1H, d), 7.4-7.3 (1H, q), 10.4 (1H, br s); m/z (ES): 283 [M+H]⁺.

### Intermediate 34: (±)-N′-[3,5-bis(trifluoromethyl)phenyl]-2-[2-fluoro-6-(methyloxy)phenyl]-2-(4-methyl-1-piperazinyl)acetohydrazide

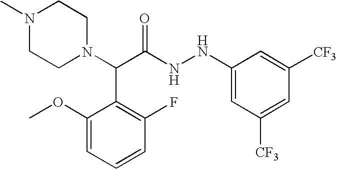

To a solution containing 175 mg of (±)-[2-fluoro-6-(methyloxy)phenyl](4-methyl-1-piperazinyl)acetic acid (intermediate 33, 0.62 mmole) and 188 mg of [3,5-bis(trifluoromethyl)phenyl]hydrazine (0.77 mmole, Lancaster) in 1.5 mL of dry DMF, were added 72 μL of NMM (Aldrich) and 288 mg of BOP (0.65 mmole, Fluka) at RT. The solution was stirred at RT for 3 h. Then 2 mL of NaOH 1N were added and the aqueous layer was extracted with AcOEt (3×3 mL). The final compound was purified on a SPE cartridge (stationary phase Si, DCM 100% to DCM/MeOH 9/1) to give 145 mg (46%) of the title compound. ¹H-NMR (500 MHz, CDCl₃): δ 2.30 (3H, m), 2.55 (6H, m), 2.85 (2H, m), 3.76 (3H, m), 4.93 (1H, m), 6.64 (1H, br s), 6.73 (2H, m), 7.27 (1H, m), 7.26 (2H, m), 7.36 (1H, m), 9.27 (1H, br s); m/z (ES): 508 [M]⁺.

### Compound 30: N′-[3,5-bis(trifluoromethyl)phenyl]-2-[2-fluoro-6-(methyloxy)phenyl]-2-(4-methyl-1-piperazinyl)acetohydrazide-enantiomer 1

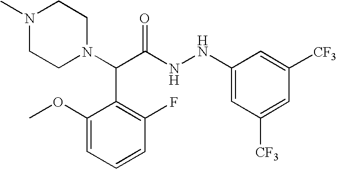

145 mg of intermediate 34 (0.28 mmole) were separated by chiral HPLC (Column: chiralpak AD-H 250×4.6 mm, Mobile phase: A: n-Hex; B:EtOH, Gradient: 25% B, Flow rate 0.8 mL/min, Rₜ=5.1 min.) to give the title compound (64 mg, 90%). ¹H-NMR (500 MHz, CDCl₃): δ 2.30 (3H, m), 2.55 (6H, m), 2.85 (2H, m), 3.76 (3H, m), 4.93 (1H, m), 6.64 (1H, br s), 6.73 (2H, m), 7.27 (1H, m), 7.26 (2H, m), 7.36 (1H, m), 9.27 (1H, br s); m/z (ES): 508 [M]⁺.

### Compound 31: N′-[3,5-bis(trifluoromethyl)phenyl]-2-[2-fluoro-6-(methyloxy)phenyl]-2-(4-methyl-1-piperazinyl)acetohydrazide-enantiomer 2

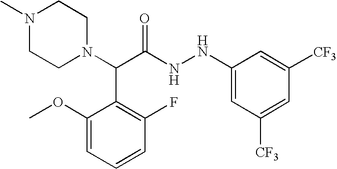

145 mg of Intermediate 34 (0.28 mmole) were separated by chiral HPLC (Column: chiralpak AD-H 250×4.6 mm, Mobile phase: A: n-Hex; B: EtOH, Gradient: 25% B, Flow rate 0.8 mL/min, Rₜ=7.2 min.) to give the title compound (60 mg, 0.12 mmole, 84%). ¹H-NMR (500 MHz, CDCl₃): δ 2.30 (3H, m), 2.55 (6H, m), 2.85 (2H, m), 3.76 (3H, m), 4.93 (1H, m), 6.64 (1H, br s), 6.73 (2H, m), 7.27 (1H, m), 7.26 (2H, m), 7.36 (1H, m), 9.27 (1H, br s); m/z (ES): 508 [M]⁺.

### Intermediate 35: 2-diazo-1-(2,3-dihydro-1,4-benzodioxin-5-yl)ethanone

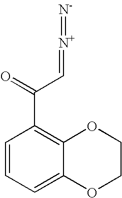

A solution of 1 g of 2,3-dihydro-1,4-benzodioxin-5-carboxylic acid (5.55 mmole, Aldrich) in 30.7 mL of SOCl₂ was stirred at 85° C. for 1 h in a dry flask under nitrogen atmosphere. The excess of SOCl₂ was removed under reduced pressure, the resulting crude oil was dissolved in 20.3 mL of MeCN and to this solution, a solution of TMSCHN₂ 2M in hexane (11.1 mmole, Aldrich) was added dropwise at 0° C. The reaction mixture was warmed to RT and stirred for 2 h. A 1M solution of citric acid was added and then extracted with AcOEt. The organic layer was washed with a saturated solution of NaHCO₃, dried over Na₂SO₄ and concentrated under reduced pressure. The crude oil was purified by flash chromatography (from CH to CH/AcOEt 80/20) to give the title compound as a yellow oil (489 mg, 42%). ¹H-NMR (500 MHz, CDCl₃): δ 4.29-4.34 (2H, m), 4.35-4.40 (2H, m), 6.25 (1H, s), 6.92 (1H, t), 7.02 (1H, dd), 7.46 (1H, s); m/z (ES): 205 [M+H]⁺.

### Intermediate 36: 2,3-dihydro-1,4-benzodioxin-5-ylacetic acid

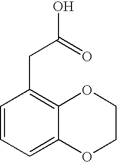

489 mg of intermediate 35 (2.37 mmole) were dissolved in dioxane/water 7.1/4.7 mL. Then 81 mg of silver benzoate (0.35 mmole, Aldrich) were added and the reaction mixture was heated at 70° C. for 2 h. After evaporation of the solvents, the crude was washed with a saturated solution of Na₂CO₃. The aqueous phase was washed with diethyl ether and acidified to pH 2 by carefully addition of concentrate hydrochloric acid. Then it was extracted with AcOEt (3×25 mL). The combined organic layers were dried over Na₂SO₄, filtered and concentrated in vacuo to give the title compound (350 mg, 76%). ¹H-NMR (500 MHz, CDCl₃): δ 3.52 (2H, s), 4.07-4.20 (4H, m), 6.61-6.73 (3H, m); m/z (ES): 195 [M+H]⁺, 217 [M+Na]⁺.

### Intermediate 37: (±)-bromo(2,3-dihydro-1,4-benzodioxin-5-ylacetic acid

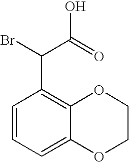

88 mg of benzoylperoxide (0.36 mmole, Aldrich) were added to a suspension of 350 mg of 2,3-dihydro-1,4-benzodioxin-5-ylacetic acid (Intermediate 36, 1.8 mmole) and 324 mg of NBS (1.82 mmole, Aldrich) in 20 mL of CCl₄. The reaction mixture was refluxed for 5 h at 85° C. After evaporation of the solvent under reduced pressure, the crude was purified by flash chromatography on silica gel (from CH to CH/AcOEt 60/40) to give the title compound (315 mg, 64%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.64 min, m/z (ES): 274 [M+H]⁺; m/z (ES): 273 [M]⁺.

### Intermediate 38: (±)-2,3-dihydro-1,4-benzodioxin-5-yl(4-methyl-1-piperazinyl)acetic acid

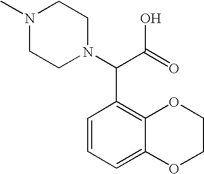

319 mg of K₂CO₃ (2.3 mmole, Fluka) were added to a solution of 315 mg of (±)-bromo(2,3-dihydro-1,4-benzodioxin-5-yl)acetic acid (Intermediate 37, 1.15 mmole) in 10 mL of THF dry. The reaction mixture was stirred for 5 min and then 141 μL of N-methylpiperazine (1.27 mmole, Aldrich) were added dropwise. The reaction mixture was stirred at room temperature for 4 h. Then, further N-methylpiperazine (1.17 mmole) was added and the reaction mixture was stirred for additional 1 h. The reaction mixture was concentrated under reduced pressure and the crude was purified by HLB cartridge (Waters Oasis® HLB 35 cc (6 g) LP Extraction Cartridges (from H₂O to H₂O/MeOH 6:4)) to give the title compound (150 mg, 47%). ¹H NMR (500 MHz, CDCl₃): δ 2.54 (3H, s), 2.85-3.17 (8H, m), 4.26 (4H, s), 4.69 (1H, s), 6.79-6.86 (2H, m), 7.02 (1H, dd); m/z (ES): 293 [M+H]⁺.

### Intermediate 39: (±)-N′-[3,5-bis(trifluoromethyl)phenyl]-2-(2,3-dihydro-1,4-benzodioxin-5-yl)-2-(4-methyl-1-piperazinyl)acetohydrazide

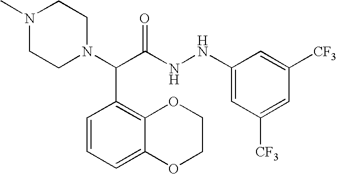

100 mg of (±)-3-dihydro-1,4-benzodioxin-5-yl(4-methyl-1-piperazinyl)acetic acid (Intermediate 38, 0.3 mmole) were dissolved in 4.5 mL of DCM and 0.66 mL of NMP. Then, 78 mg of HOBT.H₂O (0.51 mmole, Fluka), 370 mg of PL-DCC (loading: 1.59 mmole/g, 0.6 mmole, Argonaut Technologies inc.), 349 mg of PL-DIPAM (loading: 2.59 mmole/g, 0.9 mmole, Argonaut Technologies inc.) and 74 mg of 3,5-bis-(trifluoromethyl)phenylhydrazine (0.3 mmole, Alfa Aesar) were added. The reaction mixture was stirred at RT overnight. Then 192 mg of MP-isocyanate (loading 1.7 mmole/g, 0.32 mmole, Argonaut tech.) and 86 mg of PS-trisamine (loading 3.5 mmole/g, 0.3 mmole 86 mg) were added as scavengers. The mixture was stirred for 3 h at RT. The crude was filtered off, concentrated in vacuo and purified first on a SCX cartridge and then by flash chromatography on a silica gel column (from DCM to DCM/MeOH 7/3), to give the title compound (124 mg, 0.29 mmole, 97%). ¹H-NMR (400 MHz, CDCl₃): δ 2.28-2.95 (11H, m), 4.29 (4H, s), 4.59 (1H, s), 6.43 (1H, s), 6.79-6.97 (3H, m), 7.17 (2H, s), 7.37 (1H, s), 8.79 (1H, s); m/z (ES): 519 [M+H]⁺.

### Compound 32: N′-[3,5-bis(trifluoromethyl)phenyl]-2-(2,3-dihydro-1,4-benzodioxin-5-yl)-2-(4-methyl-1-piperazinyl)acetohydrazide-enantiomer 1

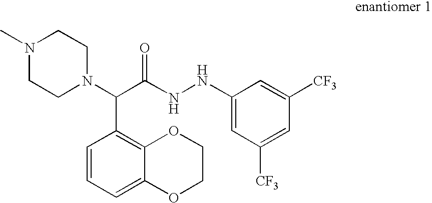

186 mg of intermediate 39 (0.36 mmole) were purified by semipreparative chiral HPLC [Chiralpak AD-H, 250×4.6 mm. Flow rate: 1.0 mL/min. UV detection: 200-400 nm. Mobile phase: A: n-Hex/B: EtOH+0.1% isopropylamine.]: Rₜ=14.69 min. Solvents were removed under reduced pressure to give the title compound as a pale yellow solid (20.9 mg, 32%). ¹H-NMR (400 MHz, CDCl₃): δ 2.28-2.95 (11H, m), 4.29 (4H, s), 4.59 (1H, s), 6.43 (1H, s), 6.79-6.97 (3H, m), 7.17 (2H, s), 7.37 (1H, s), 8.79 (1H, s) UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.61 min, m/z (ES): 519 [M+H]⁺.

### Compound 33: N′-[3,5-bis(trifluoromethyl)phenyl]-2-(2,3-dihydro-1,4-benzodioxin-5-yl)-2-(4-methyl-1-piperazinyl)acetohydrazide-enantiomer 2

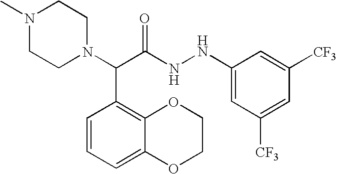

186 mg of Intermediate 39 (0.36 mmole) were purified by semipreparative chiral HPLC [Chiralpak AD-H, 250×4.6 mm. Flow rate: 1.0 mL/min. UV detection: 200-400 nm. Mobile phase: A: n-Hex/B: EtOH+0.1% isopropylamine: Rₜ=24.27 min. Solvents were removed under reduced pressure to give the title compound as a pale yellow solid (14 mg, 22%). ¹H-NMR (400 MHz, CDCl₃): δ 2.25-2.92 (11H, m), 4.28 (4H, s), 4.59 (1H, s), 6.40 (1H, s), 6.79-6.95 (3H, m), 7.17 (2H, s), 7.37 (1H, s), 8.79 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.62 min, m/z (ES): 519 [M+H]⁺.

### Intermediate 40: (±)-(4-fluoro-1-naphthalenyl)(4-methyl-1-piperazinyl)acetic acid hydrochloride salt

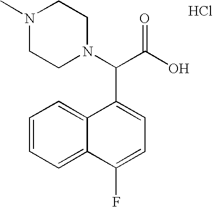

To a suspension of 500 mg of (±)-(4-fluoro-1-naphthalenyl)boronic acid (2.6 mmole, Fluorochemicals) in 5 mL of anhydrous MeCN, at RT and under N₂, were added 242 μL of glyoxylic acid hydrate (2.6 mmole, Aldrich) and 263 μL of 1-methylpiperazine (2.6 mmole, Aldrich). The mixture was subjected to microwave irradiation (120° C., 20 minutes) and was cooled to RT. The solvent was evaporated. 5 mL of HCl 1N in Et₂O were added and the suspension was stirred for 10 minutes at RT. The solvent was evaporated and the dark residue was triturated with 10 mL of DCM until a brown solid formed. The solid was filtered off, washed with 10 mL of DCM and dried under high vacuum. The crude was purified using a HLB-LP cartridge eluting with a gradient of water 100% to water/MeOH 70/30. The title compound was obtained as a brown solid (580 mg, 73%). ¹H-NMR (400 MHz, DMSO-d₆): δ 2.3 (3H, s), 2.44-2.73 (8H, m), 4.65 (1H, s), 7.29-7.41 (1H, t), 7.52-7.76 (3H, m), 8.10 (1H, d), 8.57 (1H, d); m/z (ES): 303 [MH]⁺

### Compound 34: (±)-N′-(3,5-dichlorophenyl)-2-(4-fluoro-1-naphthalenyl)-2-(4-methyl-1-piperazinyl)ethanohydrazide

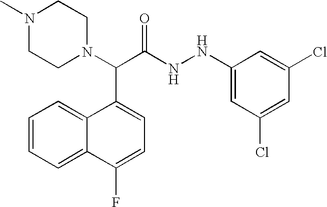

To a solution of 1.15 g of (±)-(4-fluoro-1-naphthalenyl)(4-methyl-1-piperazinyl)acetic acid hydrochloride salt (intermediate 39, 0.5 mmol) in 5 mL of anhydrous DMF, at RT and under N₂, were added 192 μL of DIPEA (1.09 mmole), 191 mg of TBTU (0.59 mmole) and 103.8 mg of (3,5-dichlorophenyl)hydrazine (0.59 mmole). The reaction mixture was stirred at RT for 24 h. It was then filtered and the solvent was evaporated. The residue was purified by Fractionlynx™ (method C). Solvents were evaporated under reduced pressure to give the title compound as a yellow foam (8 mg, 0.017 mmole, 3.5%). ¹H-NMR (400 MHz, DMSO-d₆): δ 2.18 (3H, s), 2.25-2.62 (8H, m), 4.67 (1H, s), 6.32 (2H, d), 6.72 (1H, t), 7.39 (1H, t), 7.66 (2H, m), 7.83 (1H, m), 8.09 (1H, d), 8.40 (1H, d), 8.79 (1H, d), 10.26 (1H, d); m/z (ES): 461 [M]⁺

### Intermediate 41: (±)-2,3-dihydro-1,4-benzodioxin-6-yl(4-methyl-1-piperazinyl)acetic acid

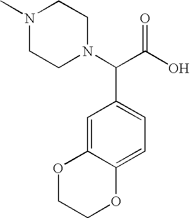

To a solution of 3.5 g of 2,3-dihydro-1,4-benzodioxin-6-ylboronic acid (19.4 mmole, Maybridge) in 5 mL of absolute EtOH, at RT, under N₂, were added 2.67 mL of glyoxylic acid hydrate (29.1 mmole, Aldrich) and 2.9 g of 1-methylpiperazine (29.1 mmole, Aldrich). The mixture was stirred at reflux temperature for 4 h and was cooled to RT. 70 mL of Et₂O were added slowly and a yellow solid precipitated. The solid was filtered and dried under high vacuum. The title compound was obtained as a yellow solid (5.3 g, 93.5%). LC-MS ES (+/−): Rₜ=0.46 min. m/z (ES): 293 [M+H]⁺

### Intermediate 42: (±)-N′-[3,5-bis(trifluoromethyl)phenyl]-2-(2,3-dihydro-1,4-benzodioxin-6-yl)-2-(4-methyl-1-piperazinyl)ethanohydrazide

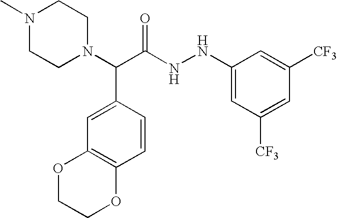

To a solution of 200 mg of (±)-2,3-dihydro-1,4-benzodioxin-6-yl(4-methyl-1-piperazinyl)acetic acid (Intermediate 41, 0.68 mmole) in 10 mL of anhydrous DCM, at RT and under N₂, were added 760 mg of PS-DIPEA (loading: 3.24 mmole/g, 0.82 mmole, Polymer Lab), 264 mg of TBTU (0.82 mmole, Aldrich) and 199 mg of 3,5-bis(trifluoromethyl)phenylhydrazine (0.82 mmole, Lancaster). The reaction mixture was stirred at RT for 24 h. It was then filtered and the solvent was evaporated. The residue was purified by flash chromatography on silica cartridge (10% MeOH/DCM) and solvents were evaporated under reduced pressure to give the title compound (120 mg, 34%) as a yellow solid. m/z (ES): 519 [M+H]⁺

### Compound 35: N′-[3,5-bis(trifluoromethyl)phenyl]-2-(2,3-dihydro-1,4-benzodioxin-6-yl)-2-(4-methyl-1-piperazinyl)ethanohydrazide-enantiomer 1

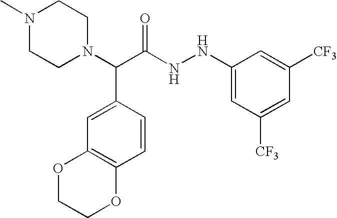

120 mg of intermediate 42 (0.23 mmole) were purified by semipreparative chiral HPLC [Chiralpak AD-H. Eluent: A: n-Hex; B: IPA+0.1% isopropylamine. Gradient: 14% B. Flow rate: 14 mL/min. UV detection: 200-400 nm]. Solvents were removed under reduced pressure to give the title compound as a white solid (49 mg, 82%, enantiomer 1). m/z (ES): 519 [M+H]⁺; Chiral HPLC [Chiralpak AD-H, 250×4.6 mm. Flow rate: 1.0 mL/min. UV detection: 200-400 nm. Mobile phase: A: n-Hex; B: IPA+0.1% isopropylamine. Gradient: 15% B]. Rₜ=10.8-14.2 min (>99.5% ee).

### Compound 36: N′-[3,5-bis(trifluoromethyl)phenyl]-2-(2,3-dihydro-1,4-benzodioxin-6-yl)-2-(4-methyl-1-piperazinyl)ethanohydrazide-enantiomer 2

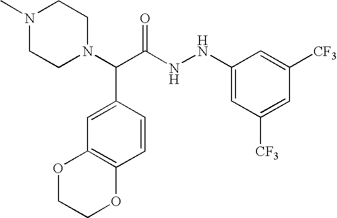

120 mg of Intermediate 42 (0.23 mmole) were purified by semipreparative chiral HPLC [Chiralpak AD-H. Eluent: A: n-Hex; B: IPA+0.1% isopropylamine. Gradient: 14% B. Flow rate: 14 mL/min. UV detection: 200-400 nm]. Solvents were removed under reduced pressure to give the title compound as a white solid (52 mg, 87%, enantiomer 2); m/z (ES): 519 [M+H]⁺; Chiral HPLC [Chiralpak AD-H, 250×4.6 mm. Flow rate: 1.0 mL/min. UV detection: 200-400 nm. Mobile phase: A: n-Hex; B: IPA+0.1% isopropylamine. Gradient: 15% B]. Rₜ=14.0-14.3 min (>98.4% ee).

### Intermediate 43: (±)-(4-methyl-1-piperazinyl){3-[(trifluoromethyl)oxy]phenyl}acetic acid

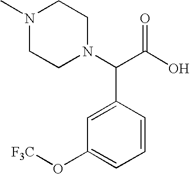

To a solution of 4.5 g of {3-[(trifluoromethyl)oxy]phenyl}boronic acid (21.8 mmole, Aldrich) in 40.0 mL of anhydrous MeCN, at RT and under N₂, were added 2 g of glyoxylic acid hydrate (21.8 mmole, Aldrich) and 2.18 g of 1-methylpiperazine (21.8 mmole, Aldrich). The mixture was stirred at 80° C. for 3 h and was cooled to RT. The solvent was evaporated under reduced pressure. The title compound was obtained as a brown solid (6.2 g, 89.0%). LC/MS-ES (+) (see general experimental part for method): Rₜ=1.29 min. m/z (ES): 319.1 [M+H]⁺

### Compound 37: (±)-N′-[3,5-bis(trifluoromethyl)phenyl]-2-(4-methyl-1-piperazinyl)-2-{3-[(trifluoromethyl)oxy]phenyl}acetohydrazide

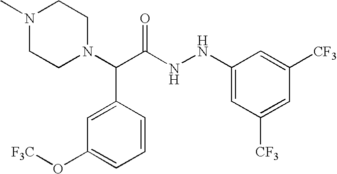

To a suspension of 1 g of (±)-(4-methyl-1-piperazinyl){3-[(trifluoromethyl)oxy]phenyl}acetic acid (intermediate 43, 3.13 mmole) in 15 mL of anhydrous DCM, at RT and under N₂, were added 811 mg of HOBT.H₂O (5.33 mmole, Aldrich), 3.9 g of PS-DCC (1.59 mmole/g: 6.26 mmole, Argonaut) and 756 mg of 3,5-bis(trifluoromethyl)phenylhydrazine (3.1 mmole, Lancaster). The reaction was stirred at RT for 3 h. The reaction mixture was then filtered and the solvent was evaporated under reduced pressure. The residue was purified by Fractionlynx™ (method C), solvents were evaporated under reduced pressure yielding the title compound as a yellow oil (62 mg, 3.6%). ¹H-NMR (400 MHz, DMSO-d₆): δ 2.34 (3H, s), 2.45-2.65 (8H, m), 4.11 (1H, s), 6.49 (1H, s), 7.04 (1H, s), 7.07 (1H, s), 7.24 (1H, s), 7.34 (2H, m), 7.45 (2H, m), 8.70 (1H, s).

### Intermediate 44: (±)-(6-methoxy-2-naphthyl)(4-methylpiperazin-1-yl)acetic acid

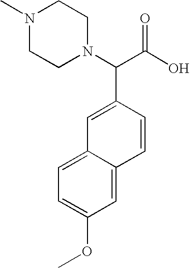

To a suspension of 175 mg of [6-(methyloxy)-2-naphthalenyl]boronic acid (0.87 mmole, Aldrich) in 1.5 mL of anhydrous MeCN, at RT and under N₂, were added 81 mg of glyoxylic acid hydrate (0.87 mmole) and 96 μL of 1-methylpiperazine (0.87 mmole, Aldrich). The mixture was subjected to microwave irradiation (120° C., 20 minutes) and was cooled to RT. The solvent was evaporated and the dark residue triturated with DCM until a brown solid formed. The solid was filtered, washed with DCM and dried under high vacuum. The title compound was obtained as a brown solid (215 mg, 78%). ¹H-NMR (400 MHz, DMSO-d₆): δ 2.15 (3H, s), 2.30-2.50 (8H, m), 3.87 (3H, s), 4.05 (1H, s), 7.09-7.22 (1H, m), 7.25-7.36 (1H, m), 7.46-7.59 (1H, m), 7.74-7.89 (3H, m); LC/MS-ES (+) (see general experimental part for method): Rₜ=0.76 min, m/z (ES)=315 [M+H]⁺

### Compound 38: (±)-N′-(3,5-dichlorophenyl)-2-[6-(methyloxy)-2-naphthalenyl]-2-(4-methyl-1-piperazinyl)acetohydrazide

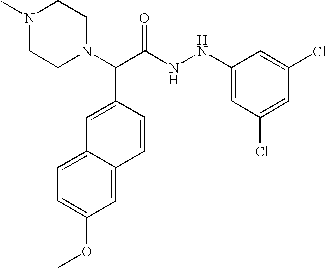

A mixture of 30 mg of (±)-(6-methoxy-2-naphthyl)(4-methylpiperazin-1-yl)acetic acid (Intermediate 44, 0.095 mmole), 13 mg of HOBT.H₂O (0.095 mmole, Fluka), 18 mg of EDCI.HCl (0.095 mmole, Aldrich) and 18 μL of DIPEA (0.095 mmole, Aldrich) in 10 mL of anhydrous DCM was stirred at RT under N₂ atmosphere for 30 minutes. 18 mg of (3,5-dichlorophenyl)hydrazine (0.103 mmole, Aldrich) were added to the mixture portion wise. The suspension was stirred at room temperature for 18 h. The suspension was diluted with AcOEt and the organic phase was washed with a saturated solution of NaHCO₃. The organic solution was dried over Na₂SO₄, filtered and evaporated to give the desired compound (6 mg, 13%). ¹H-NMR (400 MHz, DMSO-d₆): δ 2.18 (3H, br s), 2.33-2.70 (8H, m), 3.88 (3H, s), 3.99 (1H, s), 6.38 (2H, d), 6.73 (1H, t), 7.18 (1H, dd), 7.33 (1H, d), 7.64 (1H, dd), 7.83 (2H, m), 7.90 (1H, d), 8.37 (1H, d), 10.20 (1H, d); m/z (ES) 474 [M+H]⁺

### Intermediate 45: (±)-(4-methyl-1-piperazinyl)(1-naphthalenyl)acetic acid

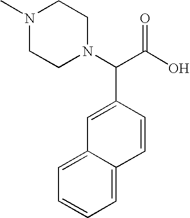

A solution of 1.1 g of glyoxylic acid monohydrate (Aldrich, 11.9 mmole) in 10 mL of EtOH at 25° C. was treated with 1.3 mL of N-methylpiperazine (Aldrich, 11.9 mmole) and the mixture was stirred at 25° C. for 5 minutes. 2.05 g of 1-Naphthylboronic acid (Matrix Scientific, 11.9 mmole) were added to the pale amber solution and the reaction vessel was sealed and heated at 100° C. under microwave irradiations for 2 h. The mixture was then allowed to cool to 25° C. and was concentrated to approximately ¼ volume. The solution was loaded onto a pre-column (silica gel) and was purified by flash chromatography (0/10 MeOH/DCM to 30/70 MeOH/DCM) to afford the acid intermediate as a brown solid (2.0 g, 59%): ¹H-NMR (400 MHz, DMSO-d₆): δ 2.13 (3H, s), 2.32 (4H, br m), 2.44-2.51 (4H, m), 4.67 (1H, br s), 7.45-7.54 (3H, m), 7.65 (1H, dd), 7.86 (1H, d), 7.91 (1H, dd), 8.52 (1H, d), 10.39 (1H, s); LC-MS (+) (see general experimental part for method): m/z 285 [M+H]⁺.

### Compound 39: (±)-N′-(3,5-Dimethylphenyl)-2-(4-methyl-1-piperazinyl)-2-(1-naphthalenyl)-acetohydrazide

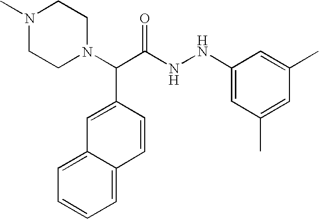

A solution of 0.4 g of (±)-(4-methyl-1-piperazinyl)(1-naphthalenyl)acetic acid (Intermediate 45, 1.4 mmole) in 10 mL of DMF at 25° C. under N₂ was treated with 0.23 g of HOBT.H₂O monohydrate (1.7 mmole, Aldrich), 0.32 g of EDCI.HCl (1.7 mmole, Aldrich) and 0.59 mL of diisopropylethylamine (3.4 mmole, Aldrich) and the reaction mixture was stirred for 25 minutes. 0.29 g of 3,5-dimethylphenylhydrazine hydrochloride (1.7 mmole, Avocado Research) were added and the mixture was stirred at 25° C. for 4 h. The reaction was quenched by the addition of saturated aqueous NaHCO₃ and diluted with AcOEt. The aqueous layer was extracted with AcOEt (3×15 mL) and the combined organic extracts were washed with brine, dried over MgSO₄, filtered and concentrated to give an orange oil. The crude material was purified by flash chromatography (12 g silica gel column, 0/10 MeOH/DCM to 0.8/9.2 MeOH/DCM) to afford the title compound as an amber solid (0.19 g, 33%). ¹H-NMR (400 MHz, DMSO-d₆): δ 1.93 (6H, s), 2.41-2.45 (2H, m), 2.73-2.74 (3H, 2 s), 2.80-2.91 (2H, m), 2.94-3.06 (2H, m), 3.29-3.41 (1H, m), 4.90 (1H, s), 6.01 (2H, s), 6.22 (1H, s), 7.50-7.60 (4H, m), 7.77 (1H, dd), 7.90-7.98 (2H, m), 8.62 (1H, d), 9.78 (1H, br s), 10.08 (1H, s);

### Compound 40: (±)-N′-[3,5-Bis(trifluoromethyl)phenyl]-2-(4-methyl-1-piperazinyl)-2-(1-naphthalenyl)acetohydrazide hydrochloride

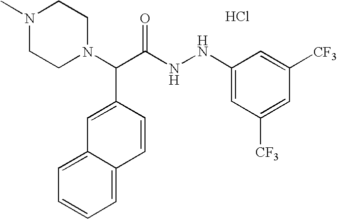

Following a similar procedure to that described for Compound 39 using 0.3 g of (±)-4-methyl-1-piperazinyl)(1-naphthalenyl)acetic acid (Intermediate 45, 1.1 mmole), 0.24 g of EDCI.HCl (1.3 mmole, Aldrich), 0.17 g of HOBT.H₂O hydrate (1.3 mmole, Aldrich) and 0.31 g of 3,5-bis(trifluoromethyl)phenylhydrazine (1.3 mmole, Alfa Aesar) in 10 mL of DMF and purifying by flash chromatography (12 g silica gel column, 0/10 MeOH/DCM to 0.8/9.2 MeOH/DCM), a pale amber solid was obtained. The solid was dissolved in a small amount of DCM and MeOH and treated with an excess of 4 N HCl in dioxane (Aldrich). The solution was then diluted with Et₂O, and the solid was collected by filtration and dried in a vacuum oven at 60° C. overnight to afford the title compound as a tan solid (0.18 g, 31%). ¹H-NMR (400 MHz, DMSO-d₆) δ: 2.45 and 2.57 (2H, m), 2.70 and 2.71 (3H, 2 s), 2.82-3.06 (4H, m), 3.25-3.39 (2H, m), 5.05 (1H, br s), 6.94 (2H, s), 7.21 (1H, s), 7.48-7.60 (3H, m), 7.74 (1H, d), 7.95 (2H, m), 8.61 (1H, br d), 8.77 (1H, br s), 10.35 (1H, br s), 10.56 (1H, s).

### Intermediate 46: (±)-1,3-Benzodioxol-5-yl(4-methyl-1-piperazinyl)acetic acid

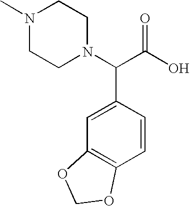

13.4 mL of N-Methylpiperazine (Aldrich, 120 mmole) were added to a solution of 11.1 g of glyoxylic acid monohydrate (Aldrich, 120 mmole) in 60 mL of EtOH at 25° C. and the mixture was stirred for 10 minutes, then it was treated with 20 g of 1,3-benzodioxol-5-ylboronic acid (Aldrich, 120 mmole). The reaction was stirred at 25° C. for 3 weeks, and then was filtered (EtOH/DCM wash). The resulting solid was suspended in Et₂O and treated with an excess of pinacol (Aldrich) and stirred at 25° C. for 1 week. The solid was collected (Et₂O wash) and dried in a vacuum oven at 55° C. overnight to afford the acid intermediate as a brown solid (20.8 g, 59%): ¹H-NMR (400 MHz, DMSO-d₆): δ 2.18 (3H, s), 2.37-2.49 (8H, br m), 3.85 (1H, s), 5.99 (2H, m), 6.85 (2H, m), 6.94 (1H, s); LC-MS (ES+) (see general experimental part for method): m/z 279 [M+H]⁺.

### Compound 41: Step 2: (±)-2-(1,3-Benzodioxol-5-yl)-N′-[3,5-bis(trifluoromethyl)phenyl]-2-(4-methyl-1-piperazinyl)acetohydrazide hydrochloride

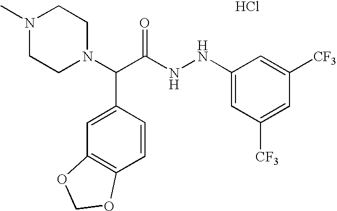

Following a similar procedure to that described for Compound 39 using 1.0 g of (±)-1,3-benzodioxol-5-yl(4-methyl-1-piperazinyl)acetic acid (Intermediate 46, 3.6 mmole), 0.83 g of EDCI.HCl (4.3 mmole, Aldrich), 0.49 g of HOBT hydrate (4.3 mmole, Aldrich) and 1.0 g of 3,5-bis(trifluoromethyl)phenylhydrazine (4.1 mmole, Alfa Aesar) in 20 mL of DMF and purifying by flash chromatography (40 g silica gel column, 0/10 MeOH/DCM to 0.8/9.2 MeOH/DCM), an amber residue was obtained. The material was dissolved in a small amount of DCM and treated with an excess of 4 N HCl in dioxane (Aldrich), then diluted with Et₂O, and the solid was collected by filtration and dried in a vacuum oven at 60° C. overnight to afford the title compound as a tan solid (0.42 g, 22%). ¹H-NMR (400 MHz, DMSO-d₆): δ 2.27-2.39 (1H, m), 2.54-2.60 (1H, m), 2.72 and 2.73 (3H, 2 s), 2.75-2.79 (1H, m), 2.94-3.15 (3H, m), 3.28-3.42 (2H, m), 4.05 (1H, br s), 6.00 (2H, d), 6.91-6.97 (4H, m), 7.02 (1H, d), 7.25 (1H, s), 8.74 (1H, br s), 10.31 (1H, br s), 10.46 (1H, s); LC-MS (ES+) (see general experimental part for method): m/z 505 [M+H]⁺.

### Compound 42: (+)-2-(3-chlorophenyl)-N′-(3,5-dichlorophenyl)-2-(4-methyl-1-piperazinyl)acetohydrazide

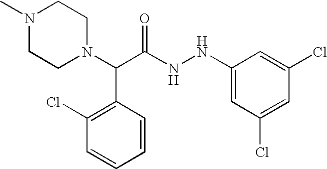

A suspension of 60 mg of (±)-(2-chlorophenyl)(4-methylpiperazin-1-yl)acetic acid hydrochloride (Intermediate 10, 0.2 mmole), 84 mg of TBTU (0.26 mmole) and 186 mg of PL-DIPAM (loading 3.24 mmole/g, 0.6 mmole) in 2 mL of anhydrous DCM was stirred at room temperature for 10 min, then 35 mg of 3,5-(dichlorophenyl)hydrazine (0.2 mmole, Aldrich) were added. The suspension was stirred at RT for 4 h. The solid was filtered and the solvent evaporated to give the title compound 25 mg (0.058 mmole, 83%). ¹H-NMR: (400 MHz, DMSO-d₆): δ 2.25 (3H, s), 2.20-2.45 (8H, br s), 4.40 (1H, s), 6.55 (2H, s), 6.80 (1H, s), 7.4 (2H, m), 7.55 (1H, d), 7.75 (1H, d), 8.10 (1H, s), 8.50 (1H, s).

### Intermediate 47: (±)-bromo(3-chlorophenyl)acetic acid

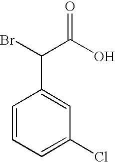

1.13 g of Benzoylperoxide (4.68 mmole) were added to a solution of 4 g of 3-(chlorophenyl)acetic acid (23.4 mmole, Fluka) and 4.1 g of NBS (23.6 mmole, Aldrich) in 120 mL of CCl₄. The reaction mixture was heated at reflux for 4 h. Then it was cooled to RT, the solid was filtered off and the filtrate concentrated in vacuo. The crude was purified by flash chromatography (from CH to CH/AcOEt 1:1) to give the title compound (2.1 g, 36%). ¹H-NMR (400 MHz, CDCl₃): δ 5.32 (1H, s), 7.22-7.52 (4H, m), 7.61 (1H, m).

### Intermediate 48: (±)-(3-chlorophenyl)(4-methyl-1-piperazinyl)acetic acid hydrochloride

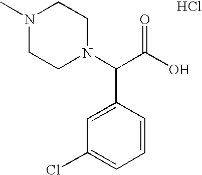

2.3 g of potassium carbonate (16.8 mmole) were added to a solution of 2.1 g of (±)-bromo(3-chlorophenyl)acetic acid (Intermediate 47, 8.4 mmole) in 80 mL of THF. The mixture was stirred for 5 min, then 0.9 mL of N-methylpiperazine (9.24 mmole, Aldrich) were added dropwise and the reaction mixture was stirred for 5 h at RT. The solid was filtered and the filtrate concentrated in vacuo. The crude was purified by HLB-LP cartridge (Waters Oasis® HLB 35 cc (6 g) LP Extraction Cartridges) (from H₂O to H₂O/MeOH 6:4) to give the title compound (1.7 g, 68%). ¹H-NMR (400 MHz, DMSO-d₆) δ 2.40-2.88 (11H, m), 3.04 (1H, m), 5.76 (1H, s), 7.32-7.55 (4H, m).

### Intermediate 49: (±)-2-(3-chlorophenyl)-N′-(3,5-dichlorophenyl)-2-(4-methyl-1-piperazinyl)acetohydrazide

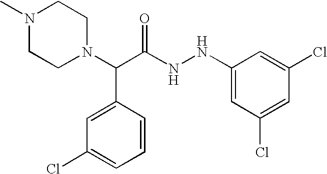

587 μL of DIPEA (3.3 mmol, Aldrich) and 344 mg of HATU (1.1 mmole, Fluka) were added to a solution of 228 mg of intermediate 48 (0.82 mmole) in 3 mL of anhydrous DCM under a nitrogen atmosphere. After stirring for 10 min, 173 mg of 3,5-(dichlorophenyl)hydrazine (1 mmole, Lancaster) were added. The solution was stirred at RT overnight, then it was diluted with water. The organic layer was dried, concentrated in vacuo and the residue was purified twice by flash chromatography (from DCM to DCM/NH₃ 0.5 M in MeOH 8:2) to give the title compound (320 mg, 91%). LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.57 min; m/z (ES): 427 [M+H]⁺

### Compound 43: 2-(3-chlorophenyl)-N′-(3,5-dichlorophenyl)-2-(4-methyl-1-piperazinyl)acetohydrazide-enantiomer 1

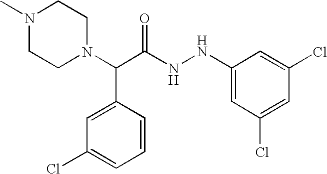

320 mg of Intermediate 49 were purified by Chiral HPLC [Chiralpak AD-H, 250×4.6 mm. Flow rate: 0.8 mL/min. UV detection: 200-400 nm. Mobile phase: A: n-Hex/B: EtOH+0.1% isopropylamine. Gradient: isocratic 20% B]: Rₜ=9.03 min (ee>99.5%, purity 98.8%). Solvents were removed under reduced pressure to give the title compound as a white solid (75.6 mg, 47%). ¹H-NMR (400 MHz, DMSO-d₆): δ 2.15 (3H, s), 2.2-2.45 (8H, br s), 3.9 (1H, s), 6.35 (2H, s), 6.8 (1H, s), 7.4 (4H, m), 7.55 (1H, s), 8.5 (1H, s).

### Compound 44: 2-(3-chlorophenyl)-N′-(3,5-dichlorophenyl)-2-(4-methyl-1-piperazinyl)-acetohydrazide-enantiomer 2

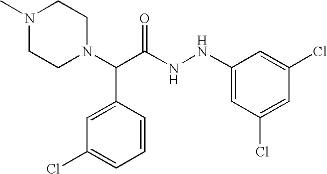

320 mg of Intermediate 49 were purified by Chiral HPLC [Chiralpak AD-H, 250×4.6 mm. Flow rate: 0.8 mL/min. UV detection: 200-400 nm. Mobile phase: A: n-Hex/B: EtOH+0.1% isopropylamine. Gradient: isocratic 20% B]: Rₜ=10.7 min (ee=98.7%, purity 99.3%). Solvents were removed under reduced pressure to give the title compound as a white solid (83 mg, 52%). ¹H-NMR (400 MHz, DMSO-d₆): δ 2.15 (3H, s), 2.2-2.45 (8H, br s), 3.9 (1H, s), 6.35 (2H, s), 6.8 (1H, s), 7.4 (4H, m), 7.55 (1H, s), 8.5 (1H, s).

### Compound 45: (±)-formic acid-N′-[3,5-bis(trifluoromethyl)phenyl]-2-(3-chlorophenyl)-2-(4-methyl-1-piperazinyl)acetohydrazide

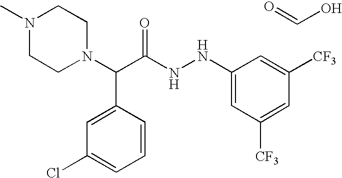

To a suspension of 250 mg of (±)-(3-chlorophenyl)(4-methyl-1-piperazinyl)acetic acid hydrochloride (Intermediate 48, 0.82 mmole) and 213 mg of HOBT.H₂O (0.9 mmole) in 4 mL of DCM were added 1 mL of NMP, 1.03 g of PL-DCC (1.08 mmole), 950 mg of PL-DIPAM (1.62 mmole) and 132 mg of 3,5-bis(trifluoromethyl)phenylhydrazine (0.54 mmole, Alfa Aesar). The reaction mixture was stirred at RT overnight, then 230 mg of MP-isocyanate (loading 1.7 mmole/g, 0.39 mmole, Argonaut tech.) and 760 mg of PS-trisamine were added as scavengers. The mixture was stirred for 8 h at RT, then the crude was filtered off, concentrated in vacuo and purified first using a SCX cartridge and then by Fractionlynx™ to give the title compound as a racemate (155 mg, 53%). ¹H NMR (400 MHz, DMSO-d₆): δ 2.15 (3H, s), 2.2-2.45 (8H, m), 3.9 (1H, s), 7.0 (2H, s), 7.25 (1H, s), 7.4 (3H, m), 7.55 (1H, s), 8.1 (1H, s), 8.8 (1H, s), 10.4 (1H, s).

### Intermediate 50: (2,2-difluoro-1,3-benzodioxol-4-yl)boronic acid

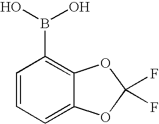

5.8 mL of BuLi 1.6 M in hexane (9.3 mmole, Aldrich) were slowly added to a solution of 2 g of 4-Bromo-2,2-difluoro-1,3-benzodioxolane (8.4 mmole, Alfa Aesar) in 25 mL of dry THF previously cooled to −78° C. under a nitrogen atmosphere. After 30 min, 2.5 mL of B(OMe)₃ (23.63 mmole, Aldrich) were carefully added keeping the temperature at −78° C. At the end of the addition the pale yellow solution was allowed to reach RT and stirred for 2 h. Then it was cooled to 0° C., quenched with 10 mL of HCl 3M and stirred for 20 minutes at RT. AcOEt was added and the organic phase was separated, dried and concentrated under reduced pressure. The crude was purified by flash chromatography on silica gel (from CH to AcOEt) to give the title compound (620 mg, 36%). UPLC-MS: Rₜ=0.63 min (see general experimental part for method), m/z (ES−): 200.9 [M−H]⁻.

### Intermediate 51: (±)-(2,2-difluoro-1,3-benzodioxol-4-yl)(4-methyl-1-piperazinyl)acetic acid hydrochloride

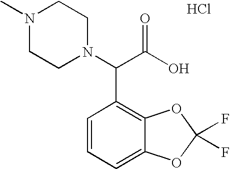

A solution of 520 mg of intermediate 50 (2.57 mmole), 288 μL of N-methylpiperazine (2.57 mmole, Aldrich) and ethyl glyoxylate (50% in toluene, 525 mg, 2.57 mmole, Aldrich) in 10 mL of toluene was heated at 80° C. for 24 h. Then the reaction mixture was cooled to RT, diluted with MeOH and purified first by SCX and then by HLB-LP cartridge (Waters Oasis® HLB 35 cc (6 g) LP Extraction Cartridges) (from H₂O to H₂O/MeOH 7:3) to give the title compound (227 mg, 25%). ¹H-NMR (400 MHz, DMSO-d₆): δ 2.3 (3H, s), 2.4-2.7 (8H, m), 4.4 (1H, s), 7.2 (2H, d), 7.35 (1H, dd); m/z MS (ES⁺): 315 [M+H]⁺

### Compound 46: (±)-N′-[3,5-bis(trifluoromethyl)phenyl]-2-(2,2-difluoro-1,3-benzodioxol-4-yl)-2-(4-methyl-1-piperazinyl)acetohydrazide

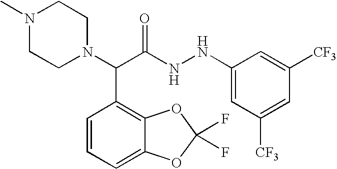

To a suspension of 70 mg of (±)-(2,2-difluoro-1,3-benzodioxol-5-yl)(4-methyl-1-piperazinyl)acetic acid hydrochloride (Intermediate 51, 0.2 mmole) and 48 mg of HOBT.H₂O (0.3 mmole) in 3 mL of DCM were added 1 mL of NMP, 226 mg of PL-DCC (0.36 mmole), 208 mg of PL-DIPAM (0.54 mmole) and 44 mg of 3,5-bis(trifluoromethyl)phenylhydrazine (0.18 mmole, Lancaster). The reaction mixture was stirred at RT overnight. Then 77 mg of MP-isocyanate (loading: 1.7 mmole/g, 0.39 mmole, Argonaut tech.) and 250 mg of PS-trisamine (loading 3.5 mmole/g, 0.87 mmole, Argonaut Tech.) were added as scavengers. The mixture was stirred for 8 h at RT. The crude was filtered off, concentrated in vacuo and purified first using a SCX cartridge and then by flash chromatography on silica gel (from DCM to DCM/NH₃ 0.5 M in MeOH 9:1) to give the title compound as racemate (80 mg, 82%). ¹H NMR (400 MHz, DMSO-d₆): δ 2.16 (3H, s), 2.44 (8H, m), 4.35 (1H, s), 7.07 (2H, s), 7.25 (1H, t), 7.30 (1H, br s), 7.39 (2H, m), 8.79 (1H, br s), 10.45 (1H, br s); m/z (ES): 541 [M+H]⁺.

### Intermediate 52: (±)-benzo[d][1,3]dioxol-5-yl(4-methyl-1-piperazinyl)acetic acid

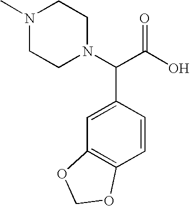

To a solution of 1 g of benzo[d][1,3]dioxol-5-ylboronic acid (6.0 mmole, Aldrich) in 10 mL of absolute EtOH at RT, were added 0.55 g of glyoxylic acid hydrate (6.0 mmole, Aldrich) and 0.665 mL of 1-methylpiperazine (6.0 mmole, Aldrich). The mixture was refluxed for 3 h during which a brown solid precipitated. The mixture was cooled to RT. 20 mL of diethyl ether were added and the solid was filtered off and dried under vacuum to give the title compound as a brown solid (1.245 g, 74%). ¹H-NMR (DMSO-d₆): δ 2.16 (3H, m), 2.40 (4H, s), 3.62 (4H, s), 3.82 (1H, m), 5.99 (2H, m), 6.85 (2H, m), 6.94 (1H, m); m/z (ES): 279 [M+H]⁺.

### Intermediate 53: (±)-benzo[d][1,3]dioxol-5-yl-N′-(3,5-dichlorophenyl)-2-(4-methyl-1-piperazinyl)acetohydrazide

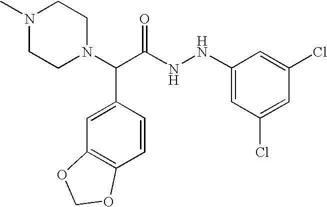

1.0 g of (±)-Benzo[d][1,3]dioxol-5-yl(4-methyl-1-piperazinyl)acetic acid (Intermediate 52, 3.59 mmole) was suspended in 10 mL of dry DMF. Then 1.25 mL of DIPEA (7.18 mmole, Aldrich) and 1.38 g of TBTU (4.31 mmole, Fluka) were added. The mixture was stirred at RT for 30 min until complete dissolution was achieved and 0.762 g of 3,5-dichlorophenylhydrazine (4.31 mmole, Aldrich) were added. The mixture was stirred at RT for 48 h. Then 20 mL of NaHCO₃ sat. were added and the mixture was extracted with AcOEt (2×30 mL). The aqueous phase was further diluted with NaHCO₃ sat. (30 mL) and again extracted with AcOEt (2×30 mL). All the organic layers were collected, dried over Na₂SO₄ and evaporated. The residue was purified by chromatography over silica (230-400 Mesh) eluting with DCM/MeOH 9/1. Solvents were removed under reduced pressure to give the title compound as a pale yellow solid (1.0 g, 63.8%). ¹H-NMR (400 MHz, DMSO-d₆,): δ 2.11 (3H, m), 2.40 (8H, m), 3.72 (1H, m), 5.99 (2H, m), 6.32 (2H, m), 6.75 (1H, m), 6.90 (2H, m), 7.03 (1H, m), 8.35 (1H, m), 10.08 (1H, m); m/z (ES): 437 [M]⁺.

### Compound 47: 2-(1,3-benzodioxol-5-yl)-N′-(3,5-dichlorophenyl)-2-(4-methyl-1-piperazinyl)-acetohydrazide hydrochloride-enantiomer 2

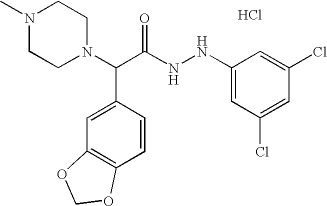

1 g (2.28 mmole) of (±)-benzo[d][1,3]dioxol-5-yl-N′-(3,5-dichlorophenyl)-2-(4-methyl-1-piperazinyl)acetohydrazide (Intermediate 53) was separated under the following Semi preparative conditions: Column: chiralcel OJ-H, 250×21 mm; Mobile phase: A: n-Hex; B: EtOH+0.1% isopropylamine; C: IPA Gradient: isocratic 80% B; 20% C; Flow rate: 14 mL/min; UV wavelength: 215 nm; Analysis time 25 min, Rₜ=17 min, to give 437 mg (0.97 mmole, 85%, enantiomer 2 as base) of a beige foam. 75 mg (0.17 mmole) of the enantiomer 2 were dissolved in 0.5 mL of dry DCM and were treated with 17 mL of HCl (1.0M in Et₂O, 0.17 mmole). The suspension was stirred for 15 min and the solvent was removed under reduced pressure. The solid was treated with Et₂O and dried under vacuum to obtain 82 mg (100%) of the title compound. UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.57 min, m/z (ES): 437/439 [M+H]⁺2Cl pattern.

### Compound 48: (±)-N′-(3,5-dichlorophenyl)-2-(4-methylpiperazin-1-yl)-2-(1-naphthyl)aceto-hydrazide diformate

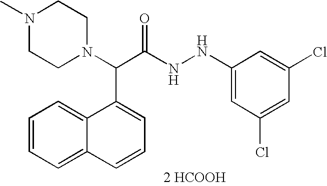

To a suspension of 30 mg of (±)-(4-methyl-1-piperazinyl)(1-naphthalenyl)acetic acid (Intermediate 45, 0.11 mmole) in 1.1 mL of anhydrous DCM, at RT and under N₂, were added 106 mg of PS-DIPEA (loading: 3.24 mmole/g, 0.32 mmole), 51 mg of TBTU (0.16 mmole) and 21 mg of 3,5-(dichlorophenyl)hydrazine (0.12 eq). The reaction mixture was stirred at RT for 1 h. It was then filtered and the solvent was evaporated. The residue was purified by flash chromatography on silica cartridge (AcOEt to 1.5/8.5 MeOH/AcOEt to 1/2/7 [0.5M NH₃/MeOH]/MeOH/AcOEt). The solid obtained was still contaminated by impurities and further purified by Fractionlynx™ to yield the title compound as a light brown solid (26 mg, 46%). ¹H-NMR (400 MHz, DMSO-d₆): δ 2.16 (3H, s), 2.38 (2H, m), 2.51 (2H, m), 3.30 (4H, m), 4.68 (1H, s), 6.32 (2H, s), 6.71 (1H, s), 7.52 (3H, m), 7.79 (1H, d), 7.88 (1H, d), 7.94 (1H, d), 8.15 (1H, s), 8.38 (1H, s), 8.67 (1H, d), 10.26 (1H, s); m/z (ES): 443 [M]⁺

### Intermediate 54: (±)-(4-methylpiperazin-1-yl)(pyridin-3-yl)acetonitrile

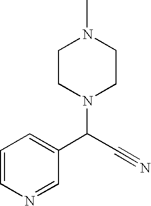

To a solution of 6.6 mL of nicotinaldehyde (70 mmole, Aldrich) in 60.7 mL of dichloroethane in a dry flask under nitrogen were added 7.4 mL of N-methylpiperazine (67 mmole, Aldrich) followed by 17.9 mL of Ti(iOPr)₄ (93.3 mmole, Aldrich). The solution was then stirred at RT for 17 h. 100 mL of a solution of Et₂AlCN 1M in toluene (100 mmole, Aldrich) were added dropwise at RT and the mixture was stirred at RT for 4 h. Then 37 mL of water were added and the mixture was stirred at RT for 1 h. The resulting precipitate was filtered on celite and the cake was washed with 700 mL of AcOEt. Solvents were removed under reduced pressure and the resulting crude was purified on flash chromatography on silica gel using first 500 mL of AcOEt and then 1250 mL of AcOEt/TEA 3% as an eluent to give the title compound as a yellow solid (13.54 g, 93%). ¹H-NMR (400 MHz, CDCl₃): δ 2.32 (3H, s), 2.48-2.65 (8H, m), 4.90 (1H, s), 7.38 (1H, dd), 7.88 (1H, d), 8.65 (1H, dd), 8.80 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=0.20 min. (100%) m/z (ES): 217 [M+H]⁺, 190 [M−CN]⁺.

### Intermediate 55: (±)-2-(4-methylpiperazin-1-yl)-2-pyridin-3-ylacetamide

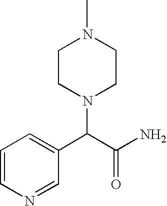

13.54 g of (±)-(4-methylpiperazin-1-yl)(pyridin-3-yl)acetonitrile (Intermediate 54, 62.6 mmole) were suspended in 142 mL of hexane. The suspension was then cooled to 0° C. and 142 mL of H₂SO₄ 96% were added dropwise. When the addition was completed the solution was allowed to reach RT and was stirred for 42 h. The crude mixture was then poured onto ice. The resulting aqueous solution was cooled to 0° C. and it was then alkalinized with 400 mL of NH₄OH 28% in water. The aqueous phase was then extracted 6 times with 150 mL of DCM. The combined organic layers were then dried over Na₂SO₄. The aqueous phase was then passed through 8 HLB cartridges (Waters Oasis® HLB 35 cc (6 g) LP Extraction Cartridges). Each cartridges was washed with 60 mL of water followed by 60 mL of MeOH. Both organic phases were mixed and evaporated under reduced pressure. The resulting crude was then purified by flash chromatography on silica gel with 200 mL of AcOEt/TEA 3% first and then with 1200 mL of AcOEt 8/MeOH 2/TEA 3% as an eluent to give the title compound as a yellow foam (11.91 g, 81%). ¹H-NMR (400 MHz, DMSO-d₆): δ 2.15 (3H, s), 2.30-2.34 (8H, m), 3.81 (1H, s), 7.18 (1H, s), 7.37 (1H, ddd), 7.61 (1H, s), 7.79 (1H, dt), 8.49 (1H, dd), 8.55 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=0.16 min. (100%) m/z (ES): 235 [M+H]⁺, 118 [M+H/2]⁺.

### Intermediate 56: (±)-2-(4-methylpiperazin-1-yl)-N′-[3,5-bis(trifluoromethyl)phenyl]-2-pyridin-3-ylacetohydrazide

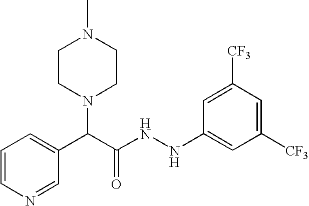

11.91 g of (±)-2-(4-methylpiperazin-1-yl)-2-pyridin-3-ylacetamide (Intermediate 55, 50.8 mmole) were dissolved in 470 mL of DCM. Then 22.74 g of (Boc)₂O (104.2 mmole, Aldrich) were added followed by 621 mg of DMAP (2,6-dimethylaminopyridine, 5.08 mmole, Aldrich). The yellow solution was then stirred for 3 h 15 min. 14.9 g of 3,5-bis(trifluoromethyl)phenylhydrazine (61.0 mmole, Lancaster) were added followed by 1.24 g of DMAP (10.2 mmole). The solution was then evaporated to the third and it was stirred at RT for 4.5 days. Solvents were removed under reduced pressure and the crude was then purified by flash chromatography on silica gel with the following eluents: 1500 mL of AcOEt 9/MeOH 1/TEA 3% and then 1500 mL of AcOEt 8/MeOH 2/TEA 3% to give the title compound as a yellow solid (11.16 g, 48%). ¹H-NMR (400 MHz, CD₃OD): δ 0.98 (3H, s), 1.21 (8H, m), 2.72 (1H, s), 5.67 (2H, s), 5.90 (1H, s), 6.16 (1H, dd), 6.71 (1H, dt), 7.24 (1H, dd), 7.36 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.63 min. (100%) m/z (ES): 462.1 [M+H]⁺, 231.6 [M+H/2]⁺.

### Compound 49: 2-(4-methylpiperazin-1-yl)-N′-[3,5-bis(trifluoromethyl)phenyl]-2-pyridin-3-ylacetohydrazide-enantiomer 1

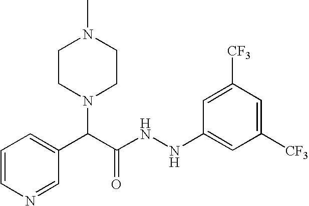

1 g of (±)-2-(4-methylpiperazin-1-yl)-N′-[3,5-bis(trifluoromethyl)phenyl]-2-pyridin-3-ylaceto-hydrazide (intermediate 56, 2.17 mmole) were purified by semipreparative chiral SFC [Chiralpak AS-H, 25×2.1 cm, pressure: 163 bar, Flow rate: 22 mL/min, UV detection: 220 nm, Injection, 40 mg each in EtOH, modifier: EtOH+0.1% isopropylamine]. Solvents were removed under reduced pressure and the crude (yellow solid, 415 mg) was triturated with 2×10 mL of Et₂O to give the title compound as a white solid (361 mg, 72%). ¹H-NMR (400 MHz, CD₃OD): δ 2.30 (3H, s), 2.53 (8H, m), 4.04 (1H, s), 6.99 (2H, s), 7.22 (1H, s), 7.49 (1H, dd), 8.04 (1H, dt), 8.56 (1H, d), 8.69 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.63 min. (100%) m/z (ES): 462.1 [M+H]⁺, 231.6 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=2.59 min. (100% ee).

### Compound 50: 2-(4-methylpiperazin-1-yl)-N′-[3,5-bis(trifluoromethyl)phenyl]-2-pyridin-3-ylacetohydrazide enantiomer 2

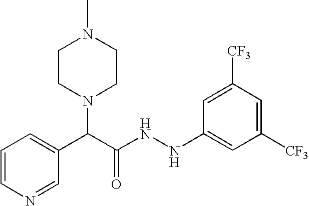

1 g of (±)-2-(4-methylpiperazin-1-yl)-N′-[3,5-bis(trifluoromethyl)phenyl]-2-pyridin-3-ylaceto-hydrazide (Intermediate 56, 2.17 mmole) were purified by semipreparative chiral SFC [Chiralpak AS-H, 25×2.1 cm, pressure: 163 bar, Flow rate: 22 mL/min, UV detection: 220 nm, Injection, 40 mg each in EtOH, modifier: EtOH+0.1% isopropylamine]. Solvents were removed under reduced pressure to give the title compound as a yellow solid (368 mg, 74%). ¹H-NMR (400 MHz, CD₃OD): δ 2.30 (3H, s), 2.53 (8H, m), 4.05 (1H, s), 6.99 (2H, s), 7.22 (1H, s), 7.49 (1H, dd), 8.04 (1H, dt), 8.56 (1H, dd), 8.68 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.63 min. (100%) m/z (ES): 462.1 [M+H]⁺, 231.6 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=5.04 min. (100% ee).

### Intermediate 57: (±)-Ethyl Hydroxy(imidazo[1,2-a]pyridin-6-yl)acetate

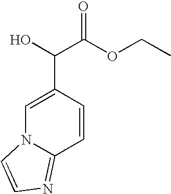

To 2.66 mL of a solution of iPrMgCl.LiCl 2.0M in THF (prepared according to the procedure of Krasovskiy A. and Knochel P., Angew. Chem. Int. Ed., 2004, 43, 3333-3336.) diluted in 2.66 mL of THF and cooled at 0° C. were added 1 g of 6-bromoimidazo[1,2-a]pyridine (5.07 mmole, Maybridge Combichem). The mixture was then allowed to reach RT and was stirred for 30 min under nitrogen atmosphere. The solution was then cooled to 0° C. and 2.07 mL of a solution of ethyl glyoxylate 50% in toluene (10.15 mmole, Fluka) were added. The thick solution was then allowed to reach RT and was stirred at RT for 15 hours. The reaction was quenched with 15 mL of water. 40 mL of AcOEt were added and the precipitate was filtered on celite. The cake was washed with 100 mL of AcOEt. The biphasic solution was transferred to a separatory funnel and the organic phase was removed. The aqueous phase was extracted 7 times with 20 mL of AcOEt. The combined organic layers were washed once with 30 mL of brine, dried over Na₂SO₄, filtered and evaporated to dryness. The resulting crude was then purified by flash chromatography (Flashmaster, columns Variant Si—NH₂) using the following eluents: 400 mL of CH, 400 mL of CH 80/AcOEt 20, 400 mL of CH 70/AcOEt 30, 400 mL of CH 30/AcOEt 70, 400 mL of CH 10/AcOEt 90, 1050 mL of AcOEt, 100 mL of AcOEt 90/MeOH 10 to give the title compound as a yellow solid (439 mg, 39%). ¹H-NMR (400 MHz, DMSO-d₆): δ 1.15 (3H, t), 4.07-4.16 (2H, m), 5.19 (1H, d), 6.26 (1H, d), 7.22 (1H, dd), 7.55 (1H, d), 7.57 (1H, d), 7.98 (1H, s), 8.61 (1H, s). LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=0.46 min. (100%) m/z (ES): 221 [M+H]⁺.

### Intermediate 58: (±)-Ethyl imidazo[1,2-a]pyridin-6-yl(4-methylpiperazinyl)acetate

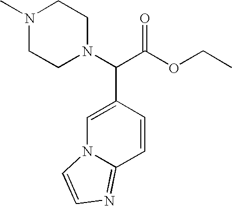

In a dry flask under nitrogen, 200 mg of (±)-ethyl hydroxy(imidazo[1,2-a]pyridin-6-yl)acetate (Intermediate 57, 0.91 mmole) were dissolved in 5.9 mL of DCM. 152 μl of TEA were added and the solution was cooled to 0° C. Then 77 μL of mesylchloride (1.00 mmole, Aldrich) were added dropwise and the solution was stirred at 0° C. for 30 min. After completion of the reaction, 222 μL of N-methylpiperazine (2.00 mmole, Aldrich) were added and the solution was allowed to reach RT. The mixture was then stirred at RT for 19 h. The reaction mixture was then transferred to a separatory funnel and 15 mL of DCM were added followed by 15 mL of a saturated solution of NaHCO₃. The aqueous phase was removed and the organic phase was washed once with 15 mL of a saturated solution of NaHCO₃, 15 mL of brine, dried over Na₂SO₄, filtered and evaporated to dryness. The crude was then purified by flash chromatography (flashmaster, column Variant Si—NH₂) with the following eluents: 200 mL of CH, 200 mL of CH 80/AcOEt 20, 200 mL of CH 70/AcOEt 30, 200 mL of CH 60/AcOEt 40, 200 mL of CH 50/AcOEt 50, 200 mL of CH 40/AcOEt 60. The fraction corresponding to the desired compound was collected and solvents were removed under reduced pressure. The resulting material was then passed through a HLB-LP cartridge (Waters Oasis® HLB 35 cc (6 g) LP Extraction Cartridges). The cartridge was then eluted with 100 mL of water, 100 mL of Water 90/MeOH 10, 50 mL of water 70/MeOH 30, 50 mL of water 60/MeOH 40, 50 mL of water 50/MeOH 50, 50 mL of water 40/MeOH 60, 100 mL of MeOH to give the title compound as a colourless oil (170 mg, 62%). ¹H-NMR (400 MHz, CDCl₃): δ 1.26 (3H, t), 2.32 (3H, s), 2.54 (8H, m), 3.98 (1H, s), 4.16-4.27 (2H, m), 7.34 (1H, dd), 7.59-7.62 (2H, m), 7.65 (1H, s), 8.27 (1H, s). LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=0.34 min. (100%) m/z (ES): 303 [M+H]⁺.

### Intermediate 59: (±)-N′-[3,5-bis(trifluoromethyl)phenyl]-2-imidazo[1,2-a]pyridin-6-yl-2-(4-methylpiperazin-1-yl)acetohydrazide

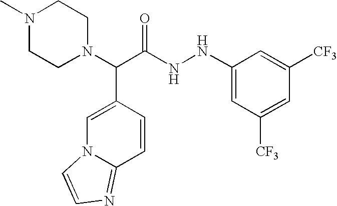

170 mg of (±)-ethyl imidazo[1,2-a]pyridin-6-yl(4-methylpiperazinyl)acetate (Intermediate 58, 0.56 mmole) were dissolved in 3.64 mL of MeOH at RT. Then 31.5 mg of KOH (0.59 mmole) were added followed by 387 μL of water. The mixture was then stirred at RT for 48 h. Solvents were removed under reduced pressure and the crude (yellow wax, 213 mg) was dried under high vacuum and used in the next step without further purification. 206 mg of this product (0.66 mmole) were then dissolved in 2.36 mL of DMF. To this solution, were added successively 169 mg of 3,5-bis(trifluoromethyl)phenylhydrazine (0.69 mmole), 76 μL of NMM (0.69 mmole, Aldrich) and 306 mg of BOP (0.69 mmole, FLUKA). The mixture was then stirred at RT for 16 h. Solvents were removed under reduced pressure and the residue was dissolved in 50 mL of AcOEt. The organic phase was then washed twice with 25 mL of NaOH 1N, once with 25 mL of a saturated solution of NaHCO₃, once with 25 mL of brine, dried over Na₂SO₄, filtered and evaporated to dryness. The crude was then purified by flash chromatography (flashmaster, column Variant Si—NH₂) with the following eluents: 200 mL of CH, 400 mL of CH 90/AcOEt 10, 200 mL of CH 80/AcOEt 20, 200 mL of CH 60/AcOEt 40, 200 mL of CH 30/AcOEt 70, 600 mL of AcOEt and 200 mL of AcOEt 90/MeOH 10 to give the title compound as a white solid (183 mg, 55%). ¹H-NMR (400 MHz, CD₃OD): δ 2.34 (3H, s), 2.60 (8H, m), 4.00 (1H, s), 7.03 (2H, s), 7.21 (1H, s), 7.55-7.62 (3H, m), 7.88 (1H, s), 8.60 (1H, s).

### Compound 51: (±)-N′-[3,5-bis(trifluoromethyl)phenyl]-2-imidazo[1,2-a]pyridin-6-yl-2-(4-methylpiperazin-1-yl)acetohydrazide enantiomer 1

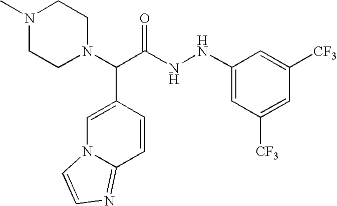

183 mg of Intermediate 59 ((±)-N′-[3,5-bis(trifluoromethyl)phenyl]-2-imidazo[1,2-a]pyridin-6-yl-2-(4-methylpiperazin-1-yl)acetohydrazide) were purified by semipreparative chiral SFC [Chiralpak AS-H, 25×2.1 cm, pressure: 163 bar, Flow rate: 22 mL/min, UV detection: 220 nm, Injection: 40 mg each in EtOH, modifier: EtOH+0.1% isopropylamine]. Solvents were removed under reduced pressure to give the title compound as a white solid (60.5 mg, 66%). ¹H-NMR (400 MHz, CD₃OD): δ 2.31 (3H, s), 2.58 (8H, m), 4.00 (1H, s), 7.03 (2H, s), 7.20 (1H, s), 7.55-7.62 (3H, m), 7.87 (1H, s), 8.60 (1H, s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.85 min. (100%) m/z (ES): 500.9 [M+H]⁺, 251.1 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=5.44 min. (100% ee).

### Compound 52: N′-[3,5-bis(trifluoromethyl)phenyl]-2-imidazo[1,2-a]pyridin-6-yl-2-(4-methylpiperazin-1-yl)acetohydrazide-enantiomer 9

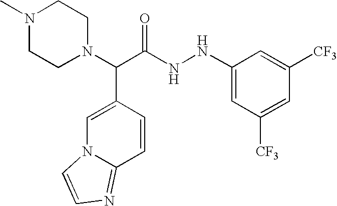

183 mg of intermediate 59 ((±)-N′-[3,5-bis(trifluoromethyl)phenyl]-2-imidazo[1,2-a]pyridin-6-yl-2-(4-methylpiperazin-1-yl)acetohydrazide) were purified by semipreparative chiral SFC [Chiralpak AS-H, 25×2.1 cm, pressure: 163 bar, Flow rate: 22 mL/min, UV detection: 220 nm, Injection, 40 mg each in EtOH, modifier: EtOH+0.1% isopropylamine]. Solvents were removed under reduced pressure to give the title compound as a white solid (60.0 mg, 66%). ¹H-NMR (400 MHz, CD₃OD): δ 2.31 (3H, s), 2.58 (8H, m), 4.01 (1H, s), 7.03 (2H, s), 7.20 (1H, s), 7.55-7.61 (3H, m), 7.87 (1H, s), 8.60 (1H, s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.85 min. (100%) m/z (ES): 500.9 [M+H]⁺, 251.1 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=10.01 min. (100% ee).

### Intermediate 60: (±)-Ethyl hydroxy(2-methylpyridin-3-yl)acetate

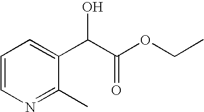

This compound was prepared following a similar procedure to that used for intermediate 57 starting from 500 mg of 3-bromo-2-methylpyridine (2.91 mmole, AsymChem) to give the title compound as a brown oil (64 mg, 11%). ¹H-NMR (400 MHz, CDCl₃): δ 1.20 (3H, t), 2.62 (3H, s), 4.15-4.26 (2H, m), 5.36 (1H, s), 7.13 (1H, dd), 7.68 (1H, d), 8.38 (1H, d). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.40 min. (83.6%); m/z (ES): 196.1 [M+H]⁺.

### Intermediate 61: (±)-Ethyl (4-methylpiperazin-1-yl)(2-methylpyridin-3-yl)acetate

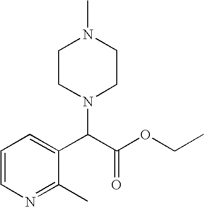

This compound was prepared following a similar procedure to that used for Intermediate 58 starting from 63 mg of (±)-ethyl hydroxyl(2-methylpyridin-3-yl)acetate (Intermediate 60, 0.32 mmole) to give the title compound as a colourless oil (40 mg, 45%). ¹H-NMR (400 MHz, CD₃OD): δ 1.21 (3H, t), 2.29 (3H, s), 2.36-2.65 (11H, m), 4.13-4.21 (2H, m), 4.42 (1H, s), 7.31 (1H, dd), 7.93 (1H, dd), 8.38 (1H, dd); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.39 min. (100%) m/z (ES): 278.2 [M+H]⁺, 139.6 [M+H/2]⁺.

### Intermediate 62: (±)-N′-[3,5-bis(trifluoromethyl)phenyl]-2-(4-methylpiperazin-1-yl)-2-(2-methylpyridin-3-yl)acetohydrazide

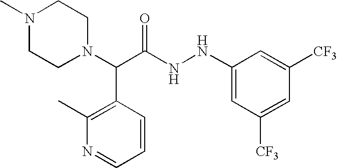

40 mg of ethyl(±)-(4-methylpiperazin-1-yl)(2-methylpyridin-3-yl)acetate (Intermediate 61, 0.14 mmole) were dissolved in 1 mL of MeOH. Then 8.5 mg of KOH were added followed by 97 μL of water. The mixture was stirred at RT for 27 h. Solvents were removed under reduced pressure and the crude (yellow solid, 45 mg) was dried under high vacuum. This product was used without further purifications in the next step. 45 mg of this product (0.16 mmole) were then dissolved in 2.36 mL of DMF. To this solution, were added successively 169 mg of 3,5-bis(trifluoromethyl)phenylhydrazine (0.69 mmole), 18 μL of NMM (0.16 mmole, Aldrich) and 73 mg of BOP (0.16 mmole, FLUKA). The mixture was then stirred at RT for 2.5 h. Solvents were removed under reduced pressure and the crude was purified by preparative HPLC. Solvents were removed and the compound was then dissolved in 3 mL of MeOH and the solution was passed through a SCX cartridge. The cartridge was washed with 15 mL of DCM, 15 mL of MeOH and 15 mL of a solution of NH₃ 2M in MeOH. Solvents were removed under reduced pressure to give the title compound as a yellow solid (22 mg, 29%). ¹H-NMR (400 MHz, CDCl₃): δ 2.33 (3H, s), 2.53-2.70 (11H, m), 4.38 (1H, s), 6.81 (1H, m), 7.02 (2H, s), 7.23 (1H, dd), 7.30 (1H, s), 7.83 (1H, dd), 8.49 (1H, dd), 8.85 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.19 min. (100%) m/z (ES): 476.1 [M+H]⁺, 238.6 [M+H/2]⁺.

### Compound 53: N′-[3,5-bis(trifluoromethyl)phenyl]-2-(4-methylpiperazin-1-yl)-2-(2-methylpyridin-3-yl)acetohydrazide enantiomer 1

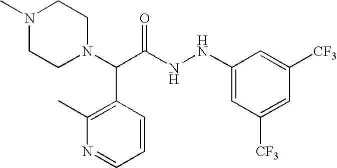

22 mg of (±)-N′-[3,5-bis(trifluoromethyl)phenyl]-2-(4-methylpiperazin-1-yl)-2-(2-methylpyridin-3-yl)acetohydrazide (Intermediate 62) were purified by semipreparative chiral SFC [Chiralpak AS-H, 25×2.1 cm, pressure: 163 bar, Flow rate: 22 mL/min, UV detection: 220 nm, Injection: 40 mg each in EtOH, modifier: EtOH+0.1% isopropylamine]. Solvents were removed under reduced pressure to give the title compound as a colourless oil (6 mg, 54%). ¹H-NMR (400 MHz, CDCl₃): δ 2.34 (3H, s), 2.50-2.64 (8H, m), 2.72 (3H, s), 4.39 (1H, s), 6.44 (1H, s), 7.05 (2H, s), 7.25 (1H, dd), 7.35 (1H, s), 7.82 (1H, dd), 8.51 (1H, dd), 8.63 (1H, s); m/z: 476.2 [M+H]⁺, 238.6 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=7.52 min. (100% ee).

### Compound 54: N′-[3,5-bis(trifluoromethyl)phenyl]-2-(4-methylpiperazin-1-yl)-2-(2-methylpyridin-3-yl)acetohydrazide-enantiomer 2

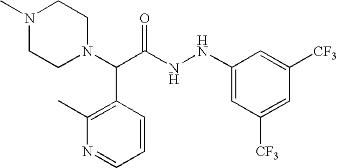

22 mg of (±)-N′-[3,5-bis(trifluoromethyl)phenyl]-2-(4-methylpiperazin-1-yl)-2-(2-methyl-pyridin-3-yl)acetohydrazide (Intermediate 62) were purified by semipreparative chiral SFC [Chiralpak AS-H, 25×2.1 cm, pressure: 163 bar, Flow rate: 22 mL/min, UV detection: 220 nm, Injection: 40 mg each in EtOH, modifier: EtOH+0.1% isopropylamine]. Solvents were removed under reduced pressure to give the title compound as a colourless oil (6.1 mg, 54%). ¹H-NMR (400 MHz, CDCl₃): δ 2.34 (3H, s), 2.54-2.64 (8H, m), 2.72 (3H, s), 4.40 (1H, s), 6.42 (1H, s), 7.06 (2H, s), 7.25 (1H, dd), 7.35 (1H, s), 7.81 (1H, dd), 8.51 (1H, dd), 8.63 (1H, s); m/z: 476.2 [M+H]⁺, 238.6 [M+H/2]⁺; Chiral SFC [Chiralpak AS-H, 25×0.46 cm, pressure: 100 bar, Flow rate: 2.0 mL/min, UV detection: 240 nm, modifier: EtOH+0.1% isopropylamine]: Rₜ=13.35 min. (100% ee).

### Intermediate 63: (3-methyl-2-pyridinyl)acetonitrile

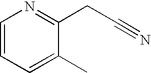

To a solution of 511 μL of dry acetonitrile (12 mmole) in 15 mL of dry THF, 7.25 mL of n-BuLi (11.6 mmole, 1.6 M solution in hexane, Aldrich) were added at −78° C. and under N₂. A solution of 0.5 g of 2-bromo-3-methylpyridine (2.9 mmole, Aldrich) in 15 mL of dry THF was added dropwise. The mixture was stirred at −78° C. for 2 h and was then warmed to RT and stirred for 2 h. 10 mL of water and 30 mL of AcOEt were added to the mixture, the organic layer was separated, dried over sodium sulphate, filtered and the solvent evaporated under reduced pressure. The crude thus obtained was purified on FlashMaster silica cartridge (CH/AcOEt 1:1 as an eluent) to give the title compound as a yellow oil (350 mg, 91%). ¹H-NMR (400 MHz, DMSO-d₆): δ 2.40 (3H, s), 3.91 (2H, s), 7.22 (1H, dd), 7.54 (1H, d), 8.45 (1H, d).

### Intermediate 64: methyl(3-methyl-2-pyridinyl)acetate

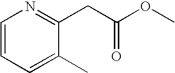

350 mg of (3-methyl-2-pyridinyl)acetonitrile (intermediate 63, 2.65 mmole) were dissolved in 5 mL of HCl 37% and stirred at 90° C. for 1 h. The solvent was then evaporated under reduced pressure to afford 0.6 g of a yellow solid that were dissolved in 5 mL of MeOH. 1.32 mL of trimethylsilyldiazomethane (2.65 mmole, 2.0M solution in n-Hex, Aldrich) were added and the reaction mixture was stirred 1 h at RT, then AcOH was added (2.65 mmole) and the reaction was stirred for 5 min at RT. The solvent was then evaporated under reduced pressure to give the title compound as a yellow foam (400 mg, 91%). ¹H-NMR (400 MHz, DMSO-d₆): δ 2.34 (3H, s), 3.74 (2H, s), 4.1 (3H, s), 7.16 (1H, dd), 7.52 (1H, dd), 8.22 (1H, d).

### Intermediate 65: (±)-methyl(4-methyl-1-piperazinyl)(3-methyl-2-pyridinyl)acetate

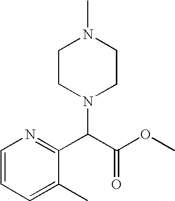

To a solution of 375 mg of (±)-methyl(3-methyl-2-pyridinyl)acetate (Intermediate 64, 2.27 mmole) in 6 mL of DCM cooled to 0° C., were added 405 mg of NBS (2.27 mmole, Aldrich). The reaction mixture was stirred at RT under nitrogen atmosphere for 3 h then 569 mg of N-methylpiperazine (5.68 mmole, Aldrich) were added. The solution was stirred at RT for 4 h. Solvents were removed under reduced pressure and the resulting crude was purified by flash chromatography on a 20 g silica gel cartridge using a gradient of DCM/MeOH 10/0 to 9/1 as an eluent. Solvents were removed and the resulting oil was dissolved in AcOEt. The organic phase was washed with NaHCO₃. The solvent was removed to give the title compound as a brown oil (210 mg, 35%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.38 min, m/z (ES): 264 [M+H]⁺.

### Intermediate 66: (±)-2-(4-methyl-1-piperazinyl)-2-(3-methyl-2-pyridinyl)acetohydrazide

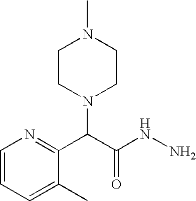

210 mg of methyl(±)-(4-methyl-1-piperazinyl)(3-methyl-2-pyridinyl)acetate (Intermediate 65) were dissolved in 2 mL of hydrazine hydrate neat. The reaction mixture was stirred at RT for 3 h. Solvents were removed to give the final compound (200 mg, 95%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.18 min, m/z (ES): 264 [M+H]⁺.

### Intermediate 67: (±)-N′-[3,5-bis(trifluoromethyl)phenyl]-2-(4-methyl-1-piperazinyl)-2-(3-methyl-2-pyridinyl)acetohydrazide

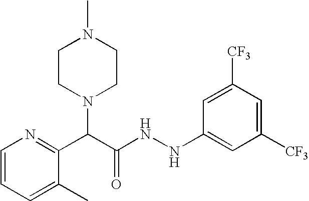

To a solution of 200 mg of (±)-2-(4-methyl-1-piperazinyl)-2-(3-methyl-2-pyridinyl)acetohydrazide (Intermediate 66, 0.76 mmole) in a 4/1 mixture of dioxane/dodecane (8 mL/2 mL) were added 134 μL of 1-iodo-3,5-bis(trifluoromethyl)benzene (0.76 mmole, Aldrich), 12 μL of diaminocyclohexane (0.08 mmole), 8.6 mg of CuI (0.045 mmole, Aldrich) and 210 mg of K₂CO₃ (1.52 mmole). The reaction mixture was flushed with argon and heated at 100° C. under argon for 3 h. It was cooled to RT and poured into H₂O/AcOEt. The organic layer was washed with brine, dried over Na₂SO₄, filtered and evaporated to dryness. The crude was purified on flash chromatography on silica gel cartridge using a gradient of DCM/MeOH 10/0 to 8/2 as an eluent. Solvents were removed under reduced pressure to give the title compound as a brown oil (29 mg, 8%). This intermediate was mixed with another batch of the same compound obtained following a similar procedure (8 mg, 0.017 mmole) to give one batch of 37 mg. ¹H-NMR (400 MHz, CDCl₃): 2.49 (3H, s), 2.57 (3H, s), 2.84 (8H, m), 4.54 (1H, s), 7.14 (2H, s), 7.30 (2H, m), 7.60 (1H, d), 8.58 (1H, d); m/z (ES): 476 [M+H]

### Compound 55: N′-[3,5-bis(trifluoromethyl)phenyl]-2-(4-methyl-1-piperazinyl)-2-(3-methyl-2-pyridinyl)acetohydrazide-enantiomer 1

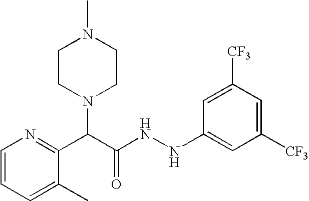

37 mg of (±)-N′-[3,5-bis(trifluoromethyl)phenyl]-2-(4-methyl-1-piperazinyl)-2-(3-methyl-2-pyridinyl)acetohydrazide (Intermediate 67) were purified by semipreparative chiral SFC. Solvents were removed under reduced pressure and the crude was triturated with Et₂O to give the title compound as a yellow oil (6.2 mg, 34%). ¹H-NMR (400 MHz, CDCl₃): δ 2.34 (3H, s), 2.44 (3H, s), 2.50 (8H, m), 4.49 (1H, s), 6.29 (1H, s), 7.20 (3H, m), 7.33 (1H, s), 7.61 (1H, d), 8.58 (1H, d), 9.35 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.62 min, m/z (ES): 476 [M+H]⁺; Chiral SFC [Chiralpak AS-H, 25×0.46 cm. Pressure: 100 bar. Flow rate: 2.0 mL/min. UV detection: 240 nm. Modifier: EtOH+0.1% isopropylamine 12%]: Rₜ=2.91 min (100% ee).

### Compound 56: N′-[3,5-bis(trifluoromethyl)phenyl]-2-(4-methyl-1-piperazinyl)-2-(3-methyl-2-pyridinyl)acetohydrazide-enantiomer 2

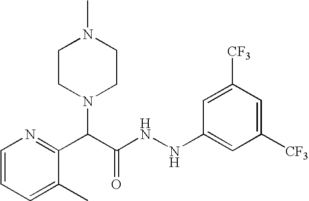

37 mg of (±)-N′-[3,5-bis(trifluoromethyl)phenyl]-2-(4-methyl-1-piperazinyl)-2-(3-methyl-2-pyridinyl)acetohydrazide (Intermediate 67) were purified by semipreparative chiral SFC. Solvents were removed under reduced pressure and the crude was triturated with Et₂O to give the title compound as a yellow oil (6.2 mg, 34%). ¹H-NMR (400 MHz, CDCl₃): δ 2.33 (3H, s), 2.49 (3H, s), 2.55 (8H, m), 4.47 (1H, s), 6.26 (1H, s), 7.24 (3H, m), 7.34 (1H, s), 7.57 (1H, d), 8.60 (1H, d), 9.35 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.62 min, m/z (ES): 476 [M+H]⁺; Chiral SFC [Chiralpak AS-H, 25×0.46 cm. Pressure: 100 bar. Flow rate: 2.0 mL/min. UV detection: 240 nm. Modifier: EtOH+0.1% isopropylamine 12%]: Rₜ=8.17 min (100% ee).

### Intermediate 68: (±)-ethyl hydroxy(6-methyl-3-pyridinyl)acetate

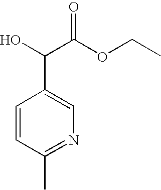

To 3.05 mL of iPrMgCl.LiCl 2.0M in THF (6.10 mmole) (prepared according to the procedure of Krasovskiy A. and Knochel P., Angew. Chem. Int. Ed., 2004, 43, 3333-3336) diluted in 3.05 mL of THF and cooled at 0° C., was added 1 g of 5-bromo-2-methylpyridine (5.81 mmole, Fluka). The mixture was allowed to reach RT and was stirred for 3 h under a nitrogen atmosphere. 1.5 mL of iPrMgCl.LiCl 2.0M in THF (2.91 mmole) were added and the reaction was left overnight. The solution was cooled to 0° C. and 2.10 mL of Ethyl glyoxylate 50% in toluene (11.63 mmole, Fluka) were added. The thick solution was allowed to reach RT and was stirred at RT for 5 h. The reaction was quenched with water. AcOEt was added and the precipitate was filtered on celite. The cake was washed with AcOEt. The biphasic solution was transferred to a separatory funnel and the organic phase was separated. The organic layers were washed with brine, dried over Na₂SO₄, filtered and evaporated to dryness. The resulting crude was purified by flash chromatography (Flashmaster, columns Variant Si—NH₂) using a gradient from 100% CH to CH/AcOEt 1/1 to give the title compound as a yellow oil (40 mg, 4%). This reaction was repeated with the same procedure on 2 g of 5-bromo-2-methylpyridine (11.6 mmole) to give the title compound (169 mg, 7.7%). The two compounds were mixed together for the next reaction. ¹H-NMR (400 MHz, CDCl₃): δ 1.23 (3H, t), 2.55 (3H, s), 3.65 (1H, s), 4.24 (2H, m), 5.17 (1H, s), 7.15 (1H, d), 7.70 (1H, dd), 8.58 (1H, d); UPLC-MS [Acquity™UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.33 min, m/z (ES): 196 [M+H]⁺.

### Intermediate 69: (±)-ethyl(4-methyl-1-piperazinyl)(6-methyl-3-pyridinyl)acetate

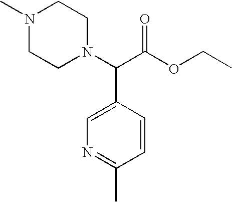

A solution of 209 mg of ethyl(±)-hydroxy(6-methyl-3-pyridinyl)acetate (Intermediate 68, 1.07 mmole) in 6 mL of DCM and 179 μL of TEA (1.28 mmole) was cooled to 0° C. and 90 μL of mesylchloride (1.18 mmole, Aldrich) were added dropwise. The solution was stirred at 0° C. for 1 h then 260 μL of N-methylpiperazine (2.35 mmole, Aldrich) were added. The mixture was allowed to reach RT and was stirred for 20 h. DCM was added and the organic phase was washed with a saturated solution of NaHCO₃, brine, dried over Na₂SO₄, filtered and evaporated to dryness to give the title compound as a yellow oil (287 mg, 94%). ¹H-NMR (400 MHz, CDCl₃): δ 1.25 (3H, t), 2.29 (3H, s), 2.55 (11H, m), 4.01 (1H, s), 4.16 (2H, m), 7.18 (1H, d), 7.75 (1H, dd), 8.55 (1H, d); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.34 min, m/z (ES): 278 [M+H]⁺.

### Intermediate 70: potassium (±)-(4-methyl-1-piperazinyl)(6-methyl-3-pyridinyl)acetate

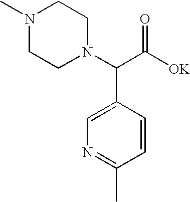

To a solution of 278 mg of ethyl(4-methyl-1-piperazinyl)(6-methyl-3-pyridinyl)acetate (Intermediate 69, 1.00 mmole) in 5 mL of MeOH were added 87 mg of KOH (1.5 mmole) followed by 500 μL of water. The mixture was stirred at RT for 72 h. Solvents were removed under reduced pressure and the crude (320 mg, quantitative) was dried and used in the next step without further purification. ¹H-NMR (400 MHz, DMSO-d₆): δ 2.10 (3H, s), 2.20 (8H, m), 2.38 (3H, s), 3.29 (1H, s), 7.05 (1H, d), 7.63 (1H, dd), 8.29 (1H, d); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.16 min, m/z (ES): 250 [M+H]⁺.

### Intermediate 71: (±)-N′-[3,5-bis(trifluoromethyl)phenyl]-2-(4-methyl-1-piperazinyl)-2-(6-methyl-3-pyridinyl)acetohydrazide

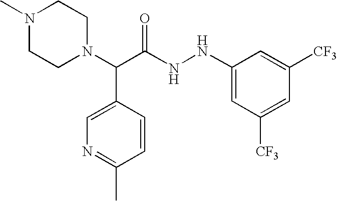

320 mg of potassium (±)-(4-methyl-1-piperazinyl)(6-methyl-3-pyridinyl)acetate (intermediate 70, 1.11 mmole) were dissolved in 5 mL of DMF. To this solution were added successively 285 mg of 3,5-bis(trifluoromethyl)phenylhydrazine (1.17 mmole, Alfa Aesar), 128 μL of NMM (1.17 mmole, Aldrich) and 517 mg of BOP (1.17 mmole, Fluka). The mixture was stirred at RT for 15 h. Solvents were removed under reduced pressure and the residue was dissolved in AcOEt. The organic phase was washed with a solution of NaOH 1N, a saturated solution of NaHCO₃, brine, dried over Na₂SO₄, filtered and evaporated to dryness. The crude was dissolved in DCM and the solution was passed through a SCX cartridge. The cartridge was washed with DCM, MeOH and NH₃ 2M/MeOH. Solvents were removed under reduced pressure and the resulting compound was purified by flash chromatography (flashmaster, column Variant Si—NH₂) using a gradient of DCM/MeOH (30% NH₃ in MeOH) 10/0 to 8/2 to give the title compound as a yellow oil (257 mg, 49%). ¹H-NMR (400 MHz, CDCl₃): δ 2.34 (3H, s), 2.58 (11H, m), 4.11 (1H, s), 6.30 (1H, s), 7.09 (2H, s), 7.19 (1H, d), 7.39 (1H, s), 7.59 (1H, s), 8.50 (1H, s), 8.75 (1H, s); m/z (ES): 476.2 [M+H]⁺, 238.6 [(M+H)/2]⁺.

### Compound 57: N′-[3,5-bis(trifluoromethyl)phenyl]-2-(4-methyl-1-piperazinyl)-2-(6-methyl-3-pyridinyl)acetohydrazide-enantiomer 1

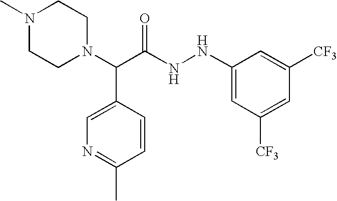

257 mg of (±)-N′-[3,5-bis(trifluoromethyl)phenyl]-2-(4-methyl-1-piperazinyl)-2-(6-methyl-3-pyridinyl)acetohydrazide (Intermediate 71, 0.54 mmole) were purified by semipreparative chiral SFC [Chiralpak AS-H, 25×2.1 cm. Pressure: 160 bar. Flow rate: 22 mL/min. UV detection: 220 nm. Injection: 20 mg each in EtOH. Modifier: EtOH+0.1% isopropylamine]. Solvents were removed under reduced pressure and the crude was triturated with Et₂O to give the title compound as a white solid (92 mg, 72%). ¹H-NMR (400 MHz, CDCl₃): δ 2.34 (3H, s), 2.55 (11H, m), 4.12 (1H, s), 6.34 (1H, s), 7.10 (2H, s), 7.21 (1H, dd), 7.37 (1H, s), 7.61 (1H, dd), 8.51 (1H, s), 8.79 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]. Rₜ=3.56 min (96.5%) m/z (ES): 476 [M+H]⁺, 238 [(M+H)/2]⁺; Chiral SFC [Chiralpak AS-H, 25×0.46 cm. Pressure: 100 bar. Flow rate: 2.0 mL/min. UV detection: 240 nm. 15% Modifier: EtOH+0.1% isopropylamine]: Rₜ=2.20 min (100% ee).

### Compound 58: N′-[3,5-bis(trifluoromethyl)phenyl]-2-(4-methyl-1-piperazinyl)-2-(6-methyl-3-pyridinyl)acetohydrazide-enantiomer 2

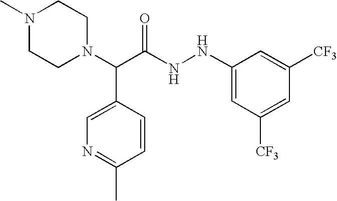

257 mg of (±)-N′-[3,5-bis(trifluoromethyl)phenyl]-2-(4-methyl-1-piperazinyl)-2-(6-methyl-3-pyridinyl)acetohydrazide (Intermediate 71, 0.54 mmole) were purified by semipreparative chiral SFC [Chiralpak AS-H, 25×2.1 cm. Pressure: 160 bar. Flow rate: 22 mL/min. UV detection: 220 nm. Injection: 20 mg each in EtOH. Modifier: EtOH+0.1% isopropylamine]. Solvents were removed under reduced pressure and the crude was triturated with Et₂O to give the title compound as a white solid (42 mg, 32%). ¹H-NMR (400 MHz, CDCl₃): δ 2.31 (3H, s), 2.54 (11H, m), 4.12 (1H, s), 6.32 (1H, s), 7.10 (2H, s), 7.21 (1H, dd), 7.38 (1H, s), 7.61 (1H, dd), 8.51 (1H, s), 8.79 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]. Rₜ=3.54 min (100%) m/z (ES): 476 [M+H]⁺, 238 [(M+H)/2]⁺; Chiral SFC [Chiralpak AS-H, 25×0.46 cm. Pressure: 100 bar. Flow rate: 2.0 mL/min. UV detection: 240 nm. 15% Modifier: EtOH+0.1% isopropylamine]: Rₜ=3.30 min (100% ee).

### Intermediate 72: (3-chloro-2-pyridinyl)acetonitrile

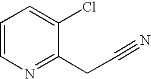

2.12 mL of MeCN (40.54 mmole, Aldrich) were added dropwise to 25.34 mL of butyllithium 1.6 M (40.541 mmole, Aldrich) in 40 mL of dry THF, previously cooled at −78° C. under nitrogen atmosphere. The temperature was maintained between −78° C. and −70° C. The reaction mixture was stirred for 45 min, and then a solution of 5 g of 2,3-dichloropyridine (33.8 mmole, 1 eq, Aldrich) in 300 mL of THF dry was carefully added dropwise. The reaction was stirred at −78° C. for 2 h, then it was allowed to reach RT and quenched with 50 mL of water. The aqueous phase was extracted with AcOEt. The combined organic phases were dried and evaporated under reduced pressure. The crude was purified by flash chromatography on silica gel (from CH to CH/AcOEt 60/40) to give the title compound (1 g, 19%). ¹H-NMR (400 MHz, CDCl₃): δ 4.05-4.12 (2H, m), 7.25-7.36 (1H, m), 7.74-7.78 (1H, m), 8.54-8.57 (1H, m); m/z (ES⁺): 153 [M+H]⁺.

### Intermediate 73: (3-chloro-2-pyridinyl)acetic acid hydrochloride salt

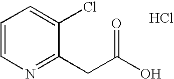

1.15 g of (3-chloro-2-pyridinyl)acetonitrile (intermediate 72, 7.57 mmole) were dissolved in 4.5 mL of concentrated hydrochloric acid. The mixture was heated at 90° C. for 4 h. The solvent was removed in vacuo to give the title compound as a pale yellow solid (1.9 g, quantitative). ¹H-NMR (400 MHz, DMSO-d₆): δ 3.89 (3H, s), 7.38 (1H, q), 7.93 (1H, dd), 8.47 (1H, dd); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.46 min, m/z (ES): 172 [M+H]⁺.

### Intermediate 74: methyl(3-chloro-2-pyridinyl)acetate

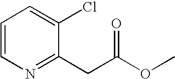

9.18 mL of TMSCHN₂ ((trimethylsilyl)diazomethane) 2M in hexanes (18.3 mmole, Aldrich) were slowly added to a solution of 1.9 g of (3-chloro-2-pyridinyl)acetic acid hydrochloride salt (Intermediate 73, 9.18 mmole) in 45 mL of dry MeOH. After stirring for 30 min, further (trimethysilyl)diazomethane (18.3 mmole) was added and the reaction mixture was stirred for additional 5 h. Then it was quenched with 2 mL of acetic acid (glacial, Aldrich), diluted with water and extracted with DCM (3×80 mL). The combined organic phases were dried and evaporated under reduced pressure to give the title compound (1.32 g, 77%). ¹H-NMR (400 MHz, CDCl₃): δ 3.75-3.78 (3H, m), 4.06 (2H, s), 7.22 (1H, q), 7.72 (1H, dd), 8.49 (1H, dd); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.57 min, m/z (ES): 186 [M+H]⁺.

### Intermediate 75: (±)-methyl(3-chloro-2-pyridinyl)(4-methyl-1-piperazinyl)acetate

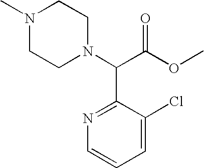

577 mg of NBS (3.2 mmole, Aldrich) were added to a solution of 600 mg of methyl(3-chloro-2-pyridinyl)acetate (intermediate 74, 3.2 mmole) in 10 mL of dry DCM at 0° C. and the reaction mixture was stirred at RT overnight. Then, 902 μL of N-methylpiperazine (8.1 mmole, Aldrich) were added and the reaction mixture was stirred at RT for 7 h. Then water was added, the phases were separated and aqueous phase was extracted with DCM (3×20 mL). The combined organic phases were dried and evaporated under reduced pressure. The crude was purified by flash chromatography on silica gel (from DCM to DCM/MeOH 80/20) to give the title compound (524 mg, 57%). ¹H-NMR (400 MHz, CDCl₃): δ 2.33 (3H, s), 2.54-2.59 (4H, br s), 2.73-2.90 (4H, br s), 3.73 (3H, s), 5.06 (1H, s), 7.21 (1H, q), 7.73 (1H, dd), 8.52 (1H, dd); m/z (ES/+): 284 [M+H]⁺.

### Intermediate 76: (±)-2-(3-chloro-2-pyridinyl)-2-(4-methyl-1-piperazinyl)acetohydrazide

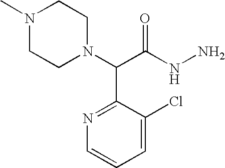

524 mg of methyl(±)-(3-chloro-2-pyridinyl)(4-methyl-1-piperazinyl)acetate (intermediate 75, 1.85 mmole) were dissolved in 1.15 mL of hydrazine hydrate (37.03 mmole, Aldrich). The reaction mixture was stirred at RT for 2 h. Then, 5 mL of hydrazine hydrate were added and the mixture was stirred overnight at RT. The reaction mixture was concentrated under reduced pressure to give the title compound (450 mg, 86%) which was used in the next step without further purification. ¹H-NMR (400 MHz, CDCl₃): δ 2.21-2.31 (3H, m), 2.33-2.74 (8H, m), 3.95 (2H, s), 4.98 (1H, s), 7.14-7.30 (1H, m), 7.73 (1H, dd), 8.54 (1H, dd), 8.61 (1H, s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.24 min, m/z (ES): 284 [M+H]⁺; m/z (ES/+): 284 [M+H]⁺.

### Intermediate 77: potassium (±)-(3-chloro-2-pyridinyl(4-methyl-1-piperazinyl)carboxylate

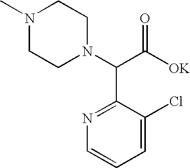

206 mg of methyl(±)-(3-chloro-2-pyridinyl)(4-methyl-1-piperazinyl)acetate (intermediate 75, 0.7 mmole) were suspended in 1.5 mL of a 1.5 M water solution of KOH (2.2 mmole, Fluka) and stirred at RT overnight. Then the mixture was concentrated under reduced pressure, to give the title compound as a pale yellow solid (339 mg, quantitative). ¹H-NMR (400 MHz, DMSO-d₆): δ 2.11 (3H, s), 2.16-2.75 (8H, m), 4.23 (1H, s), 7.21 (1H, q), 7.79 (1H, dd), 8.42 (1H, dd); m/z (ES): 270 [M+H]⁺.

### Intermediate 78: (±)-N′-[3,5-bis(trifluoromethyl)phenyl]-2-(3-chloro-2-pyridinyl)-2-(4-methyl-1-piperazinyl)acetohydrazide-procedure A

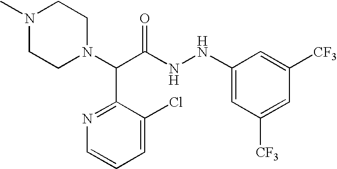

In a dry round bottom flask (5 cycles vacuum/argon) to a mix of 225 mg of N′-[3,5-bis (trifluoromethyl)phenyl]-2-(3-chloro-2-pyridinyl)-2-(4-methyl-1-piperazinyl)acetohydrazide (intermediate 76, 0.79 mmole), 117 μL of 1-iodo-3,5-bis(trifluoromethyl)benzene (0.66 mmole), 8.8 μL of trans 1,2-cyclohexanediamine (0.08 mmole), 7.5 mg of CuI (0.04 mmole, Aldrich) and 183 mg of K₂CO₃ (1.3 mmole) was added a 4/1 mixture of dioxane/dodecane (1 mL/235 μL). The reaction mixture was flushed with argon, and heated at 110° C. under argon for 2 h. It was cooled to RT and poured into H₂O. The organic layer was extracted with DCM, dried over Na₂SO₄, filtered and concentrated to dryness. The crude was purified on flash chromatography on silica gel cartridge using a gradient of DCM/MeOH 10/0 to 8/2 as an eluent. Solvents were removed under reduced pressure to give the title compound (30 mg, 7%). ¹H-NMR (400 MHz, CDCl₃): δ 2.31-2.35 (3H, m), 2.38-2.83 (8H, m), 4.97 (1H, s), 6.42 (1H, s), 7.25-7.40 (4H, m), 7.78 (1H, dd), 8.61 (1H, dd), 9.27 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., run time 4.5 min., flow rate: 2 mL/min]. Rₜ=1.62 min m/z (ES): 496 [M+H]⁺.

### Intermediate 78: (±)-N′-[3,5-bis(trifluoromethyl)phenyl]-2-(3-chloro-2-pyridinyl)-2-(4-methyl-1-piperazinyl)acetohydrazide-procedure B

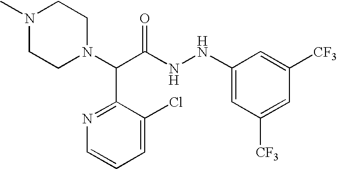

100 mg of potassium-(±)-(3-chloro-2-pyridinyl)(4-methyl-1-piperazinyl)carboxylate (intermediate 77, 0.3 mmole) were dissolved in 2 mL of DMF. 79 mg of 3,5-bis(trifluoromethyl)phenylhydrazine (0.34 mmole), 38 μL of NMM (0.34 mmole, Aldrich) and 288 mg of BOP (0.65 mmole., Fluka) were added and the mixture was stirred at RT for 5 h. Then AcOEt was added, and the organic phase was washed with a saturated solution of Na₂CO₃ first and then with brine, dried over Na₂SO₄, and concentrated under reduced pressure. The crude was purified by flash chromatography on silica gel (from DCM to DCM/MeOH 80/20) to give 63 mg of the desired product. This compound was re-dissolved in DCM and washed with a solution of NaOH 1N, dried over Na₂SO₄, and concentrated under reduced pressure to give the title compound (48 mg, 30%). ¹H-NMR (400 MHz, CDCl₃): δ 2.46-2.55 (3H, m), 2.60-2.95 (8H, m), 4.98 (1H, s), 6.48 (1H, s), 7.23-7.39 (4H, m), 7.79 (1H, dd), 8.61 (1H, dd), 9.29 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., run time 4.5 min., flow rate: 2 mL/min]: Rₜ=1.63 min m/z (ES): 496 [M+H]⁺.

### Compound 59: N′-[3,5-bis(trifluoromethyl)phenyl]-2-(3-chloro-2-pyridinyl)-2-(4-methyl-1-piperazinyl)acetohydrazide-enantiomer 1

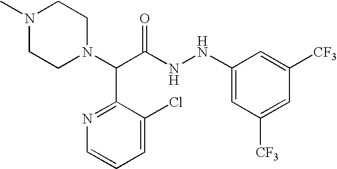

80 mg of (±)-N′-[3,5-bis(trifluoromethyl)phenyl]-2-(3-chloro-2-pyridinyl)-2-(4-methyl-1-piperazinyl)acetohydrazide (intermediate 78) were purified by preparative chiral HPLC [Chiralpak AD-H, 250×4.6 mm. Flow rate: 1 mL/min. UV detection: 200-400 nm. Mobile phase: A: n-Hex/B: EtOH+0.1% isopropylamine. Gradient: 18% B]: Rₜ=4.7 min. Solvents were removed under reduced pressure to give the title compound as a pink solid (27.8 mg, 68%). ¹H-NMR (400 MHz, CDCl₃): δ 2.26-2.96 (11H, m), 4.97 (1H, s), 6.41 (1H, s), 7.24-7.41 (4H, m), 7.76-7.80 (1H, m), 8.58-8.67 (1H, m), 9.28 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., run time 4.5 min., flow rate: 2 mL/min]: Rₜ=1.62 min m/z (ES): 496 [M+H]⁺.

### Compound 60: N′-[3,5-bis(trifluoromethyl)phenyl]-2-(3-chloro-2-pyridinyl)-2-(4-methyl-1-piperazinyl)acetohydrazide-enantiomer 2

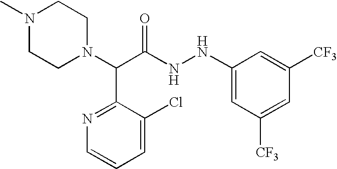

80 mg of (±)-N′-[3,5-bis(trifluoromethyl)phenyl]-2-(3-chloro-2-pyridinyl)-2-(4-methyl-1-piperazinyl)acetohydrazide (intermediate 78) were purified by semipreparative chiral HPLC [Chiralpak AD-H, 250×4.6 mm. Flow rate: 1 mL/min. UV detection: 200-400 nm. Mobile phase: A: n-Hex/B: EtOH+0.1% isopropylamine. Gradient: 18% B]: Rₜ=12.08 min. Solvents were removed under reduced pressure to give the title compound as a yellow solid (31.1 mg, 77%). ¹H-NMR (400 MHz, CDCl₃): δ 2.23-2.89 (11H, m), 4.97 (1H, s), 6.41 (1H, br s), 7.25-7.39 (4H, m), 7.78 (1H, dd), 8.63 (1H, dd), 9.27 (1H, br s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.61 min m/z (ES): 496 [M+H]⁺.

### Intermediate 79: ethyl(±)-bromo(pyridin-2-yl)acetate

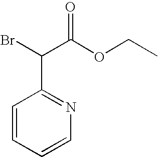

100 mg of benzoyl peroxide (0.41 mmole, Fluka) were added in one portion to a solution of 5 g of ethyl pyridin-2-ylacetate (30 mmole, Aldrich) and 5.36 g of NBS (30.28 mmole) in 30 mL of CCl₄. The mixture was heated at reflux for 5 h. The mixture was cooled to RT. The solid was filtered and the solvent was evaporated. The crude was purified using a SI cartridge (50 g) eluting with CH:AcOEt 95:5 for the first collected fraction and then CH: AcOEt 90:10. After removal of the solvents under reduced pressure, the title compound was obtained as a yellow oil (5.9 g, 80%). ¹H-NMR (400 MHz, CDCl₃): δ 1.31 (3H, t), 4.23-4.36 (2H, m), 5.53 (1H, s), 7.24-7.31 (1H, m), 7.69-7.81 (2H, m), 8.58 (1H, m); LC-MS (ES+) (see general experimental part for method): Rₜ=4.49 min m/z (ES): 244/246 [M+H]⁺ Br pattern.

### Intermediate 80: (±)-1,1-dimethylethyl 4-[2-(ethyloxy)-2-oxo-1-(2-pyridinyl)ethyl]-1-piperazinecarboxylate

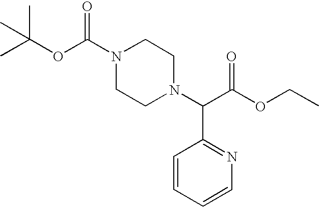

1.13 g of K₂CO₃ (8.19 mmole, Fluka) were added to a solution of 1 g of ethyl(±)-bromo(2-pyridinyl)acetate (intermediate 79, 4.09 mmole) in 4 mL of MeCN followed by 763 mg of tert-butyl-1-piperazine-carboxylate (4.09 mmole, Aldrich). The mixture was stirred overnight at RT, then the solids were filtered off and the filtrate was concentrated under reduced pressure. The crude material was purified by flash chromatography on silica gel (from CH to CH/AcOEt 40/60) to give the title compound (1.28 g, 89%). ¹H-NMR (400 MHz, CDCl₃): δ 1.24 (3H, t), 1.45 (9H, s), 2.56 (4H, br s), 3.47 (4H, br s), 4.14-4.30 (2H, m), 4.40 (1H, s), 7.22-7.27 (1H, m), 7.53 (1H, d), 7.68-7.75 (1H, m), 8.59 (1H, d); m/z (ES/+): 350 [M+H]⁺.

### Intermediate 81: (±)-potassium-(4-{[(1,1-dimethylethyl)oxy]carbonyl}-1-piperazinyl)(2-pyridinyl)carboxylate

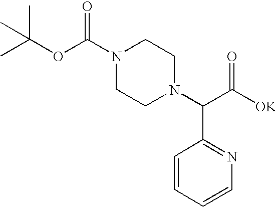

A solution of 209 mg of KOH (3.72 mmole, Fluka) in 4 mL of H₂O was added to a solution of 650 mg of (±)-1,1-dimethylethyl 4-[2-(ethyloxy)-2-oxo-1-(2-pyridinyl)ethyl]-1-piperazinecarboxylate (Intermediate 80, 1.86 mmole) in 5 mL of MeOH. The mixture was stirred at RT overnight, then concentrated under reduced pressure to give a crude compound that was dried again with toluene and THF dry to give the title compound (770 mg, quantitative) which was used in the next step without further purification. ¹H-NMR (400 MHz, DMSO-d₆): δ 1.37 (9H, s), 2.07-2.16 (2H, m), 2.39-2.48 (2H, m), 3.23-3.34 (4H, m), 3.56 (1H, s), 7.12-7.17 (1H, m), 7.48 (1H, d), 7.60-7.66 (1H, m), 8.35-8.41 (1H, m); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.48 min, m/z (ES): 322 [M+H]⁺.

### Intermediate 82: (±)-1,1-dimethylethyl 4-[2-{2-[3,5-bis(trifluoromethyl)phenyl]hydrazino}-2-oxo-1-(2-pyridinyl)ethyl]-1-piperazinecarboxylate

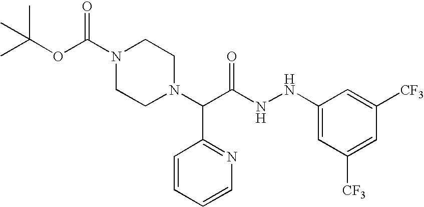

1.94 g of Py-BOP (3.7 mmole, Aldrich) were added to a suspension of 668 mg (1.7 mmole) of (±)-potassium-(4-{[(1,1-dimethylethyl)oxy]carbonyl}-1-piperazinyl)(2-pyridinyl)carboxylate (intermediate 81, 1.86 mmole), 215 μL of NMM (Aldrich) and 467 mg of 3,5-bis-(trifluoromethyl)phenylhydrazine (1.9 mmole, Aldrich) in 8 mL of DMF. The mixture was stirred at RT for 7 h. Then 37.5 mg of NMM (0.37 mmole), 3,5-bis-(trifluoromethyl)phenylhydrazine (100 mg, 0.4 mmole) and 210 mg of Py-BOP (0.4 mmole) were added and the reaction stirred overnight. The reaction mixture was diluted with AcOEt, the organic phase was washed with 1N NaOH (100 mL), a saturated solution of NaHCO₃ (100 mL) and finally with brine (100 mL). The organic phase was dried under reduced pressure and the crude was purified by flash chromatography (from CH to CH/AcOEt 40/60) to give the title compound (487 mg, 47%). ¹H-NMR (400 MHz, CDCl₃): 1.46-1.48 (9H, m), 2.37-2.58 (4H, m), 3.52 (4H, t), 4.15 (1H, s), 6.34 (1H, d), 7.22 (2H, s), 7.31-7.42 (3H, m), 7.72-7.79 (1H, m), 8.74 (1H, dd), 9.22 (1H, s).

### Intermediate 83: (±)-N′-[3,5-bis(trifluoromethyl)phenyl]-2-(1-piperazinyl)-2-(2-pyridinyl)acetohydrazide

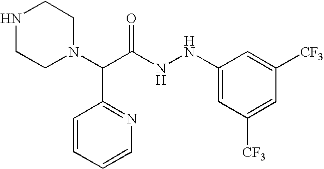

A solution of 3.4 mL of TFA in 18 mL of DCM was added dropwise to a stirred solution of 483 mg of (±)-1,1-dimethylethyl-4-[2-{2-[3,5-bis(trifluoromethyl)phenyl]hydrazino}-2-oxo-1-(2-pyridinyl)ethyl]-1-piperazinecarboxylate (intermediate 82, 0.833 mmole) in 4.4 mL of DCM previously cooled to 0° C. Then the reaction mixture was warmed at RT and stirred for 9 h. After evaporation of the volatiles under reduced pressure, DCM and a saturated solution of NaHCO₃ (100 mL) were added, the two phases were separated, the organic layer was dried over Na₂SO₄ and concentrated in vacuo. The crude was purified by flash chromatography on a SCX column (from DCM to NH₃ 0.5M in MeOH), to give the title compound (288 mg, 73%). ¹H-NMR (400 MHz, CDCl₃): δ 2.42-2.63 (4H, m), 3.01 (4H, br s), 4.15 (1H, s), 6.45 (1H, s), 7.20-7.25 (2H, m), 7.30-7.36 (2H, m), 7.39 (1H, d), 7.74-7.74 (1H, m), 8.73 (1H, d), 9.27 (1H, s).

### Compound 61: N′-[3,5-bis(trifluoromethyl)phenyl]-2-(1-piperazinyl)-2-(2-pyridinyl)acetohydrazide-enantiomer 1

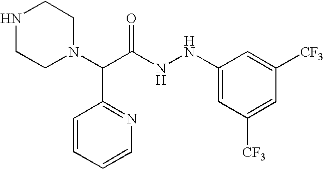

214 mg of (±)-N′-[3,5-bis(trifluoromethyl)phenyl]-2-(1-piperazinyl)-2-(2-pyridinyl)-acetohydrazide (intermediate 83) were purified by analytical chiral HPLC [Chiralpak AD-H, 250×4.6 mm. Flow rate: 0.8 mL/min. UV detection: 200-400 nm. Mobile phase: A: n-Hex/B: EtOH+0.1% IPA. Gradient: 20% B]: Rₜ=7.5 min. Solvents were removed under reduced pressure to give the title compound as a yellow solid (48.1 mg, 44%). ¹H-NMR (400 MHz, CDCl₃): δ 2.37-2.61 (4H, m), 2.90-3.03 (4H, m), 4.12 (1H, s), 6.45 (1H, s), 7.22 (2H, s), 7.29-7.36 (2H, m), 7.39 (1H, d), 7.69-7.78 (1H, m), 8.73 (1H, d), 9.25 (1H, s); LC-MS [Supelcosil ABZ+Plus, 33×4.6 mm, 3 μm, gradient: A: H₂O+0.1% HCOOH/B: MeCN: 0% B for 1 min. 0% to 95% B in 5 min., 95% B for 1.5 min., 95% to 0% B in 0.1 min., run time 8.5 min., flow rate: 1 mL/min]: Rₜ=4.15 min m/z (ES): 448 [M+H]⁺.

### Compound 62: N′-[3,5-bis(trifluoromethyl)phenyl]-2-(1-piperazinyl)-2-(2-pyridinyl)aceto-hydrazide-enantiomer 2

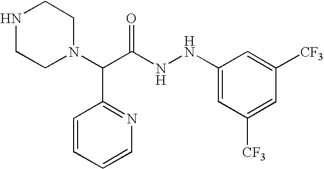

214 mg of (±)-N′-[3,5-bis(trifluoromethyl)phenyl]-2-(1-piperazinyl)-2-(2-pyridinyl)aceto-hydrazide (intermediate 83) were purified by analytical chiral HPLC [Chiralpak AD-H, 250×4.6 mm. Flow rate: 0.8 mL/min. UV detection: 200-400 nm. Mobile phase: A: n-Hex/B: EtOH+0.1% IPA. Gradient: 20% B]: Rₜ=9.4 min. Solvents were removed under reduced pressure to give the title compound as a white solid (41.7 mg, 38%). ¹H-NMR (400 MHz, CDCl₃): δ 2.38-2.62 (4H, m), 2.91-3.05 (4H, m), 4.13 (1H, s), 6.39 (1H, s), 7.23 (2H, s), 7.30-7.36 (2H, m), 7.39 (1H, d), 7.70-7.78 (1H, m), 8.73 (1H, d), 9.24 (1H, s); LC-MS [Supelcosil ABZ+Plus, 33×4.6 mm, 3 μm, gradient: A: H₂O+0.1% HCOOH/B: MeCN: 0% B for 1 min. 0% to 95% B in 5 min., 95% B for 1.5 min., 95% to 0% B in 0.1 min., run time 8.5 min., flow rate: 1 mL/min]: Rₜ=4.15 min m/z (ES): 448 [M+H]⁺.

### Intermediate 84: ethyl(4-methyl-2-pyridinyl)acetate

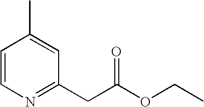

18.7 mL of LDA 1.8M (33.6 mmole, Aldrich) were added dropwise to a solution of 3.2 mL of 2,4-dimethyllutidine (28 mmole, Aldrich) and 4.2 mL of TMEDA (28 mmole, Aldrich) in 50 mL of dry THF previously cooled to −78° C. The reaction mixture was stirred for 1 h and then a solution of 2.7 mL of chloroethyl formate (28 mmole, Fluka) in 20 mL of dry THF was added. The solution was allowed to reach RT and then quenched with water and extracted with DCM. The organic phase was dried and concentrated in vacuo. The crude was purified by flash chromatography (CH/AcOEt 7:3) to give the title compound (1 g, 20%). ¹H-NMR (400 MHz, CDCl₃): δ 1.25 (3H, t), 2.35 (3H, s), 3.80 (2H, s), 4.20 (2H, q), 7.05 (1H, d), 7.15 (1H, s), 8.45 (1H, d).

### Intermediate 85: (±)-ethyl bromo(4-methyl-2-pyridinyl)acetate

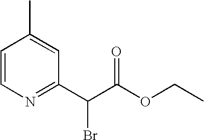

148 mg of Benzoylperoxide (0.61 mmole, Fluka) were added to a suspension of 548 mg of ethyl(4-methyl-2-pyridinyl)acetate (intermediate 84, 3.06 mmole) and 541 mg of NBS (3.04 mmole, Aldrich) in 16 mL of CCl₄. The reaction mixture was refluxed for 1 h at 85° C. Then, cyclohexane was added and solids were filtered off. Mother liquors were concentrated under reduced pressure and the crude was purified by flash chromatography on silica gel (from CH to CH/AcOEt 80/20) to give the title compound (220 mg, 27%). ¹H-NMR (400 MHz, CDCl₃): δ 1.31 (3H, t), 2.41 (3H, s), 4.24-4.35 (2H, m), 5.50 (1H, s), 7.06-7.12 (1H, m), 7.54 (1H, s), 8.43 (1H, d).

### Intermediate 86: (±)-ethyl(4-methyl-1-piperazinyl)(4-methyl-2-pyridinyl)acetate

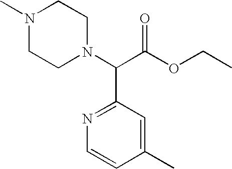

236 mg of K₂CO₃ (1.7 mmole, Fluka) were added to a solution of 220 mg of (±)-ethyl bromo(4-methyl-2-pyridinyl)acetate (intermediate 85, 0.853 mmole) in 8.5 mL of THF dry, and stirred for 5 min. Then 104 μL of N-methylpiperazine (0.94 mmole, Aldrich) were added dropwise. The reaction mixture was stirred at RT for 3 h. The organic layer was filtered and concentrated under reduced pressure. The crude was purified by flash chromatography (from DCM to DCM/MeOH 90/10) to give the title compound (150 mg, 63%). ¹H-NMR (400 MHz, CDCl₃): δ 1.23 (3H, t), 2.29 (3H, s), 2.37 (3H, s), 2.39-2.70 (8H, m), 4.12-4.29 (2H, m), 4.29 (1H, s), 7.00-7.09 (1H, m), 7.37 (1H, s), 8.43 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., run time 4.5 min., flow rate: 2 mL/min]: Rₜ=0.25 min; m/z (ES): 278 [M+H]⁺.

### Intermediate 87: (±)-potassium(4-methyl-1-piperazinyl)(4-methyl-2-pyridinyl)carboxylate

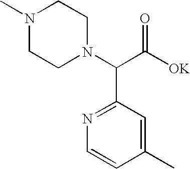

To a solution of 150 mg of (±)-ethyl(4-methyl-1-piperazinyl)(4-methyl-2-pyridinyl)acetate (intermediate 86, 0.541 mmole) in 5 mL of MeOH were added 60 mg of KOH (1.08 mmole, Fluka) in 1 mL of H₂O. The mixture was stirred at RT overnight. Then it was concentrated under reduced pressure and the crude was dried again with toluene to provide potassium(4-methyl-1-piperazinyl)(4-methyl-2-pyridinyl)carboxylate (170 mg, quantitative). ¹H-NMR (400 MHz, CDCl₃): δ 2.02-2.60 (14H, m), 3.46 (1H, s), 6.94-6.98 (1H, m), 7.31 (1H, s), 8.21 (1H, d); m/z (ES⁺): 250 [M+H]⁺.

### Intermediate 88: (±)-N′-[3,5-bis(trifluoromethyl)phenyl]-2-(4-methyl-1-piperazinyl)-2-(4-methyl-2-pyridinyl)acetohydrazide

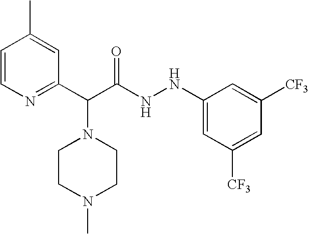

463 mg of BOP (1 mmol, Aldrich) were added to a stirred mixture of 150 mg of (±)-potassium(4-methyl-1-piperazinyl)(4-methyl-2-pyridinyl)carboxylate (intermediate 87, 0.523 mmole), 60 μL of NMM (0.55 mmole, Aldrich), 134 mg of 3,5-bis-(trifluoromethyl)phenylhydrazine (0.55 mmole, Aldrich) in 3 mL of dry DMF. The mixture was stirred at RT for 2 h. Then AcOEt was added and the organic layer was washed with 1N NaOH, saturated solution of NaHCO₃ and brine. The organic phase was evaporated under reduced pressure and the resulting crude material was purified by flash chromatography (from DCM to DCM/MeOH 8/2), to give the title compound (88 mg, 35%). ¹H-NMR (400 MHz, CDCl₃): δ 2.20-2.74 (14H, m), 4.05 (1H, s), 6.57 (1H, s), 7.06-7.42 (5H, m), 8.56 (1H, d), 9.25 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., run time 4.5 min., flow rate: 2 mL/min]: Rₜ=1.58 min; m/z (ES): 476 [M+H]⁺.

### Compound 63: N′-[3,5-bis(trifluoromethyl)phenyl]-2-(4-methyl-1-piperazinyl)-2-(4-methyl-2-pyridinyl)acetohydrazide-enantiomer 1

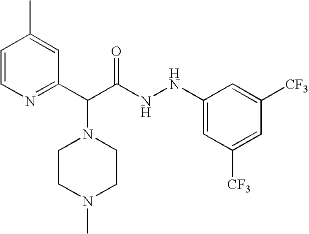

88 mg of racemic of (±)-N′-[3,5-bis(trifluoromethyl)phenyl]-2-(4-methyl-1-piperazinyl)-2-(4-methyl-2-pyridinyl)acetohydrazide (intermediate 88) were purified by analytical chiral HPLC [Chiralpak AD-H, 250×4.6 mm. Flow rate: 2.0 mL/min. UV detection: 210-340 nm. Mobile phase: 2-propanol+0.1% isopropylamine 12%. Rₜ=4.81 min. Solvents were removed under reduced pressure to give the title compound as a white solid (21.5 mg, 48%). ¹H-NMR (400 MHz, CDCl₃): 2.25-2.74 (14H, m), 4.05 (1H, s), 6.30 (1H, s), 7.12-7.35 (5H, m), 8.58 (1H, d), 9.20 (1H, s); LC-MS [Supelcosil ABZ+Plus, 33×4.6 mm, 3 μm, gradient: A: H₂O+0.1% HCOOH/B: MeCN: 0% B for 1 min. 0% to 95% B in 5 min., 95% B for 1.5 min., 95% to 0% B in 0.1 min., run time 8.5 min., flow rate: 1 mL/min]: Rₜ=4.20 min; m/z (ES): 476 [M+H]⁺.

### Compound 64: N′-[3,5-bis(trifluoromethyl)phenyl]-2-(4-methyl-1-piperazinyl)-2-(4-methyl-2-pyridinyl)acetohydrazide-enantiomer 2

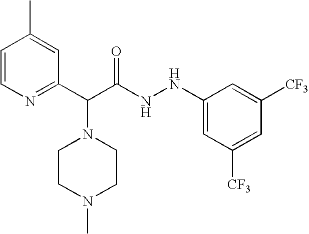

88 mg of racemic of (±)-N′-[3,5-bis(trifluoromethyl)phenyl]-2-(4-methyl-1-piperazinyl)-2-(4-methyl-2-pyridinyl)acetohydrazide (intermediate 88) were purified by analytical chiral HPLC [Chiralpak AD-H, 250×4.6 mm. Flow rate: 2.0 mL/min. UV detection: 210-340 nm. Mobile phase: 12% 2-propanol+0.1% isopropylamine]: Rₜ=7.15 min. Solvents were removed under reduced pressure to give the title compound as a white solid (22.6 mg, 50%). ¹H NMR (400 MHz, CDCl₃): δ 2.16-2.88 (14H, m), 4.04 (1H, s), 6.32 (1H, s), 7.11-7.35 (5H, m), 8.58 (1H, d), 9.20 (1H, s); LC-MS [Supelcosil ABZ+Plus, 33×4.6 mm, 3 μm, gradient: A: H₂O+0.1% HCOOH/B: MeCN: 0% B for 1 min. 0% to 95% B in 5 min., 95% B for 1.5 min., 95% to 0% B in 0.1 min., run time 8.5 min., flow rate: 1 mL/min]: Rₜ=4.20 min m/z (ES): 476 [M+H]⁺.

### Intermediate 89: (±)-diethyl(6-methyl-2-pyridinyl)propanedioate

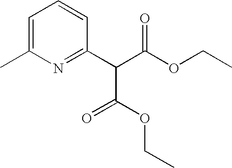

55.3 mL of LDA 1.8 M (99.6 mmole, Aldrich) were added dropwise to a solution of 9.6 mL of 2,6-dimethyllutidine (83 mmole, Aldrich) and 12.6 mL of TMEDA (83 mmole, Aldrich) in 150 mL of dry THF previously cooled to −78° C. The reaction mixture was stirred for 1 hour and then a solution of 8.1 mL of chloro ethylformate (83 mmole, Fluka) in 60 mL of dry THF was added. The solution was allowed to reach RT and was quenched with water and extracted with DCM. The organic phase was dried and concentrated in vacuo. The crude was purified by flash chromatography (CH/AcOEt 7:3) to give the title compound (1 g, 5%). ¹H-NMR (400 MHz, CDCl₃): δ 1.25 (6H, t), 2.55 (3H, s), 4.25 (4H, q), 4.9 (1H, s), 6.95 (1H, d), 7.12 (1H, d), 7.65 (1H, t); m/z (ES): 252 [M+H]⁺

### Intermediate 90: methyl(6-methyl-2-pyridinyl)acetate

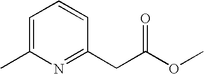

668 mg of potassium hydroxide (11.9 mmole) were added to a solution of 300 mg of intermediate 89 (1.19 mmole) in 40 mL of EtOH. The mixture was heated at reflux for 1 h, then it was allowed to cool to RT and concentrated in vacuo. The crude was passed trough a SCX column, the collected fractions were concentrated and the resulted potassium salt was dissolved in 5 mL of water, cooled to 0° C. and treated with HCl 1N. The solvent was removed in vacuo to give 160 mg of a crude that was dissolved in 8 mL of dry MeOH, then 850 μL of (trimethysilyl)diazomethane 2M in hexans (1.7 mmole, Aldrich) were added dropwise. After stirring for 3 h, further (trimethysilyl)diazomethane (1.7 mmole) was added and the reaction mixture was stirred for additional 2 h. Then it was quenched with acetic acid (glacial, Aldrich) and the solvents were evaporated in vacuo. The reaction was repeated using 360 mg of intermediate 20 and the combined crude were purified by flash chromatography (CH/AcOEt from 9:1 to 8:2) to give the title compound (160 mg, 35%). ¹H-NMR (400 MHz, CDCl₃): δ 2.55 (3H, s), 3.8 (3H, s), 3.9 (2H, s), 7.1 (2H, dd), 7.55 (1H, t); m/z (ES): 166 [M+H]⁺.

### Intermediate 91: (±)-methyl bromo(6-methyl-2-pyridinyl)acetate

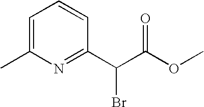

This compound was prepared in a similar manner as described for intermediate 85 starting from 160 mg of intermediate 90 (0.97 mmole) to give the title compound as a colourless oil (120 mg, 48%). ¹H-NMR (400 MHz, CDCl₃): δ 2.55 (3H, s), 3.8 (3H, s), 5.5 (1H, s), 7.1 (1H, d), 7.55 (1H, d), 7.65 (1H, t); m/z (ES): 244 [M]⁺.

### Intermediate 92: (±)-methyl(4-methyl-1-piperazinyl)(6-methyl-2-pyridinyl)acetate

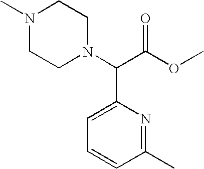

124 mg of potassium carbonate (0.9 mmole) were added to a solution of 110 mg of intermediate 91 (0.45 mmole) in 5 mL of THF and then 55 μL of N-methyl piperazine (0.5 mmole, Aldrich) were added dropwise and the reaction mixture was stirred for 2 h at RT. The solid was filtered off and mother liquors were concentrated in vacuo. The crude was passed trough a SCX column to give the title compound (115 mg, 97%). ¹H-NMR (400 MHz, CDCl₃): δ 1.6 (3H, m), 2.3 (3H, s), 2.55 (3H, s), 2.76 (5H, m), 3.8 (3H, s), 5.5 (1H, s), 7.1 (1H, d), 7.55 (1H, d), 7.65 (1H, t); m/z (ES): 264 [M+H]⁺.

### Intermediate 93: (±)-potassium (4-methyl-1-piperazinyl)(6-methyl-2-pyridinyl)acetate

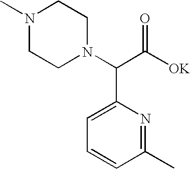

A solution of 32 mg of KOH (0.57 mmole, Fluka) in 0.2 mL of water was added to a solution of 115 mg of intermediate 92 (0.44 mmole) in 0.6 mL of MeOH and the mixture was stirred at RT overnight. Further KOH (0.1 mmole) in water (0.2 mL) was added and the mixture was stirred for 3 h and then concentrated under reduced pressure to give the title compound (150 mg, quantitative). ¹H-NMR (400 MHz, CDCl₃): δ 1.6 (3H, m), 2.3 (3H, s), 2.55 (3H, s), 2.76 (5H, m), 3.8 (3H, s), 5.5 (1H, s), 7.1 (1H, d), 7.55 (1H, d), 7.65 (1H, t); m/z (ES): 288 [M+K]⁺, 250 [M+H]⁺.

### Intermediate 94: (±)-N′-[3,5-bis(trifluoromethyl)phenyl]-2-(4-methyl-1-piperazinyl)-2-(6-methyl-2-pyridinyl)acetohydrazide

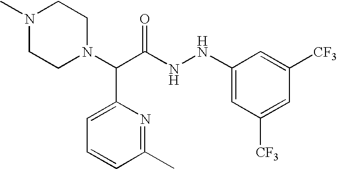

71.7 mg of intermediate 93 (0.25 mmole) were dissolved in 2 mL of DMF. 67 mg of 3,5-bis(trifluoromethyl)phenylhydrazine (0.27 mmole), 30 μL of NMM (0.27 mmole, Aldrich) and 122 mg of BOP (0.27 mmole, Fluka) were added and the mixture was stirred at RT for 1 h, then NaOH 1M and AcOEt were added, the organic phase was separated, dried and concentrated under reduced pressure. The crude was purified first by flash chromatography on SCX and then on silica gel (from DCM to DCM/NH₃ 0.5 M in MeOH 90/10) to give the title compound (24 mg, 20%). The reaction was repeated using 120 mg of intermediate 93 to give the title compound (76 mg). ¹H-NMR (400 MHz, CDCl₃): δ 2.35 (3H, s), 2.40-2.65 (8H, m), 2.65 (3H, s), 4.1 (1H, s), 6.5 (1H, br s), 7.2 (2H, m), 7.25 (2H, d), 7.65 (2H, t), 9.3 (1H, br s); m/z (ES): 476 [M+H]⁺.

### Compound 65: N′-[3,5-bis(trifluoromethyl)phenyl]-2-(4-methyl-1-piperazinyl)-2-(6-methyl-2-pyridinyl)acetohydrazide-enantiomer 1

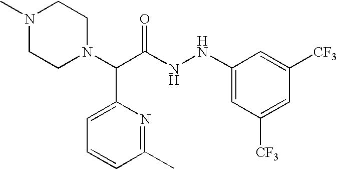

76 mg of intermediate 94 (±)-N′-[3,5-bis(trifluoromethyl)phenyl]-2-(4-methyl-1-piperazinyl)-2-(6-methyl-2-pyridinyl)acetohydrazide were separated by Chiral SFC [Chiralpak AS-H, 25×0.46 cm. Pressure: 100 bar. Flow rate: 2.0 mL/min. DAD: 210-340 nm. CD: 240 nm. 12% Modifier: EtOH+0.1% isopropylamine]: Rₜ=2.72 min. Solvents were removed under reduced pressure to give the title compound as a white solid (33 mg, 87%). ¹H-NMR (400 MHz, CDCl₃): δ 2.31 (3H, s), 2.4-2.7 (8H, br s), 2.65 (3H, s), 4.1 (1H, s), 6.35 (1H, br s), 7.15 (4H, m), 7.3 (1H, s), 7.55 (1H, t), 9.25 (1H, br s).

### Compound 66: N′-[3,5-bis(trifluoromethyl)phenyl]-2-(4-methyl-1-piperazinyl)-2-(6-methyl-2-pyridinyl)acetohydrazide-enantiomer 2

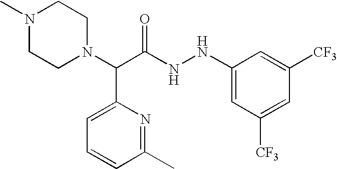

76 mg of the intermediate 94 (±)-N′-[3,5-bis(trifluoromethyl)phenyl]-2-(4-methyl-1-piperazinyl)-2-(6-methyl-2-pyridinyl)acetohydrazide were separated by Chiral SFC [Chiralpak AS-H, 25×0.46 cm. Pressure: 100 bar. Flow rate: 2.0 mL/min. DAD: 210-340 nm. CD: 240 nm. 12% Modifier: EtOH+0.1% isopropylamine]: Rₜ=4.67 min. Solvents were removed under reduced pressure to give the title compound as a white solid (28 mg, 74%). m/z (ES): 476 [M+H]⁺

### Intermediate 95: (±)-ethyl(4-methyl-1-piperazinyl)(2-pyridinyl)acetate

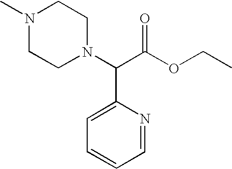

To a solution of 2.20 g of ethyl 2-pyridinylacetate (13.3 mmole.) in 30 mL of CCl₄ were added at RT, 2.37 g of NBS (13.3 mmole, Aldrich). The reaction mixture was stirred at reflux and under nitrogen atmosphere for 2 h and then it was allowed to cool to RT and 3.7 mL of N-methylpiperazine (33.3 mmole, Aldrich) were added. The solution was heated at 75° C. for 1 h. Solvents were removed under reduced pressure and the resulting crude was purified by flash chromatography on a 50 g silica gel cartridge using a gradient of DCM/MeOH 10/0 to 9/1 as an eluent. Solvents were removed to give the title compound as a brown oil (3.5 g, quantitative). ¹H-NMR (400 MHz, DMSO-d₆): δ 1.14 (3H, t), 2.15 (3H, s), 2.39 (8H, m), 4.10 (2H, m), 4.46 (1H, s), 7.34 (1H, ddd), 7.49 (1H, dt), 7.83 (1H, td), 8.50 (1H, ddd); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.39 min, m/z (ES): 264 [M+H]⁺.

### Intermediate 96: potassium (±)-(4-methyl-1-piperazinyl)(2-pyridinyl)acetate

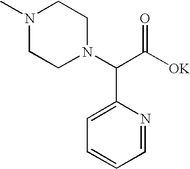

To a solution of 3.5 g of (±)-ethyl(4-methyl-1-piperazinyl)(2-pyridinyl)acetate (Intermediate 95, 13.3 mmole) in 30 mL of MeOH was added a solution of 2.24 g of KOH (39.9 mmoles) in water (10 mL). The mixture was stirred at RT overnight. Solvents were removed under reduced pressure to give the title compound (6 g, quantitative). ¹H-NMR (400 MHz, DMSO-d₆): 2.09 (3H, s), 2.32 (8H, m), 3.50 (1H, s), 7.12 (1H, dd), 7.47 (1H, d), 7.60 (1H, m), 8.34 (1H, d).

### Intermediate 97: (±)-N′-[3,5-bis(trifluoromethyl)phenyl]-2-(4-methyl-1-piperazinyl)-2-(2-pyridinyl)acetohydrazide

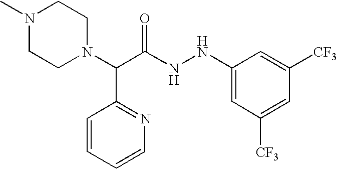

A mixture of 400 mg of potassium (±)-(4-methyl-1-piperazinyl)(2-pyridinyl)acetate (intermediate 96, 1.70 mmole), 240 mg of HOBT.H₂O (1.70 mmole), 300 mg of EDC.HCl (1.70 mmole) and 0.3 mL of DIPEA, in 5 mL of anhydrous DCM was stirred at RT under N₂ atmosphere for 30 min. 415 mg of 3,5-bis(trifluoromethylphenyl)hydrazine (1.70 mmole) were added to the mixture. The suspension was stirred at RT for 2 days. Solvents were removed under reduced pressure and the crude was purified first by SCX cartridge then by flash chromatography on silica gel cartridge using a gradient of DCM/MeOH 10/0 to 8/2 as an eluent. Solvents were removed to give the title compound as an orange solid (60 mg, 13%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.59 min, m/z (ES): 462 [M+H]⁺.

### Compound 67: N′-[3,5-bis(trifluoromethyl)phenyl]-2-(4-methyl-1-piperazinyl)-2-(2-pyridinyl)acetohydrazide-enantiomer 1

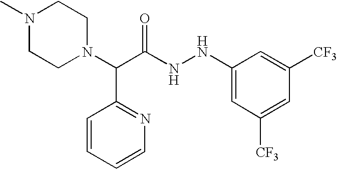

60 mg of intermediate 97 were separated by Chiral HPLC [Chiralpak AD-H, 250×4.6 mm. Flow rate: 1.0 mL/min. UV detection: DAD at 225 nm. Mobile phase: A: n-Hex/B: 2-Propanol 85:15]: Rₜ=4.9 min. Solvents were removed under reduced pressure to give the title compound as a white solid (24 mg, 80%). m/z (ES): 461 [M]⁺

### Compound 68: N′-[3,5-bis(trifluoromethyl)phenyl]-2-(4-methyl-1-piperazinyl)-2-(2-pyridinyl)acetohydrazide-enantiomer 2

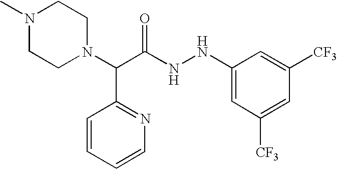

60 mg of intermediate 97 were purified by Chiral HPLC [Chiralpak AD-H, 250×4.6 mm. Flow rate: 1.0 mL/min. UV detection: DAD at 225 nm. Mobile phase: A: n-Hex/B: 2-Propanol 85:15]: Rₜ=7.9 min. Solvents were removed under reduced pressure to give the title compound as a white solid (17 mg, 57%). m/z (ES): 461 [M+H]⁺.

### Intermediate 98: 1,1-dimethylethyl 4-[1,3-benzodioxol-5-yl(cyano)methylidene]-1-piperidinecarboxylate

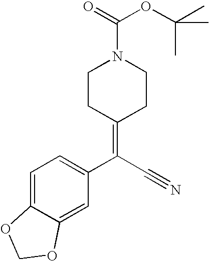

288 mg of sodium (12.5 mmole, 99% grade Aldrich) were added to 10 mL of dry MeOH under nitrogen atmosphere. After complete dissolution, a solution of 3 g of 1,1-dimethylethyl 4-oxo-1-piperidinecarboxylate (15 mmole, Aldrich) and 2.01 g of 1,3-benzodioxol-5-ylacetonitrile (12.5 mmole, Aldrich) in 10 mL of dry MeOH was added dropwise. When the addition was completed the reaction mixture was heated to reflux overnight. The solution was allowed to reach RT and concentrated in vacuo to eliminate the MeOH. At 0° C., AcOEt and water were added and the organic layer was separated, dried and concentrated in vacuo. The resulting crude was then purified by flash chromatography (CH/AcOEt 8:2) to give the title compound (3.4 g, 79%). ¹H-NMR (400 MHz, CDCl₃): δ 1.5 (9H, s), 2.4 (2H, br s), 2.7 (2H, br s), 3.4 (2H, br s), 3.6 (2H, br s), 6.0 (2H, s), 6.75-6.85 (3H, m); m/z (ES): 366 [M+Na]⁺, 287 [M-56]⁺, 243 [M-Boc]⁺.

### Intermediate 99: (±)-1,1-dimethylethyl 4-[1,3-benzodioxol-5-yl(cyano)methyl]-1-piperidinecarboxylate

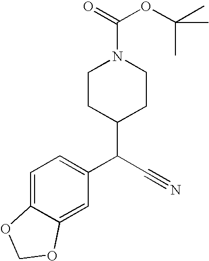

To a solution of 1.5 g of 1,1-dimethylethyl 4-[1,3-benzodioxol-5-yl(cyano)methylidene]-1-piperidinecarboxylate (intermediate 98, 4.38 mmole) in 10 mL of isopropyl alcohol, 332 mg of NaBH₄ (8.76 mmole, Aldrich) were added portionwise at 40° C. under a nitrogen atmosphere. When the addition was completed the reaction mixture was heated to reflux overnight and then allowed to reach RT. Water was added and the aqueous phase was extracted with DCM. The organic layer was dried and concentrated in vacuo giving the title compound (1.55 g, quantitative). ¹H-NMR (400 MHz, CDCl₃): δ 1.2-1.4 (4H, m), 1.50 (9H, s), 1.85 (2H, m), 2.65 (2H, m), 4.2 (2H, m), 6.0 (2H, s), 6.75-6.85 (3H, m).

### Intermediate 100: (±)-1,3-benzodioxol-5-yl(4-piperidinyl)acetonitrile

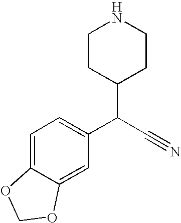

32 mL of TFA (Aldrich) were added to a solution of 1.45 g of (±)-1,1-dimethylethyl 4-[1,3-benzodioxol-5-yl(cyano)methyl]-1-piperidinecarboxylate (Intermediate 99, 4.22 mmole) in 40 mL of anhydrous DCM previously cooled to 0° C. under a nitrogen atmosphere. The solution was stirred for 2 h at RT, then it was concentrated in vacuo. The residue was dissolved in DCM and washed with a saturated sodium hydrogen carbonate solution. The aqueous layer was extracted with further DCM. The organic extract was dried and concentrated in vacuo. The crude was purified by flash chromatography (from CH/AcOEt 1:1 to DCM/NH₃ 0.5M in MeOH 9:1) to give the title compound (680 mg, 66%). ¹H-NMR (400 MHz, CDCl₃): δ 1.20-1.50 (2H, m), 1.60 (1H, d), 1.80-2.00 (3H, m), 2.60 (2H, m), 3.15 (2H, m), 3.50 (1H, d), 6.00 (2H, s), 6.75 (3H, m).

### Intermediate 101: (±)-1,3-benzodioxol-5-yl(1-methyl-4-piperidinyl)acetonitrile

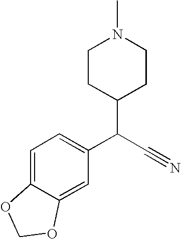

417 μL of formaldehyde in water (37% w/w; 5.6 mmole, Aldrich) were added to a stirred solution of 680 mg of (±)-1,3-benzodioxol-5-yl(4-piperidinyl)acetonitrile (intermediate 100, 2.8 mmole) in 7 mL of MeCN under a nitrogen atmosphere. After 30 min, 890 mg of sodium triacetoxyborohydride (4.2 mmole, Aldrich) were added. The mixture was stirred for further 4 h then it was quenched with a saturated sodium hydrogen carbonate solution and extracted with DCM. The organic phase was dried and concentrated in vacuo. The crude was purified by flash chromatography (from DCM to DCM/MeOH 8:2) to give the title compound (660 mg, 94%). ¹H-NMR (400 MHz, CDCl₃): δ 1.30-2.00 (7H, m), 2.25 (3H, s), 2.90 (2H, dd), 3.50 (1H, d), 6.00 (2H, s), 6.80 (3H, m); m/z (ES): 259 [M+H]⁺.

### Intermediate 102: (±)-1,3-benzodioxol-5-yl(1-methyl-4-piperidinyl)acetic acid hydrochloride

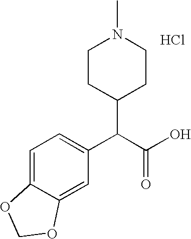

1.3 mL of a 5M aqueous solution of KOH were added to a solution of 335 mg of (±)-1,3-benzodioxol-5-yl(1-methyl-4-piperidinyl)acetonitrile (intermediate 101, 1.3 mmole) in 6 mL of ethylene glycol. The reaction mixture was heated at 130° C. for 5 days. Then it was cooled to 0° C. and the solution was acidified with HCl 6N to pH 1-2. The water was evaporated and the ethylene glycol distilled by kugelrohr (high vacuum and T=160° C.). The residue was purified by HLB-LP cartridge (Waters Oasis® HLB 35 cc (6 g) LP Extraction Cartridges) (from H₂O to H₂O/MeOH 6:4) to give the title compound (256 mg, 63%). ¹H-NMR: (400 MHz, DMSO-d₆): δ 1.10-1.60 (3H, m), 1.60-2.00 (2H, m), 2.40 (3H, s), 2.90-3.80 (5H, m), 6.00 (2H, s), 6.85 (1H, dd), 6.95 (2H, m), 11.3 (1H, br s).

### Compound 69: (±)-2-(1,3-benzodioxol-5-yl)-N′-(3,5-dichlorophenyl)-2-(1-methyl-4-piperidinyl)acetohydrazide

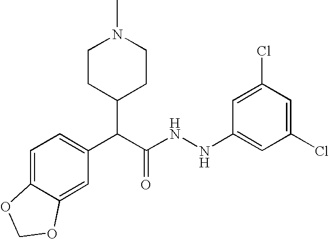

308 μL of DIPEA (1.72 mmole, Aldrich) and 181 mg of TBTU (0.56 mmole, Fluka) were added to a solution of 120 mg of (±)-1,3-benzodioxol-5-yl(1-methyl-4-piperidinyl)acetic acid hydrochloride (intermediate 102, 0.43 mmole) in 3 mL of anhydrous DCM under a nitrogen atmosphere. After stirring for 10 minutes, 91 mg of 3,5-(dichlorophenyl)hydrazine (0.52 mmole, Lancaster) were added. The solution was stirred at RT overnight, then it was diluted with water. The organic layer was dried, concentrated in vacuo and the residue was purified twice by flash chromatography (from DCM to DCM/NH₃ 0.5 M in MeOH 8:2) to give a first batch of the pure title compound as racemate (23 mg). A second batch was further purified by Fractionlynx™ (method D) to give additional 20 mg of the title compound as white solid (43 mg, 23%). ¹H-NMR (400 MHz, DMSO-d₆): δ 0.80-1.47 (3H, m), 1.54-2.09 (3H, m), 2.10 (3H, s), 2.63 (2H, dd), 3.20 (1H, d), 6.00 (2H, d), 6.40 (2H, s), 6.62-7.05 (4H, m), 8.2 (1H, s), 10.00 (1H, s); m/z (ES): 436 [M]⁺.

### Compound 70: 2-(1,3-benzodioxol-5-yl)-N′-(3,5-dichlorophenyl)-2-(1-methyl-4-piperidinyl)-acetohydrazide-enantiomer 1

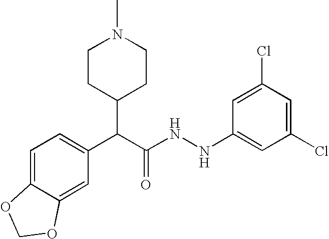

20 mg of compound 69 were purified by Chiral HPLC [Chiralpak AD-H, 250×4.6 mm. Flow rate: 0.8 mL/min. UV detection: 200-400 nm. Mobile phase: A: n-Hex/B: EtOH. Gradient: isocratic 30% B]: Rₜ=13.7 min. Solvents were removed under reduced pressure to give the title compound as a white solid (3.8 mg, 40%). m/z (ES): 436 [M+H]⁺

### Compound 71: 2-(1,3-benzodioxol-5-yl)-N′-(3,5-dichlorophenyl)-2-(1-methyl-4-piperidinyl)-acetohydrazide-enantiomer 2

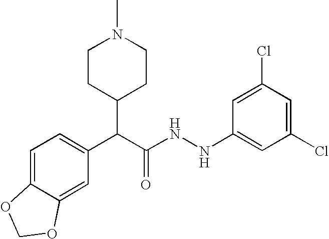

20 mg of compound 69 were purified by Chiral HPLC [Chiralpak AD-H, 250×4.6 mm. Flow rate: 0.8 mL/min. UV detection: 200-400 nm. Mobile phase: A: n-Hex/B: EtOH. Gradient: isocratic 30% B]: Rₜ=28.9 min. Solvents were removed under reduced pressure to give the title compound as a white solid (10 mg, 100%). m/z (ES): 436 [M+H]⁺

### Intermediate 103: 1,1-dimethylethyl 4-[cyano(1-naphthalenyl)methylidene]-1-piperidinecarboxylate

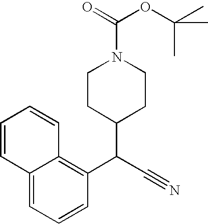

288 mg of sodium (12.5 mmole, 99% grade Aldrich) were added to 10 mL of dry MeOH under nitrogen atmosphere. After complete dissolution, a solution of 3 g of 1,1-dimethylethyl 4-oxo-1-piperidinecarboxylate (15 mmole, Aldrich) and 2 g of 1-naphthalenylacetonitrile (12.5 mmole, Aldrich) in 20 mL of dry MeOH were added dropwise. When the addition was completed the reaction mixture was heated to reflux overnight. The solution was allowed to reach RT and concentrated in vacuo to eliminate the MeOH. At 0° C., AcOEt and water were added and the organic layer was separated, dried and concentrated in vacuo. The resulting crude was then purified by flash chromatography (Horizon, columns 65M Si) using CH/AcOEt (from 9:1 to 8:2) as an eluent to give the title compound (3.1 g, 72%). ¹H-NMR (400 MHz, CDCl₃): δ 1.50 (9H, s), 2.15 (2H, m), 2.90 (2H, m), 3.35 (2H, m), 3.75 (2H, m), 7.50-7.70 (4H, m), 7.80-8.00 (3H, m); m/z (ES): 349 [M+H]⁺, 293 [M-55]⁺, 249 [M-Boc]⁺.

### Intermediate 104: (±)-1-naphthalenyl(4-piperidinyl)acetonitrile

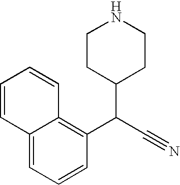

To a solution of 300 mg of (±)-1,1-dimethylethyl 4-[cyano(1-naphthalenyl)methylidene]-1-piperidinecarboxylate (intermediate 103, 0.86 mmole) in 2 mL of IPA, 65 mg of NaBH₄ (1.72 mmole, Aldrich) were added portionwise at 40° C. under nitrogen atmosphere. When the addition was completed the reaction mixture was heated to reflux overnight and then allowed to reach RT. Water was added and the aqueous phase was extracted with DCM. The organic layer was dried and concentrated in vacuo giving 300 mg of a solid which was dissolved in 2 mL of IPA, then 65 mg of NaBH₄ (1.72 mmole, Aldrich) were added portionwise at 40° C. under nitrogen atmosphere. When the addition was completed the reaction mixture was heated to reflux overnight and then allowed to reach RT. Water was added and the aqueous phase was extracted with DCM. The organic layer was dried and concentrated in vacuo 5 mL of DCM were added, followed by 5 mL of TFA (Aldrich) at 0° C. and under a nitrogen atmosphere. The solution was stirred for 2 h at RT, then it was concentrated in vacuo. The residue was dissolved in DCM and washed with saturated sodium hydrogen carbonate solution. The aqueous layer was extracted with further DCM. The organic extract was dried and concentrated in vacuo. The crude was purified by flash chromatography (from CH/AcOEt 1:1 to DCM/NH₃ 0.5M in MeOH 9:1) to give the title compound (200 mg, quantitative). ¹H-NMR (400 MHz, CDCl₃): δ 1.50-2.50 (6H, m), 2.70 (2H, m), 3.35 (2H, dd), 4.40 (1H, d), 7.50-7.70 (4H, m), 7.80-8.00 (3H, m); m/z (ES): 251 [M+H]⁺.

### Intermediate 105: (±)-(1-methyl-4-piperidinyl)(1-naphthalenyl)acetonitrile

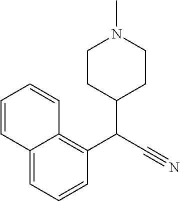

119 μL of formaldehyde in water (37% w/w; 1.6 mmole, Aldrich) were added to a stirred solution of 200 mg of (±)-1-naphthalenyl(4-piperidinyl)acetonitrile (intermediate 104, 0.8 mmole) in 2 mL of MeCN under a nitrogen atmosphere. After 30 min, 255 mg of sodium triacetoxyborohydride (1.2 mmole, Aldrich) were added. The mixture was stirred for further 4 h then it was quenched with a saturated sodium hydrogen carbonate solution and extracted with DCM. The organic phase was dried and concentrated in vacuo to give the title compound (170 mg, 80%). ¹H-NMR (400 MHz, CDCl₃): δ 1.50-2.10 (7H, m), 2.25 (3H, s), 2.90 (2H, dd), 4.40 (1H, d), 7.50-7.70 (4H, m), 7.80-8.00 (3H, m); m/z (ES): 265 [M+H]⁺.

### Intermediate 106: (±)-(1-methyl-4-piperidinyl)(1-naphthalenyl)acetic acid hydrochloride

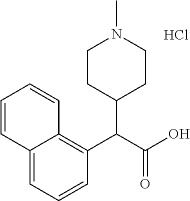

200 mg of (±)-(1-methyl-4-piperidinyl)(1-naphthalenyl)acetonitrile (intermediate 105, 0.76 mmole) dissolved in 5.4 mL of HCl conc were heated to reflux. Then it was concentrated in vacuo and the residue was triturated with Et₂O to give the title compound (240 mg, quantitative) as white solid. ¹H-NMR (400 MHz, CDCl₃): δ 1.17 (1H, m), 1.33 (1H, m), 1.76 (1H, m), 2.07 (1H, d), 2.35 (1H, m), 2.62 (3H, d), 2.78 (1H, m), 2.97 (1H, m), 3.18 (1H, d), 3.41 (1H, d), 4.18 (1H, d), 7.55 (4H, m), 7.86 (1H, d), 7.96 (1H, d), 8.25 (1H, d), 10.24 (1H, br s).

### Compound 72: (±)-N′-(3,5-dichlorophenyl)-2-(1-methyl-4-piperidinyl)-2-(1-naphthalenyl)-acetohydrazide

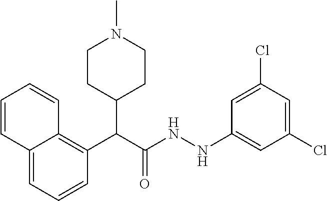

215 μL of DIPEA (1.2 mmole, Aldrich) and 166 mg of TBTU (0.52 mmole, Fluka) were added to a solution of 127 mg of (±)-(1-methyl-4-piperidinyl)(1-naphthalenyl)acetic acid hydrochloride (intermediate 106, 0.4 mmole) in 2 mL of anhydrous DCM under a nitrogen atmosphere. After stirring for 10 min, 85 mg of 3,5-(dichlorophenyl)hydrazine (0.48 mmole, Lancaster) were added. The solution was stirred at RT overnight, then it was diluted with water. The organic layer was dried, concentrated in vacuo and the residue was purified by flash chromatography (from DCM to DCM/NH₃ 0.5 M in MeOH 8:2) to give 62 mg that were further purified by Fractionlynx™ (LC2 ultra) to give the title compound racemate as a white foam (27 mg, 15%). ¹H-NMR (400 MHz, DMSO-d₆): δ 1.06 (2H, m), 1.51 (1H, m), 1.70 (1H, t), 1.80 (1H, m), 1.89 (1H, t), 2.10 (4H, m), 2.57 (1H, m), 2.82 (1H, m), 4.16 (1H, d), 6.37 (2H, m), 6.72 (1H, m), 7.51 (2H, m), 7.61 (1H, m), 7.68 (1H, d), 7.84 (1H, d), 7.95 (1H, d), 8.37 (1H, m), 8.45 (1H, d), 10.12 (1H, m).

### Compound 73: N′-(3,5-dichlorophenyl)-2-(1-methyl-4-piperidinyl)-2-(1-naphthalenyl)-acetohydrazide-enantiomer 1

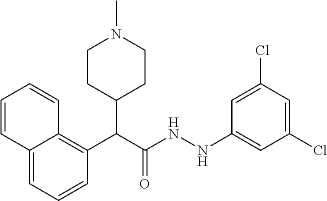

20 mg of (±)-N′-(3,5-dichlorophenyl)-2-(1-methyl-4-piperidinyl)-2-(1-naphthalenyl)-acetohydrazide (compound 72) were purified by semipreparative Chiral HPLC [Chiralpak AD-H, 250×4.6 mm. Flow rate: 0.8 mL/min. UV detection: 200-400 nm. Mobile phase: A: n-Hex/B: EtOH+0.1% isopropylamine. Gradient: isocratic 30% B]: Rₜ=9.8 min. Solvents were removed under reduced pressure to give the title compound (4 mg, 40%).

### Compound 74: N′-(3,5-dichlorophenyl)-2-(1-methyl-4-piperidinyl)-2-(1-naphthalenyl)-acetohydrazide-enantiomer 2

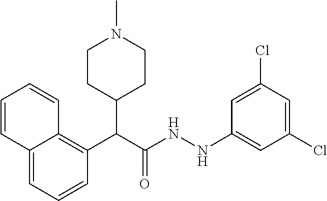

20 mg of (±)-N′-(3,5-dichlorophenyl)-2-(1-methyl-4-piperidinyl)-2-(1-naphthalenyl)-acetohydrazide (compound 72) were purified by semipreparative Chiral HPLC [Chiralpak AD-H, 250×4.6 mm. Flow rate: 0.8 mL/min. UV detection: 200-400 nm. Mobile phase: A: n-Hex/B: EtOH+0.1% isopropylamine. Gradient: isocratic 30% B]: Rₜ=13.1 min. Solvents were removed under reduced pressure to give the title compound as a white solid (10 mg, 100%).

### Compound 75: (+)-N′-[3,5-bis(trifluoromethyl)phenyl]-2-(2-chlorophenyl)-2-(1-piperazinyl)acetohydrazide hydrochloride-enantiomer 2

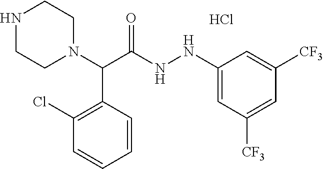

218 mg of N′-[3,5-bis(trifluoromethyl)phenyl]-2-(2-chlorophenyl)-2-(1-piperazinyl)-acetohydrazide enantiomer 2 (Compound 27, 0.45 mmole) were dissolved in 5 mL of dry DCM in a dry flask under nitrogen. Then 450 μL of HCl 1M solution in Et₂O (0.45 mmole, Aldrich) were added. The solution was then stirred for 30 min. The solvents were removed under reduced pressure and the resulting crude solid was triturated with Et₂O. The solid was filtered, dried under reduced pressure to give the title compound as a sand color solid (196 mg, 84%). ¹H-NMR (400 MHz, DMSO-d₆): δ 2.67 (4H, m), 3.12 (4H, m), 4.65 (1H, s), 7.12 (1H, s), 7.31 (1H, s), 7.40 (1H, m), 7.51 (1H, m), 7.69 (1H, m), 8.80 (2H, br s), 10.54 (1H, s); Chiral HPLC [Chiralpak AS-H, 25×4.6 cm, n-Hex/EtOH+0.1% isopropylamine 85/15; flow rate: 1 mL/min; DAD=225 nm, CD=245 nm]: Rₜ=8.55 min. (ee=99%). [α]_{D}=40.17 de.g., concentration: 1.0325% Weight/Volume.

### Compound 76: (+)-N′-[3,5-bis(trifluoromethyl)phenyl]-2-(2-chlorophenyl)-2-(4-methyl-1-piperazinyl)acetohydrazide hydrochloride-enantiomer 1

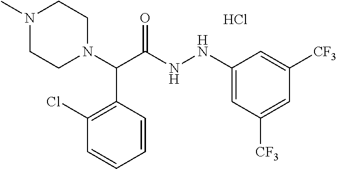

To a solution of 10.1 g of compound 9 N′-[3,5-bis(trifluoromethyl)phenyl]-2-(2-chlorophenyl)-2-(4-methyl-1-piperazinyl)acetohydrazide-enantiomer 1 (obtained from preparative chiral HPLC) in 100 mL of Et₂O at 0° C. under N₂, 20.4 mL of a 1M HCl solution in Et₂O (Aldrich) were added in about 15 min. A white precipitate was formed immediately and the slurry was left stirring at 0° C. for 1 h. 30 mL of pentane were then added and the solid recovered by filtration in a dry box apparatus. The collected material was dried at high vacuum overnight at room temperature to get 9.5 g (17.8 mmol, 87%) of the title compound as an off white solid. ¹H-NMR (600 MHz, DMSO-d₆): δ 2.45 (1H, m), 2.74 (3H, d), 2.76 (2H, m), 3.04 (3H, m), 3.33 (1H, m), 3.42 (1H, m), 4.64 (1H, s), 7.11 (2H, br s), 7.29 (1H, br s), 7.38 (2H, m), 7.52 (1H, m), 7.71 (1H, m), 8.82 (1H, br s), 10.51 (1H, br s), 10.58 (1H, br s); chiral HPLC conditions: column: Chiralpak AD-H (25×4.6 cm), mobile phase: n-Hex/EtOH+0.1% isopropylamine 95/5, flow: 1 mL/min, detector: CD=245 nm, Rₜ: 8.93 min, ee=99.5%. [α]_{D}=37.37 de.g., concentration: 1.0320% Weight/Volume.

### Intermediate 107: Ethyl(±)-(2,6-dimethyl-3-pyridinyl)(4-methyl-1-piperazinyl)acetate

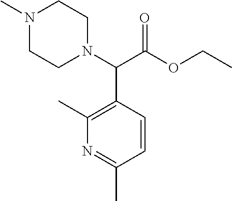

To 2.85 mL of iPrMgCl.LiCl 2.0M in THF (5.37 mmole) (prepared according to the procedure of Krasovskiy A. and Knochel P., Angew. Chem. Int. Ed., 2004, 43, 3333-3336) diluted in 3.05 mL of dry THF and cooled at 0° C., was added 1 g of 3-bromo-2,6-dimethylpyridine (5.37 mmole, Fluka). The mixture was allowed to reach RT and was stirred for 30 min under nitrogen atmosphere. Then, 2.15 mL of ethyl glyoxylate 50% in toluene (10.8 mmole, Fluka) were added. The solution was stirred at RT for 2 h. The reaction was quenched with water. AcOEt was added and the precipitate was filtered on celite. The cake was washed with AcOEt. The biphasic solution was transferred to a separatory funnel and the organic phase was separated. The organic layers were washed with brine, dried over Na₂SO₄, filtered and evaporated to dryness to give 1.2 g of a crude. 320 mg of this crude were dissolved in 20 mL of DCM. 250 μL of TEA (1.83 mmole, Aldrich) were added, then the solution was cooled at 0° C. and 130 μL of mesylchloride (1.1.68 mmole, Aldrich) were added dropwise. The solution was stirred at 0° C. for 0.5 h then 370 μL of N-methylpiperazine (3.34 mmole, Aldrich) were added. The mixture was allowed to reach RT and was stirred for 16 h. 20 mL of AcOEt were added and the organic phase was washed with 20 mL of water, dried over Na₂SO₄, filtered and evaporated to dryness. The crude was purified by Flashmaster (eluent: AcOEt/MeOH with 5% of NH₃ 0.5 M in water 9:1 to 7:3) to give the title compound as a yellow oil (280 mg, 59%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.32 min, m/z (ES): 292.18 [M+H]⁺.

### Intermediate 108: (±)-N′-[3,5-bis(trifluoromethyl)phenyl]-2-(2,6-dimethyl-3-pyridinyl)-2-(4-methyl-1-piperazinyl)acetohydrazide

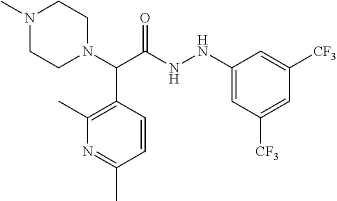

To a solution of 280 mg of ethyl(±)-(2,6-dimethyl-3-pyridinyl)(4-methyl-1-piperazinyl)acetate (intermediate 107, 0.96 mmole) in 5 mL of MeOH and 1 mL of water, were added 65 mg of KOH (1.16 mmole). The mixture was stirred at RT for 16 h. Solvents were removed under reduced pressure and the solid thus obtained was dissolved in dry THF (15 mL). To this solution were added successively 283 mg of 3,5-bis(trifluoromethyl)phenylhydrazine (1.16 mmole, Alfa Aesar), 723 μL of DIPEA (3.9 mmole, Aldrich) and 551 mg of PyBOP (1.06 mmole, Fluka). The mixture was stirred at RT for 24 h. Solvents were removed under reduced pressure and the residue was dissolved in DCM. The organic phase was washed with a saturated solution of NaHCO₃, dried over Na₂SO₄, filtered and evaporated to dryness. The crude was dissolved in DCM and the solution was passed through a silica cartridge. The cartridge was eluted with DCM/MeOH with 1% of aqueous 0.5 M ammonia solution 9:1). Solvents were removed to give 28 mg of the title compound (6%). UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 mL/min]: Rₜ=0.54 min, m/z (ES): 490.03 [M+H]⁺.

### Compound 77: N′-[3,5-bis(trifluoromethyl)phenyl]-2-(2,6-dimethyl-3-pyridinyl)-2-(4-methyl-1-piperazinyl)acetohydrazide-enantiomer 2

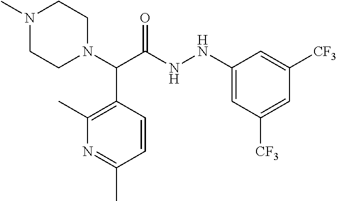

28 mg of (±)-N′-[3,5-bis(trifluoromethyl)phenyl]-2-(2,6-dimethyl-3-pyridinyl)-2-(4-methyl-1-piperazinyl)acetohydrazide (intermediate 108, 0.06 mmole) were purified by semipreparative chiral SFC [Chiralpak AS-H, 25×2.1 cm. Pressure: 160 bar. Flow rate: 22 mL/min. UV detection: 220 nm. Injection: 20 mg each in EtOH. Modifier: EtOH+0.1% isopropylamine]. Solvents were removed under reduced pressure to give the title compound (6 mg, 43%). ¹H-NMR (CDCl₃, 400 MHz): δ 2.37 (3H, s), 2.55 (3H, s), 2.60 (11H, m), 4.39 (1H, s), 6.43 (1H, s), 7.04 (2H, s), 7.11 (1H, d), 7.34 (1H, s), 7.71 (1H, d), 8.67 (1H, s); Chiral SFC [Chiralpak AS-H, 25×0.46 cm. Pressure: 100 bar. Flow rate: 2.0 mL/min. UV detection: 210-340 nm. 8% Modifier: 2-propanol+0.1% isopropylamine]: R=7.6 min (100% ee).

### Intermediate 109: (±)-1,1-Dimethylethyl 4-[cyano(5-pyrimidinyl)methyl]-1-piperazinecarboxylate

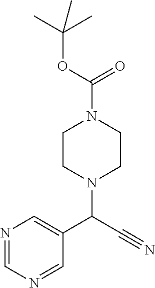

To a solution of 1 g of 5-pyrimidinecarbaldehyde (9.2 mmole, Aldrich) in 10 mL of dichloroethane in a dry flask under nitrogen were added 1.7 g of 1,1-dimethylethyl 1-piperazinecarboxylate (9.2 mmole, Fluka) followed by 3.8 mL of Ti(iOPr)₄ (12.8 mmole, Aldrich). The solution was then stirred at RT overnight. 11 mL of a solution of Et₂AlCN 1M in toluene (11 mmole, Aldrich) were added dropwise at RT and the mixture was stirred at RT for 4 h. Then 3×10 mL of a saturated solution of NaHCO₃ in water was added dropwise and the mixture was stirred at RT overnight. The resulting precipitate was filtered on celite and the cake was washed with AcOEt. The organic phase was washed with a saturated solution of NaHCO₃ in water and the layers were separated on a phase separator cartridge. Solvents were removed under reduced pressure and the resulting crude compound was purified by flash chromatography on a 25 g silica gel cartridge with AcOEt/CH 1:1 as an eluent to give the title compound as a sand color solid (1.72 g). ¹H-NMR (400 MHz, CDCl₃): δ 1.46 (9H, s), 2.56 (4H, m), 3.41 (4H, m), 5.11 (1H, s), 8.81 (2H, s), 8.94 (1H, s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.64 min. m/z (ES): 326 [M+Na]⁺, 248 [M−N]⁺.

### Intermediate 110: (±)-1,1-Dimethylethyl 4-[2-amino-2-oxo-1-(5-pyrimidinyl)ethyl]-1-piperazinecarboxylate

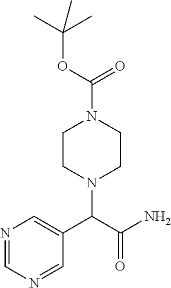

To a solution of 1.7 g of (±)-1,1-dimethylethyl 4-[cyano(5-pyrimidinyl)methyl]-1-piperazinecarboxylate (intermediate 109, 5.6 mmole) in 15 mL of MeOH were added 477 μL of DMSO (6.7 mmol, Aldrich), 6.1 mL of NaOH 2M in water (12.3 mmol) and 2.1 mL of H₂O₂ 11% (6 mmol). The mixture was stirred at RT overnight. The mixture was then neutralized with a saturated solution of NH₄Cl. The aqueous phase was then extracted 2 times with 20 mL of DCM. The aqueous phase was separated on a phase separator cartridge. The aqueous phase was then passed through HLB (Waters Oasis® HLB 35 cc (6 g) LP Extraction Cartridges) washed with 50 mL of water followed by 60 mL of MeOH. MeOH was removed under reduced pressure. The resulting crude was then purified by flash chromatography on a 20 g silica gel using a gradient of AcOEt/CH 1:1 to 10:0 as an eluent to give the title compound as a white solid (380 mg, 21%). ¹H-NMR (400 MHz, DMSO-d₆): δ 1.37 (9H, s), 2.32 (4H, m), 3.32 (4H, m), 4.09 (1H, s), 7.39 (1H, s), 7.75 (1H, s), 8.75 (2H, s), 9.13 (1H, s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.51 min. m/z (ES): 322 [M+H]⁺, 266 [M−CN]⁺.

### Intermediate 111: (±)-1,1-Dimethylethyl 4-[2-{2-[3,5-bis(trifluoromethyl)phenyl]hydrazino}-2-oxo-1-(5-pyrimidinyl)ethyl]-1-piperazinecarboxylate

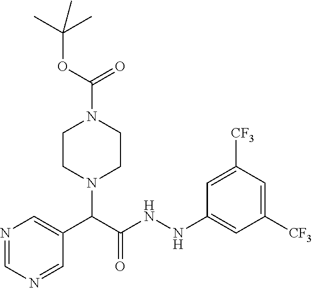

380 mg of (±)-1,1-dimethylethyl 4-[2-amino-2-oxo-1-(5-pyrimidinyl)ethyl]-1-piperazinecarboxylate (intermediate 110, 1.2 mmole) were dissolved in 15 mL of DCM.

Then 516 mg of (Boc)₂O (2.4 mmole, Aldrich) were added followed by 15 mg of DMAP (2,6-dimethylaminopyridine, 0.12 mmole, Aldrich). The solution was then stirred at RT overnight. 345 mg of 3,5-bis(trifluoromethyl)phenylhydrazine (1.4 mmole, Lancaster) were added followed by 30 mg of DMAP (0.24 mmole). The solution was stirred at RT for 2 days. Solvents were removed under reduced pressure and the crude was then purified by flash chromatography on a 20 g silica gel cartridge using a gradient of AcOEt/CH 1:1 to 10:0 as an eluent to give the title compound as a white solid (176 mg, 26%). ¹H-NMR (400 MHz, CD₃OD): δ 1.43 (9H, s), 2.49 (4H, m), 5.40 (4H, m), 4.32 (1H, s), 6.36 (1H, s), 7.16 (1H, br s), 7.39 (1H, br s), 8.68 (1H, d); 8.80 (1H, d); 8.22 (1H, d).

### Intermediate 112: (±)-N′-[3,5-bis(trifluoromethyl)phenyl]-2-(1-piperazinyl)-2-(5-pyrimidinyl)-acetohydrazide

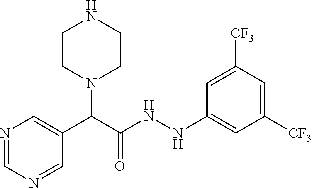

A solution of 1 mL of TFA in 5 mL of DCM was added dropwise to a stirred solution of 176 mg of (±)-1,1-dimethylethyl 4-[2-{2-[3,5-bis(trifluoromethyl)phenyl]hydrazino}-2-oxo-1-(5-pyrimidinyl)ethyl]-1-piperazinecarboxylate (intermediate 111, 0.32 mmole) in 4.4 mL of DCM. Then the reaction mixture was stirred at RT overnight. After evaporation of the volatiles under reduced pressure, the crude was purified by flash chromatography on a 10 g SCX column (from DCM, MeOH to NH₃ 0.5M in MeOH), to give the title compound as a sand colour solid (125 mg, 87%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.56 min. m/z (ES): 449 [M+H]⁺.

### Intermediate 113: (±)-N′-[3,5-bis(trifluoromethyl)phenyl]-2-(4-methyl-1-piperazinyl)-2-(5-pyrimidinyl)acetohydrazide

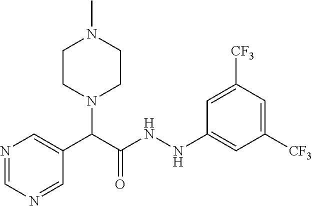

To a solution of 125 mg of (±)-N′-[3,5-bis(trifluoromethyl)phenyl]-2-(1-piperazinyl)-2-(5-pyrimidinyl)acetohydrazide (intermediate 112, 0.28 mmole) in 3 mL of CH₃CN, 34 μL of formaldehyde 37% in water (0.42 mmol, Aldrich) were added. The mixture was stirred under nitrogen at RT for 1 h, then 89 mg of Na(OAc)₃BH (0.42 mmol, Aldrich) were added and the mixture was stirred for 3 h. Then water was added and the aqueous phase was extracted with DCM. The organic phase was separated on a phase separator cartridge and evaporated under reduced pressure. The crude was then purified by flash chromatography on a 5 g silica gel cartridge using a gradient of AcOEt/CH 6:4 to 10:0, then AcOEt/MeOH 9:1 to 7:3 as an eluent to give the title compound as a sand colour solid (28 mg). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.56 min. m/z (ES): 463 [M+H]⁺; The aqueous phase was passed through a 5 g SCX cartridge (from DCM, MeOH to NH₃ 0.5M in MeOH), to give the title compound as a sand colour solid (78 mg). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.56 min. m/z (ES): 463 [M+H]⁺.

### Compound 78: N′-[3,5-bis(trifluoromethyl)phenyl]-2-(4-methyl-1-piperazinyl)-2-(5-pyrimidinyl)acetohydrazide-enantiomer 1

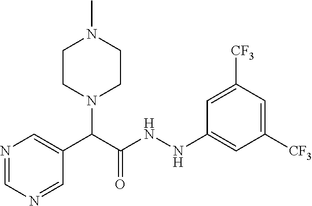

94 mg of (±)-N′-[3,5-bis(trifluoromethyl)phenyl]-2-(4-methyl-1-piperazinyl)-2-(5-pyrimidinyl)acetohydrazide (intermediate 113, 0.2 mmole) were purified by semipreparative chiral SFC [Chiralpak AD-H, 25×2.1 cm, pressure: 163 bar, Flow rate: 14 mL/min, UV detection: 245 nm, Injection 900 μL of 20 mg/mL solution in mobile phase each, modifier: n-Hex/EtOH 85/15]. Solvents were removed under reduced pressure to give the title compound as a sand colour solid (37.2 mg, 0.08 mmole). ¹H-NMR (400 MHz, CDCl₃): δ 2.30 (3H, s), 2.57 (8H, m), 4.32 (1H, s), 6.58 (1H, s), 7.15 (2H, s), 7.39 (2H, s), 8.69 (1H, s), 8.96 (1H, s), 9.23 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.55 min. (100%) m/z (ES): 463.1 [M+H]⁺; Chiral SFC [Chiralpak AS-H, 25×4.6 cm, pressure: 100 bar, Flow rate: 2.0 mL/min, UV detection: 245 nm, modifier: n-Hex/EtOH 85/15]: Rₜ=6.64 min. (99% ee).

### Compound 79: N′-[3,5-bis(trifluoromethyl)phenyl]-2-(4-methyl-1-piperazinyl)-2-(5-pyrimidinyl)acetohydrazide-enantiomer 2

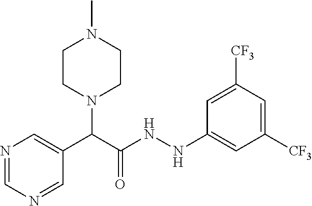

94 mg of (±)-N′-[3,5-bis(trifluoromethyl)phenyl]-2-(4-methyl-1-piperazinyl)-2-(5-pyrimidinyl)acetohydrazide (intermediate 113, 0.2 mmole) were purified by semipreparative chiral SFC [Chiralpak AD-H, 25×2.1 cm, pressure: 163 bar, Flow rate: 14 mL/min, UV detection: 245 nm, Injection 900 μL of 20 mg/mL solution in mobile phase each, modifier: n-Hex/EtOH 85/15]. Solvents were removed under reduced pressure to give the title compound as a sand colour solid (36.9 mg, 0.08 mmole). ¹H-NMR (400 MHz, CDCl₃): δ 2.30 (3H, s), 2.57 (8H, m), 4.32 (1H, s), 6.58 (1H, s), 7.15 (2H, s), 7.39 (2H, s), 8.69 (1H, s), 8.96 (1H, s), 9.23 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.55 min. (100%) m/z (ES): 463.1 [M+H]⁺; Chiral SFC [Chiralpak AS-H, 25×4.6 cm, pressure: 100 bar, Flow rate: 2.0 mL/min, UV detection: 245 nm, modifier: n-Hex/EtOH 85/15]: Rₜ=9.22 min. (99% ee).

### Intermediate 114: (8aS)-Octahydropyrrolo[1,2-a]pyrazine dihydrochloride

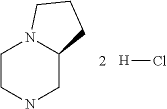

To a stirred solution of 13 g of (8aS)-hexahydropyrrolo[1,2-a]pyrazin-6(2H)-one (prepared as set out in WO03066635A1, 93 mmole) in 250 mL of dry THF under nitrogen atmosphere and at 0° C., were added 280 mL of 1M solution of BH₃.THF (278 mmole, Aldrich) over 20 min. The solution was then heated at 55° C. overnight. The solution was cooled to 0° C. and 100 mL of MeOH were added followed by 500 mL of HCl 1M in Et₂O. The solution was heated at reflux overnight. Solvents were removed under reduced pressure to give the title compound (18.8 g, quantitative). ¹H-NMR (400 MHz, DMSO-d₆): δ 1.25-4.15 (13H, m), 9.58-10.15 (2H, m), 11.84-12.24 (1H, br s).

### Intermediate 115: (8aR)-Octahydropyrrolo[1,2-a]pyrazine dihydrochloride

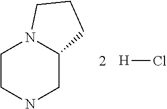

To a stirred solution of 10 g of (8aR)-hexahydropyrrolo[1,2-a]pyrazin-6(2H)-one (prepared as set out in WO03066635A1, 71.4 mmole) in 150 mL of dry THF under Argon atmosphere and at 0° C., were added 214 mL of 1M solution of BH₃.THF (214 mmole, Aldrich). The solution was then stirred at RT for 3 h, heated at 50° C. for 3 h, left at RT overnight and then heated again at 50° C. for 2 h. The reaction mixture was then cooled to 0° C. and further 71 mL of BH₃*THF (71 mmole, Aldrich) were added dropwise. The solution was heated at 50° C. to allow the reaction to be completed. At 0° C., 80 mL of MeOH were added followed by 300 mL of HCl 1M in Et₂O. The solution was heated at reflux for 5 h and then left at RT over the weekend. Solvents were removed under reduced pressure to give the title compound (13 g, 93%). ¹H-NMR (400 MHz, DMSO-d₆): δ 1.15-4.05 (13H, m), 9.2-9.9 (2H, br s), 11.84-12.04 (1H, br s).

### Intermediate 116: Ethyl(±)-(2,6-dimethyl-3-pyridinyl)(hydroxy)acetate

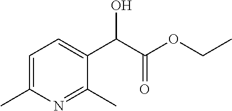

In a 50 mL round-bottomed flask, 0.67 mL of isopropylmagnesium chloride (1.34 mmole, Fluka) were dissolved in 5 mL of dry THF to give a colorless solution. This solution was cooled to 0° C. and 1.76 mL of BuLi 1.6 M (2.82 mmole, Aldrich) were added. The yellow pale solution was stirred for 15 min and then cooled to −10° C. A solution of 500 mg of 3-bromo-2,6-dimethylpyridine (2.69 mmole, ABCR) in 7 mL of dry THF was added and the solution was stirred for 30 min at −10° C. 1.06 mL of ethyl glyoxylate (5.37 mmole, Fluka) were added and the mixture was slowly warmed to 0° C. and stirred for 2 h. A saturated solution of NaHCO₃ was added at 0° C., the aqueous layer was backextracted with AcOEt (2×20 mL). The combined organic layers were dried over Na₂SO₄, filtered and concentrated. The crude product was purified by flash chromatography eluting with a gradient CH/AcOEt. Collected fractions gave the title compound (200 mg, 35%). ¹H-NMR (400 MHz, CDCl₃): δ 1.26 (3H, t), 2.59 (3H, s), 2.70 (3H, s), 4.11-4.35 (2H, m), 5.38 (1H, br s), 7.00 (1H, d), 7.48 (1H, d); m/z (ES): 210 [M+H]⁺.

### Intermediate 117: Ethyl (2,6-dimethyl-3-Pyridinyl)[(8aR)-hexahydropyrrolo[1,2-a]pyrazin-2(1H)-yl]acetate-mixture of diastereoisomers

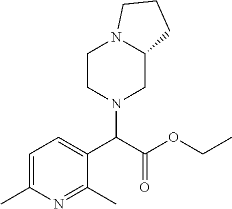

In a 250 mL round-bottomed flask, 1.65 g of ethyl(2,6-dimethyl-3-pyridinyl)(hydroxy)acetate (intermediate 116, 7.89 mmole) were dissolved in 40 mL of dry DCM to give a colorless solution. 4.40 mL of TEA (31.5 mmole, Aldrich) were added and the solution was cooled to 0° C. 0.67 mL of Mesyl-Cl (8.67 mmole, Fluka) were added and the pale yellow solution was stirred at 0° C. for 45 min. 1.57 g of (8aR)-octahydropyrrolo[1,2-a]pyrazine dihydrochloride (intermediate 115, 7.89 mmole) were added and the mixture was stirred at RT for 48 h. Further TEA (3 eq) and 1.57 g of (8aR)-octahydropyrrolo[1,2-a]pyrazine dihydrochloride (7.89 mmole) were added and the reaction mixture was stirred for additional 15 h. The suspension was washed with a saturated NaHCO₃ solution. The organic phase was dried over Na₂SO₄, filtered and concentrated. The crude was purified by flash chromatography eluting with a gradient of DCM/MeOH to give the title compound (1.2 g, 48%). ¹H-NMR (400 MHz, CDCl₃): δ 1.25 (3H, t), 1.70-2.50 (9H, m), 2.48 (3H, s), 2.60 (3H, s), 2.80 (1H, d), 2.90 (1H, d), 3.20 (2H, m), 4.07-4.24 (2H, m), 4.30 (1H, s), 7.00 (1H, d), 7.70 (1H, d); m/z (ES): 318 [M+H]⁺, 159 [(M+H)/2]⁺

### Intermediate 118: Potassium (2,6-dimethyl-3-pyridinyl)[(8aR)-hexahydropyrrolo[1,2-a]pyrazin-2(1H)-yl]acetate

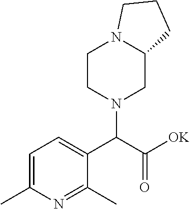

In a 50 mL round-bottomed flask, 800 mg ethyl(2,6-dimethyl-3-pyridinyl)[(8aR)-hexahydropyrrolo[1,2-a]pyrazin-2(1H)-yl]acetate (intermediate 117, 2.52 mmole) were dissolved in 10 mL of MeOH to give a yellow solution. Water was added followed by 175 mg of potassium hydroxide (2.65 mmole, Fluka). The solution was stirred at RT for 45 h. 28 mg of potassium hydroxide (0.5 mmole) were added and the solution was stirred for additional 24 h. 28 mg of potassium hydroxide (0.5 mmole) were added and the solution stirred for additional 24 h. The solution was concentrated under vacuum to give the title compound (1.12 g, quantitative). ¹H-NMR (400 MHz, DMSO-d₆): δ 1.00-1.25 (1H, m), 1.50-2.20 (7H, m), 2.25-2.00 (1H, m), 2.30 (3H, s), 2.48 (3H, s), 2.69-2.96 (2H, m), 3.04-3.28 (2H, m), 3.70 (1H, d), 6.80 (1H, d), 7.70 (1H, d).

### Intermediate 119: N′-[3,5-Bis(trifluoromethyl)phenyl]-2-(2,6-dimethyl-3-pyridinyl)-2-[(8aR)-hexahydropyrrolo[1,2-a]pyrazin-2(1H)-yl]acetohydrazide-mixture of diastereoisomers

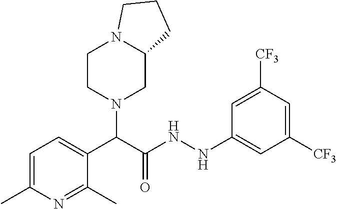

In a 50 mL round-bottomed flask, 550 mg of potassium (2,6-dimethyl-3-pyridinyl)[(8aR)-hexahydropyrrolo[1,2-a]pyrazin-2(1H)-yl]acetate (intermediate 118, 1.68 mmole) were dissolved in 10 mL of dry DCM to give a white suspension. 0.88 mL of DIPEA (5.04 mmole, Aldrich) were added followed by 992 mg of TOTU (3.02 mmole, Fluka). The resulted red solution was stirred at RT for 15 min. Then 451 mg of 3,5-bis(trifluoromethyl)phenylhydrazine (1.84 mmole, Lancaster) were added and the solution became dark. The reaction mixture was washed with a saturated solution of NaHCO₃. The organic phase was dried over Na₂SO₄, filtrated and concentrated. The crude product was purified by flash chromatography eluting with DCM/MeOH 9:1. Solvents were removed under reduced pressure to give the title compound as a mixture of 60:40 of 2 diastereoisomers (300 mg, 35%). LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.28 min.

### Compound 80: N′-[3,5-Bis(trifluoromethyl)phenyl]-2-(2,6-dimethyl-3-pyridinyl)-2-[(8aR)-hexahydropyrrolo[1,2-a]pyrazin-2(1H)-yl]acetohydrazide-diastereoisomer 1

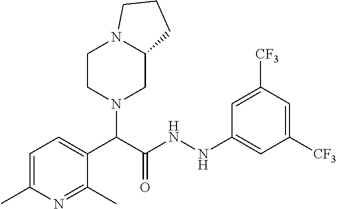

70 mg of N′-[3,5-bis(trifluoromethyl)phenyl]-2-(2,6-dimethyl-3-pyridinyl)-2-[(8aR)-hexahydropyrrolo[1,2-a]pyrazin-2(1H)-yl]acetohydrazide (intermediate 119, 0.13 mmole) was purified by semipreparative chiral SFC [Chiralpak AD-H, 25×0.46 cm, pressure: 100 bar, Flow rate: 2.0 mL/min, UV detection: CD 245 nm, 13% modifier: 2-propanol+0.1% isopropylamine]. Solvents were removed under reduced pressure to give the title compound as a white solid (23 mg, 55%). ¹H-NMR (400 MHz, CDCl₃): δ 1.50-2.20 (8H, m), 2.50 (3H, s), 2.60 (3H, s), 2.80 (1H, m), 3.10 (3H, t), 3.30 (1H, m), 4.60 (1H, br s), 6.70 (1H, br s), 7.10 (3H, m), 7.40 (1H, s), 7.60 (1H, d), 9.20 (1H, br s); Chiral SFC [Chiralpak AD-H, 25×0.46 cm, pressure: 100 bar, Flow rate: 2.0 mL/min, UV detection: 240 nm, 13% modifier: 2-propanol+0.1% isopropylamine]: Rₜ=8.06 min. (100% d.e.).

### Compound 81: N′-[3,5-bis(trifluoromethyl)phenyl]-2-(2,6-dimethyl-3-pyridinyl)-2-[(8aR)-hexahydropyrrolo[1,2-a]pyrazin-2(1H)-yl]acetohydrazide-diastereoisomer 2

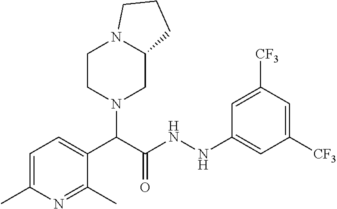

70 mg of N′-[3,5-bis(trifluoromethyl)phenyl]-2-(2,6-dimethyl-3-pyridinyl)-2-[(8aR)-hexahydropyrrolo[1,2-a]pyrazin-2(1H)-yl]acetohydrazide (intermediate 119, 0.13 mmole) were purified by semipreparative chiral SFC [Chiralpak AD-H, 25×0.46 cm, pressure: 100 bar, Flow rate: 2.0 mL/min, UV detection: CD 245 nm, 13% modifier: 2-propanol+0.1% isopropylamine]. Solvents were removed under reduced pressure to give the title compound as a white solid (12 mg, 43%). ¹H-NMR (400 MHz, CDCl₃): δ 1.70-2.00 (4H, m), 2.01-2.41 (6H, m), 2.50 (3H, s), 2.65 (3H, s), 2.70 (1H, d), 2.94-3.15 (2H, m), 3.20 (1H, d), 4.40 (1H, s), 6.30 (1H, br s), 7.10 (3H, m), 7.30 (1H, s), 7.60 (1H, d); Chiral SFC [Chiralpak AD-H, 25×0.46 cm, pressure: 100 bar, Flow rate: 2.0 mL/min, UV detection: 240 nm, 13% modifier: 2-propanol+0.1% isopropylamine]: Rₜ=9.78 min. (100% d.e.).

### Intermediate 120 (2-chlorophenyl)[(8aS)-hexahydropyrrolo[1,2-a]pyrazin-2(1H)-yl]acetic acid dihydrochloride-mixture of diastereoisomers

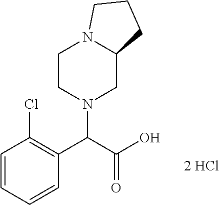

540 mg of potassium carbonate (3.88 mmole, Fluka) were added to a solution of 323 mg of (±)-bromo(2-chlorophenyl)acetic acid (1.3 mmole, Apollo Scientific Ltd) in 2 mL of THF. The mixture was stirred for 10 min, then 210 mg of (8aS)-octahydropyrrolo[1,2-a]pyrazine hydrochloride (intermediate 114, 1.3 mmole) in THF/TEA (5 mL/0.3 mL) were added dropwise and the suspension was stirred at RT overnight. The solid was filtered and the filtrate concentrated under vacuum. The crude was passed twice through a HLB cartridge (Waters Oasis® HLB 35 cc (6 g) LP Extraction Cartridges) to give the title compound (37 mg, 8%) as mixture of two diastereisomers. LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.05 min; m/z (ES): 295 [M+H]⁺.

### Intermediate 121: N′-[3,5-bis(trifluoromethyl)phenyl]-2-(2-chlorophenyl)-2-[(8aS)-hexa-hydropyrrolo[1,2-a]pyrazin-2(1H)-yl]acetohydrazide-mixture of diastereoisomers

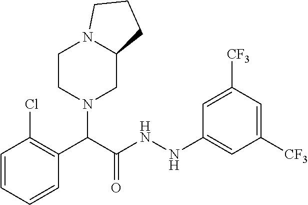

36 mg of (2-chlorophenyl)[(8aS)-hexahydropyrrolo[1,2-a]pyrazin-2(1H)-yl]acetic acid dihydrochloride (intermediate 120, 0.1 mmole) were dissolved in 1 mL of dry DCM. To this solution, were added successively 61 μL of DIPEA (0.35 mmole, Aldrich), 17 mg of HOBT.H₂O (0.13 mmole, Fluka) and 24 mg of EDCI.HCl (0.13 mmole, Fluka) and the reaction mixture was stirred for 15 min at RT. Then 33 mg of 3,5-bis(trifluoromethyl)phenylhydrazine (0.13 mmole, Lancaster) were added. Further 3,5-bis(trifluoromethyl)phenylhydrazine (38 mg, 0.15 mmole), HOBT.H₂O (6.5 mg, 0.05 mmole), EDCI.HCl (9 mg, 0.05 mmole) and DIPEA (17 μL, 0.1 mmole) were added and stirring was continued overnight. The organic phase was then washed with a saturated solution of NaHCO₃, dried over Na₂SO₄, filtered and evaporated to dryness. The crude was then purified by flash chromatography on silica gel eluting from DCM 100% to DCM/MeOH 80:20 to give the title compound (32 mg, 62%) as a mixture of two diastereoisomers. ¹H-NMR (400 MHz, CDCl₃): δ 1.12-3.31 (13H, m), 4.82-4.94 (1H, m), 6.55-6.69 (1H, s), 7.07-7.19 (2H, s), 7.39-7.59 (5H, m), 8.68-8.89 (1H, m); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.96 min.

### Compound 82: N′-[3,5-bis(trifluoromethyl)phenyl]-2-(2-chlorophenyl)-2-[(8aS)-hexahydro-pyrrolo[1,2-a]pyrazin-2(1H)-yl]acetohydrazide-diastereoisomer 1

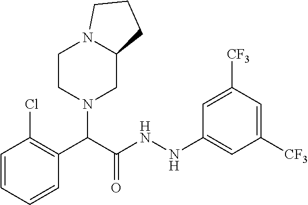

32 mg of N′-[3,5-bis(trifluoromethyl)phenyl]-2-(2-chlorophenyl)-2-[(8aS)-hexahydro-pyrrolo[1,2-a]pyrazin-2(1H)-yl]acetohydrazide (intermediate 121, 0.06 mmole) were purified by Chiral SFC [Chiralpak AD-H, 25×2.1 cm, pressure: 168 bar, Flow rate: 22 mL/min, UV detection: 220 nm, 15% modifier: EtOH+0.1% isopropylamine]. Solvents were removed under reduced pressure to give the title compound as a white solid (12 mg, 75%). ¹H-NMR (400 MHz, CDCl₃): δ 1.20-3.20 (13H, m), 4.80 (1H, s), 6.50 (1H, s), 7.10-7.60 (7H, m), 8.70 (1H, s); Chiral SFC [Chiralpak AD-H, 25×0.46 cm, pressure: 100 bar, Flow rate: 2.0 mL/min, UV detection: 240 nm, 15% modifier: EtOH+0.1% isopropylamine]: Rₜ=3.6 min.

### Compound 83: N′-[3,5-bis(trifluoromethyl)phenyl]-2-(2-chlorophenyl)-2-[(8aS)-hexahydro-pyrrolo[1,2-a]pyrazin-2(1H)-yl]acetohydrazide-diastereoisomer 2

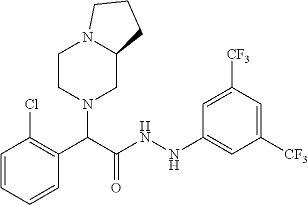

32 mg of N′-[3,5-bis(trifluoromethyl)phenyl]-2-(2-chlorophenyl)-2-[(8aS)-hexahydro-pyrrolo[1,2-a]pyrazin-2(1H)-yl]acetohydrazide (intermediate 121, 0.06 mmole) were purified by Chiral SFC [Chiralpak AD-H, 25×2.1 cm, pressure: 168 bar, Flow rate: 22 mL/min, UV detection: 220 nm, 15% modifier: EtOH+0.1% isopropylamine]. Solvents were removed under reduced pressure to give the title compound as a white solid (5 mg, 16%). Chiral SFC [Chiralpak AD-H, 25×0.46 cm, pressure: 100 bar, Flow rate: 2.0 mL/min, UV detection: 240 nm, 15% modifier: EtOH+0.1% isopropylamine]: Rₜ=4.07 min.

### Intermediate 122: (2-chlorophenyl)[(8aR)-hexahydropyrrolo[1,2-a]pyrazin-2(1H)-yl]acetic acid dihydrochloride-mixture of diastereoisomers

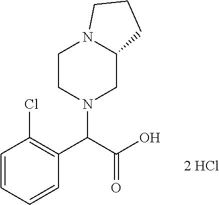

330 mg of potassium carbonate (2.48 mmole, Fluka) were added to a solution of 206 mg of (±)-bromo(2-chlorophenyl)acetic acid (0.83 mmole, Apollo Scientific Ltd) in 3 mL of THF. The mixture was stirred for 10 min, then a solution of 134 mg of (8aR)-octahydropyrrolo[1,2-a]pyrazine dihydrochloride (intermediate 115, 0.83 mmole) in 1 mL of DMF was added dropwise and the suspension was stirred at RT overnight. The solid was filtered and mother liquors were concentrated under vacuum. The crude was passed through a HLB cartridge (Waters Oasis® HLB 35 cc (6 g) LP Extraction Cartridges) to give the title compound (141 mg, 0.53 mmole) as a mixture of two diastereoisomers. This batch was used in the next step without further purification although it was not clean. LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.04 min; m/z (ES): 295 [M+H]⁺.

### Intermediate 123: N′-[3,5-bis(trifluoromethyl)phenyl]-2-(2-chlorophenyl)-2-[(8aR)-hexahydropyrrolo[1,2-a]pyrazin-2(1H)-yl]acetohydrazide-mixture of diastereoisomers

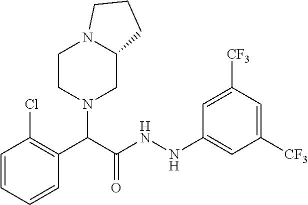

141 mg of (2-chlorophenyl)[(8aR)-hexahydropyrrolo[1,2-a]pyrazin-2(1H)-yl]acetic acid dihydrochloride (intermediate 122, 0.38 mmole) were dissolved in 4 mL of dry DCM. To this solution, were added successively 201 μL of DIPEA (1.16 mmole, Aldrich), 52 mg of HOBT.H₂O (0.38 mmole, Fluka) and 74 mg of EDCI.HCl (0.38 mmole, Fluka) and the reaction mixture was stirred for 15 min at RT. Then 103 mg of 3,5-bis(trifluoromethyl)phenylhydrazine (0.42 mmole, Lancaster) were added. Further 3,5-bis(trifluoromethyl)phenylhydrazine (196 mg, 0.76 mmole), HOBT.H₂O (52 mg, 0.38 mmole), EDCI.HCl (74 mg, 0.38 mmole) and DIPEA (177 μL, 0.76 mmole) were added and stirring was continued overnight. The organic phase was then washed with a saturated solution of NaHCO₃, dried over Na₂SO₄, filtered and evaporated to dryness. The crude was then purified by flash chromatography on silica gel eluting from DCM 100% to DCM/MeOH 80:20 to give the title compound (65 mg, 33%) as mixture of two diastereoisomers. ¹H-NMR (400 MHz, CDCl₃): δ 1.07-3.35 (13H, m), 4.82-4.93 (1H, m), 6.59-6.74 (1H, br s), 7.03-7.18 (2H, s), 7.18-7.59 (5H, m), 8.71-8.96 (1H, br s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.98 min.

### Compound 84: N′-[3,5-bis(trifluoromethyl)phenyl]-2-(2-chlorophenyl)-2-[(8aR)-hexahydropyrrolo[1,2-a]pyrazin-2(1H)-yl]acetohydrazide-diastereoisomer 1

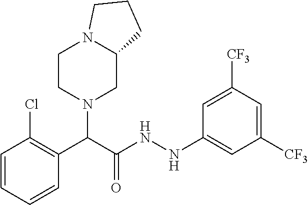

65 mg of N′-[3,5-bis(trifluoromethyl)phenyl]-2-(2-chlorophenyl)-2-[(8aR)-hexahydro-pyrrolo[1,2-a]pyrazin-2(1H)-yl]acetohydrazide (intermediate 123, 0.12 mmole) were purified by Chiral SFC [Chiralpak AD-H, 25×2.1 cm, pressure: 168 bar, Flow rate: 22 mL/min, UV detection: 220 nm, 15% modifier: EtOH+0.1% isopropylamine]. Solvents were removed under reduced pressure to give the title compound as a white solid (16.6 mg, 52%). Chiral SFC [Chiralpak AD-H, 25×0.46 cm, pressure: 100 bar, Flow rate: 2.0 mL/min, UV detection: 240 nm, 15% modifier: EtOH+0.1% isopropylamine]: Rₜ=3.93 min.

### Compound 85: N′-[3,5-bis(trifluoromethyl)phenyl]-2-(2-chlorophenyl)-2-[(8aR)-hexahydro-pyrrolo[1,2-a]pyrazin-2(1H)-yl]acetohydrazide-diastereoisomer 2

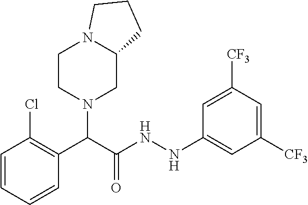

65 mg of N′-[3,5-bis(trifluoromethyl)phenyl]-2-(2-chlorophenyl)-2-[(8aR)-hexahydro-pyrrolo[1,2-a]pyrazin-2(1H)-yl]acetohydrazide (intermediate 123, 0.12 mmole) were purified by Chiral SFC [Chiralpak AD-H, 25×2.1 cm, pressure: 168 bar, Flow rate: 22 mL/min, UV detection: 220 nm, 15% modifier: EtOH+0.1% isopropylamine]. Solvents were removed under reduced pressure to give the title compound as a white solid (18 mg, 57%). ¹H-NMR (400 MHz, CDCl₃): δ 1.25-3.25 (13H, m), 4.80 (1H, s), 6.40 (1H, br s), 7.15 (2H, s), 7.20-7.40 (3H, m), 7.59 (2H, m), 8.71 (1H, br s); Chiral SFC [Chiralpak AD-H, 25×0.46 cm, pressure: 100 bar, Flow rate: 2.0 mL/min, UV detection: 240 nm, 15% modifier: EtOH+0.1% isopropylamine]: Rₜ=4.68 min.

### Intermediate 124: N′-(3,5-dichlorophenyl)-2-[(8aS)-hexahydropyrrolo[1,2-a]pyrazin-2(1H)-yl]-2-(1-naphthalenyl)acetohydrazide-mixture of diastereoisomers

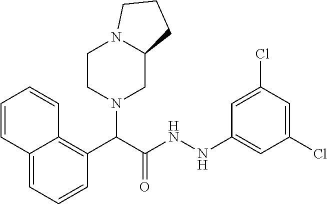

A solution of 259 mg of 1-naphthalenylboronic acid (1.5 mmole, Lancaster), 190 mg of (8aS)-octahydropyrrolo[1,2-a]pyrazine dihydrochloride (intermediate 114, 1.5 mmole) and 139 mg of glyoxylic acid monohydrate (1.5 mmole, Aldrich) in 1.5 mL of MeCN was heated at 120° C. under MW irradiation (2×10 min). Then it was allowed to cool to RT, diluted with DCM, basified with NaOH 2N and the aqueous phase treated with HCl 1N and passed through HLB cartridge (Waters Oasis® HLB 35 cc (6 g) LP Extraction Cartridges) (from H₂O to H₂O/MeOH 4:6). Solvents were removed under reduced pressure and the compound (244 mg, 0.64 mmole) was dried under high vacuum and used in the next step. This product was then suspended in 3 mL of DCM, and 460 μL of DIPEA (2.56 mmole, Aldrich) and 266 mg of TBTU (0.83 mmole, Fluka) were added successively. The solution was stirred for 20 min and then 136 mg of 3,5-bis(trifluoromethyl)phenylhydrazine (0.77 mmole, Lancaster) were added. The red mixture was then stirred at RT overnight. Water was added, the organic phase was dried and concentrated under vacuum. The crude material was purified by flash chromatography with the following eluents: DCM 100%, DCM/MeOH 95:5, 90:10, 85:15, 80:20, DCM/NH₃ 0.5M in MeOH 90:10 to give the title compound as a mixture of diastereoisomers (263 mg, 88%). m/z (ES): 469.1 [M]⁺

### Compound 86: N′-(3,5-dichlorophenyl)-2-[(8aS)-hexahydropyrrolo[1,2-a]pyrazin-2(1H)-yl]-2-(1-naphthalenyl)acetohydrazide-diastereoisomer 1

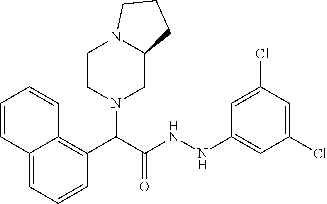

263 mg of N′-(3,5-dichlorophenyl)-2-[(8aS)-hexahydropyrrolo[1,2-a]pyrazin-2(1H)-yl]-2-(1-naphthalenyl)acetohydrazide (intermediate 124, 0.56 mmole) were purified by Chiral HPLC [Chiralpak AD-H, 250×21 mm, Flow rate: 13 mL/min, UV detection: 225 nm, modifier: n-Hex/IPA+0.1% isopropylamine]. Solvents were removed under reduced pressure to give the title compound as a white solid (105 mg). ¹H-NMR (400 MHz, DMSO-d₆): δ 1.15-3.00 (13H, m), 4.80 (1H, s), 6.35 (2H, s), 6.70 (1H, s), 7.15-7.45 (3H, m), 7.80 (1H, d), 7.85-8.00 (2H, dd), 8.35 (1H, s), 8.60 (1H, s), 10.25 (1H, br s).

### Compound 87: N′-(3,5-dichlorophenyl)-2-[(8aS)-hexahydropyrrolo[1,2-a]pyrazin-2(1H)-yl]-2-(1-naphthalenyl)acetohydrazide-diastereoisomer 2

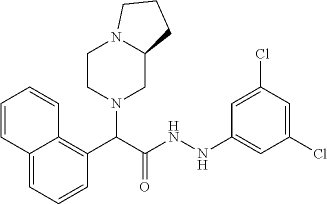

263 mg of N′-(3,5-dichlorophenyl)-2-[(8aS)-hexahydropyrrolo[1,2-a]pyrazin-2(1H)-yl]-2-(1-naphthalenyl)acetohydrazide (intermediate 124, 0.56 mmole) were purified by chiral HPLC [Chiralpak AD-H, 250×21 mm, Flow rate: 13 mL/min, UV detection: 225 nm, modifier: n-Hex/IPA+0.1% isopropylamine]. Solvents were removed under reduced pressure to give the title compound as a white solid (94 mg). ¹H-NMR (400 MHz, DMSO-d₆): δ 1.20-3.10 (13H, m), 4.77 (1H, s), 6.40 (2H, s), 6.72 (1H, s), 7.46-7.62 (3H, m), 7.82 (1H, d), 7.84-8.00 (2H, dd), 8.40 (1H, s), 8.65 (1H, s), 10.28 (1H, br s).

### Intermediate 125: (±)-Ethyl hydroxy(3-pyridinyl)acetate

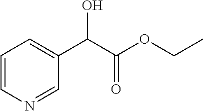

19.78 mL of nBuLi 1.6M in hexanes (31.65 mmole, ALDRICH) were added to 7.91 mL of iPrMgCl 2M diluted in 66 mL of THF at 0° C. (7° C. int. T°). After stirring for 15 min at 0° C., the mixture was cooled to −10° C. (−8° C. int. T°) and a solution of 3.05 mL of 3-bromopyridine (31.65 mmole, ALDRICH) in 31.65 mL of THF was added. The mixture was then stirred at −10° C. for 30 min. Then 12.92 mL of a solution of ethyl glyoxylate 50% in toluene (63.29 mmole, FLUKA) were added and the mixture was stirred at 0° C. for 2 h. The reaction mixture was then partitioned between 100 mL of AcOEt and 100 mL of a saturated solution of K₂CO₃. The precipitate was filtered and the resulting biphasic solution was transferred to a separatory funnel. The aqueous phase was extracted with 50 mL of AcOEt. The combined organic layers were dried over Na₂SO₄, filtered and evaporated to dryness. The resulting crude was then purified by flash chromatography on silica gel with the following gradient: A: CH+3% TEA/AcOEt+3% TEA: 0% B for 2 min, 0% to 100% B in 24 min, 100% B for 6 min. Evaporation of the solvents under reduced pressure and further purification gave the title compound as a yellow oil (2.75 g, 48%). ¹H-NMR (400 MHz, CDCl₃): δ 1.23 (3H, t), 4.14-4.33 (2H, m), 5.23 (1H, s), 7.31 (1H, dd), 7.79 (1H, dt), 8.55 (1H, dd), 8.68 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=0.24 min and 0.36 min. (96.8%) m/z (ES): 182.0 [M+H]⁺.

### Intermediate 126: Ethyl (8aR)-hexahydropyrrolo[1,2-a]pyrazin-2(1H)-yl(3-pyridinyl)acetate-mixture of diastereoisomers

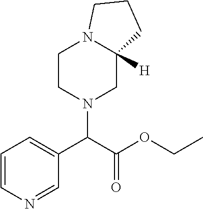

1.5 g of (±)-ethyl hydroxy(3-pyridinyl)acetate (intermediate 125, 8.28 mmole) were dissolved in 52.5 mL of DCM. The solution was cooled to 0° C. (+4° C. int. T°) and 4.05 mL of TEA (28.97 mmole, Aldrich) were added followed by 704 μL of MsCl (9.11 mmole, Aldrich). The mixture was then stirred at 0° C. for 1.3 h. Then 1.41 g of (8aR)-octahydropyrrolo[1,2-a]pyrazine dihydrochloride (intermediate 115, 8.7 mmole) were added and the solution was warmed to RT and stirred at RT for 13.5 h. 50 mL of a saturated solution of K₂CO₃ were added and the biphasic solution was transferred to a separatory funnel. The aqueous phase was removed and the organic phase was dried over Na₂SO₄, filtered and evaporated to dryness. The resulting crude was then purified by flash chromatography on silica gel using the following gradient: A: CH+3% TEA/AcOEt+3% TEA: 0% B for 2 min, 0% to 100% B in 25 min, 100% B for 5 min. Evaporation of the solvents under reduced pressure and further purification gave the title compound as an orange oil (896.3 mg, 37%). ¹H-NMR (400 MHz, CDCl₃): δ 1.22-1.41 (5H, m), 1.68-1.90 (3H, m), 2.05-2.20 (2H, m), 2.22-2.50 (2H, m), 2.69-2.83 (1H, m), 2.90-3.10 (3H, m), 4.12-4.29 (3H, m), 7.30-7.32 (1H, m), 7.82-7.84 (1H, m), 8.57-8.58 (1H, m), 8.64 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.35 min (82.8%) m/z (ES): 290.1 [M+H]⁺.

### Intermediate 127: N′-[3,5-bis(trifluoromethyl)phenyl]-2-[(8aR)-hexahydropyrrolo[1,2-a]pyrazin-2(1H)-yl]-2-(3-pyridinyl)acetohydrazide-mixture of diastereoisomers

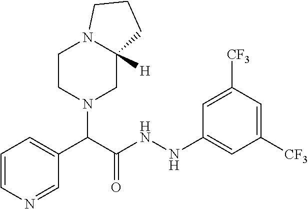

896 mg of ethyl(8aR)-hexahydropyrrolo[1,2-a]pyrazin-2(1H)-yl(3-pyridinyl)acetate (intermediate 126, 3.1 mmole) were dissolved in 10.1 mL of MeOH. 191 mg of KOH (3.4 mmole, FLUKA) were added followed by 2.23 mL of water. The mixture was then stirred at RT for 43.5 h. Solvents were removed under reduced pressure and the resulting compound was dried under high vacuum for a couple of hours to give an orange oil (984 mg). This compound was dissolved in 20.5 mL of DMF at RT. Then 883 mg of 3,5-bis(trifluoromethyl)phenylhydrazine (3.62 mmole, LANCASTER) were added followed by 1.60 g of BOP (3.62 mmole, FLUKA). The mixture was stirred at RT for 3.5 h. Solvents were removed under reduced pressure and the residue was dissolved in MeOH. The solution was then passed through a SCX cartridge (25 g). The cartridge was washed with 100 mL of DCM, 100 mL of MeOH and the compound was released with 60 mL of NH₃ 2M in MeOH. Solvents were removed under reduced pressure and the resulting crude was then purified by flash chromatography on silica gel with the following gradient: A: AcOEt+3% TEA/B: MeOH+3% TEA: 0% B for 2 min, 0% to 20% B in 20 min, 20% B for 4 min. Solvents were removed under reduced pressure and the resulting compound was then triturated twice with 10 mL of Et₂O. Supernatant was removed and the resulting solid was dried under high vacuum for a couple of hours to give the title compound as a yellow solid (673 mg, 44%). ¹H-NMR (400 MHz, CD₃OD): δ 1.40-1.60 (1H, m), 1.70-2.00 (3H, m), 2.22-2.59 (5H, m), 2.73-2.80 (1H, m), 3.06-3.33 (3H, m), 4.23-4.25 (1H, 2 s), 7.13 (2H, d), 7.34 (1H, s), 7.58-7.61 (1H, m), 8.14-8.16 (1H, m), 8.66 (1H, d), 8.79 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.52 min (100%) m/z (ES): 488 [M+H]⁺, 244.5 [(M+H)/2]⁺.

### Compound 88: (2S)—N′-[3,5-bis(trifluoromethyl)phenyl]-2-[(8aR)-hexahydropyrrolo[1,2-a]pyrazin-2(1H)-yl]-2-(3-pyridinyl)acetohydrazide (diastereoisomer 1)

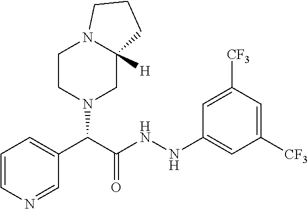

673 mg of N′-[3,5-bis(trifluoromethyl)phenyl]-2-[(8aR)-hexahydropyrrolo[1,2-a]pyrazin-2(1H)-yl]-2-(3-pyridinyl)acetohydrazide (intermediate 127, 1.38 mmole) were purified by semipreparative chiral SFC [Chiralpak AD-H, 25×2.0 cm, pressure: 182 bar, Flow rate: 22 mL/min, UV detection: 220 nm, Injection: 20 mg each in EtOH, modifier: EtOH+0.1% isopropylamine]. Solvents were removed under reduced pressure to give the title compound as a yellow solid (258.4 mg, 38%). ¹H-NMR (400 MHz, CDCl₃): δ 1.31-1.36 (2H, m), 1.64-1.84 (3H, m), 2.12-2.22 (2H, m), 2.41 (1H, td), 2.58 (1H, dt), 2.88 (1H, dt), 3.00-3.14 (3H, m), 4.31 (1H, s), 6.45 (1H, br s), 7.14 (2H, s), 7.34-7.38 (2H, m), 7.69 (1H, dt), 8.60 (1H, s), 8.63 (1H, dd), 8.91 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.52 min (100%) m/z (ES): 488 [M+H]⁺, 244.5 [(M+H)/2]⁺; Chiral SFC [Chiralpak AD-H, 25×0.46 cm, pressure: 100 bar, Flow rate: 2.0 mL/min, UV detection: 240 nm, modifier: EtOH+0.1% isopropylamine]: Rₜ=9.61 min. (100% d.e.).

### Compound 89: (2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide (diastereoisomer 2)

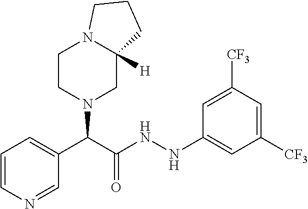

673 mg of N′-[3,5-bis(trifluoromethyl)phenyl]-2-[(8aR)-hexahydropyrrolo[1,2-a]pyrazin-2(1H)-yl]-2-(3-pyridinyl)acetohydrazide (intermediate 127, 1.38 mmole) were purified by semipreparative chiral SFC [Chiralpak AD-H, 25×2.0 cm, pressure: 182 bar, Flow rate: 22 mL/min, UV detection: 220 nm, Injection: 20 mg each in EtOH, modifier: EtOH+0.1% isopropylamine]. Solvents were removed under reduced pressure to give the title compound as a yellow solid (285 mg, 42%). ¹H-NMR (400 MHz, CDCl₃): δ 1.42-1.46 (1H, m), 1.75-1.87 (3H, m), 2.09-2.23 (4H, m), 2.33-2.37 (1H, t), 2.71 (1H, d), 3.02-3.08 (2H, m), 3.19 (1H, d), 4.27 (1H, s), 6.38 (1H, brs), 7.15 (2H, s), 7.33-7.39 (2H, m), 7.70 (1H, dt), 8.61-8.63 (2H, m), 8.86 (1H, s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.53 min (100%) m/z (ES): 488 [M+H]⁺, 244.5 [(M+H)/2]⁺; Chiral SFC [Chiralpak AD-H, 25×0.46 cm, pressure: 100 bar, Flow rate: 2.0 mL/min, UV detection: 240 nm, modifier: EtOH+0.1% isopropylamine]: Rₜ=13.08 min. (100% d.e.).

### Compound 90: (2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide hydrochloride-diastereoisomer 2

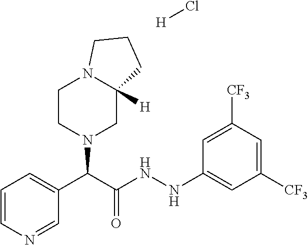

285 mg of N′-[3,5-bis(trifluoromethyl)phenyl]-2-[(8aR)-hexahydropyrrolo[1,2-a]pyrazin-2(1H)-yl]-2-(3-pyridinyl)acetohydrazide (diastereoisomer 2) (Compound 89) were dissolved in 8 mL of MeOH and 585 μL of HCl 1N/Et₂O were added. The mixture was stirred at RT for 5 min and solvents were removed. 5 mL of Et₂O were added and solvents were removed. The resulting solid was then dried under high vacuum for a couple of hours to give the title compound as a white solid (313.7 mg, quantitative). ¹H-NMR (400 MHz, CD₃OD): δ 1.70-1.80 (0.5H, m), 2.11-2.27 (3.5H, m), 2.56-2.94 (4H, m), 3.05-3.25 (2H, m), 3.37-3.52 (3H, m), 3.60-3.70 (1H, m), 4.43 (0.5H, s), 4.58 (0.5H, s), 7.05-7.11 (2H, m), 7.29 (1H, m), 7.75 (1H, m), 8.28 (1H, m), 8.72 (1H, m), 8.83 (1H, s); LC-MS [Supelcosil ABZ+Plus, 33×4.6 mm, 3 uM, gradient: A: H2O+0.1% HCOOH/B: MeCN: 0% to 95% B in 3 min, 95% B for 1 min, 95% to 0% B in 0.1 min, flow rate: 2 mL/Min]: Rₜ=1.53 min (100%) m/z (ES): 488 [M+H]⁺, 244.5 [(M+H)/2]⁺; Chiral SFC [Chiralpak AD-H, 25×0.46 cm, pressure: 100 bar, Flow rate: 2.0 mL/min, UV detection: 240 nm, modifier: EtOH+0.1% isopropylamine]: Rₜ=13.08 min. (100% d.e.).

### Compound 91: N′-[3,5-bis(trifluoromethyl)phenyl]-2-(2-chlorophenyl)-2-(1-piperazinyl)-acetohydrazide hydrochloride-enantiomer 2

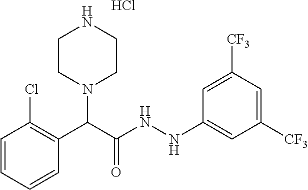

To a solution of 218 mg of N′-[3,5-bis(trifluoromethyl)phenyl]-2-(2-chlorophenyl)-2-(1-piperazinyl)acetohydrazide enantiomer 2 (compound 27, 0.45 mmole) in 5 mL of dry DCM under nitrogen, were added 450 μL of HCl 1M solution in Et₂O (0.45 mmole, Aldrich). The solution was then stirred for 30 min. The solvents were removed under reduced pressure and the resulting crude solid was triturated with dry Et₂O to give the title compound (196 mg, 84%). ¹H-NMR (400 MHz, DMSO-d₆): δ 2.67 (4H, m), 3.12 (4H, m), 4.65 (1H, s), 7.12 (2H, s), 7.31 (1H, s), 7.40 (2H, m), 7.51 (1H, m), 7.69 (1H, m), 8.7 (1H br s), 8.8 (1H, br s), 10.54 (1H, s). LC-MS [Chiralpak AS-H, 25×4.6 cm, n-Hex/EtOH+0.1% ispropylamine 85/15; flow rate: 1 mL/min; DAD=225 nm, CD=245 nm]: Rₜ=8.55 min. (98.96% e.e.).

### Intermediate 128: Ethyl(±)-hydroxy(6-methyl-3-pyridinyl)acetate

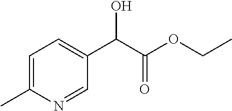

7.27 mL of nBuLi 1.6M in hexanes (11.63 mmole, ALDRICH) were added to 2.91 mL of isopropylmagnesium chloride 2.0M in THF (5.81 mmole, ALDRICH) diluted in 24 mL of THF at 0° C. The mixture was stirred at 0° C. for 15 min. Then the solution was cooled to −10° C. and a solution of 2 g of 5-bromo-2-methylpyridine (11.63 mmole, ALDRICH) in 11.5 mL of THF was added dropwise. The mixture was stirred at −10° C. for 0.5 h. Then, 4.75 mL of ethyl oxoacetate 50% in toluene (23.25 mmole, FLUKA) were added and the solution was stirred for 2.5 h at 0° C. The mixture was then poured into 100 mL of a saturated K₂CO₃ solution. The resulting precipitate was then filtered off and the cake was washed with 100 mL of AcOEt. The resulting biphasic solution was transferred to a separatory funnel and the aqueous phase was extracted 3 times with 50 mL of AcOEt. The combined organic layers were dried over Na₂SO₄, filtered and evaporated to dryness. The resulting crude was then purified by flash chromatography on silica gel with the following eluent: A: CH+3% TEA/B: AcOEt+3% TEA: 0% B for 2.5 min, 0% to 80% B in 25 min, 80% B for 10 min. Evaporation of the solvents under reduced pressure gives a yellow oil (210 mg). This compound was purified by a second flash chromatography on silica gel with the following eluent: A: CH+3% TEA/B: AcOEt+3% TEA: 0% B for 1 min, 0% to 75% B in 11 min, 75% B for 6 min. Only the fraction corresponding to the target material was collected. Evaporation of the solvents under reduced pressure gives the title compound as a yellow oil (146 mg, 6%). ¹H-NMR (400 MHz, CDCl₃): δ 1.26 (3H, t), 2.58 (3H, s), 4.13-4.35 (2H, m), 5.19 (1H, s), 7.15-7.21 (1H, m), 7.66 (1H, dd), 8.57 (1H, d). LC-MS [Xterra MS C18, 30×4.6 mm, 2.5 μm, gradient: A: NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.24 min (79.7%) m/z (ES)=196.0 [M+H]⁺.

### Intermediate 129: Ethyl (8aR)-hexahydropyrrolo[1,2-a]pyrazin-2(1H)-yl(6-methyl-3-pyridinyl)acetate-mixture of diastereoisomers

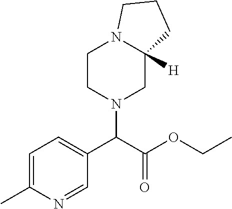

146 mg of ethyl hydroxy(6-methyl-3-pyridinyl)acetate (intermediate 128, 0.748 mmole) were dissolved in 4.74 mL of DCM. The solution was cooled to 0° C. and 365 μL of triethylamine (2.62 mmole, Aldrich) were added followed by 64 μL of Mesyl-Cl (0.821 mmole, Aldrich). The mixture was stirred at 0° C. for 2 h 20. Then, 175 mg of (8aR)-octahydropyrrolo[1,2-a]pyrazine dihydrochloride (intermediate 115, 1.076 mmole) were added. The solution was warmed to RT and stirred for 17 h. Then, 128 mg of (8aR)-octahydropyrrolo[1,2-a]pyrazine dihydrochloride (intermediate 115, 0.643 mmole) and 207 μL of triethylamine (1.49 mmole, Aldrich) were added. The mixture was stirred at RT for 75 h. The mixture was then poured into 15 mL of a saturated K₂CO₃ solution. 50 mL of AcOEt were added and the biphasic solution was transferred to a separatory funnel. The aqueous phase was extracted twice with 25 mL of AcOEt. The combined organic layers were dried over Na₂SO₄, filtered and evaporated to dryness. The resulting crude was then purified by flash chromatography on silica gel with the following eluent: A: CH+3% TEA/B: AcOEt+3% TEA: 0% B for 1 min, 0% to 100% B in 10.5 min, 100% B for 5.5 min. Evaporation of the solvents under reduced pressure gives the title compound as a yellow oil (82.4 mg, 36%). ¹H-NMR (400 MHz, CDCl₃): δ 1.20-1.45 (5H, m), 1.67-1.95 (3H, m), 2.04-2.48 (4H, m), 2.55-2.58 (3H, m), 2.65-2.84 (1H, m), 2.89-3.11 (3H, m), 4.05-4.27 (3H, m), 7.16 (1H, dd), 7.72 (1H, dd), 8.51 (1H, d). LC-MS [XTerra MC C18, 30×4.6 mm, 2.5 μm, gradient: A NH₄CO₃ 5 mM, pH 10; (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.38 min (100%) m\z (ES)=304 [M+H]⁺.

### Intermediate 130: N′-[3,5-bis(trifluoromethyl)phenyl]-2-[(8aR)-hexahydropyrrolo[1,2-a]pyrazin-2(1H)-yl]-2-(6-methyl-3-pyridinyl)acetohydrazide-mixture of diastereoisomers

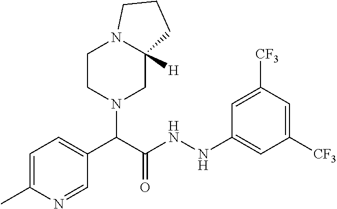

82.4 mg of ethyl(8aR)-hexahydropyrrolo[1,2-a]pyrazin-2(1H)-yl(6-methyl-3-pyridinyl)acetate (intermediate 129, 0.272 mmole) were dissolved in 900 μL of MeOH. 17 mg of potassium hydroxide (0.299 mmole) were added followed by 200 μL of water. The mixture was then stirred at RT for 19.5 h. The solution was then evaporated to dryness and the crude was dried under high vacuum for a couple of hours to give the corresponding potassium salt as an orange oil (76.6 mg). This compound was dissolved in 5 mL of DMF at RT. Then, 66 mg of 3,5-bis(trifluoromethyl)phenylhydrazine (0.269 mmole, Alfa Aesar) were added followed by 119 mg of BOP (0.269 mmole, Fluka). The mixture was stirred at RT for 19 h. The solution was passed through a SCX cartridge. Then the cartridge was washed with 25 mL of DCM, 25 mL of MeOH, and the compound was released with 25 mL of NH₃ 2M in MeOH. Solvents were removed under reduced pressure and the resulting crude was purified by flash chromatography on silica gel with the following eluent:A: AcOEt+3% TEA/B: MeOH+3% TEA: 0% B for 1 min, 0% to 10% B in 9 min, 10% B for 5 min. Evaporation of the solvents under reduced pressure gives the title compound as a yellow foam (69.2 mg, 56%). ¹H-NMR (400 MHz, CD₃OD): δ 1.28-1.54 (2H, m), 1.74-1.97 (3H, m), 2.11-2.34 (2H, m), 2.36-2.53 (2H, m), 2.57 (3H, d), 2.63-2.85 (1H, m), 2.97-3.24 (3H, m), 4.10 (1H, s), 7.00 (2H, d), 7.23 (1H, s), 7.36 (1H, d), 7.91-7.97 (1H, m), 8.56 (1H, s). LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.78 min (100%) m/z (ES)=502 [M+H]⁺.

### Compound 92: N′-[3,5-bis(trifluoromethyl)phenyl]-2-[(8aR)-hexahydropyrrolo[1,2-a]pyrazin-2(1H)-yl]-2-(6-methyl-3-pyridinyl)acetohydrazide-diastereoisomer 1

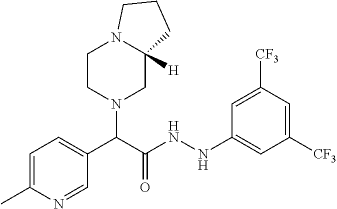

69.2 mg of N′-[3,5-bis(trifluoromethyl)phenyl]-2-[(8aR)-hexahydropyrrolo[1,2-a]pyrazin-2(1H)-yl]-2-(6-methyl-3-pyridinyl)acetohydrazide (intermediate 130, 0.137 mmole) were purified by preparative chiral HPLC [Chiralpak AD-H, 25×0.46 cm, Mobile phase: n-Hex/2-Propanol 90/10% v/v, Flow: 1 mL/min]. Solvents were removed under reduced pressure to give the title compound as a colourless oil (34 mg, 49%). ¹H-NMR (400 MHz, CD₃OD): δ 1.27-1.40 (1H, m), 1.72-1.87 (4H, m), 2.17-2.30 (2H, m), 2.43-2.50 (2H, m), 2.57 (3H, s), 2.79 (1H, d), 3.06 (3H, dd), 4.09 (1H, s), 6.98 (2H, s), 7.22 (1H, s), 7.36 (1H, d), 7.93 (1H, dd), 8.55 (1H, d). LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.77 min (100%) m/z (ES)=502 [M+H]⁺; Chiral HPLC [Chiralpak AD-H, 25×0.46 cm, mobile phase: n-Hex/2-Propanol 90/10% v/v, flow: 1 mL/min]: Rₜ=5.90 min (100% d.e.).

### Compound 93: N′-[3,5-bis(trifluoromethyl)phenyl]-2-[(8aR)-hexahydropyrrolo[1,2-a]pyrazin-2(1H)-yl]-2-(6-methyl-3-pyridinyl)acetohydrazide-diastereoisomer 2

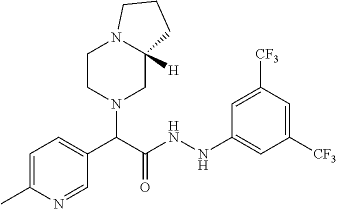

69.2 mg of N′-[3,5-bis(trifluoromethyl)phenyl]-2-[(8aR)-hexahydropyrrolo[1,2-a]pyrazin-2(1H)-yl]-2-(6-methyl-3-pyridinyl)acetohydrazide (intermediate 130, 0.137 mmole) were purified by preparative chiral HPLC [Chiralpak AD-H, 25×0.46 cm, Mobile phase: n-Hex/2-Propanol 90/10% v/v, Flow: 1 mL/min]. Solvents were removed under reduced pressure to give the title compound as a colourless oil (27 mg, 39%). ¹H-NMR (400 MHz, CD₃OD): δ 1.40-1.52 (1H, m), 1.78-1.93 (3H, m), 2.10-2.27 (3H, m), 2.30-2.42 (2H, m), 2.57 (3H, s), 2.66 (1H, dd), 2.93-3.09 (2H, m), 3.22 (1H, d), 4.08 (1H, s), 7.00 (2H, s), 7.24 (1H, s), 7.36 (1H, d), 7.94 (1H, dd), 8.56 (1H, d); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.79 min (100%) m/z (ES)=502 [M+H]⁺; Chiral HPLC [Chiralpak AD-H, 25×0.46 cm; Mobile phase: n-Hex/2-Propanol 90/10% v/v; flow: 1 mL/min]: Rₜ=12.50 min (100% d.e.).

### Intermediate 131: (±)-Ethyl hydroxy(2-methyl-3-pyridinyl)acetate

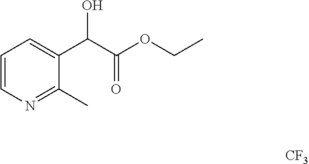

7.27 mL of nBuLi solution in hexanes (11.63 mmole, Aldrich) were added to 2.91 mL of isopropylmagnesium chloride 2.0M in diethylether (5.81 mmole) diluted in 24 mL of THF at 0° C. (int. T° C.=3° C.). The mixture was stirred at 0° C. for 15 min. Then the solution was cooled to −10° C. (int. T° C.=−15° C.) and a solution of 2 g of 3-bromo-2-methylpyridine (11.63 mmole, Aldrich) in 11.5 mL of THF was added dropwise. The mixture was stirred at −10° C. for 0.5 h. Then 4.75 mL of ethyl glyoxylate 50% in toluene (23.25 mmole, Fluka) were added and the solution was stirred for 2.5 h at 0° C. (int. T° C.=2° C.). The mixture was then poured into 100 mL of a saturated K₂CO₃ solution. The resulting precipitate was then filtered off and the cake was washed with 100 mL of AcOEt. The resulting biphasic solution was transferred to a separatory funnel and the aqueous phase was extracted twice with 50 mL of AcOEt. The combined organic layers were dried over Na₂SO₄, filtered and evaporated to dryness. The resulting crude was then purified by flash chromatography on silica gel with the following eluent: A: CH+3% TEA/B: AcOEt+3% TEA: 0% B for 1 min, 0% to 75% B in 14 min, 75% B for 7 min. Evaporation of the solvents under reduced pressure gives the title compound as a yellow oil (1.136 g, 50%). ¹H-NMR (400 MHz, CDCl₃): δ 1.25 (3H, t), 2.68 (3H, s), 4.16-4.35 (2H, m), 5.37 (1H, s), 7.18 (1H, dd), 7.67 (1H, dd), 8.48 (1H, dd); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.23 min (100%) m/z (ES)=196 [M+H]⁺.

### Intermediate 132: Ethyl (8aR)-hexahydropyrrolo[1,2-a]pyrazin-2(1H)-yl(2-methyl-3-pyridinyl)acetate-mixture of diastereoisomers

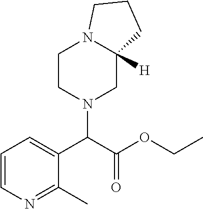

600 mg of ethyl(±)-hydroxy(2-methyl-3-pyridinyl)acetate (intermediate 131, 3.07 mmole) were dissolved in 19.45 mL of DCM. The solution was cooled to 0° C. and 1.5 mL of TEA (10.76 mmole) were added followed by 263 μL of mesyl-Cl (3.37 mmole, Aldrich). The mixture was stirred at 0° C. for 1 h 20. Then 407 mg (8aR)-octahydropyrrolo[1,2-a]pyrazine dihydrochloride (intermediate 115, 3.23 mmole) were added and the solution was warmed to RT and stirred for 73 h. 193 mg of (8aR)-octahydropyrrolo[1,2-a]pyrazine dihydrochloride (intermediate 115, 1.535 mmole) and 428 μL of TEA (3.07 mmole) were added and the mixture was stirred at RT for 19.5 h. Again 193 mg of (8aR)-octahydropyrrolo[1,2-a]pyrazine dihydrochloride (intermediate 115, 1.535 mmole) and 428 μL of TEA (3.07 mmole) were added and the mixture was stirred at RT for 20 h. The mixture was then poured into 30 mL of a saturated K₂CO₃ solution. 75 mL of AcOEt were added and the biphasic solution was transferred to a separatory funnel. The aqueous phase was extracted twice with 50 mL of AcOEt. The combined organic layers were dried over Na₂SO₄, filtered and evaporated to dryness. The resulting crude was then purified by flash chromatography on silica gel with the following eluent: A: CH+3% TEA/B: AcOEt+3% TEA: 0% B for 1 min, 0% to 100% B in 10 min, 100% B for 7 min. After evaporation of the solvents under reduced pressure, the resulting residue was dissolved in 10 mL of DCM and 1.2 g of PS-Trisamine (loading: 4.11 mmol/g) were added. The mixture was then stirred for 21 h at RT. The reaction mixture was filtered and the resin was washed with 50 mL of DCM. Solvents were removed under reduced pressure to give the title compound as a yellow oil (380 mg, 40%). ¹H-NMR (400 MHz, CDCl₃): δ 1.20-1.46 (5H, m), 1.64-1.97 (3H, m), 2.09-2.39 (4H, m), 2.62-2.82 (4H, m), 2.89-3.12 (3H, m), 4.09-4.26 (2H, m), 4.28-4.39 (1H, m), 7.13-7.22 (1H, m), 7.84-7.94 (1H, m), 8.39-8.50 (1H, m); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.42 min (100%) m/z (ES)=304 [M+H]⁺.

### Intermediate 133: N′-[3,5-bis(trifluoromethyl)phenyl]-2-[(8aR)-hexahydropyrrolo[1,2-a]pyrazin-2(1H)-yl]-2-(2-methyl-3-pyridinyl)acetohydrazide-mixture of diastereoisomers

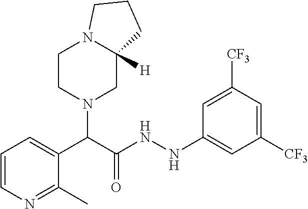

380 mg of ethyl(8aR)-hexahydropyrrolo[1,2-a]pyrazin-2(1H)-yl(2-methyl-3-pyridinyl)acetate (intermediate 132, 1.252 mmole) were dissolved in 4.14 mL of MeOH. 77 mg of potassium hydroxide (1.378 mmole, Fluka) were added followed by 920 μL of water. The mixture was then stirred at RT for 71 h. 35 mg of potassium hydroxide (0.626 mmole, Fluka) were added and the mixture was stirred at RT for 24 h. Solvents were removed under reduced pressure and the resulting crude was then dried under high vacuum for a couple of hours to give 446 mg of a yellow oil. The corresponding potassium salt was dissolved in 29 mL of DMF at RT. Then 382 mg of [3,5-bis(trifluoromethyl)phenyl]hydrazine (1.565 mmole, Alfa Aesar) were added followed by 692 mg of BOP (1.565 mmole, Fluka) and the mixture was stirred at RT for 18 h. The solution was passed through a SCX cartridge (10 g) and the cartridge was washed with 50 mL of DCM, 50 mL of MeOH, and the compound was released with 100 mL of NH₃ 2M in MeOH. Solvents were removed under reduced pressure and the resulting crude was then purified by flash chromatography on silica gel with the following eluent: A: AcOEt+3% TEA/B: MeOH+3% TEA: 0% B for 1 min, 0% to 10% B in 10 min, 10% B for 7 min. Evaporation of the solvents under reduced pressure gives a yellow solid that was triturated once with 3 mL of Et₂O and twice with 2 mL of Et₂O, Supernatant was removed and the compound was dried a couple of hours under high vacuum to give the title compound as a clear yellow solid (309 mg, 43%). ¹H-NMR (400 MHz, DMSO-d₆): δ 1.24 (1H, s), 1.59-1.86 (4H, m), 1.91-2.04 (2H, m), 2.12 (2H, d), 2.61-2.72 (4H, m), 2.94 (3H, s), 4.27-4.29 (1H, m), 7.04 (2H, s), 7.23 (1H, dd), 7.27-7.32 (1H, m), 7.89-7.97 (1H, m), 8.35-8.41 (1H, m), 8.78 (1H, d), 10.42 (1H, d); LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.36 min (100%) m/z (ES)=502 [M+H]⁺.

### Compound 94: N′-[3,5-bis(trifluoromethyl)phenyl]-2-[(8aR)-hexahydropyrrolo[1,2-a]pyrazin-2(1H)-yl]-2-(2-methyl-3-pyridinyl)acetohydrazide-diastereoisomer 1

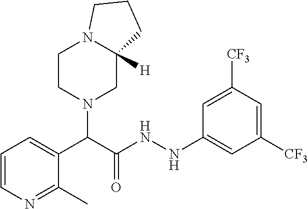

309 mg of (±)-N′-[3,5-bis(trifluoromethyl)phenyl]-2-[(8aR)-hexahydropyrrolo[1,2-a]pyrazin-2(1H)-yl]-2-(2-methyl-3-pyridinyl)acetohydrazide (intermediate 133, 0.62 mmole) were purified by preparative chiral HPLC [Chiralpak AD-H, 25×0.46 cm, Mobile phase: n-Hex/2-Propanol 93/7% v/v, flow: 1 mL/min]. Solvents were removed under reduced pressure to give the title compound as a yellow solid (149 mg, 48%). ¹H-NMR (400 MHz, CD₃OD): δ 1.30-1.40 (1H, m), 1.72-1.85 (3H, m), 1.97 (1H, t), 2.17-2.28 (2H, m), 2.37-2.47 (1H, m), 2.58-2.68 (1H, m), 2.73 (3H, s), 2.83 (1H, d), 2.98-3.12 (3H, m), 4.40 (1H, s), 7.05 (2H, s), 7.23 (1H, s), 7.32 (1H, dd), 8.15 (1H, dd), 8.41 (1H, dd); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.77 min (100%) m/z (ES)=502 [M+H]⁺; Chiral HPLC [Chiralpak AD-H, 25×0.46 cm, Mobile phase: n-Hex/2-Propanol 93/7% v/v; flow: 1 mL/min]: Rₜ=8.91 min (100% d.e.).

### Compound 95: N′-[3,5-bis(trifluoromethyl)phenyl]-2-[(8aR)-hexahydropyrrolo[1,2-a]pyrazin-2(1H)-yl]-2-(2-methyl-3-pyridinyl)acetohydrazide-diastereoisomer 2

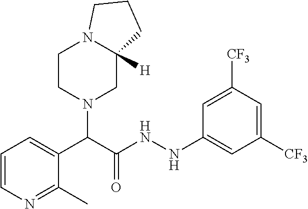

309 mg of (±)-N′-[3,5-bis(trifluoromethyl)phenyl]-2-[(8aR)-hexahydropyrrolo[1,2-a]pyrazin-2(1H)-yl]-2-(2-methyl-3-pyridinyl)acetohydrazide (intermediate 133, 0.62 mmole) were purified by preparative chiral HPLC [Chiralpak AD-H, 25×0.46 cm, Mobile phase: n-Hex/2-Propanol 93/7% v/v, flow: 1 mL/min]. Solvents were removed under reduced pressure to give the title compound as a yellow solid (138 mg, 45%). ¹H-NMR (400 MHz, CD₃OD): δ 1.41-1.53 (1H, m), 1.76-1.93 (3H, m), 2.13-2.38 (5H, m), 2.64-2.76 (4H, m), 2.94-3.10 (2H, m), 3.17 (1H, d), 4.41 (1H, s), 7.07 (2H, s), 7.24 (1H, s), 7.33 (1H, dd), 8.18 (1H, d), 8.41 (1H, dd); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.82 min (100%) m/z (ES)=502 [M+H]⁺; Chiral HPLC [Chiralpak AD-H, 25×0.46 cm, Mobile phase: n-Hex/2-Propanol 93/7% v/v; flow: 1 mL/min]: Rₜ=11.06 min (100% d.e.).

### Intermediate 134: 2,3-Dihydro-1,4-benzodioxin-6-yl[8aR)-hexahydropyrrolo[1,2-a]pyrazin-2(1H)-yl]acetic acid-mixture of diastereoisomers

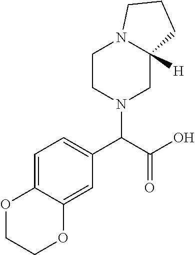

181 mg of (8aR)-octahydropyrrolo[1,2-a]pyrazine dihydrochloride (intermediate 115, 1.111 mmole) and 3 mL of acetonitrile were placed in a microwave vial. 310 μL of triethylamine (2.223 mmole) were added and the mixture was stirred. Then 200 mg of 2,3-dihydro-1,4-benzodioxin-6-ylboronic acid (1.111 mmole, Aldrich) and 104 mg of glyoxylic acid hydrate (1.4 mmole, Aldrich) were added and the mixture was heated at 120° C. for 20 min under microwaves irradiation. The solution was passed through a HLB cartridge. Then the cartridge was washed with H₂O (20 mL), 95% H₂O/5% MeOH (20 mL), 90% H₂O/10% MeOH (20 mL), 85% H₂O/15% MeOH (20 mL), 80% H₂O/20% MeOH (20 mL), 75% H₂O/25% MeOH (20 mL), 70% H₂O/30% MeOH (20 mL), 65% H₂O/35% MeOH (20 mL), 60% H₂O/40% MeOH (20 mL), 55% H₂O/45% MeOH (20 mL). Solvents were removed under reduced pressure to give the title compound as a yellow oil (117 mg, 33%). ¹H-NMR (400 MHz, CD₃OD): δ 1.92 (1H, s), 2.10 (3H, d), 2.42-3.26 (6H, m), 3.37-3.56 (3H, m), 4.09 (1H, d), 4.98 (4H, s), 6.81-6.84 (1H, m), 6.87-6.91 (1H, m), 6.94-6.97 (1H, m); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=1.14 min (100%) m/z (ES)=319 [M+H]⁺.

### Intermediate 135: N′-[3,5-bis(trifluoromethyl)phenyl]-2-(2,3-dihydro-1,4-benzodioxin-6-yl)-2-[(8aR)-hexahydropyrrolo[1,2-a]pyrazin-2(1H)-yl]acetohydrazide-mixture of diastereoisomers

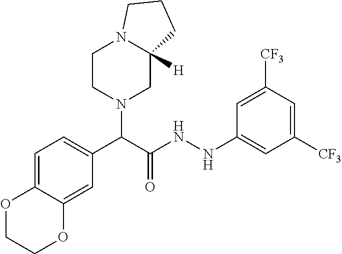

116 mg of 2,3-dihydro-1,4-benzodioxin-6-yl[(8aR) hexahydropyrrolo[1,2-a]pyrazin-2(1H)-yl]acetic acid (intermediate 134, 0.296 mmole) were dissolved in 6 mL of DMF at RT. Then 124 μL of TEA (0.889 mmole) were added followed by 80 mg of [3,5-bis(trifluoromethyl)phenyl]hydrazine (0.326 mmole) and 144 mg of BOP (0.326 mmole). The mixture was stirred at RT for 19 h. The solution was passed through a SCX cartridge (5 g). Then the cartridge was washed with 50 mL of DCM, 50 mL of MeOH, and the compound was released with 100 mL of NH₃ 2M in MeOH. Solvents were removed under reduced pressure and the resulting crude was purified by flash chromatography on silica gel with the following eluent: 95% AcOEt/5% MeOH+3% TEA. Evaporation of the solvents gives the title compound as a yellow oil (143 mg, 89%). ¹H-NMR (400 MHz, CD₃OD): δ 1.23-1.51 (1H, m), 1.71-1.91 (3H, m), 2.04-2.56 (5H, m), 2.69-3.24 (4H, m), 3.84-3.88 (1H, m), 4.21-4.28 (4H, m), 6.85 (3H, m), 7.00 (3H, m), 7.09 (1H, t), 7.21 (1H, s); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=2.01 min (100%) m/z (ES)=545 [M+H]⁺.

### Compound 96: N′-[3,5-bis(trifluoromethyl)phenyl]-2-(2,3-dihydro-1,4-benzodioxin-6-yl)-2-[(8aR)-hexahydropyrrolo[1,2-a]pyrazin-2(1H)-yl]acetohydrazide-diastereoisomer 1

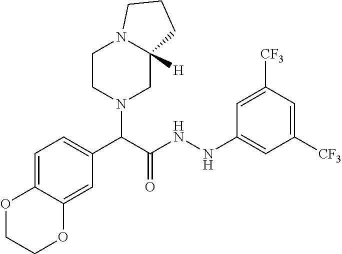

143 mg of N′-[3,5-bis(trifluoromethyl)phenyl]-2-(2,3-dihydro-1,4-benzodioxin-6-yl)-2-[(8aR)-hexahydropyrrolo[1,2-a]pyrazin-2(1H)-yl]acetohydrazide (intermediate 135, 0.26 mmole) were purified by preparative chiral HPLC [Chiralpak AD-H, Mobile phase=n-Hex/IPA 80/20 v/v, flow=1 mL/min]. Solvents were removed under reduced and the residue was triturated with Et₂O, Supernatant was removed and the resulting solid was dried under high vacuum to give the title compound as a yellow solid (18.7 mg, 13%). ¹H-NMR (400 MHz, CD₃OD): δ 1.40-1.52 (1H, m), 1.80-1.97 (3H, m), 2.39-2.73 (4H, m), 2.83-3.26 (5H, m), 3.89 (1H, s), 4.23-4.30 (4H, m), 6.87 (1H, d), 6.96-7.02 (3H, m), 7.08 (1H, d), 7.21 (1H, s); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=2.05 min (100%) m/z (ES)=545.0 [M+H]⁺; Chiral HPLC [Chiralpak AD-H, Mobile phase=n-Hex/IPA 80/20 v/v, flow=1 mL/min]: Rₜ=7.54 min (>99.5% d.e.).

### Compound 97: N′-[3,5-bis(trifluoromethyl)phenyl]-2-(2,3-dihydro-1,4-benzodioxin-6-yl)-2-[(8aR)-hexahydropyrrolo[1,2-a]pyrazin-2(1H)-yl]acetohydrazide-diastereoisomer 2

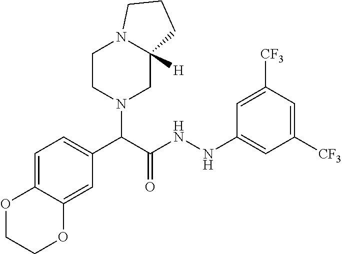

143 mg of N′-[3,5-bis(trifluoromethyl)phenyl]-2-(2,3-dihydro-1,4-benzodioxin-6-yl)-2-[(8aR)-hexahydropyrrolo[1,2-a]pyrazin-2(1H)-yl]acetohydrazide (intermediate 135, 0.26 mmole) were purified by preparative chiral HPLC [Chiralpak AD-H, Mobile phase=n-Hex/IPA 80/20 v/v, flow=1 mL/min]. Solvents were removed under reduced and the resulting solid was dried under high vacuum to give the title compound as a white solid (54.6 mg, 38%). ¹H-NMR (400 MHz, CD₃OD): δ 1.39-1.55 (1H, m), 1.75-1.93 (3H, m), 2.02-2.45 (5H, m), 2.70-2.77 (1H, m), 2.92-3.12 (2H, m), 3.19 (1H, d), 3.86 (1H, s), 4.25 (4H, s), 6.85 (1H, d), 6.97-7.03 (3H, m), 7.09 (1H, d), 7.22 (s, 1H); LC-MS [Supelcosil ABZ+Plus 33×4.6 mm, 3 μm, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.78 min (100%) m/z (ES)=545.0 [M+H]⁺; Chiral HPLC [Chiralpak AD-H, Mobile phase=n-Hex/IPA 80/20 v/v, flow=1 mL/min]: Rₜ=10.70 min (>99.5% d.e.).

### Alternative Preparation of Compound 89

### Intermediate 115: (8aR)-Octahydropyrrolo[1,2-a]pyrazine dihydrochloride

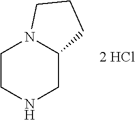

62 g of (8aR)-hexahydropyrrolo[1,2-a]pyrazin-6(2H)-one (prepared as set out in WO03066635A1, 442 mmole) were suspended in 1 L of anhydrous THF (15 vol.) and the mixture was cooled to 7° C. (internal). 1.33 L of Borane 1M sol. in THF (1327 mmol, Aldrich) were added dropwise in 20 min. The mixture was heated at 50° C. (internal) for 5 h. Then the solution was cooled to 7° C. (internal) and 500 mL of MeOH were added. The solution was left at 25° C. overnight. 1 L of HCl 1M in ether (16 vol.) were added and the mixture was heated at 50° C. (internal) for 4 h. Further 200 mL of HCl 1M in ether were added and the mixture was heated at 50° C. for another hour. The mixture was cooled to 25° C. and the solvent was evaporated. MeOH (2×1250 ml) was added and evaporated to give a white solid (89.7 g, 100%). ¹H-NMR (400 MHz, DMSO-d₆): δ 1.15-4.05 (13H, m), 9.2-9.9 (2H, br s), 11.84-12.04 (1H, br s).

### Intermediate 136: (8aR)-hexahydropyrrolo[1,2-a]pyrazin-2(1H)-yl(3-pyridinyl)acetonitrile-mixture of diastereoisomers

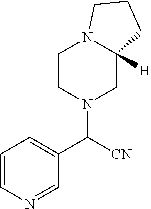

To a solution of 30.8 mL of 3-pyridinecarbaldehyde (326 mmole, Aldrich) in 52.5 mL of diethyl ether (1.5 vol.) were added 52.5 mL of TMSCN (392 mmole, Aldrich) and 5.21 g of zinc iodide (16.32 mmole, Aldrich) at RT under nitrogen. The orange reaction mixture was cooled to 10° C. (internal) and it was stirred for 5 min. Then a solution of 70 g of (8aR)-octahydropyrrolo[1,2-a]pyrazine dihydrochloride (intermediate 115, 352 mmole) and 95 mL of TEA (685 mmole) in 286 mL of methanol (8.1 vol.) was added dropwise to the reaction mixture and it was refluxed for 3 h. The solution was cooled to RT and 700 mL of a saturated solution of K₂CO₃ in water and 700 mL of AcOEt were added. The phases were separated and the water phase was back extracted twice with AcOEt (700 mL and 350 mL). The combined organic layers were dried over Na₂SO₄, filtered and evaporated to dryness. The crude was purified by flash chromatography on silica gel eluting with AcOEt+TEA 2% v/v to give the title compound as an orange liquid that solidified at +5° C. (71.3 g, 90%) ¹H-NMR (400 MHz, CDCl₃): δ 1.30-1.58 (1H, m), 1.64-2.05 (4H, m), 2.05-2.26 (2H, m), 2.35-2.54 (2H, m), 2.56-3.02 (2H, m), 3.05-3.18 (2H, m), 4.88-4.99 (1H, m), 7.34-7.41 (1H, m), 7.89 (1H, d), 8.65 (1H, ddd), 8.77-8.83 (1H, m). TLC plate (DCM/MeOH 9:1), R_{f}=0.58.

### Intermediate 137: 2-[(8aR)-hexahydropyrrolo[1,2-a]pyrazin-2(1H)-yl]-2-(3-pyridinyl)-acetamide-mixture of diastereoisomers

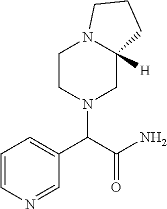

71 g of (8aR)-hexahydropyrrolo[1,2-a]pyrazin-2(1H)-yl(3-pyridinyl)acetonitrile (intermediate 136, 293 mmole) were suspended in 710 mL of n-Hexane and cooled down to 0° C. Then 437 mL of concentrated sulfuric acid (8204 mmole) were added dropwise and the obtained mixture was allowed to reach RT and was stirred mechanically for 2 days. The mixture was cooled down to 0° C. and 1 volume of ice was added. The mixture was slowly alkalinized with 1.4 L of a 28% solution of ammonia in water. The aqueous phase was extracted twice with 1.5 L of DCM, The organic phase was dried over Na₂SO₄, filtered and evaporated to dryness to give a first batch of the title compound as a yellow solid (39 g). The aqueous phase was extracted twice with 2 L of DCM. The combined organic layers were then dried over Na₂SO₄, filtered and evaporated to dryness to give a second batch of the title compound as a yellow solid (15 g). Finally, the aqueous layer was concentrated under reduced pressure to approximately 1 L, solids were removed by filtration and mother liquors were extracted twice with 1.5 L of DCM. The combined organic layers were then dried over Na₂SO₄, filtered and evaporated to dryness to give the third batch of the title compound as a yellow solid (11.7 g) (global yield: 86%). ¹H-NMR (400 MHz, CD₃OD): δ 1.24-1.50 (1H, m), 1.70-2.28 (6H, m), 2.53 (2H, m), 2.76 (1H, m), 2.89-3.24 (3H, m), 4.01-4.09 (1H, m), 7.47 (1H, dd), 7.92-8.01 (1H, m), 8.52 (1H, m), 8.58-8.64 (1H, m); TLC Plate R_{f}=0.15 (AcOEt/MeOH 9/1+TEA 3% v/v)

### Compound 89: (2R)—N′-[3,5-bis(trifluoromethyl)phenyl]-2-[(8aR)-hexahydropyrrolo[1,2-a]pyrazin-2(1H)-yl]-2-(3-pyridinyl)ethanohydrazide-diastereoisomer 2

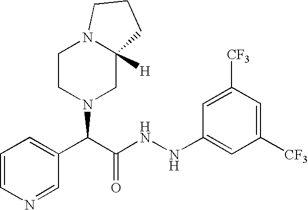

60 g of 2-[(8aR)-hexahydropyrrolo[1,2-a]pyrazin-2(1H)-yl]-2-(3-pyridinyl)acetamide (intermediate 137, 230 mmole) were dissolved in 2.1 L of DCM. Then 107 mL of Boc₂O (461 mmole) and 2.81 g of DMAP (23.03 mmole) were added. The mixture was stirred at RT for 5.5 h. The solution was concentrated to the third and 67.5 g of [3,5-bis(trifluoromethyl)phenyl]hydrazine (277 mmole) were added followed by 5.63 g of DMAP (46.06 mmole). The solution was stirred at RT for 64 h. The solid was filtered, washed with diethyl ether and dried in vacuo to give 38.7 g of a white solid (batch 1). Mother liquors were concentrated to a minimum volume and were charged on a silica pad. The compound was eluted with AcOEt/MeOH 9:1+3% of TEA. Removal of the solvent gave 50.9 g of a solid. This solid was suspended in 500 mL of diisopropyl ether and 57.5 g of 1,4-diazabicyclo[2.2.2]octane (513 mmole, Aldrich) were added. The suspension was heated at 80° C. overnight. The mixture was allowed to reach RT and filtered through a sintered glass funnel to obtain 20 g of a beige solid (batch 2). Batch 1 and batch 2 were mixed together (53.7 g, 110 mmole) and were suspended in 540 mL of acetone and heated to 70° C. for 45 min. Then the mixture was allowed to reach RT and stirred for 1 h. The reaction mixture was filtered through a sintered glass funnel, the solid was washed with 150 mL of acetone, and dried in vacuo to give the title compound as a white solid (49 g, 43%). ¹H-NMR (400 MHz, DMSO-d₆): δ 1.13-1.37 (1H, m), 1.58-1.76 (3H, m), 1.90-2.20 (5H, m), 2.51-2.56 (1H, m), 2.75-3.08 (3H, m), 4.07 (1H, s), 7.03 (2H, s), 7.29 (1H, s), 7.40 (1H, dd), 7.88 (1H, dt), 8.54 (1H, dd), 8.64 (1H, d), 8.77 (1H, s), 10.46 (1H, s). Chiral HPLC [Chiralpak AD-H, mobile phase: n-Hexane/Isopropanol 92/8 v/v, flow: 1 mL/min]: Rₜ=20.07 min (d.e. >99%).

X-Ray Structure of compound 89:

A spatula tip of compound 2 (ca. 2 mg) was suspended in water (ca. 2 mL) in a soda glass sample vial (50×12/13 mm). The solution was heated to 40° C. while being stirred. At 40° C. acetonitrile was added dropwise until the solid just dissolved (ca 0.35 mL). The solution was then cooled to ambient temperature and filtered through a 0.45 μm syringe tip filter into a soda glass sample vial, which had been blown through with compressed air to remove any particulates. The vial was sealed with a plastic lid, into which 2 holes had previously been pierced with a needle. The solution was allowed to evaporate slowly before isolation of the crystals.

X-ray experiment was performed on a Nonius KappaCCD diffractometer.

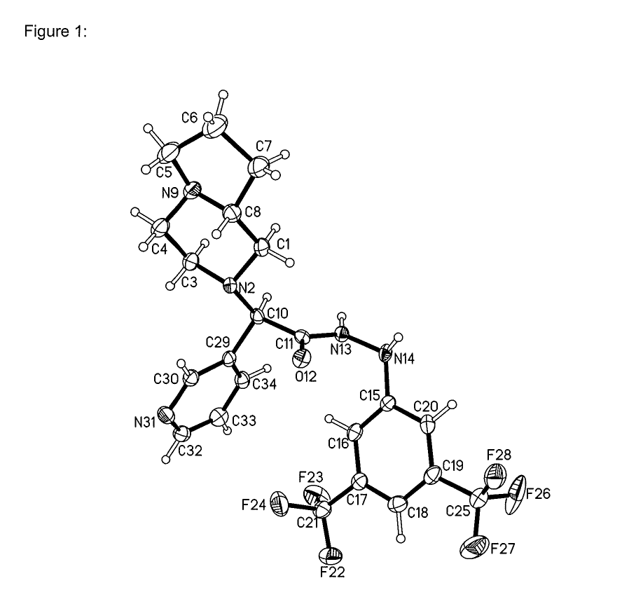

The ORTEP diagram of compound 2 is shown in FIG. 1: a view of a crystal structure of a molecule of compound 2, showing the numbering scheme employed. Anisotropic atomic displacement ellipsoids for the non-hydrogen atoms are shown at the 50% probability level. Hydrogen atoms are displayed with an arbitrarily small radius. The minor component of the trifluoromethyl disorder is omitted for clarity.

The diffraction experiment unambiguously gave the relative stereochemistry (C8, R; C10, R) and the modelled enantiomer was chosen based on the ‘known’ stereocentre (C8, see WO03066635A1).

### Intermediate 138: (8aR)-hexahydropyrrolo[1,2-a]pyrazin-2(1H)-yl(1,3-thiazol-2-yl)-acetonitrile (diastereomeric mixture)

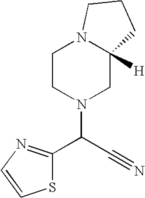

To a solution of 315 mg of thiazole-2-carbaldehyde (2.8 mmole) in 15 mL of diethyl ether were added 410 μL of trimethylsilyl cyanide (3 mmole) and 41 mg of zinc iodide (0.13 mmole) at RT under nitrogen. The mixture was cooled to 0° C. and it was stirred at 0° C. for 5 min. Then a solution of 500 mg of (R)-octahydropyrrolo[1,2-a]pyrazine dihydrochloride (intermediate 115, 2.5 mmole) in 15 mL of MeOH was added to the mixture. Then, 730 μL of TEA (5 mmole) were added to the reaction. The reaction was refluxed overnight. The mixture was cooled to RT and treated with 20 mL of saturated solution of K₂CO₃. The mixture was extracted with AcOEt (3×20 mL). The combined organic layers were washed with a saturated solution of NaHCO₃ (20 mL), and dried over Na₂SO₄ filtered and evaporated to dryness. The crude product was purified by flash chromatography on silica gel to afford the title compound (400 mg, 64.2%) as a brown solid (diastereomeric mixture). ¹H-NMR (CDCl₃): δ 1.40-1.60 (1H, m), 1.80-2.00 (4H, m), 2.00-2.40 (2H, m), 2.50-2.80 (2H, m), 2.90-3.20 (4H, m), 5.12-5.16 (1H, d), 7.42 (1H, d), 7.78 (1H, d); TLC (DCM/MeOH 10:1) R_{f}=0.4.

### Intermediate 139: 2-[(8aR)-hexahydropyrrolo[1,2-a]pyrazin-2(1H)-yl]-2-(1,3-thiazol-2-yl)acetamide (diastereomeric mixture)

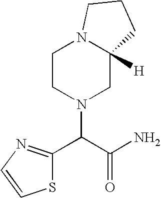

At 0° C., to a solution of 380 mg of (8aR)-hexahydropyrrolo[1,2-a]pyrazin-2(1H)-yl(1,3-thiazol-2-yl)acetonitrile (intermediate 138, 1.53 mmole) in 10 mL of n-hexane were added 10 mL of H₂SO₄ (200 mmole). The mixture was stirred overnight at RT. The mixture was cooled to 0° C., then ice was added. The acid was neutralized with NH₃H₂O 28%. The mixture was freeze-dried. The residue was washed by DCM/MeOH (10/1). The organic layer was concentrated in vacuo to afford a crude product. The crude was purified by preparative HPLC [Sample Prep: DMSO, mobile phase: A: MeCN B: H₂O (0.05% NH₃.H₂O) Column: Gemini 250*21.2 5u C18. Wavelength: 220 nm Flow rate: 15 mL/min Injection volume: 3 mL. Run time: 20 min, Equilibration Time: 3 min. Gradient Profile Description: Time: 0 (% A:10, % B:90); 20 (% A:40, % B:60)] to afford the title compound (110 mg, 27%) as a brown solid (diastereomeric mixture). ¹H-NMR (CD₃OD) δ: 1.20-1.47 (1H, m), 1.70-1.86 (3H, m), 1.95-2.30 (3H, m), 2.31-2.60 (2H, m), 2.66-2.85 (1H, m), 2.90-3.14 (3H, m), 4.60 (1H, s), 7.59-7.60 (1H, d), 7.76-7.77 (1H, d); m/z (ES): 267 [M+H]⁺, 555 [2M+Na]⁺.

### Compound 98: N′-[3,5-bis(trifluoromethyl)phenyl]-2-[(8aR)-hexahydropyrrolo[1,2-a]pyrazin-2(1H)-yl]-2-(1,3-thiazol-2-yl)acetohydrazide (Diastereomeric mixture)

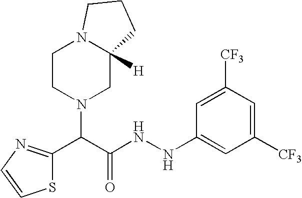

110 mg (0.413 mmole) of 2-[(8aR)-hexahydropyrrolo[1,2-a]pyrazin-2(1H)-yl]-2-(1,3-thiazol-2-yl)acetamide (intermediate 139) and 185 mg (0.847 mmole) of Boc-anhydride were dissolved in 4.13 mL of DCM to give a yellow solution. 5.05 mg (0.041 mmole) of DMAP were added and the reaction mixture was stirred at RT for 2 days. 0.2 eq of DMAP and 121 mg of [3,5-bis(trifluoromethyl)phenyl]hydrazine (0.496 mmole) were added and the solvent was partially evaporated. The reaction mixture was stirred at RT for 25 h. The solvent was removed and the crude material was added to a silica gel cartridge and eluted with AcOEt 100%, AcOEt/MeOH/NH₃ 2.0 M in MeOH 95/5/3% and AcOEt/MeOH/NH₃ 2.0 M in MeOH 90/10/3%. 42 mg of the title compound were obtained as a crude and purified by MDAP FractionLynx Autopurification System™ [Column: Gemini C18, 50×4.6 mm, 5 μm; Mobile phase: A: NH₄HCO₃ sol. 10 mM, pH 10/B: CH₃CN; Gradient: 35% B for 0.5 min, 35% to 95% B in 4.5 min, 95% B for 1.5 min; Flow rate: 2 ml/min; UV range: 210-350 nm; Ionization: ES⁺; Mass range: 100-900 amu]. Preparative chromatographic conditions Column: Gemini C18 AXIA, 50×21 mm, 5 μm; Mobile phase: A: NH₄HCO₃ sol. 10 mM, pH 10; B: CH₃CN; Gradient: 30% to 35% (B) in 1 min, 35% to 65% (B) in 7 min, 65% to 100% (B) in 1 min, 100% (B) for 1.5 min; Flow rate: 17 ml/min; UV range: 210-350 nm; Ionization: ES⁺; Mass range: 100-900 amu) to give the title compound (as diastereomeric mixture) (4 mg, 2%). ¹H-NMR (CDCl₃, 400 MHz) δ: 1.4-3.15 (m, 13H), 4.71 (s, 1H), 6.58 (s, 1H), 7.29, (s, 1H), 7.36 (s, 1H), 7.42 (d, 1H), 7.88 (t, 1H), 9.57 (br s, 1H); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min, 99% to 3% B in 0.35 min, flow rate: 1 ml/min]: Rₜ=0.64 min, m/z (ES): 494.10 [M+H]⁺.

### Intermediate 140: (±)-1,4-bis{[(1,1-dimethylethyl)oxy]carbonyl}-2-piperazinecarboxylic acid

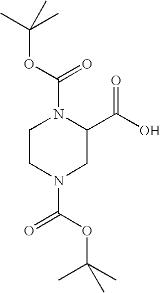

In a 1 L round-bottomed flask 15.76 g of NaOH (394 mmole) were poured in 390 mL of water, then 20 g of 2-piperazinecarboxylic acid (98 mmole) were added portionwise to give a colourless solution. This addition was performed keeping the temperature below +5° C. A solution of 50 mL of Boc₂O (215 mmole) dissolved in 1.4 dioxane was added dropwise and the solution was allowed to stir on. The day after the reaction was finished. The mixture was cooled to +5° C., then HCl 3N was added until pH=3. The suspension was then extracted by 300 mL of AcOEt, then the organic phase was dried over sodium sulfate and evaporated in vacuo to obtain 22.7 g of desired product as a white solid. The aqueous phase was acidified with 3N HCl until pH=3, then extracted with 2×100 mL of AcOEt. The organic phase was dried over sodium sulfate and evaporated in vacuo to obtain a second batch of 7.9 g of desired product The two batches were combined together to give the title compound (30.6 g, 94%). ¹H-NMR (400 MHz, DMSO-d₆): δ 1.33 (18H, m), 2.70-4.50 (8H, m), 12.9 (1H, s); m/z (ES) 330 [M+H]⁺.

### Intermediate 141: (±)-1,4-bis(1,1-dimethylethyl) 2-methyl 1,2,4-piperazinetricarboxylate

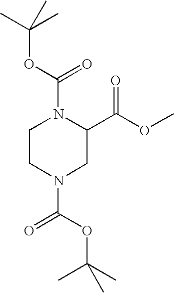

In a 500 mL round-bottomed flask, 22.6 g of intermediate 140 (68.4 mmole) were dissolved in 160 mL of DCM and 40 mL of methanol to give a colorless solution. 58 mL of Trimethylsilyl diazomethane (116 mmole) were added dropwise keeping the internal temperature below +2° C. The solution was allowed to reach RT and was stirred at RT for 2 h. The mixture was carefully evaporated under reduced pressure (Tbath=35° C.) and the solid residue was triturated with 100 mL of pentane, filtered and dried in vacuo to obtain 21 g of desired product as a white solid. The mother liquor was concentrated in vacuo to give 2.5 g of the title compound. UPLC-MS [Acquity™ UPLC BEH C18, 50×21 mm, 1.7 μm, Mobile phases: A: H₂O+0.1% HCOOH/B: MeCN+0.06% HCOOH. Gradient: t=0 min: 97% A, 3% B, t=0.1 min 94% A, 6% B t=0.6 min 30% A, 70% B t=1.10 min 1% A, 99% B t=1.45 min 97% A, 3% B t=1.50 min 97% A, 3% B flow rate: 1 ml/min, UV range wavelength 210-350 nm]: Rₜ=0.80 min, m/z (ES): 344 [M+H]⁺, 367 [M+Na]⁺.

### Intermediate 142: (±)-1,4-bis(1,1-dimethylethyl) 2-methyl 2-methyl-1,2,4-piperazinetricarboxylate

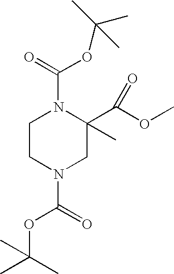

In a 500 ml round-bottomed flask, 12 g of intermediate 141 (34.8 mmole) were dissolved in 240 mL of THF to give a colourless solution. 38.3 mL of LiHMDS 1M in THF (38.3 mmole) were added dropwise to the solution keeping the mixture to −78° C. The reaction was allowed to stir at this temperature for 1 h. 2.40 mL of iodomethane (38.3 mmole) were added dropwise at −78° C. and the reaction was allowed to stir at this temperature for 1 h. The mixture was warmed to −20° C. and it was allowed to react at this temperature for 2 h. The reaction was almost complete and was cooled to −78° C. and 12 mL of LiHMDS 1M in THF (12 mmole, 1 M sol. In THF) and 1.2 mL of iodomethane (19 mmole) were added successively at this temperature. Then the mixture was warmed to −20° C. and after 1.5 h a TLC control showed then the reaction was finished. The mixture was quenched at 0° C. with 150 mL of a saturated solution of ammonium chloride. Then the mixture was allowed to reach RT. The two phases were separated and the aqueous was extracted with 2×200 mL of AcOEt. The resulting organic layer was dried over Na₂SO₄, filtered and evaporated under reduced pressure to obtain 12.2 g of desired product as a crude oil. UPLC-MS [Acquity™ UPLC BEH C18, 50×21 mm, 1.7 μm, Mobile phases: A: H₂O+0.1% HCOOH/B: MeCN+0.06% HCOOH; Gradient: t=0 min: 97% A, 3% B t=0.1 min 94% A, 6% B t=0.6 min 30% A, 70% B t=1.10 min 1% A, 99% B t=1.45 min 97% A, 3% B t=1.50 min 97% A, 3% B flow rate: 1 mL/min, UV range wavelength 210-350 nm] Rₜ=0.80 min, m/z (ES): 359 [M+H]⁺.

### Intermediate 143: (±)-bis(1,1-dimethylethyl) 2-(hydroxymethyl)-2-methyl-1,4-piperazine-dicarboxylate

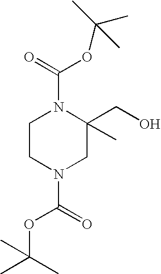

In a 500 mL round-bottomed flask, 10.2 g of (±)-1,4-bis(1,1-dimethylethyl) 2-methyl 2-methyl-1,2,4-piperazinetricarboxylate (intermediate 142, 28.5 mmole) were dissolved in 250 mL of THF to obtain a colourless solution. 28.5 mL of LiAlH₄ 1M in THF (28.5 mmole) were added dropwise at this solution keeping the internal temperature between −5 and 0° C. The reaction was stirred at 0° C. for 1.5 h. Additional 7 mL of LiAlH₄ were added and the mixture was stirred at 0° C. for 45 min. Additional 6 mL of LiAlH₄ were then added and the mixture was stirred at 0° C. for 45 min. The reaction was quenched by careful addition of 70 mL of a saturated aqueous solution of sodium sulfate keeping T below +3° C. The resulting suspension was filtered, then the two phases were separated and the aqueous layer was extracted with 2×100 mL of ethyl acetate. The resulting organic layer was dried over Na₂SO₄, filtered and evaporated in vacuo to obtain 9 g of a crude oil. The crude product was purified by flash chromatography on silica gel (250 g) and was eluted with CH/AcOEt from 8/2 to 7/3. Collected fractions were evaporated in vacuo to obtain 5.8 g of the title compound (5.8 g, 62%). UPLC-MS [Acquity™ UPLC BEH C18, 50×21 mm, 1.7 μm, Mobile phases: A: H₂O+0.1% HCOOH/B: MeCN+0.06% HCOOH; Gradient: t=0 min: 97% A, 3% B t=0.1 min 94% A, 6% B t=0.6 min 30% A, 70% B t=1.10 min 1% A, 99% B t=1.45 min 97% A, 3% B t=1.50 min 97% A, 3% B flow rate: 1 ml/min, UV range wavelength 210-350 nm]:Rₜ=0.74 min m/z (ES): 331 [M+H]⁺.

### Intermediate 144: (±)-bis(1,1-dimethylethyl) 2-formyl-2-methyl-1,4-piperazinedicarboxylate

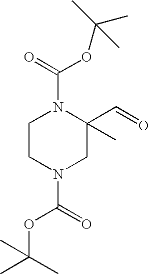

In a 2000 mL round-bottomed flask were dissolved 5.8 g of (±)-bis(1,1-dimethylethyl) 2-(hydroxymethyl)-2-methyl-1,4-piperazinedicarboxylate (intermediate 143, 17.55 mmole) dissolved in 700 mL of DCM to obtain a colorless solution. 2.95 g of NaHCO₃ (35.1 mmole) and 8.93 g of Dess-Martin periodinane (21.06 mmole) were added and the mixture was stirred at RT for 3 h. The reaction was quenched by addition of 250 mL of a 5% solution of Na₂S₂O₃. The two phases were separated and the organic layer was washed with 2×200 mL of NaHCO₃ sat solution. The organic phase was dried over Na₂SO₄, filtered and concentrated in vacuo to give the title compound as a white solid (5.1 g, 88%). ¹H-NMR (400 MHz, CDCl₃): δ 1.30 (3H, s), 1.44 (18H, m), 3.0-3.95 (6H, m), 9.5 (1H, s). TLC(CH/AcOEt 7:3) R_{f}=0.31.

### Intermediate 145: (±)-bis(1,1-dimethylethyl) 2-[(1E)-3-(ethyloxy)-3-oxo-1-propen-1-yl]-2-methyl-1,4-piperazinedicarboxylate

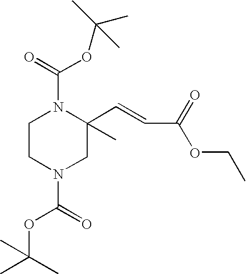

In a 100 mL round-bottomed flask were dissolved 5.1 g of (±)-bis(1,1-dimethylethyl) 2-formyl-2-methyl-1,4-piperazinedicarboxylate (intermediate 144, 15.53 mmole) in 80 mL of toluene to obtain a colourless solution. 8.12 g of ethyl(triphenyl-5-phosphanylidene)acetate (23.29 mmole) were added. The reaction was heated at an internal T of 80° C. for 24 h. Additional 6.6 g of ethyl(triphenyl-5-phosphanylidene)acetate were added and the mixture was heated at an internal T of 80° C. for 7 h. The mixture was allowed to cool to RT, then 50 mL of water were added dropwise. The two phases were separated, then the aqueous was extracted with 2×100 mL of AcOEt. The combined organic layers were dried over Na₂SO₄, filtered and concentrated in vacuo to obtain 20.5 g of crude product. This residue was purified by flash chromatography on silica gel (500 g of silica) and was eluted with CH/AcOEt from 8/2 to 7/3. Collected fractions were concentrated in vacuo to give the title compound (4.7 g, 76%). ¹H-NMR (400 MHz, CDCl₃): δ 1.25 (3H, t), 1.44 (18H, m), 1.50 (3H, s), 3.30-3.90 (6H, m), 4.20 (2H, q), 5.8 (1H, d), 7.0 (1H, d); m/z (ES): 399 [M+H]⁺.

### Intermediate 146: (±)-bis(1,1-dimethylethyl) 2-[3-(ethyloxy)-3-oxopropyl]-2-methyl-1,4-piperazinedicarboxylate

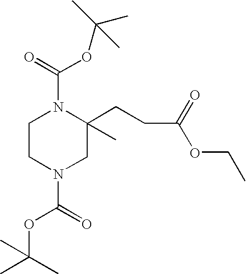

In a 500 mL pear flask, 3.0 g of (±)-bis(1,1-dimethylethyl) 2-[(1E)-3-(ethyloxy)-3-oxo-1-propen-1-yl]-2-methyl-1,4-piperazinedicarboxylate (intermediate 145, 7.53 mmole) were dissolved in 110 mL of Ethanol to give a colourless solution. 0.5 g of Pd/C (4.70 mmole) were added and the hydrogenation was performed at atmospheric pressure. The day after the reaction was finished. The mixture was filtered over a pad of celite and the cake was washed with ethanol (3×200 mL). The filtrate was concentrated in vacuo to obtain 2.93 g of the title compound as a colourless oil. Another batch of the same compound was prepared following the same procedure (1.4 g obtained starting from 1.4 g of title compound. ¹H-NMR (400 MHz, CDCl₃): δ 1.25 (3H, t), 1.35 (3H, s), 1.44 (18H, m), 1.90-3.90 (10H, m), 4.25 (2H, q). Rₜ=0.88 min m/z (ES): 401 [M+H]⁺.

### Intermediate 147: (±)-ethyl 3-(2-methyl-2-piperazinyl)propanoate bis-trifluoroacetic acid salt

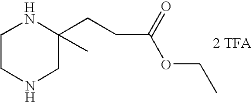

In a 250 mL pear flask 1.4 g of (±)-bis(1,1-dimethylethyl) 2-[3-(ethyloxy)-3-oxopropyl]-2-methyl-1,4-piperazinedicarboxylate were dissolved (intermediate 146, 3.50 mmole) in 50 mL of DCM to give a yellow solution. 11.85 mL of TFA (154 mmole) were added dropwise keeping the internal temperature below +2° C. with an ice bath. At the end of the addition the bath was removed and the mixture was allowed to stir at RT for 4 h. The reaction mixture was concentrated in vacuo to give a crude containing the title compound (3.6 g) and an excess of trifluoroacetic acid. UPLC-MS [Acquity™ UPLC BEH C18, 50×21 mm, 1.7 μm, Mobile phases: A: H₂O+0.1% HCOOH/B: MeCN+0.06% HCOOH. Gradient: t=0 min: 97% A, 3% B t=0.1 min 94% A, 6% B t=0.6 min 30% A, 70% B t=1.10 min 1% A, 99% B t=1.45 min 97% A, 3% B t=1.50 min 97% A, 3% B flow rate: 1 ml/min, UV range wavelength 210-350 nm]: Rₜ=0.21 min; m/z (ES): 201 [M+H]⁺.

### Intermediate 148: 8a-methylhexahydropyrrolo[1,2-a]pyrazin-6(2H)-one-enantiomer 1

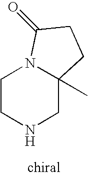

In a 500 mL pear flask 701 mg of (±)-ethyl 3-(2-methyl-2-piperazinyl)propanoate bis-trifluoroacetic acid salt (crude intermediate 147, 3.5 mmole) were dissolved in 90 mL of Ethanol to give a yellow solution. The mixture was heated to reflux for 1.5 h. The mixture was evaporated in vacuo to obtain 1.2 g of a crude (still containing a small amount of TFA). This crude was diluted with 20 mL of methanol and the desired product was isolated by SCX chromathography. The product was recovered eluting with 130 mL of 2M ammonia in methanol end evaporating in vacuo the basic methanolic solution to give 520 mg of the title compound as a yellow oil. Other two batches of the same compound were prepared following the same procedure. All batches were then combined to give 1.5 g of a racemic mixture that was separated by chiral preparative HPLC [Column: Chiralpack AS-H (25*2.0 cm), 5u, Mobile phase: n-hexane/ethanol 70/30 v/v, Flow rate 14 ml/min, UV=225 nm, 50 mg/uinj in ethanol/n hexane, UV at 225 nm: enantiomer 1: Rₜ=7.78 min, enantiomer 2: Rₜ=11.07 min] to give the title compound (enantiomer 1) (700 mg). ¹H-NMR (400 MHz, CDCl₃): δ 1.35 (3H, s), 1.7-1.8 (1H, m), 1.9-1.95 (1H, m), 2.3-2.6 (4H, m), 2.8-3.05 (3H, m), 3.9-3.95 (1H, m).

### Intermediate 149: 8a-methyloctahydropyrrolo[1,2-a]pyrazine dihydrochloride pure enantiomer

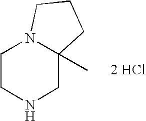

5.253 mL (5.25 mmole) of BH₃.THF complex was added dropwise to a solution of 270 mg (1.751 mmole) of 8a-methylhexahydropyrrolo[1,2-a]pyrazin-6(2H)-one (intermediate 148, 1.75 mmole) in 9 mL of THF cooled at 0° C. using a ice bath. The reaction mixture was stirred at 50° C. for 24 h and 1 eq of Borane was added. The reaction mixture was stirred at 50° C. for additional 3 h. The reaction mixture was cooled to 0° C. and 5 mL of MeOH and HCl (1.0 N in Et₂O) were added (caution). The reaction mixture was refluxed for 4 h. The suspension was cooled to RT and the volatiles were evaporated under reduced pressure. The crude was suspended in MeOH and the solution was treated with HCl (1.0 N in Et₂O). The resulting solution was concentrated under reduced pressure (these operations were done three times) to give the title compound as a white solid (320 mg, 86%). ¹H-NMR (400 MHz, DMSO-d₆) δ: 1.65 (s, 3H), 1.70-2.25 (m, 4H), 3.10-3.80 (m, 8H), 10.0 (br s, 2H), 12.0 (br s, 1H); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% B to 70% B in 0.5 min, 70% B to 99% B in 0.5 min., 99% B to 3% B in 0.35 min, flow rate: 1 ml/min]: R_{f}=0.16 min, m/z (ES): 141.06 [M+H]⁺ and Rₜ=0.45 min, m/z (ES): 141.06 [M+H]⁺.

### Intermediate 150: (8a-methylhexahydropyrrolo[1,2-a]pyrazin-2(1H)-yl)(3-methylphenyl)acetic acid hydrochloride-diastereomeric mixture

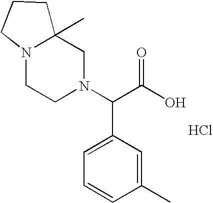

150 mg of 8a-methyloctahydropyrrolo[1,2-a]pyrazine (dihydrochloride salt) (intermediate 149, 0.704 mmole), 96 mg of (3-methylphenyl)boronic acid (0.704 mmole), 64.8 mg of glyoxylic acid (0.704 mmole) and 195 mg of K₂CO₃ (1.407 mmole) were suspended in 2.5 mL of acetonitrile to give a white suspension. The reaction mixture was heated at 120° C. for 20 min (3 cycles under microwave conditions). The volatiles were removed and the crude was solubilised in 2 mL of HCl 1.0 M in water. The solution was passed onto a HLB Oasis Extraction Cartridge (column size 6 g) and the compound was eluted with water and methanol (linear gradient starting from 100% water to 100% methanol) to give the title compound (diastereomeric mixture) as a brown gum (55 mg, 24%). UPLC-MS [Acquity™UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 ml/min]: Rₜ=0.47 min, m/z (ES): 289.15 [M+H]⁺.

### Compound 99: N′-[3,5-bis(trifluoromethyl)phenyl]-2-(8a-methylhexahydropyrrolo[1,2-a]pyrazin-2(1H)-yl)-2-(3-methylphenyl)acetohydrazide-diastereomeric mixture

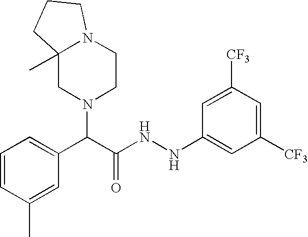

50 mg of (8a-methylhexahydropyrrolo[1,2-a]pyrazin-2(1H)-yl)(3-methylphenyl)acetic acid hydrochloride (intermediate 150, 0.154 mmole), 37.6 mg of [3,5-bis(trifluoromethyl)phenyl]hydrazine (0.154 mmole, Fluka), and 0.020 mL of NMM (0.185 mmole, Aldrich) were dissolved in 3 mL of DMF to give an orange solution. 82 mg of BOP (0.185 mmole, Fluka) were added and the reaction mixture was stirred at RT overnight. NaOH (1.0 M in water) was added and the aqueous layer was extracted with AcOEt. The organic phase was washed with a saturated aqueous solution of NaHCO₃, H₂O, and brine. The organic phase was dried over Na₂SO₄, filtered and concentrated. The crude material was purified by filtration on a SCX cartridge (column size 2 g) eluting with methanol and ammonia 0.5 M in methanol. The ammoniacal fractions were collected and the solvent evaporated. 80 mg of the title compound were obtained as a crude. The crude was purified by flash chromatography on silica gel (column size 5 g) eluting with DCM 100% and DCM/MeOH 20% to give the title compound as a diastereomeric mixture (40 mg, 50%). ¹H-NMR: (500 MHz, DMSO-d₆) δ 1.29-1.59 (1H, br s), 1.40 (3H, s), 1.60-1.81 (1H, br s), 1.77-2.16 (3H, br s), 2.29-2.44 (1H, br s), 2.31 (3H, s), 2.62 (2H, m), 2.96-3.20 (1H, br s), 4.03-4.09 (1H, 2s), 6.97 (2H, s), 7.17 (1H, s), 7.29 (3H, m), 8.73 (1H, s), 10.39 (1H, s), 10.64 (1H, br s); UPLC-MS [Acquity™ UPLC BEH C18, 50×21 mm, 1.7 μm, gradient: A: H₂O+0.1% HCOOH/B: MeCN+0.075% HCOOH: 3% to 6% B in 0.1 min., 6% to 70% B in 0.5 min, 70% to 99% B in 0.5 min., 99% to 3% B in 0.35 min, flow rate: 1 ml/min]: Rₜ=0.72 min, m/z (ES): 515.19 [M+H]⁺.

### Intermediate 151: (2-ethylphenyl)[(8aR)-hexahydropyrrolo[1,2-a]pyrazin-2(1H)-yl]acetic acid hydrochloride

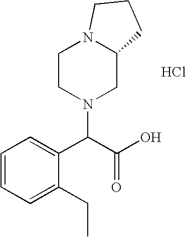

A mixture of 188 mg of (2-ethylphenyl)boronic acid (1.25 mmole, Aldrich), 250 mg of (8aR)-octahydropyrrolo[1,2-a]pyrazine di-hydrochloride (intermediate 115, 1.25 mmole), 116 mg of glyoxylic acid monohydrate (1.25 mmole, Aldrich) and 347 mg of K₂CO₃ (2.51 mmole) in 6 mL of CH₃CN was heated under microwave irradiation at 120° C. for 20 min. Then, the solvent was removed and the residue was treated with HCl 1N, charged on a HLB column and eluted with a gradient from 100% H₂O to 100% MeOH. Solvents were removed under reduced pressure to give the title compound as a mixture of two diastereoisomers (260 mg, 57%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.44 min. (91%) m/z (ES): 289.1 [M+H]⁺.

### Compound 100: N′-[3,5-bis(trifluoromethyl)phenyl]-2-(2-ethylphenyl)-2-[(8aR)-hexahydro-pyrrolo[1,2-a]pyrazin-2(1H)-yl]acetohydrazide-diastereomeric mixture

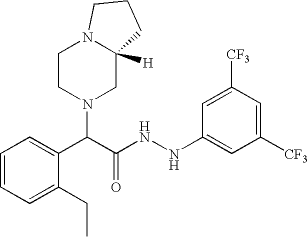

A solution of 260 mg of (2-ethylphenyl)[(8aR)-hexahydropyrrolo[1,2-a]pyrazin-2(1H)-yl]acetic acid hydrochloride (intermediate 151, 0.80 mmole), 260 μL of NMM (2.40 mmole, Aldrich), 425 mg of BOP (0.96 mmole, Fluka) and 215 mg of 3,5-bis(trifluoromethyl)phenylhydrazine (0.88 mmole, Lancaster) in 5 mL of DMF was stirred at RT overnight. Then it was diluted with AcOEt, washed with NaOH 1M, a saturated aqueous solution of NaHCO₃ and brine, dried over Na₂SO₄, filtered and concentrated in vacuo. The crude was purified by flash chromatography eluting with a gradient DCM/MeOH from DCM 100% to DCM/MeOH 80:20. Solvents were removed under reduced pressure to give the title compound as a mixture of two diastereoisomers (334 mg, 75%). ¹H-NMR (400 MHz, CDCl₃): δ 1.25 (3H, t), 1.26-1.40 (1H, m), 1.50-1.95 (4H, m), 2.2-2.5 (4H, m), 2.5-3.00 (4H, m), 3.05-3.30 (2H, m), 4.45 (1H, s), 6.30 (1H, s), 7.05 (2H, s), 7.25-7.38 (4H, m), 7.60 (1H, m), 8.60 (1H, bs); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.65-0.67 min. (99%)

### Compound 101: N′-[3,5-bis(trifluoromethyl)phenyl]-2-(2-ethylphenyl)-2-[(8aR)-hexahydropyrrolo[1,2-a]pyrazin-2(1H)-yl]acetohydrazide-diastereoisomer 1

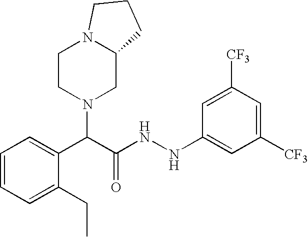

328 mg of N′-[3,5-bis(trifluoromethyl)phenyl]-2-(2-ethylphenyl)-2-[(8aR)-hexahydro-pyrrolo[1,2-a]pyrazin-2(1H)-yl]acetohydrazide-diastereomeric mixture (Compound 100) were purified by chiral chromatography [Chiralcel AD-H, 25×0.46 cm, Flow rate: 1.0 ml/min, UV detection: CD 240 nm, mobile phase: n-hexane/2-propanol 87/13% v/v]. Solvents were removed under reduced pressure to give the title compound (diastereoisomer 1) as a white solid (140 mg, 85%). ¹H-NMR (400 MHz, CDCl₃): δ 1.25 (3H, t), 1.50 (1H, m), 1.75-2.00 (3H, m), 2.05-2.45 (5H, m), 2.7-3.05 (4H, m), 3.10 (1H, m), 3.3 (1H, d), 4.45 (1H, s), 6.30 (1H, s), 7.05 (2H, s), 7.25-7.38 (4H, m), 7.60 (1H, m), 8.60 (1H, bs); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.73 min. (94%).

### Compound 102: N′-[3,5-bis(trifluoromethyl)phenyl]-2-(2-ethylphenyl)-2-[(8aR)-hexahydropyrrolo[1,2-a]pyrazin-2(1H)-yl]acetohydrazide-diastereoisomer 2

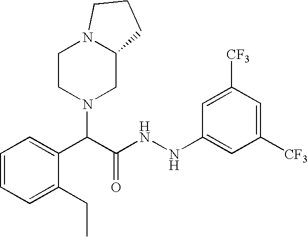

328 mg of N′-[3,5-bis(trifluoromethyl)phenyl]-2-(2-ethylphenyl)-2-[(8aR)-hexahydropyrrolo-[1,2-a]pyrazin-2(1H)-yl]acetohydrazide-diastereomeric mixture (Compound 100) were purified by chiral chromatography [Chiralcel AD-H, 25×0.46 cm, Flow rate: 1.0 ml/min, UV detection: CD 240 nm, mobile phase: n-hexane/2-propanol 87/13% v/v]. Solvents were removed under reduced pressure to give the title compound (diastereoisomer 2) as a white solid (120 mg, 73%). ¹H-NMR (400 MHz, CDCl₃): δ 1.25 (3H, t), 1.26-1.40 (1H, m), 1.70-1.95 (4H, m), 2.03-2.30 (2H, m), 2.35-2.50 (1H, m), 2.52-2.82 (2H, m), 2.82-3.00 (2H, m), 3.04-3.24 (3H, m), 4.45 (1H, s), 6.30 (1H, s), 7.05 (2H, s), 7.25-7.38 (4H, m), 7.60 (1H, m), 8.60 (1H, bs); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.71 min (96%).

### Intermediate 152: [2-(ethyloxy)phenyl][(8aR)-hexahydropyrrolo[1,2-a]pyrazin-2(1H)-yl]-acetic acid hydrochloride-mixture of two diastereoisomers

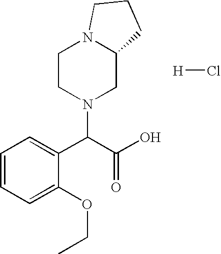

A mixture of 208 mg of [2-(ethyloxy)phenyl]boronic acid (1.25 mmole, Aldrich), 250 mg of (8aR)-octahydropyrrolo[1,2-a]pyrazine di-hydrochloride (intermediate 115, 1.25 mmole), 116 mg of glyoxylic acid monohydrate (1.25 mmole, Aldrich) and 347 mg of K₂CO₃ (2.51 mmole) in 6 mL of CH₃CN was heated under microwaves irradiation at 120° C. for 20 min. Then, the solvent was removed and the residue was treated with HCl 1N, charged on a HLB cartridge and eluted with a gradient from 100% H₂O to 100% MeOH. Solvents were removed under reduced pressure to give the title compound as mixture of two diastereoisomers (150 mg, 29%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.40 min (83%) m/z (ES+): 305 [M+H]⁺.

### Compound 103: N′-[3,5-bis(trifluoromethyl)phenyl]-2-[2-(ethyloxy)phenyl]-2-[(8aR)-hexahydropyrrolo[1,2-a]pyrazin-2(1H)-yl]acetohydrazide-mixture of diastereoisomers

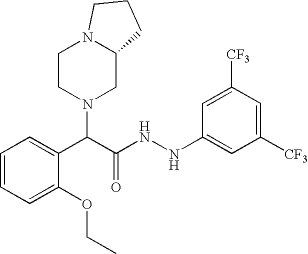

A solution of 150 mg of [2-(ethyloxy)phenyl][(8aR)-hexahydropyrrolo[1,2-a]pyrazin-2(1H)-yl]acetic acid hydrochloride (intermediate 152, 0.44 mmole), 145 μL of NMM (1.32 mmole, Aldrich), 234 mg of BOP (0.52 mmole, Fluka) and 118 mg of 3,5-bis(trifluoromethyl)phenylhydrazine (0.48 mmole, Lancaster) in 5 mL of DMF was stirred at RT overnight. Then it was diluted with AcOEt, washed with NaOH 1M, a saturated aqueous solution of NaHCO₃ and brine, dried and concentrated in vacuo. The crude was purified by flash chromatography eluting with a gradient starting from DCM 100% to DCM/MeOH 80:20. After removal of the solvents the product was not pure enough and was further purified by flash chromatography eluting with a gradient starting from AcOEt 100% to AcOEt/MeOH 80:20. Solvents were removed under reduced pressure to give the titled compound as a mixture of two diastereoisomers (120 mg, 51.4%). ¹H-NMR (400 MHz, CDCl₃): δ 1.35 (3H, t), 1.5-3.25 (14H, m), 4.1 (2H, q), 4.2 (1H, s), 6.4 (1H, d), 6.86-7.05 (2H, m), 7.18 (2H, s), 7.25-7.40 (2H, m), 8.80 (1H, bs).

### Intermediate 153: (±)-bromo(2-chloro-3-pyridinyl)acetic acid

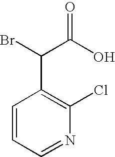

To a mixture of 3 g of ice, 476 mg of potassium hydroxide (8.48 mmole Fluka), and 3 mL of 1,4-dioxane (Aldrich) at 0° C., was added a mixture of 300 mg 2-chloro-3-pyridinecarbaldehyde (2.119 mmole, Aldrich) and 589 mg of tribromomethane (2.331 mmole, Aldrich) in 3 mL of 1,4-dioxane. After 1 h at 0° C., 3 mL of water were added and the stirring was continued at RT for additional 3 h. The aqueous solution was washed with AcOEt (2×10 mL) treated with a saturated aqueous solution of NH₄Cl and washed again with AcOEt (10 mL). The water was evaporated under vacuum and the title compound was used without any further purification (800 mg). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.50 min. (61%) m/z (ES): 250 [M]⁺, 252 [M+2]⁺.

### Intermediate 154: (±)-methyl bromo(2-chloro-3-pyridinyl)acetate

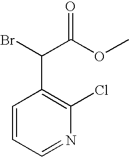

To a solution of 800 mg of bromo(2-chloro-3-pyridinyl)acetic acid (intermediate 153, 8.48 mmole) 10 mL of in methanol, was added 1 mL of sulphuric acid (18.76 mmole, Fluka) at RT and the mixture stirred at 85° C. for 3 h. The mixture was treated with NaHCO₃ saturated solution to reach a basic pH and extracted with DCM (2×10 mL). The organic phase was dried over Na₂SO₄, filtered and evaporated under reduced pressure to give the title compound (330 mg, 42%). ¹H-NMR (400 MHz, CDCl₃): δ 3.83-3.84 (d, 3H), 5.81-5.83 (d, 1H), 7.36-7.38 (m, 1H), 8.05-8.20 (dd, 1H), 8.38-8.43 (m, 1H).

### Intermediate 155: methyl(2-chloro-3-pyridinyl)[(8aR)-hexahydropyrrolo[1,2-a]pyrazin-2(1H)-yl]acetate-mixture of diastereoisomers

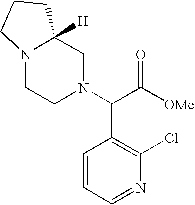

To a mixture of 320 mg of (±)-methyl bromo(2-chloro-3-pyridinyl)acetate (intermediate 154, 1.210 mmole) and 356 mg of sodium bicarbonate (4.23 mmole, Fluka) in 10 mL of methanol, were added 241 mg of (8aR)-octahydropyrrolo[1,2-a]pyrazine (1.210 mmole) and the reaction stirred at 80° C. for 3 h. The solvent was removed under vacuum And the residue was purified by flash chromatography on Si SPE cartridge with a gradient starting from DCM/MeOH 99/1 to 9/1 to give the title compound (170 mg, 45.4%). ¹H-NMR (400 MHz, CDCl₃): δ 1.31-1.48 (m, 1H), 1.65-1.91 (m, 3H), 2.06-2.20 (m, 3H), 2.26-2.41 (m, 1H), 2.45-2.59 (m, 1H), 2.67-2.86 (m, 1H), 2.88-3.00 (m, 1H), 3.01-3.13 (m, 2H), 3.75 (s, 3H), 4.67-4.69 (d, 1H), 7.29-7.31 (m, 1H), 8.02-8.04 (m, 1H), 8.35-8.37 (m, 1H); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.41 min. (88%) m/z (ES): 310 [M+H]⁺.

### Intermediate 156: (2-chloro-3-pyridinyl)[(8aR)-hexahydropyrrolo[1,2-a]pyrazin-2(1H)-yl]acetate ammonium salt-mixture of diastereoisomers

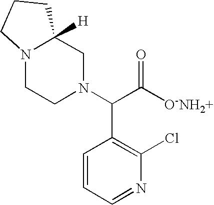

To 170 mg of methyl(2-chloro-3-pyridinyl)[(8aR)-hexahydropyrrolo[1,2-a]pyrazin-2(1H)-yl]acetate (intermediate 155, 0.549 mmole) dissolved in 5 mL of ethanol, were added 46.2 mg of potassium hydroxide (0.823 mmole, Fluka) and the reaction stirred at RT overnight. Then, 3 additional eq of sodium hydroxide were added over 3 h and the mixture was stirred at 40° C. The methanol solution was then eluted through a SPE-SCX cartridge with a gradient starting from MeOH 100 to MeOH/NH₃ 1M in MeOH 8/2 to give the title compound (160 mg, 93%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.42 min. (88%) m/z (ES): 310 [M+H]⁺.

### Compound 104: N′-[3,5-bis(trifluoromethyl)phenyl]-2-(2-chloro-3-pyridinyl)-2-[(8aR)-hexahydropyrrolo[1,2-a]pyrazin-2(1H)-yl]acetohydrazide-diastereomeric mixture

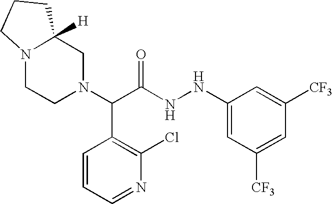

To a solution of 160 mg of (2-chloro-3-pyridinyl)[(8aR)-hexahydropyrrolo[1,2-a]pyrazin-2(1H)-yl]acetic acid (intermediate 156, 0.541 mmole), 267 mg of HATU (0.703 mmole, Fluka) and 196 μL of TEA (1.407 mmole, Aldrich) in 10 mL of N,N-dimethylformamide, were added 158 mg of [3,5-bis(trifluoromethyl)phenyl]hydrazine (0.649 mmole, Alfa Aesar). The mixture was stirred for 3 h at RT. The solvent was evaporated under reduced pressure and the mixture was partitioned between 5 mL of water and 10 mL of DCM. The aqueous phase was extracted one more time with 10 mL of DCM. The combined organic layers were dried over Na₂SO₄, filtered and evaporated to dryness. The resulting residue was purified by Si SPE cartridge with a gradient starting from DCM/MeOH 99/1 to 9/1 to give the title compound (67 mg, 23.73%). ¹H-NMR (400 MHz, DMSO-d₆): δ 1.05-3.19 (m, 13H), 4.36-4.88 (m, 1H), 7.15 (s, 2H), 7.32 (s, 1H), 7.49 (m, 1H), 8.12 (bs, 1H), 8.39 (bs, 1H), 8.86 (s, 1H), 10.63 (s, 1H); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.63 min. (96%) m/z (ES): 522 [M+H]⁺.

### Intermediate 157: 4-Fluoro-2-methyl-benzaldehyde

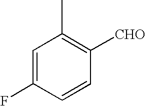

A solution of 10 g of 5-Bromo-3-fluorotoluene (0.05 mole) in 500 mL of THF was cooled to −78° C. Then, 32 mL of a solution of n-Butyl lithium in hexanes (2.5 M, 0.08 mole) were added to the mixture keeping the internal temperature between −75° C. and −78° C. The resulting mixture was stirred at −78° C. for 50 min. 43 mL of Dry DMF (0.53 mole) were added over 1 h to the mixture. The mixture was allowed to warm to RT (about 18° C.) and stirred overnight. The reaction was quenched with 20 mL of a saturated solution of NH₄Cl. The mixture was washed with water (5×100 mL) and the organic layer was washed with a 50 mL of a saturated aqueous solution of NaHCO₃, dried over Na₂SO₄, filtered and evaporated to dryness. The residue was purified by a column chromatography on silica gel (PE/AcOEt=50/1) to give the title compound as light yellow liquid (3.00 g, 41.09%).

### Intermediate 158, first preparation: (4-fluoro-2-methylphenyl)[(8aR)-hexahydropyrrolo[1,2-a]pyrazin-2(1H)-yl]acetonitrile-diastereomeric mixture

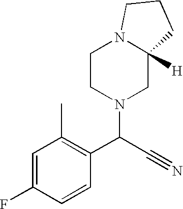

To a solution of 50 mg of 4-Fluoro-2-methyl-benzaldehyde (intermediate 157, 0.36 mmole) in 2 mL of diethyl ether, were added 59 μL of trimethylsilyl cyanide (0.43 mmole) and 5.8 mg of zinc iodide (0.02 mmole) at RT under nitrogen. The reaction mixture was cooled to 0° C. and it was stirred at 0° C. for 5 min. Then a solution of 72 mg of (R)-octahydropyrrolo[1,2-a]pyrazine (2HCl salt, intermediate 115, 0.36 mmole) in 2 mL of MeOH was added dropwise to the reaction mixture. Then, 105 μL of TEA (0.72 mmole) were added dropwise to the reaction. The mixture was refluxed overnight. The solution was cooled to RT and the mixture was purified by preparative TLC to afford the title compound as a brown oil (60 mg, 50.55%). ¹H-NMR (CDCl₃) δ: 1.20-1.50 (1H, m), 1.55-2.82 (3H, m), 2.15-2.30 (3H, s), 2.30 (3H, m), 2.30-2.51 (2H, m), 2.71-3.06 (4H, m), 4.72-4.80 (1H, d), 6.83-6.88 (2H, m), 7.43-7.46 (1H, m); TLC (PE/AcOEt 10:1): R_{f}=0.2.

### Intermediate 158, second preparation: (4-fluoro-2-methylphenyl)[(8aR)-hexahydropyrrolo[1,2-a]pyrazin-2(1H)-yl]acetonitrile-diastereomeric mixture

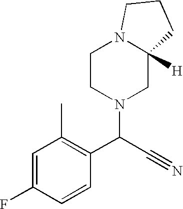

To a solution of 347 mg of 4-Fluoro-2-methyl-benzaldehyde (intermediate 157, 2.5 mmole) in 14 mL of diethyl ether, were added 410 μL of trimethylsilyl cyanide (3 mmole) and 41 mg of zinc iodide (0.13 mmole) at RT under nitrogen. The reaction mixture was cooled to 0° C. and it was stirred at 0° C. for 5 min. Then a solution of 500 mg of (R)-octahydropyrrolo[1,2-a]pyrazine dihydrochloride (intermediate 115, 2.5 mmole) in 14 mL of MeOH was added dropwise to the reaction mixture. Then, 730 μL of TEA (5 mmole) were added dropwise to the reaction. The mixture was refluxed overnight and the reaction was cooled to RT. 20 mL of a saturated aqueous solution of K₂CO₃ were added, followed by 20 mL of AcOEt. The mixture was extracted twice with 50 mL of AcOEt. The combined organic layers were washed twice with 20 mL of a saturated aqueous solution of NaHCO₃, and dried over Na₂SO₄, filtered and evaporated to dryness. The resulting crude compound was purified by a column chromatography on silica gel (PE/AcOEt=3/1) to afford the title compound as a brown oil (450 mg, 66%). ¹H-NMR (CDCl₃): δ: 1.10-1.50 (4H, m), 1.70-1.90 (2H, m), 1.95-2.10 (3H, m), 2.30 (3H, s), 2.35-2.52 (2H, m), 2.72-2.79 (1H, m), 2.85-3.06 (2H, m), 4.72-4.80 (1H, d), 6.83-6.88 (2H, m), 7.43-7.46 (1H, m); TLC (PE/AcOEt 10:1): R_{f}=0.15.

### Intermediate 159: 2-(4-fluoro-2-methylphenyl)-2-[(8aR)-hexahydropyrrolo[1,2-a]pyrazin-2(1H)-yl]acetamide-diastereomeric mixture

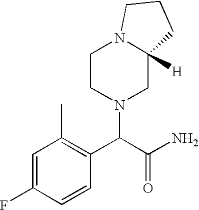

At 0° C., to a solution of 480 mg of (4-fluoro-2-methylphenyl)[(8aR)-hexahydropyrrolo[1,2-a]pyrazin-2(1H)-yl]acetonitrile (intermediate 158, 1.76 mmole) in 10 mL of n-hexane, were added 10 mL of H₂SO₄ 98% (0.2 mole). The mixture was stirred overnight at RT. The mixture was cooled to 0° C., and ice was added. Then the acid was neutralized with NH₃H₂O 28%. The mixture was extracted twice with 20 mL of AcOEt. The organic layers were combined and dried over Na₂SO₄, filtered and evaporated to dryness. The resulting residue was purified by a flash chromatography on silica gel to afford the title compound as a brown solid (350 mg, 68%). ¹H-NMR (CDCl₃): δ 1.05-1.40 (2H, m), 1.70-1.90 (2H, m), 1.90-2.30 (4H, m), 2.35 (3H, s), 2.40-2.70 (2H, m), 2.80-3.18 (3H, m), 4.14 (1H, s), 5.44 (1H, s), 6.71-6.89 (3H, m), 7.27-7.35 (1H, m); m/z (ES): 292[M+H]⁺.

### Intermediate 160: N′-[3,5-bis(trifluoromethyl)phenyl]-2-(4-fluoro-2-methylphenyl)-2-[(8aR)-hexahydropyrrolo[1,2-a]pyrazin-2(1H)-yl]acetohydrazide-diastereomeric mixture

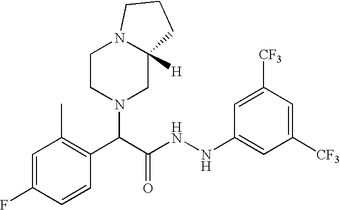

351 mg of 2-(4-fluoro-2-methylphenyl)-2-[(8aR)-hexahydropyrrolo[1,2-a]pyrazin-2(1H)-yl]acetamide (intermediate 159, 1.205 mmole) were dissolved in 12 mL of DCM. Then 0.573 mL of BOC₂O (2.470 mmole) were added followed by 15 mg of DMAP (0.120 mmole). The mixture was stirred at RT for 4 h. Then 29 mg of DMAP (0.241 mmole) were added followed by 353 mg of [3,5-bis(trifluoromethyl)phenyl]hydrazine (1.446 mmole). The mixture was evaporated to the third and was stirred for 51 h. The mixture was evaporated to dryness and the crude was then purified by flash chromatography (ISCO COMPANION) on a 40 g silica gel column with the following gradient: A: AcOEt+3% TEA/B:MeOH+3% TEA: 0% B for 1 min, 0% to 2.5% B in 23 min, 2.5% B for 6 min. All the fractions collected contained DMAP as impurity. The compound was dissolved in H₂O containing drops of HCl conc. and MeOH and passed through a HLB cartridge (6 g). Then the cartridge was washed with H₂O (20 mL), 95% H₂O/5% MeOH (20 mL), 90% H₂O/10% MeOH (20 mL), 85% H₂O/15% MeOH (20 mL), 80% H₂O/20% MeOH (20 mL), 75% H₂O/25% MeOH (20 mL). The target compound was then released with 60 mL of MeOH. The hydrochloride salt was then passed through a SCX (3 g). Then the cartridge was washed with 15 mL of DCM, 15 mL of MeOH, and the compound was released with 20 mL of NH₃ 2M/MeOH. The solvents were removed under reduced pressure to give the title compound as a yellow oil (111.6 mg, 17%). ¹H-NMR (400 MHz, CD₃OD): δ 1.27-1.52 (m, 2H), 1.73-1.92 (m, 4H), 2.16-2.26 (m, 3H), 2.27-2.48 (m, 2H), 2.50-2.54 (m, 3H), 2.68-2.88 (m, 1H), 2.92-3.22 (m, 3H), 4.31 (d, 1H), 6.94-7.05 (m, 4H), 7.23 (s, 1H), 7.69-7.76 (m, 1H); LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.91 min (100%) m/z (ES): 519.2 [M+H]⁺.

### Compound 105: N′-[3,5-bis(trifluoromethyl)phenyl]-2-(4-fluoro-2-methylphenyl)-2-[(8aR)-hexahydropyrrolo[1,2-a]pyrazin-2(1H)-yl]acetohydrazide-diastereoisomer 2

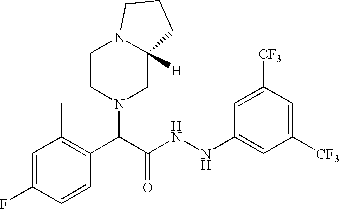

111.6 mg of N′-[3,5-bis(trifluoromethyl)phenyl]-2-(4-fluoro-2-methylphenyl)-2-[(8aR)-hexahydropyrrolo[1,2-a]pyrazin-2(1H)-yl]acetohydrazide (intermediate 160) were purified by preparative chiral HPLC [Column: Chiralpak AD-H (25×0.46 cm); Mobile phase: n-Hexane/2-Propanol 90/10% v/v; Flow rate: 1.0 mL/min; DAD: 210-340 nm; CD: 245 nm]. Solvents were removed under reduced pressure to give the title compound as a yellow solid (50 mg, 45%). ¹H-NMR (400 MHz, MeOD): δ 1.41-1.53 (m, 1H), 1.76-1.93 (m, 3H), 2.13-2.40 (m, 5H), 2.52 (s, 3H), 2.71 (dd, 1H), 2.91-2.99 (m, 1H), 3.00-3.11 (m, 1H), 3.19 (d, 1H), 4.32 (s, 1H), 6.94-7.02 (m, 2H), 7.03 (s, 2H), 7.23 (s, 1H), 7.73 (dd, 1H); LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.92 min (100%) m/z (ES): 519.2 [M+H]⁺. Chiral HPLC [Column: Chiralpak AD-H (25×0.46 cm); Mobile phase: n-Hexane/2-Propanol 90/10% v/v; Flow rate: 1.0 mL/min; DAD: 210-340 nm; CD: 245 nm]: Rₜ=14.79 min (100% e.e.).

### Intermediate 161: 5-methyl-2-pyridinecarbaldehyde

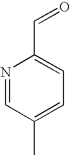

5 g of 5-bromo-2-methylpyridine (29.1 mmole, Aldrich), dissolved in 60 mL of dry THF. The solution was cooled to −78° C. To this solution, were added 15.99 mL of BuLi (32.0 mmole) and the reaction mixture was stirred at −78° C. for 0.5 h. Then 4.50 mL of dry DMF (58.1 mmole) were added and stirring was continued for 0.5 h. 30 mL of water were added followed by 100 mL of AcOEt. The organic layer was separated, dried over Na₂SO₄, filtered and evaporated under vacuum to give a crude that was purified on Isco-Companion (CH/AcOEt 1:1 to 1:9) to give the title compound as a yellow oil (1.6 g, 45% yield). ¹H-NMR (400 MHz, CDCl₃): δ 1.99 (s, 3H), 7.22 (d, 1H), 7.43 (d, 1H), 8.16 (s, 1H), 9.59 (s, 1H); m/z (ES): 121.99 [M+H]⁺.

### Intermediate 162: (8aR)-hexahydropyrrolo[1,2-a]pyrazin-2(1H)-yl(5-methyl-2-pyridinyl)-acetonitrile-diastereomeric mixture

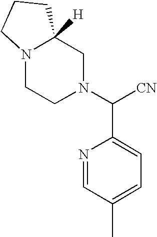

To a solution of 1.6 g of 5-methyl-3-pyridinecarbaldehyde (intermediate 161, 13.21 mmole) in 2.5 mL of diethyl ether, were added 2.125 mL of TMSCN (15.85 mmole) and 211 mg of zinc iodide (0.660 mmole) at RT under nitrogen. The reaction mixture was cooled to 0° C. and stirred 5 min at this temperature. Then a solution of 2.60 g of (8aR)-octahydropyrrolo[1,2-a]pyrazine di-hydrochloride (intermediate 115, 13.21 mmole) and 3.87 mL of TEA (27.7 mmole) in 13 mL of MeOH was added dropwise to the reaction mixture. The solution was then refluxed for 3.5 h. The mixture was cooled to RT and 50 mL of a saturated solution of K₂CO₃ were added followed by 100 mL of AcOEt. The aqueous layer was extracted twice with 50 mL of AcOEt. The combined organic layers were dried over Na₂SO₄, filtered and evaporated under vacuum to give a crude that was purified on Isco-Companion (AcOEt TEA 3%/CH 1:9 to 1:1 as eluent) to give the title compound as a yellow oil (2.52 g, 63%). ¹H-NMR (400 MHz, CDCl₃): δ 1.60-1.90 (m, 5H), 2.13-2.27 (m, 2H), 2.38 (s, 3H), 2.40-2.55 (m, 2H), 2.71-2.98 (m, 2H), 2.99-3.13 (m, 2H), 4.98 (d, 1H), 7.47-7.58 (dd, 2H), 8.50 (s, 1H); m/z (ES): 257.17 [M+H]⁺.

### Intermediate 163: 2-[(8aR)-hexahydropyrrolo[1,2-a]pyrazin-2(1H)-yl]-2-(5-methyl-2-pyridinyl)acetamide-diastereomeric mixture

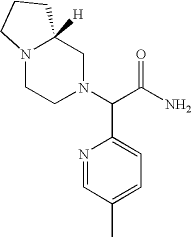

2.5 g of (8aR)-hexahydropyrrolo[1,2-a]pyrazin-2(1H)-yl(5-methyl-2-pyridinyl)acetonitrile (intermediate 162, 9.11 mmole) were suspended in 22 mL of cyclohexane. The solution was cooled to 0° C. and 22 mL of sulfuric acid were added dropwise. The resulting slurry was then allowed to reach RT and then stirred at RT for 25 h. The solution was poured into a flask with ice. Then 83 mL of NH₄OH 28% were added slowly. Then the solution was poured into a separatory funnel and extracted four times with 150 mL of DCM. The aqueous phase was reduced at third under vacuum and extracted three times with 50 mL of DCM. The combined organic layers were dried over Na₂SO₄, filtered and evaporated to dryness. The resulting crude compound was then purified by flash chromatography (ISCO COMPANION) on silica gel (80 g column) with the following gradient: A: AcOEt+3% TEA/B: MeOH+3% TEA: 0% B for 2.5 min, 0% to 20% B in 28.5 min, 20% B for 6 min. Solvents were removed under reduced pressure to give the title compound as a yellow foam (1.961 g, 72%). ¹H-NMR (CDCl₃, 400 MHz) δ 1.61-1.92 (m, 5H), 2.09-2.21 (m, 2H), 2.25-2.42 (m, 4H), 2.49-2.64 (m, 1H), 2.71-2.98 (m, 1H), 2.99-3.29 (m, 3H), 4.12 (d, 1H), 5.54 (s, 1H), 7.26 (d, 1H), 7.33 (s, 1H), 7.47-7.52 (m, 1H), 8.46 (s, 1H). LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]:Rₜ=0.229 min (75.73%) m/z (ES): 275.2 [M+H]⁺; Rₜ=0.300 min (21.6%) m/z (ES): 275.2 [M+H]⁺.

### Intermediate 164: N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide-diastereomeric mixture

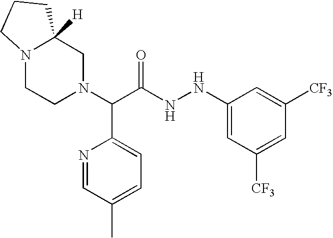

1.96 g of 2-[(8aR)-hexahydropyrrolo[1,2-a]pyrazin-2(1H)-yl]-2-(5-methyl-2-pyridinyl)acetamide (intermediate 163, 3.91 mmole) were dissolved in 72 mL of DCM. Then 3.40 mL of BOC₂O (14.64 mmole) were added followed by 87 mg of DMAP (0.714 mmole). The mixture was stirred at RT for 19 h. Then 175 mg of DMAP (1.430 mmole) were added followed by 2.093 g of [3,5-bis(trifluoromethyl)phenyl]hydrazine (8.57 mmole). The mixture was evaporated to the third and was stirred for other 25.5 h. The solution was evaporated to dryness and the crude compound was then purified by flash chromatography (Isco Companion) on a 80 g silica gel column with the following gradient: A:AcOEt+3% TEA/B:MeOH+3% TEA: 0% B for 2 min, 0% to 2.5% B in 26 min, 2.5% B for 20 min. The fractions collected contained the target material with other impurities. The compound was dissolved into a mixture of H₂O, HCl 2N and MeOH and passed through a HLB cartridge (6 g). Then the cartridge was washed with H₂O (20 mL), 95% H₂O/15% MeOH (20 mL), 90% H₂O/10% MeOH (20 mL), 85% H₂O/15% MeOH (20 mL), 80% H₂O/20% MeOH (20 mL), 75% H₂O/25% MeOH (20 mL), 70% H₂O/30% MeOH (20 mL), MeOH (60 mL). Only the fractions containing the target compound were collected. Then, the hydrochloride salt of the compound was passed through a SCX cartridge (10 g). Then, the cartridge was washed with 50 mL of DCM, 50 mL of MeOH, and the compound was released with 100 mL of NH₃ 2M/MeOH. Solvents were removed under reduced pressure to give the title compound as a yellow solid (980 mg, 26%). ¹H-NMR (400 MHz, CDCl₃): δ 1.20-1.46 (m, 1H), 1.60-1.89 (m, 3H), 2.06-2.26 (m, 3H), 2.27-2.56 (m, 5H), 2.67 (d, 1H), 2.88-3.21 (m, 3H), 4.13 (d, 1H), 7.06 (s, 2H), 7.13-7.23 (m, 2H), 7.25-7.33 (m, 1H), 7.51 (d, 1H), 8.45-8.54 (m, 1H), 9.37 (s, 1H); LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.74 min (97.20%) m/z (ES): 502.1 [M+H]⁺.

### Compound 106: N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide-diastereoisomer 2

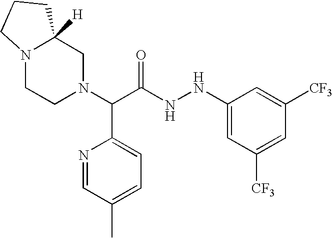

980 mg of N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide (intermediate 164) were purified by preparative Chiral HPLC [Column: Chiralpak AD-H (25×0.46 cm); Mobile phase: n-Hexane/2-Propanol 85/15% v/v; Flow rate: 1.0 mL/min; DAD: 210-340 nm; CD: 245 nm]. Solvents were removed under reduced pressure to give the title compound as a yellow solid (310 mg, 32%). ¹H-NMR (400 MHz, MeOD): δ 1.46 (d, 1H), 1.73-1.94 (m, 3H), 2.10-2.48 (m, 8H), 2.57-2.73 (m, 1H), 2.88-3.10 (m, 2H), 3.21 (d, 1H), 4.22 (s, 1H), 7.12 (s, 2H), 7.23 (s, 1H), 7.56-7.62 (m, 1H), 7.69 (dd, 1H), 8.45 (d, 1H). LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=1.74 min (96.82%) m/z (ES): 502.2 [M+H]⁺. Chiral HPLC [Chiralpak AD-H (25×0.46 cm); Mobile phase: n-Hexane/2-Propanol 85/15% v/v; Flow rate: 1.0 mL/min; DAD: 210-340 nm; CD: 245 nm]: Rₜ=17.24 min (100% e.e.).

### Intermediate 165: (8aR)-hexahydropyrrolo[1,2-a]pyrazin-2(1H)-yl(6-methyl-2-pyridinyl)-acetonitrile-diastereomeric mixture

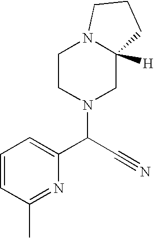

1.5 g of 6-methyl-2-pyridinecarbaldehyde (12.38 mmole, ALDRICH) were dissolved in 2 mL of diethyl ether. Then, 1.992 mL of trimethylsilyl cyanide (14.86 mmole, ALDRICH) and 0.198 g of zinc iodide (0.619 mmole, ALDRICH) were added at RT under nitrogen. The reaction was cooled to 0° C. and it was stirred at 0° C. for 5 min. Then a solution of 2.466 g of (8aR)-octahydropyrrolo[1,2-a]pyrazine dihydrochloride (intermediate 115, 12.38 mmole) and 3.62 mL of TEA (26.0 mmole, FLUKA) in 8.95 mL of Methanol was added dropwise to the reaction mixture and it was then refluxed for 3 h. The solution was cooled to RT and 75 mL of a saturated solution of K₂CO₃ were added followed by 100 mL of AcOEt. The mixture was then transferred to a separatory funnel and the aqueous phase was extracted two more times with 50 mL of AcOEt. The combined organic layers were then dried over Na₂SO₄, filtered and evaporated to dryness. The resulting crude compound was then purified by flash chromatography (ISCO COMPANION, 80 g silica gel column) with the following gradient: A: Cyclohexane+3% TEA/B: AcOEt+3% TEA: 0% B for 2 min, 0% to 90% B in 19 min, 90% B for 6 min. Solvents were removed under reduced pressure to give the title compound as a yellow oil (2.418 g, 74%). ¹H-NMR (400 MHz, CDCl₃): δ 1.35-1.56 (m, 1H), 1.61-1.94 (m, 3H), 1.97-2.31 (m, 3H), 2.36-2.64 (m, 5H), 2.68-3.02 (m, 2H), 3.04-3.14 (m, 2H), 4.79-5.01 (m, 1H), 7.15 (d, 1H), 7.37 (d, 1H), 7.63 (td, 1H). UPLC-MS [Acquity™ UPLC BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]: Rₜ=0.39 min (100%) m/z (ES): 257.19 [M+H]⁺, 230.20 [M−CN]⁺.

### Intermediate 166: 2-[(8aR)-hexahydropyrrolo[1,2-a]pyrazin-2(1H)-yl]-2-(6-methyl-2-pyridinyl)acetamide-diastereomeric mixture

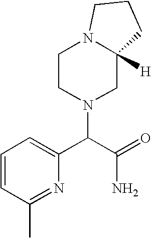

2.4184 g of (8aR)-hexahydropyrrolo[1,2-a]pyrazin-2(1H)-yl(6-methyl-2-pyridinyl)-acetonitrile (intermediate 165, 9.43 mmole) was suspended in 21 mL of n-Hexane. The solution was cooled to 0° C. and 21 mL of sulfuric acid were added dropwise. The resulting slurry was then allowed to reach RT and was stirred at RT for 18 h. The mixture was cooled to 0° C. and ice was added. Then the acid was neutralized with 80 mL of NH₄OH 28%. The aqueous phase was extracted with DCM (4×75 mL). Then the aqueous phase was evaporated to the third and was extracted with DCM (3×75 mL). The combined organic layers were dried over Na₂SO₄, filtered and evaporated to dryness. The resulting crude compound was purified by flash chromatography on silica gel (ISCO COMPANION, 80 g silica gel column) with the following gradient: A: AcOEt+3% TEA/B: MeOH+3% TEA: 0% B for 2 min, 0% to 15% B in 20 min, 20% B for 5 min. Solvents were removed under reduced pressure to give the title compound as a yellow foam (2.275 g, 88%). ¹H-NMR (400 MHz, CD₃OD): δ 1.19-1.51 (m, 1H), 1.66-2.40 (m, 7H), 2.41-2.51 (m, 1H), 2.55 (s, 3H), 2.57-2.77 (m, 1H), 2.87-3.25 (m, 3H), 4.10 (s, 1H), 7.24 (d, 1H), 7.41 (d, 1H), 7.71 (t, 1H). UPLC-MS [Acquity™ UPLC BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]: Rₜ=0.29 min and 0.31 min (100%) m/z (ES): 275.17 [M+H]⁺, 138.03 [M+H/2]⁺.

### Intermediate 167: methyl(8aR)-hexahydropyrrolo[1,2-a]pyrazin-2(1H)-yl(6-methyl-2-pyridinyl)acetate-diastereomeric mixture

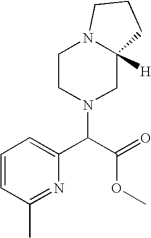

0.98 g of 2-[(8aR)-hexahydropyrrolo[1,2-a]pyrazin-2(1H)-yl]-2-(6-methyl-2-pyridinyl)-acetamide (intermediate 166, 3.57 mmole) were dissolved in 39 mL of dry Methanol. Then, 2.381 mL of N,N-dimethylformamide dimethyl acetal (DMF-DMA) (17.86 mmole, ALDRICH) were added and the mixture was stirred at RT for three days. Then 1.634 mL of a solution of sodium methoxide 25 wt % in Methanol (7.14 mmole, ALDRICH) were added and the mixture was stirred for 24 h. 1.634 mL of a solution of sodium methoxide 25 wt % in Methanol (7.14 mmole) were added and the mixture was stirred for 2 days. 1.634 mL of a solution of sodium methoxide 25 wt % in Methanol (7.14 mmole) were added and the mixture was stirred at RT for additional 27.5 h. The solution was evaporated to dryness under vacuum (important: the compound must not be heated). The crude was then purified by flash chromatography (ISCO COMPANION) on a 80 g silica gel column with the following gradient: A:Cyclohexane+3% TEA/B:AcOEt+3% TEA: 0% B for 2 min, 0% to 70% B in 21 min, 70% B for 5 min. Solvents were removed under reduced pressure to give the title compound as a yellow foam (197.6 mg, 18%). ¹H-NMR (400 MHz, CDCl₃): δ 1.24-1.45 (m, 1H), 1.67-1.88 (m, 3H), 2.10-2.45 (m, 4H), 2.48-2.76 (m, 4H), 2.80-3.06 (m, 4H), 3.70 (d, 3H), 4.41 (d, 1H), 7.21 (d, 1H), 7.38 (d, 1H), 7.70 (t, 1H); m/z (ES): 290.7 [M+H]⁺

### Intermediate 168: methyl 2-[(8aR)-hexahydropyrrolo[1,2-a]pyrazin-2(1H)-yl]-2-(6-methyl-2-pyridinyl)propanoate-diastereomeric mixture

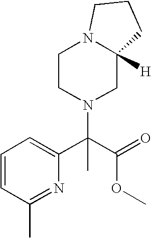

197 mg of methyl(8aR)-hexahydropyrrolo[1,2-a]pyrazin-2(1H)-yl(6-methyl-2-pyridinyl)acetate (intermediate 167, 0.681 mmole) were dissolved in 4.5 mL of THF. The solution was cooled to −78° C. and 1.513 mL of a solution of LiHMDS 0.9 M in hexane (1.362 mmole, ACROS ORGANICS) were added dropwise. The solution was stirred at −78° C. for 1.5 h. Then 0.064 mL of iodomethane (1.021 mmole) were added dropwise and the mixture was allowed to reach RT slowly. It was stirred for 2.5 h. The solution was evaporated to dryness under vacuum and the crude was purified by flash chromatography (ISCO COMPANION) on a 12 g silica gel column with the following gradient: A: Cyclohexane+3% TEA/B: AcOEt+3 TEA: 60% B for 3 min, 60% to 70% B in 3 min, 70% B for 9 min. Solvents were removed under reduced pressure to give the title compound as a yellow oil (41.5 mg, 16%). ¹H-NMR (400 MHz, CD₃OD): δ 1.17-1.47 (m, 1H), 1.65 (s, 3H), 1.73-1.89 (m, 3H), 2.16-2.41 (m, 4H), 2.46-2.51 (m, 3H), 2.56-2.78 (m, 1H), 2.85-3.15 (m, 4H), 3.70-3.75 (m, 3H), 7.12 (d, 1H), 7.46 (t, 1H), 7.65 (t, 1H); LC-MS [Supelcosil ABZ+Plus 33×4.6 mm, 3μ, gradient: A: H₂O+0.1% HCOOH, B: CH₃CN: 0% to 95% B in 3 min, 95% B for 1 min, 95% to 0% B in 0.1 min, flow: 2 mL/min]: Rₜ=0.241 min (22.06%) m/z (ES): 304 [M+H]⁺, 0.746 min (57.54%) m/z (ES): 304.3 [M+H]⁺.

### Compound 107: N′-[3,5-bis(trifluoromethyl)phenyl]-2-[(8aR)-hexahydropyrrolo[1,2-a]pyrazin-2(1H)-yl]-2-(6-methyl-2-pyridinyl)propanohydrazide-diastereomeric mixture

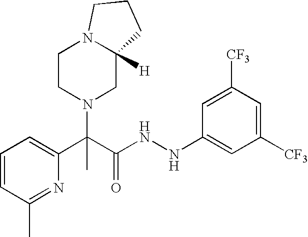

36.9 mg of methyl 2-[(8aR)-hexahydropyrrolo[1,2-a]pyrazin-2(1H)-yl]-2-(6-methyl-2-pyridinyl)propanoate (intermediate 168, 0.122 mmole) were dissolved in 0.5 mL of MeOH. 8.19 mg of potassium hydroxide (0.146 mmole) were added followed by 0.1 mL of water. The mixture was stirred at RT for 4 weeks. Solvents were removed under reduced pressure and the resulting crude compound was dried under high vacuum for a couple of hours to give 22.20 mg of (±) potassium 2-[(8aR)-hexahydropyrrolo[1,2-a]pyrazin-2(1H)-yl]-2-(6-methyl-2-pyridinyl)propanoate (0.068 mmole) which were dissolved in 2 mL of DMF. Then 18.21 mg of [3,5-bis(trifluoromethyl)phenyl]hydrazine (0.075 mmole) were added followed by 45 mg of BOP (0.102 mmole). The mixture was stirred at RT for 3 h. The solution was passed through a SCX cartridge (1 g). Then the cartridge was washed with 3 mL of DCM, 3 mL of MeOH, and the compound was released with 6 mL of NH₃ 2M/MeOH. Solvents were removed under reduced pressure. The crude was then purified by preparative HPLC [Column: Gemini C18 AXIA, 50×21 mm, 5 μm; Mobile phase: A: NH₄HCO₃ sol. 10 mM, pH 10/B: CH₃CN; Gradient: 40% to 45% B in 1 min, 45% to 80% B in 7 min, 80% to 100% B in 1 min, 100% B for 2 min; Flow rate: 17 mL/min; UV range: 210-350 nm; Ionization: ES+; Mass range: 100-900 amu]: Rₜ=4.06 min (37.76%) m/z (ES): 516 [M+H]⁺, Rₜ=4.26 min (1.56%) m/z (ES): 516 [M+H]⁺. Solvents were removed under reduced pressure to give the title compound as colourless oil (6 mg, 16%). ¹H-NMR (400 MHz, CD₃OD): δ 1.38-1.69 (m, 1H), 1.73 (d, 3H), 1.82-2.17 (m, 3H), 2.31-2.88 (m, 9H), 2.93-3.22 (m, 3H), 7.03 (d, 2H), 7.18-7.24 (m, 2H), 7.64 (d, 1H), 7.73 (t, 1H); LC-MS [XTerra MC C18, 30×4.6 mm, 2.5 μm, gradient: A NH₄CO₃ 5 mM, pH 10 (NH₄OH)/B: CH₃CN: 0 to 50% B in 0.4 min, 50% to 95% B in 3.6 min, 95% B for 1 min, 95% to 0% B in 0.1 min, flow: 1.5 mL/min]: Rₜ=2.51 min (100%) m/z (ES): 516.0 [M+H]⁺.

### Intermediate 169: (8aR)-hexahydropyrrolo[1,2-a]pyrazin-2(1H)-yl[2-methyl-4-(methyloxy)-phenyl]acetonitrile-diastereomeric mixture

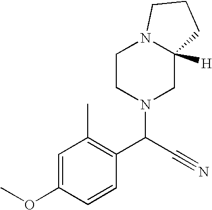

To a solution of 412 mg of 4-methoxy-2-methylbenzaldehyde (2.8 mmole) in 15 mL of diethyl ether, were added 410 μL of trimethylsilyl cyanide (3 mmole) and 41 mg of zinc iodide (0.13 mmole) at RT under nitrogen. The mixture was cooled to 0° C. and stirred 5 min at 0° C. Then the mixture was treated with a solution of 500 mg of (R)-octahydropyrrolo[1,2-a]pyrazine dihydrochloride (intermediate 115, 2.5 mmole) in 15 mL of MeOH. Then, 730 μL of TEA (5 mmole) were added to the reaction. The reaction was refluxed overnight. The reaction was cooled to RT. Then 20 mL of a saturated aqueous solution of K₂CO₃ were added to the reaction. The mixture was extracted with AcOEt (3×20 mL). The combined organic layers were dried over Na₂SO₄, filtered and evaporated to dryness. The crude product was purified by a column chromatography on silica gel to give the title compound as a brown solid (530 mg, 77%). ¹H-NMR (CDCl₃): δ 1.30-1.50 (1H, m), 1.55-1.83 (4H, m), 2.00-2.15 (3H, m), 2.25 (3H, s), 2.32-2.45 (2H, m), 2.70-3.06 (3H, m), 3.74 (3H, s), 4.71-4.80 (1H, d), 6.66-6.70 (2H, m), 7.38-7.40 (1H, d); TLC (DCM/MeOH 3:1):R_{f}=0.7

### Intermediate 170: 2-[(8aR)-hexahydropyrrolo[1,2-a]pyrazin-2(1H)-yl]-2-[2-methyl-4-(methyloxy)phenyl]acetamide-diastereomeric mixture

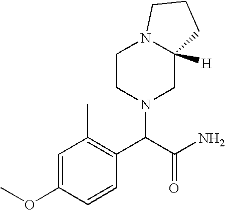

A mixture of 470 mg of (8aR)-hexahydropyrrolo[1,2-a]pyrazin-2(1H)-yl[2-methyl-4-(methyloxy)phenyl]acetonitrile (intermediate 169, 1.65 mmole), 30 mL of a solution of KOH 25% in water (0.13 mole) and 50 mL of H₂O₂3% (4.4 mmole) was warmed to 45° C. The mixture was stirred overnight at 45° C. for 40 h. The mixture was dried by lyophilization. The crude was purified by preparative HPLC (P-HPLC condition see HPLC condition A) to give the title compound as a white solid (130 mg, 26%). ¹H-NMR (CD₃OD): δ 1.20-1.45 (1H, m), 1.60-1.80 (4H, m), 2.08-2.21 (2H, m), 2.20-2.30 (1H, m), 2.39 (3H, s), 2.40 (1H, m), 2.55-2.70 (1H, m), 2.88-3.20 (3H, m), 3.74 (3H, s), 4.08-4.10 (1H, d), 6.73-6.75 (2H, m), 7.41-4.45 (1H, t); m/z (ES): 304 [M+H]⁺.

### Compound 108: N′-[3,5-bis(trifluoromethyl)phenyl]-2-[(8aR)-hexahydropyrrolo[1,2-a]-pyrazin-2(1H)-yl]-2-[2-methyl-4-(methyloxy)phenyl]acetohydrazide-diastereomeric mixture

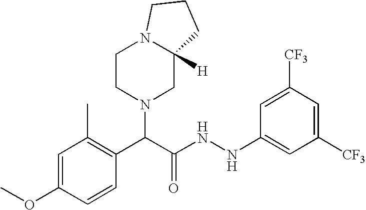

In a 10 mL round-bottomed flask, were added 130 mg of 2-[(8aR)-hexahydropyrrolo[1,2-a]pyrazin-2(1H)-yl]-2-[2-methyl-4-(methyloxy)phenyl]acetamide (intermediate 170, 0.428 mmole) in 4 mL of DCM to give a colorless solution. Then, 209 μL of BOC₂O (0.900 mmole, Aldrich) were added followed by 5.2 mg of DMAP (0.043 mmole, Aldrich) and the mixture was stirred at RT for 4 h. Then, 126 mg of [3,5-bis(trifluoromethyl)phenyl]hydrazine (0.514 mmole, Aldrich) were added and the mixture was stirred at RT overnight. The residue was purified via Biotage (5:1 CH₂Cl₂/MeOH; 12M column) and then by MDAP to give the title compound (30 mg, 13.20%). ¹H-NMR (400 MHz, CDCl₃): δ 2.08-2.10 (4H, m), 2.39 (3H, s), 3.04 (5H, m), 3.41 (4H, m), 3.81 (3H, s), 4.49 (1H, m), 6.77 (2H, s), 6.99 (2H, d), 7.25 (1H, s), 7.49 (1H, m), 8.41 (1H, br s), 9.37 (1H, d); m/z (ES): 531.2 [M+H]⁺

### Compound 109: N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide-diastereoisomer 2

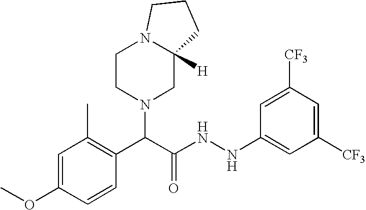

30 mg of Compound 109 (0.057 mmole) were separated by chiral HPLC to give the title compound. Chiral HPLC [Column: Chiralpak AD-H (25×0.46 cm); Mobile phase: n-Hexane/2-Propanol 85/15% v/v; Flow rate: 1.0 ml/min; DAD: 210-340 nm; CD: 245 nm]. Rₜ=12.31 min. (100% ee).

### Intermediate 171: (8aR)-hexahydropyrrolo[1,2-a]pyrazin-2(1H)-yl(3-methylphenyl)-acetonitrile-diastereomeric mixture

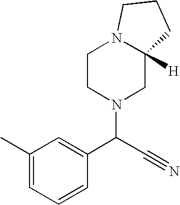

To a solution of 300 mg of 3-methylbenzaldehyde (2.8 mmole) in 15 mL of diethyl ether were added 410 μL trimethylsilyl cyanide (3 mmole) and 41 mg of zinc iodide (0.13 mmole) at RT under nitrogen. The mixture was cooled to 0° C. and stirred for 5 min at 0° C. Then, the mixture was treated with a solution of 500 mg of (R)-octahydropyrrolo[1,2-a]pyrazine dihydrochloride (intermediate 115, 2.5 mmole) in 15 mL of MeOH. Then, 730 μL of TEA (5 mmole) were added to the mixture. The reaction was refluxed overnight. The solution was cooled to RT and 20 mL of a saturated aqueous solution of K₂CO₃ were added. The mixture was extracted with AcOEt (3×20 mL). The combined organic layers were dried over Na₂SO₄, filtered and evaporated to dryness. The resulting crude was purified by a flash chromatography on silica gel to give the title compound as a brown oil (450 mg, 70%). ¹H-NMR (CDCl₃): δ: 1.30-1.52 (1H, m), 1.61-1.92 (4H, m), 2.00-2.30 (2H, m), 2.38 (3H, s), 2.40-2.50 (1H, m), 2.50-2.65 (1H, m), 2.80-2.90 (1H, m), 3.00-3.15 (3H, m), 4.80-4.85 (1H, d), 7.12-7.16 (1H, m), 7.25-7.35 (3H, m); TLC (MeOH/DCM 1:10): R_{f}=0.8.

### Intermediate 172: 2-[(8aR)-hexahydropyrrolo[1,2-a]pyrazin-2(1H)-yl]-2-(3-methylphenyl)-acetamide-diastereomeric mixture

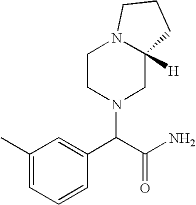

A mixture of 430 mg of (8aR)-hexahydropyrrolo[1,2-a]pyrazin-2(1H)-yl(3-methylphenyl)acetonitrile (intermediate 171, 1.7 mmole), 50 mL of H₂O₂ 3% (44 mmole) and 30 mL of KOH 25% (0.13 mole) was heated at 45° C. for 72 h. The mixture was freeze-dried and purified by a flash chromatography on silica gel to afford crude product. The resulting product was further purified by preparative HPLC (condition A) to give the title compound as white solid (35 mg). ¹H-NMR (CD₃OD) δ: 1.21-1.45 (1H, m), 1.62-1.82 (4H, m), 1.91-2.22 (3H, m), 2.31-2.35 (4H, m), 2.50-2.72 (1H, m), 2.91-3.20 (3H, m), 3.78 (1H, d), 7.08-7.12 (1H, s), 7.15-7.30 (3H, m); LC-MS: Rₜ=2.08 min (93.29%) m/z (ES): 274 [M+H]⁺.

### Compound 110: N′-[3,5-bis(trifluoromethyl)phenyl]-2-[(8aR)-hexahydropyrrolo[1,2-a]-pyrazin-2(1H)-yl]-2-(3-methylphenyl)acetohydrazide-diastereomeric mixture

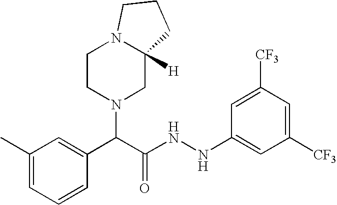

In a 10 mL round-bottomed flask, 102 mg of 2-[(8aR)-hexahydropyrrolo[1,2-a]pyrazin-2(1H)-yl]-2-(3-methylphenyl)acetamide (intermediate 172, 0.373 mmole) were dissolved in 4 mL DCM to give a colorless solution. Then, 9.11 mg of DMAP (0.037 mmole, Aldrich) were added followed by 93 mg of bis(1,1-dimethylethyl) dicarbonate (0.765 mmole, Aldrich) and the mixture was stirred at RT overnight. More bis(1,1-dimethylethyl) dicarbonate was added and stirring was continued at room temperature for 4 h. Then 98 mg of [3,5-bis(trifluoromethyl)phenyl]hydrazine (0.448 mmole, Aldrich) and 18 mg of DMAP (0.037 mmole, Aldrich) were added and the solution was concentrated to a third and stirred at RT overnight. Solvent was then evaporated and the residue was purified via Biotage (from DCM to 30% MeOH in DCM). The obtained product was further purified by MDAP to give the title compound as a mixture of diastereoisomers. (5 mg, 2.68%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% B to 60% B in 1.05 min, 60% B to 100% B in 0.5 min., 100% B for 0.2 min, flow rate: 1 ml/min]: R_{f}=0.72 min. (67%) m/z (ES): 501.16 [M+H]⁺; MS: m/z (ES): 501.2 [M+H]

### Intermediate 173: (6-(trifluoromethyl)pyridin-3-yl)methanol

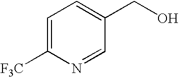

To a solution of 2 g of 6-(trifluoromethyl)nicotinic acid (10.5 mmole) in 100 mL of THF (100 ml) cooled at 0° C., were added 780 mg of LAH (21.1 mmole). The mixture was warmed to RT and stirred overnight. The reaction was quenched with Na₂SO₄.10H₂O and the precipitate was filtered. Mother liquors were evaporated under reduced pressure to give the crude product which was purified by a flash chromatography on silica gel to give the title compound as a brown oil (1.08 g, 58.3%). ¹H-NMR (CDCl₃): δ 4.82-4.85 (2H, s), 7.64-7.71 (1H, d), 7.91-7.93 (1H, d), 8.64-8.72 (1H, s); TLC (DCM/MeOH 10:1): R_{f}=0.8.

### Intermediate 174: 6-(trifluoromethyl)nicotinaldehyde

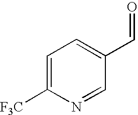

To a solution of 800 mg of 6-(trifluoromethyl)pyridin-3-yl)methanol (intermediate 173, 4.52 mmole) in 50 mL of DCM cooled at 0° C., were added 2 g of MnO₂ (23 mmole). The mixture was stirred at RT overnight. The solution was filtered and concentrated on standing at RT to give the title compound (750 mg, 95%) as white solid. ¹H-NMR (CDCl₃) δ: 7.88-7.90 (1H, d), 8.36-8.38 (1H, d), 9.20 (1H, s), 10.22 (1H, s); TLC (DCM/MeOH 10:1): R_{f}=0.9.

### Intermediate 175: 2-[(8aR)-hexahydropyrrolo[1,2-a]pyrazin-2(1H)-yl]-2-[6-(trifluoromethyl)-3-pyridinyl]acetamide-diastereomeric mixture

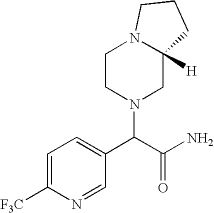

To a solution of 450 mg of 6-(trifluoromethyl)nicotinaldehyde (intermediate 174, 2.57 mmole) in 20 mL of diethyl ether, were added 410 μL of trimethylsilyl cyanide (3 mmole) and 41 mg of zinc iodide (0.1 mmole) at RT under nitrogen. The mixture was cooled to 0° C. and stirred for 5 min at 0° C. The mixture was treated with a solution of 500 mg of (R)-octahydropyrrolo[1,2-a]pyrazine dihydrochloride (intermediate 115, 2.51 mmole) in 20 mL of MeOH. Then, 730 μL of TEA (5 mmole) were added to the reaction. The mixture was refluxed overnight. Then, the solution was cooled to RT and 20 mL of a saturated aqueous solution of K₂CO₃ were added to the mixture. The resulting aqueous phase was extracted with AcOEt (3×20 mL). The combined organic layers were dried over Na₂SO₄, filtered and evaporated to dryness. The resulting crude was purified by a flash chromatography on silica gel to give 480 mg of a yellow oil. 460 mg (1.48 mmol) of this oil were dissolved in 10 mL of n-hexane at 0° C. 10 mL of H₂SO₄ 98% (200 mmole) were added and the mixture was stirred 16 h at RT. The mixture was cooled to 0° C., then ice was added. The acid was neutralized with NH₃H₂O 28% and the solution was freeze-dried. The residue was washed by DCM/MeOH (10/1) to afford crude product. The crude was purified by preparative HPLC (condition A) to give the title compound as a white solid (173 mg, 36%). ¹H-NMR (CD₃OD): δ 1.21-1.35 (1H, m), 1.70-1.89 (4H, m), 2.00-2.25 (2H, m), 2.30-2.45 (2H, m), 2.55-2.72 (1H, m), 2.90-3.10 (3H, m), 4.18 (1H, d), 7.80-7.82 (2H, d), 8.09-8.11 (2H, d), 8.73 (1H, s); LC/MS: m/z (ES): 329[M+H]⁺.

### Compound 111: N′-[3,5-bis(trifluoromethyl)phenyl]-2-[(8aR)-hexahydropyrrolo[1,2-a]pyrazin-2(1H)-yl]-2-[6-(trifluoromethyl)-3-pyridinyl]acetohydrazide-diastereomeric mixture

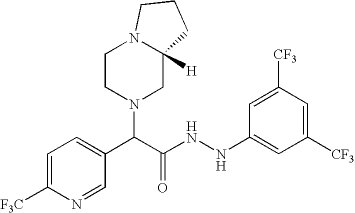

In a 10 mL round-bottomed flask, 173 mg of 2-[(8aR)-hexahydropyrrolo[1,2-a]pyrazin-2(1H)-yl]-2-[6-(trifluoromethyl)-3-pyridinyl]acetamide (intermediate 175, 0.527 mmole) were dissolved in 4 mL of DCM to give a colorless solution. Then, 12.86 mg of DMAP (0.053 mmole, Aldrich) were added followed by 132 mg of bis(1,1-dimethylethyl) dicarbonate (1.080 mmole, Aldrich) and the mixture was stirred at RT overnight. More bis(1,1-dimethylethyl) dicarbonate (0.52 mmole) was added and stirring was continued at RT for 4 h. Then 138 mg of [3,5-bis(trifluoromethyl)phenyl]hydrazine (0.632 mmole, Aldrich) and 26 mg of DMAP (0.106 mmole, Aldrich) were added and the solution was concentrated to the third and stirred at RT overnight. Solvent was then evaporated under reduced pressure and the residue was purified via Biotage (from DCM to 30% MeOH in DCM); 12M column to give the title compound (42 mg, 14.35%). ¹H-NMR (400 MHz, CDCl₃): δ 1.27-1.47 (2H, m), 1.72-1.85 (2H, m), 2.09-2.20 (2H, m), 2.30-2.60 (2H, m), 2.70 (1H, d), 2.87 (1H, d), 3.01-3.11 (2H, m), 3.23 (1H, d), 4.41 (1H, m), 6.53 (1H, d), 7.11 (2H, s), 7.34 (1H, s), 7.72 (1H, d), 7.95 (1H, m), 8.22 (1H, s), 8.70 (1H, s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: R_{f}=0.71 min. (84%) m/z (ES): 555.0 [M]⁺;

### Intermediate 176: (8aR)-hexahydropyrrolo[1,2-a]pyrazin-2(1H)-yl{2-[(trifluoromethyl)oxy]-phenyl}acetic acid dihydrochloride-diastereomeric mixture

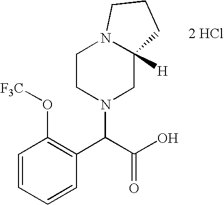

In a 20 mL microwave vial were suspended 259 mg of {2-[(trifluoromethyl)oxy]phenyl}boronic acid (1.256 mmole, Aldrich), 250 mg of (8aR)-octahydropyrrolo[1,2-a]pyrazine dihydrochloride (intermediate 115, 1.256 mmole), 93 mg of oxoacetic acid (1.256 mmole, Aldrich) and 347 mg of potassium carbonate (2.51 mmole, Aldrich) in 10 mL of acetonitrile. The mixture was then heated at 120° C. for 20 minutes under microwave irradiation. The solvent was then evaporated and the product purified via HLB (from water to MeOH, loading the crude dissolved into few mL of 2N HCl) to give the title compound (39 mg, 7.44%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.50 min. (37%) m/z (ES): 345.16 [M+H]⁺.

### Compound 112: N′-[3,5-bis(trifluoromethyl)phenyl]-2-[(8aR)-hexahydropyrrolo[1,2-a]-pyrazin-2(1H)-yl]-2-{2-[(trifluoromethyl)oxy]phenyl}acetohydrazide-diastereomeric mixture

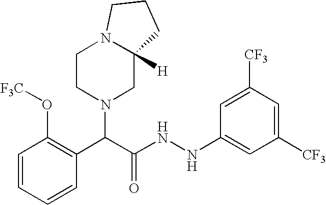

In a 100 mL round-bottomed flask, 39 mg of (8aR)-hexahydropyrrolo[1,2-a]pyrazin-2(1H)-yl{2-[(trifluoromethyl)oxy]phenyl}acetic acid dihydrochloride (intermediate 176, 0.093 mmole) were dissolved in 2 mL of DMF to give a colorless solution to which were added 4 of N-Methylmorpholine (0.280 mmole, Aldrich), 25.1 mg of [3,5-bis(trifluoromethyl)phenyl]hydrazine (0.103 mmole, Aldrich) and 45.5 mg of BOP (0.103 mmole, Aldrich). The resulting mixture was stirred at RT overnight. The mixture was diluted with 1N NaOH and transferred to a separatory funnel. The aqueous phase was extracted with AcOEt, washed with a saturated aqueous solution of NaHCO₃, brine, dried over Na₂SO₄, filtered and concentrated. The residue was purified via Biotage (DCM/MeOH from 100/0 to 70/30) to give the title compound (40 mg, 75%). ¹H-NMR (400 MHz, CDCl₃): δ 1.51 (1H, m), 1.83-2.02 (3H, m), 2.28-2.46 (4H, m), 2.54 (2H, t), 2.78 (1H, d), 2.89-3.17 (3H, m), 4.72 (1H, s), 4.76 (1H, s), 6.88 (1H, s), 7.08 (3H, s), 7.39-7.43 (1H, m), 7.53 (1H, t), 9.00 (1H, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.76 min (90%) m/z (ES): 571.13 [M+H]⁺.

### Intermediate 177: (8aR)-hexahydropyrrolo[1,2-a]pyrazin-2(1H)-yl[2-(methyloxy)phenyl]-acetic acid dihydrochloride-diastereomeric mixture

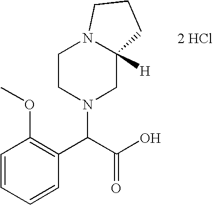

In a 20 mL microwave vial, were suspended 191 mg of [2-(methyloxy)phenyl]boronic acid (1.256 mmole, Aldrich), 93 mg of glyoxilic acid (1.256 mmole, Aldrich), 250 mg of (8aR)-octahydropyrrolo[1,2-a]pyrazine dihydrochloride (intermediate 115, 1.256 mmol) and 347 mg of potassium carbonate (2.51 mmol, Aldrich) in 10 mL of acetonitrile. The reaction was then heated at 120° C. for 20 minutes under microwave irradiation. The solvent was then removed and the crude dissolved into 5 mL of 2M HCl and purified via a HLB Oasis Extraction Cartridge (from water to methanol) to give the title compound that was used without any further purification (190 mg, 52.1%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.36 min. (99%) m/z (ES): 291.16 [M+H]⁺; MS: m/z (ES): 290.9 [M+H]⁺.

### Intermediate 178: N′-[3,5-bis(trifluoromethyl)phenyl]-2-[(8aR)-hexahydropyrrolo[1,2-a]-pyrazin-2(1H)-yl]-2-[2-(methyloxy)phenyl]acetohydrazide-diastereomeric mixture

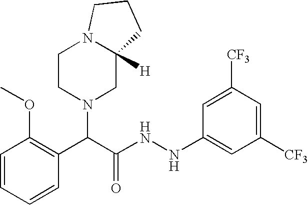

In a 100 mL round-bottomed flask, 190 mg of (8aR)-hexahydropyrrolo[1,2-a]pyrazin-2(1H)-yl[2-(methyloxy)phenyl]acetic acid dihydrochloride (intermediate 177, 0.523 mmole) were dissolved in 10 mL of DMF to give a colorless solution to which were added 173 μL of N-methylmorpholine (1.569 mmole, Aldrich), 140 mg of [3,5-bis(trifluoromethyl)phenyl]hydrazine (0.575 mmole, Aldrich) and 254 mg of BOP (0.575 mmole, Aldrich). The resulting mixture was stirred at RT overnight. Then the mixture was diluted with 1N NaOH and the aqueous phase was extracted with AcOEt. The combined organic layers were washed with a saturated aqueous solution of NaHCO₃, brine, dried over Na₂SO₄, filtered and concentrated under reduced pressure. The residue was purified via Biotage (DCM/MeOH from 100/0 to 70/30) to give the title compound (180 mg, 67%). ¹H-NMR (400 MHz, CDCl₃): δ 1.15-1.29 (1H, m), 1.73-1.89 (4H, m), 2.11-2.44 (4H, m), 2.65 (2H, d), 2.77 (1H, d), 2.92-3.18 (4H, m), 3.05 (3H, s), 4.72 (1H, m), 6.97 (2H, m), 7.08 (2H, s), 7.21 (1H, d), 8.98 (1H, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.68 min. (67%) m/z (ES): 517.15 [M+H]⁺.

### Compound 113: N′-[3,5-bis(trifluoromethyl)phenyl]-2-[(8aR)-hexahydropyrrolo[1,2-a]-pyrazin-2(1H)-yl]-2-[2-(methyloxy)phenyl]acetohydrazide-diastereoisomer 2

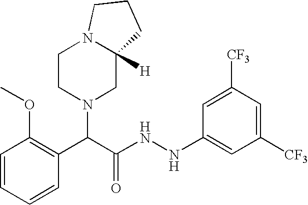

180 mg of N′-[3,5-bis(trifluoromethyl)phenyl]-2-[(8aR)-hexahydropyrrolo[1,2-a]pyrazin-2(1H)-yl]-2-[2-(methyloxy)phenyl]acetohydrazide-diastereomeric mixture (intermediate 178, 0.349 mmole) were separated by chiral preparative HPLC to give the title compound (diastereoisomer 2) (45 mg, 25%). Chiral HPLC [Column: Chiralpak AD-H (25×0.46 cm); Mobile phase: n-Hexane/2-Propanol 90/10; Flow rate: 1.0 mL/min; DAD: 210-340 nm; CD: 245 nm]: Rₜ=11.85 min. (100% ee).

### Intermediate 179: (8aR)-hexahydropyrrolo[1,2-a]pyrazin-2(1H)-yl(2-methylphenyl)acetic acid dihydrochloride

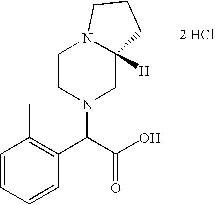

In a 20 mL microwave vial were suspended 171 mg of (2-methylphenyl)boronic acid (1.256 mmole, Aldrich), 93 mg of glyoxilic acid (1.256 mmole, Aldrich), 250 mg of (8aR)-octahydropyrrolo[1,2-a]pyrazine dihydrochloride (intermediate 115, 1.256 mmole) and 347 mg of potassium carbonate (2.51 mmole, Aldrich) in Acetonitrile. The mixture was then heated at 120° C. for 20 minutes under microwave irradiation. The solvent was then removed under reduced pressure and the crude was dissolved into 5 mL of HCl 2M in water. The solution was purified via a HLB Oasis Extraction Cartridge (from water to methanol) to give the title compound which was used in the next step without any further purification (300 mg, 87%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.42 min. (99%) m/z (ES): 275.17 [M+H]⁺; MS: m/z (ES): 275.0 [M+H]⁺.

### Compound 114: N′-[3,5-bis(trifluoromethyl)phenyl]-2-[(8aR)-hexahydropyrrolo[1,2-a]-pyrazin-2(1H)-yl]-2-(2-methylphenyl)acetohydrazide-diastereomeric mixture

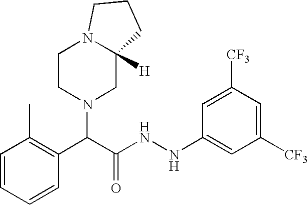

In a 100 mL round-bottomed flask, 300 mg of (8aR)-hexahydropyrrolo[1,2-a]pyrazin-2(1H)-yl(2-methylphenyl)acetic acid dihydrochloride (intermediate 179, 0.864 mmole) were dissolved in 10 mL of DMF to give a colorless solution to which were added 285 μL N-methylmorpholine (2.59 mmole, Aldrich), 232 mg of [3,5-bis(trifluoromethyl)phenyl]hydrazine (0.950 mmole, Aldrich) and 420 mg of BOP (0.950 mmole, Aldrich). The resulting mixture was stirred at RT overnight. The mixture was diluted with 1N NaOH and the aqueous phase was extracted with AcOEt The combined organic layers were washed with a saturated solution of NaHCO₃, brine, dried over Na₂SO₄, filtered and evaporated under reduced pressure. The residue was purified via Biotage (10:1 CH₂Cl₂/MeOH; 12M column) to give the title compound (155 mg, 36%). ¹H-NMR (400 MHz, CDCl₃): δ 1.33-1.48 (1H, m), 1.72-1.90 (4H, m), 2.12-2.29 (4H, m), 2.44 (3H, s), 2.58 (1H, m), 2.93-3.21 (4H, m), 3.47 (3H, s), 4.42 (1H, s), 6.86 (1H, d), 6.97 (2H, s), 7.26 (1H, m), 7.52 (2H, d), 8.72 (1, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.72 min. (91%) m/z (ES): 501.16 [M+H]⁺.

### Compound 115: N′-[3,5-bis(trifluoromethyl)phenyl]-2-[(8aR)-hexahydropyrrolo[1,2-a]-pyrazin-2(1H)-yl]-2-(2-methylphenyl)acetohydrazide-diastereoisomer 2

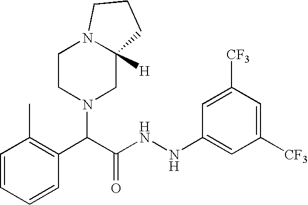

155 mg of N′-[3,5-bis(trifluoromethyl)phenyl]-2-[(8aR)-hexahydropyrrolo[1,2-a]pyrazin-2(1H)-yl]-2-(2-methylphenyl)acetohydrazide-diastereomeric mixture (compound 114) were purified by chiral preparative HPLC to give isomer 2 (53 mg, 34%). Chiral HPLC [Column: Chiralpak AD-H (25×0.46 cm); Mobile phase: n-Hexane/2-Propanol 88/12% v/v, Flow rate: 1.0 ml/min, DAD: 210-340 nm, CD: 250 nm]: Rₜ=12.37 min. (100% ee).

### Intermediate 180: (8aR)-hexahydropyrrolo[1,2-a]pyrazin-2(1H)-yl[2-(1-methylethyl)-phenyl]acetic acid dihydrochloride-diastereomeric mixture

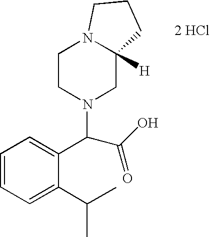

In a 20 mL microwave vial, were suspended 206 mg of [2-(1-methylethyl)phenyl]boronic acid (1.256 mmole, Aldrich), 93 mg of glyoxilic acid (1.256 mmole, Aldrich), 250 mg of (8aR)-octahydropyrrolo[1,2-a]pyrazine dihydrochloride (intermediate 115, 1.256 mmole) and 347 mg of potassium carbonate (2.51 mmole, Aldrich) in 10 mL of CH₃CN. The reaction was then heated at 120° C. for 20 minutes under microwave irradiation. The solvent was then removed and the crude was dissolved into 5 mL of 2M HCl and purified via a HLB Oasis Extraction Cartridge (from water to methanol) to give the title compound which was used in the next step without any further purification (275 mg, 72%). UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.52 min. (97%) m/z (ES): 303.16 [M+H]⁺; MS: m/z (ES): 302.9 [M+H]⁺.

### Compound 116: N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide-diastereomeric mixture

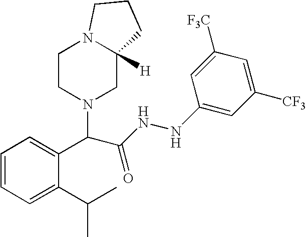

In a 100 mL round-bottomed flask, 275 mg of (8aR)-hexahydropyrrolo[1,2-a]pyrazin-2(1H)-yl[2-(1-methylethyl)phenyl]acetic acid dihydrochloride-diastereomeric mixture (intermediate 180, 0.733 mmole) were dissolved in 10 mL of DMF to give a colorless solution to which were added 242 μL of N-methylmorpholine (2.198 mmole, Aldrich), 197 mg of [3,5-bis(trifluoromethyl)phenyl]hydrazine (0.806 mmole, Aldrich) and 356 mg of BOP (0.806 mmole, Aldrich). The resulting mixture was stirred at RT overnight. The mixture was then diluted with 1N NaOH. The resulting aqueous phase was extracted with AcOEt. The combined organic layers were washed with a saturated aqueous solution of NaHCO₃, brine, dried over Na₂SO₄, filtered and evaporated under reduced pressure. The residue was purified via Biotage (10:1 CH₂Cl₂/MeOH; 12M column) to give the title compound (180 mg, 46.5%). ¹H-NMR (400 MHz, CDCl₃): δ 1.24 (6H, t), 1.50 (1H, m), 1.80-1.92 (4H, m), 2.11 (1H, t), 2.19-2.36 (4H, m), 2.83 (1H, d), 3.01 (1H, d), 3.10 (1H, t), 3.26 (1H, d), 3.49 (1H, m), 4.54 (1H, s), 6.55 (1H, s), 7.01 (2H, s), 7.37 (2H, m), 7.57 (2H, d), 8.62 (1H, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.75 min. (90%) m/z (ES): 529.22 [M+H]⁺; MS: m/z (ES): 529.0 [M+H]⁺.

### Compound 117: N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide-diastereoisomer 2

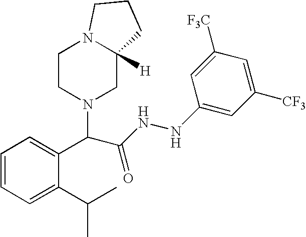

180 mg of N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide-diastereomeric mixture (compound 116) were purified by chiral HPLC to give the title compound (diastereoisomer 2) (71 mg, 18%). Chiral HPLC [Column: Chiralpak AD-H (25×0.46 cm); Mobile phase: n-Hexane/2-Propanol 88/12% v/v, Flow rate: 1.0 ml/min, DAD: 210-340 nm, CD: 250 nm]: Rₜ=10.14 min. (100% ee).

### Intermediate 181: (8aR)-hexahydropyrrolo[1,2-a]pyrazin-2(1H)-yl(1H-pyrazol-3-yl)-acetonitrile-mixture of diastereoisomers

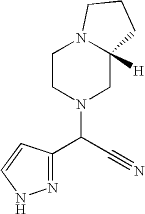

To a solution of 240 mg of 1H-pyrazole-4-carbaldehyde (2.8 mmole) in 15 mL of diethyl ether were added 410 μL trimethylsilyl cyanide (3 mmole) and 41 mg of zinc iodide (0.13 mmole) at RT under nitrogen. The mixture was cooled to 0° C. and stirred 5 min at 0° C. Then the mixture was treated with a solution of 500 mg of (8aR)-octahydropyrrolo[1,2-a]pyrazine dihydrochloride (intermediate 115, 2.5 mmole) in 15 mL of MeOH. Then, 730 μL of TEA (5 mmole) were added and the solution was refluxed overnight. The reaction was cooled to RT and 20 mL of a saturated aqueous solution of K₂CO₃ were added. The mixture was extracted with AcOEt (3×20 mL). The combined organic layers were dried over Na₂SO₄, filtered, and evaporated to dryness. The crude was purified by a flash chromatography on silica gel to give the title compound as a white solid (330 mg, 57%). ¹H-NMR (CDCl₃): δ 1.40-1.50 (1H, m), 1.80-1.90 (3H, m), 2.12-2.25 (2H, m), 2.39-2.52 (2H, m), 2.77-2.85 (2H, m), 2.89-3.12 (3H, m), 4.96-4.99 (1H, d), 6.44-6.45 (1H, d), 7.63-7.64 (1H, d). TLC (DCM/MeOH 9:1): R_{f}=0.6.

### Intermediate 182: 2-[(8aR)-hexahydropyrrolo[1,2-a]pyrazin-2(1H)-yl]-2-(1H-pyrazol-3-yl)-acetamide-diastereomeric mixture

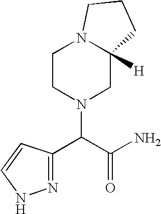

To a solution of 310 mg of (8aR)-hexahydropyrrolo[1,2-a]pyrazin-2(1H)-yl(1H-pyrazol-3-yl)acetonitrile-mixture of diastereoisomers (intermediate 181, 1.34 mmole) in 15 mL of n-hexane cooled at 0° C., were added 15 mL of H₂SO₄ (0.3 mole). The mixture was stirred for 18 h at RT. Then, the solution was cooled to 0° C. and ice was added. The acid was neutralized with NH₃H₂O 28% and the mixture was freeze-dried. The residue was washed with DCM/MeOH (10/1) to get the crude product. The crude was purified by preparative HPLC (see HPLC condition A) to give the title compound as a pale yellow solid (204 mg, 61%). ¹H-NMR (CD₃OD): δ 1.21-1.42 (1H, m), 1.65-1.85 (4H, m), 2.00-2.30 (3H, m), 2.33-2.41 (1H, m), 2.60-2.75 (1H, m), 2.90-3.10 (3H, m), 4.12 (1H, d), 6.32 (1H, s), 7.58 (1H, s); LC-MS: Rₜ=2.5 min (99.74%) m/z (ES): 250 [M+H]⁺, 521 [2M+Na]⁺.

### Compound 118: N′-[3,5-bis(trifluoromethyl)phenyl]-2-[(8aR)-hexahydropyrrolo[1,2-a]-pyrazin-2(1H)-yl]-2-(1H-pyrazol-3-yl)acetohydrazide-mixture of diastereoisomers

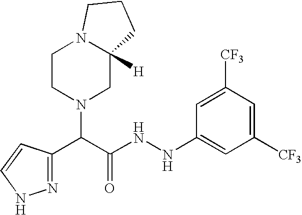

In a 10 mL round-bottomed flask, 204 mg of 2-[(8aR)-hexahydropyrrolo[1,2-a]pyrazin-2(1H)-yl]-2-(1H-pyrazol-3-yl)acetamide-diastereomeric mixture (intermediate 182, 0.818 mmole) were dissolved in 4 mL of DCM to give a colorless solution. Then, 19.98 mg of DMAP (0.082 mmole, Aldrich) were added followed by 310 mg of bis(1,1-dimethylethyl) dicarbonate (2.54 mmole, Aldrich). The mixture was stirred at RT overnight. More bis(1,1-dimethylethyl) dicarbonate was added and stirring was continued at RT for 4 h, then 214 mg of [3,5-bis(trifluoromethyl)phenyl]hydrazine (0.982 mmole, Aldrich) and 40 mg of DMAP (0.164 mmole, Aldrich) were added and the solution was concentrated to the third. The mixture was stirred at RT overnight. Solvent was then evaporated and the residue was purified via Biotage (10:1 CH₂Cl₂/MeOH; 12M column). The obtained product was then dissolved in 4 mL of DCM and treated with 2 mL of TFA. The reaction was stirred at RT overnight. Solvents were removed under reduced pressure. The resulting residue was, first, purified via SCX cartridge (from DCM to MeOH and then 0.5M NH₃ in MeOH) and then by MDAP to give the title compound (87 mg, 22%). ¹H-NMR (400 MHz, CDCl₃): 1.40 (1H, m), 1.69-1.87 (2H, m), 2.09-2.27 (3H, m), 2.36 (1H, t), 2.54 (1H, t), 2.67 (1H, d), 2.84 (1H, d), 2.97 (1H, d), 3.05 (2H, m), 4.38 (1H, m), 5.87 (1H, d), 6.33 (1H, d), 7.09 (2H, s), 7.25 (1H, s), 7.45 (1H, s), 7.50 (1H, dd), 9.45 (1H, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.59 min. (82%) m/z (ES): 477.15 [M+H]⁺.

### Compound 119: N′-[3,5-bis(trifluoromethyl)phenyl]-2-[(8aR)-hexahydropyrrolo[1,2-a]-pyrazin-2(1H)-yl]-2-(1H-pyrazol-3-yl)acetohydrazide-diastereoisomers 2

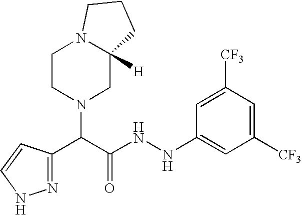

87 mg of N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide-mixture of diastereoisomers (compound 118) were separated by chiral HPLC to give the title compound (27 mg, 6.93%). Chiral HPLC [Column: Chiralpak AD-H (25×0.46 cm); Mobile phase: n-Hexane/2-Propanol 85/15% v/v, Flow rate: 1.0 ml/min, DAD: 210-340 nm, CD: 245 nm]: Rₜ=10.14 min. (100% ee).

### Intermediate 183: (2-fluorophenyl)[(8aR)-hexahydropyrrolo[1,2-a]pyrazin-2(1H)-yl]-acetonitrile-diastereomeric mixture

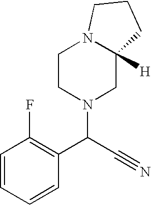

To a solution of 318 mg of 2-fluorobenzaldehyde (2.8 mmole) in 15 mL of diethyl ether were added 410 μL of trimethylsilyl cyanide (3 mmole) and 41 mg of zinc iodide (0.13 mmole) at RT under nitrogen. The mixture was cooled to 0° C. and stirred 5 min at 0° C. Then the mixture was treated with a solution of 500 mg of (8aR)-octahydropyrrolo[1,2-a]pyrazine dihydrochloride (intermediate 115, 2.5 mmole) in 15 mL of MeOH. Then, 730 μL of TEA (5 mmole) were added to the mixture. The solution was refluxed overnight. The mixture was cooled to RT and 20 mL of a saturated aqueous solution of K₂CO₃ were added. The aqueous phase was extracted with AcOEt (4×20 mL). The combined organic layers were dried over Na₂SO₄, filtered and evaporated to dryness. The crude was purified by a flash chromatography on silica gel to give the title compound as a brown oil (450 mg, 69%). ¹H-NMR (CDCl₃): δ 1.30-1.52 (1H, m), 1.60-2.02 (4H, m), 2.11-2.24 (2H, m), 2.30-2.41 (1H, m), 2.60-2.90 (2H, m), 2.91-3.15 (3H, m), 5.08-5.10 (1H, d), 7.09-7.12 (1H, m), 7.18-7.20 (1H, m), 7.35-7.41 (1H, m), 7.51-7.61 (1H, td); TLC (DCM/MeOH 10:1) R_{f}=0.7.

### Intermediate 184: 2-(2-fluorophenyl)-2-[(8aR)-hexahydropyrrolo[1,2-a]pyrazin-2(1H)-yl]acetamide-diastereomeric mixture

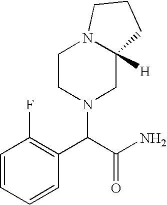

A mixture of 430 mg of (2-fluorophenyl)[(8aR)-hexahydropyrrolo[1,2-a]pyrazin-2(1H)-yl]acetonitrile-diastereomeric mixture (intermediate 183, 1.66 mmole), 50 mL of H₂O₂ 3% (44 mmole) and 30 mL of KOH 25% (0.13 mole) was heated at 45° C. for 24 h. The mixture was freeze-dried and purified by a flash chromatography on silica gel to give the title compound as a white solid (250 mg, 54%). ¹H-NMR (CD₃OD): δ 1.10-1.20 (1H, m), 1.30-2.80 (4H, m), 1.90-2.20 (2H, m), 2.20-2.41 (2H, m), 2.58-2.80 (1H, m), 2.81-3.10 (3H, m), 4.32 (1H, s), 7.01-7.10 (2H, m), 7.20-7.30 (1H, m), 7.38-7.42 (1H, m); HPLC: Rₜ=3.5 min (99.09%). m/z (ES) 278 [M+H]⁺, 577 [2M+Na]⁺.

### Compound 120: N′-[3,5-bis(trifluoromethyl)phenyl]-2-(2-fluorophenyl)-2-[(8aR)-hexahydro pyrrolo[1,2-a]pyrazin-2(1H)-yl]acetohydrazide-diastereomeric mixture

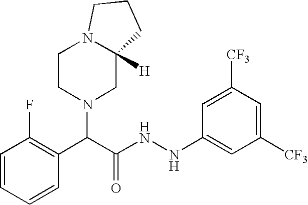

In a 10 mL round-bottomed flask, 250 mg of 2-(2-fluorophenyl)-2-[(8aR)-hexahydropyrrolo[1,2-a]pyrazin-2(1H)-yl]acetamide-diastereomeric mixture (intermediate 184, 0.901 mmole) were dissolved in 4 mL of DCM to give a colorless solution. Then, 440 μL of BOC₂O (1.893 mmole, Aldrich) were added followed by 11,01 mg of DMAP (0.090 mmole, Aldrich) and the mixture was stirred at RT for 4 h. Then, 22 mg of DMAP (0.18 mmole, Aldrich) and 264 mg of [3,5-bis(trifluoromethyl)phenyl]hydrazine (1.082 mmole, Aldrich) were added and the reaction was stirred at RT overnight. The residue was purified via Biotage (5:1 CH₂Cl₂/MeOH; 12M column) to give the title compound (320 mg, 70%). ¹H-NMR (400 MHz, CDCl₃): δ 1.44 (1H, m), 1.75-1.89 (5H, m), 2.23-2.48 (2H, m), 2.64 (1H, t), 2.83 (1H, d), 2.96-3.19 (3H, m), 3.51 (1H, s), 4.65 (1H, m), 6.62 (1H, d), 7.10-7.20 (3H, m), 7.31-7.38 (3H, m), 9.05 (1H, br s); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 ml/min]: Rₜ=0.65 min. (66%) m/z (ES): 505.17 [M+H]⁺.

### Compound 121 N′-[3,5-bis(trifluoromethyl)phenyl]-2-(2-fluorophenyl)-2-[(8aR)-hexahydro pyrrolo[1,2-a]pyrazin-2(1H)-yl]acetohydrazide-diastereoisomer 2

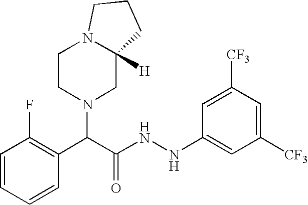

320 mg of N′-[3,5-bis(trifluoromethyl)phenyl]-2-(2-fluorophenyl)-2-[(8aR)-hexahydro pyrrolo[1,2-a]pyrazin-2(1H)-yl]acetohydrazide-diastereomeric mixture (compound 120) were separated by chiral HPLC to give the title compound (isomer 2) (115 mg, 36%). Chiral HPLC [Column: Chiralpak AD-H (25×0.46 cm); Mobile phase: n-Hexane/2-Propanol 88/12% v/v, Flow rate: 1.0 ml/min, DAD: 210-340 nm, CD: 245 nm]: Rₜ=9.17 min. (100% ee).

### Intermediate 185: (8aR)-hexahydropyrrolo[1,2-a]pyrazin-2(1H)-yl(5-pyrimidinyl)acetonitrile-mixture of diastereoisomers

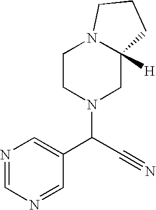

To a solution of 278 mg of pyrimidine-5-carbaldehyde (2.6 mmole) in 15 mL of diethyl ether were added 410 μL of trimethylsilyl cyanide (3 mmole) and 41 mg of zinc iodide (0.13 mmole). The mixture was cooled to 0° C. and stirred for 5 min at 0° C. Then, a solution of 500 mg of (8aR)-octahydropyrrolo[1,2-a]pyrazine dihydrochloride (intermediate 115, 2.5 mmole) in 15 mL of MeOH was added dropwise at 0° C. followed by 730 μL of TEA (5 mmole). The mixture was refluxed overnight. The solution was cooled to RT and 20 mL of a saturated aqueous solution of K₂CO₃ were added. The mixture was extracted with AcOEt (3×20 mL). The combined organic layers were dried over Na₂SO₄, filtered and evaporated to dryness. The resulting crude compound was purified by a flash chromatography on silica gel to give the title compound as a brown solid (410 mg, 67%). ¹H-NMR (CDCl₃) δ: 1.60-1.70 (4H, m), 1.80-2.00 (2H, m), 2.20-2.38 (1H, m), 2.55-2.68 (2H, m), 2.90-3.01 (1H, bs), 3.02-3.25 (3H, m), 4.86-4.95 (1H, d), 8.80-8.85 (2H, s), 9.13-9.22 (1H, s); TLC: DCM/MeOH (10:1): R_{f}=0.3.

### Intermediate 186: 2-[(8aR)-hexahydropyrrolo[1,2-a]pyrazin-2(1H)-yl]-2-(5-pyrimidinyl)-acetamide-mixture of diastereoisomers

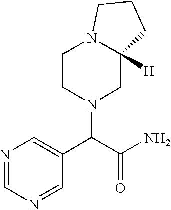

To a solution of 410 mg of (8aR)-hexahydropyrrolo[1,2-a]pyrazin-2(1H)-yl(5-pyrimidinyl)acetonitrile-mixture of diastereoisomers (intermediate 185, 1.7 mmole), in 10 mL of n-hexane at 0° C., were added 10 mL of H₂SO₄ (200 mmole). The mixture was stirred overnight at RT. The mixture was cooled to 0° C. and ice was added. The acid was neutralized with a 28% aqueous solution of NH₄OH and freeze-dried. The residue was extracted with DCM/MeOH (10/1). The solvents were removed under reduced pressure and the crude was purified by preparative HPLC to give the title compound as a white solid (193 mg, 44%). ¹H-NMR (CD₃OD): δ 1.20-1.47 (1H, m), 1.70-1.8 (3H, m), 1.95-2.22 (3H, m), 2.31-2.45 (2H, m), 2.60-2.80 (1H, m), 2.91-3.18 (3H, m), 4.20 (1H, s), 8.82 (2H, s), 9.11 (1H, s); m/z (ES): 262 [M+H]⁺.

### Compound 122: N′-[3,5-bis(trifluoromethyl)phenyl]-2-[(8aR)-hexahydropyrrolo[1,2-a]pyrazin-2(1H)-yl]-2-(5-pyrimidinyl)acetohydrazide-diastereomeric mixture

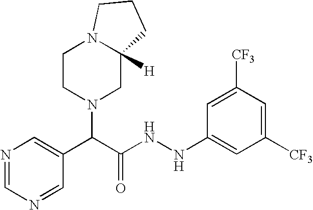

In a 10 mL round-bottomed flask, 193 mg of 2-[(8aR)-hexahydropyrrolo[1,2-a]pyrazin-2(1H)-yl]-2-(5-pyrimidinyl)acetamide-mixture of diastereoisomers (intermediate 186, 0.739 mmole) were dissolved in 6 mL of DCM to give a colorless solution. Then, 352 μL of BOC₂O (1.514 mmole, Aldrich) were added followed by 9.02 mg of DMAP (0.074 mmole, Aldrich). The mixture was stirred at RT for 4 h. Then, 18 mg of DMAP (0.148 mmole, Aldrich) were added followed by 216 mg of [3,5-bis(trifluoromethyl)phenyl]hydrazine (0.886 mmole, Aldrich). The volume of the mixture was reduced to the third and the reaction mixture was left under nitrogen for the weekend. Solvents were removed under reduced pressure and the residue was purified, first, via Biotage (5:1 CH₂Cl₂/MeOH; 12M column), then by MDAP (Method B) and, finally, with a SCX cartridge (from DCM to MeOH and then 0.5M NH₃ in MeOH) to give the title compound (120 mg, 33%). ¹H-NMR (400 MHz, CDCl₃): δ 1.74-1.84 (5H, m), 2.12-2.19 (3H, m), 2.40 (1H, m), 2.50-2.71 (1H, m), 2.89 (1H, d), 2.97-3.17 (3H, m), 4.43 (1H, m), 6.88 (1H, s), 7.15 (2H, s), 7.37 (1H, s), 8.69 (2H, d), 9.20 (1H, m); MS: m/z (ES): 489.16 [M+H]⁺;

### Compound 123: N′-[3,5-bis(trifluoromethyl)phenyl]-2-[(8aR)-hexahydropyrrolo[1,2-a]pyrazin-2(1H)-yl]-2-(5-pyrimidinyl)acetohydrazide-diastereoisomer 2

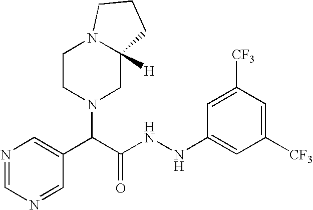

120 mg of N′-[3,5-bis(trifluoromethyl)phenyl]-2-[(8aR)-hexahydropyrrolo[1,2-a]pyrazin-2(1H)-yl]-2-(5-pyrimidinyl)acetohydrazide-diastereomeric mixture (compound 122) were separated by chiral preparative HPLC to give the title compound (diastereoisomer 2) (21 mg, 17%). Chiral HPLC [Column: Chiralpak AD-H (25×0.46 cm); Mobile phase: n-Hexane/2-Propanol 80/20% v/v, Flow rate: 1.0 ml/min, DAD: 210-340 nm, CD: 245 nm]: Rₜ=10.35 min. (100% ee).

### Intermediate 187: (8aR)-hexahydropyrrolo[1,2-a]pyrazin-2(1H)-yl(1-methyl-1H-imidazol-5-yl)acetonitrile-diastereomeric mixture

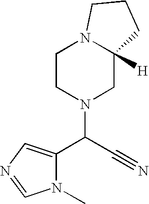

To a solution of 306 mg of 1-methyl-1H-imidazole-5-carbaldehyde (2.78 mmole) in 15 mL of diethyl ether were added 410 μL of trimethylsilyl cyanide (3 mmole) and 41 mg of zinc iodide (0.13 mmole) at RT under nitrogen. The mixture was cooled to 0° C. and was stirred for 5 min. Then, a solution of 500 mg of (8aR)-octahydropyrrolo[1,2-a]pyrazine dihydrochloride (intermediate 115, 2.5 mmole) in 15 mL of MeOH was added followed by 730 μL of TEA (5 mmole). The mixture was refluxed overnight. The solution was cooled to RT and 20 mL of a saturated aqueous solution of K₂CO₃ were added. The resulting aqueous solution was extracted with AcOEt (3×20 mL). The combined organic layers were washed with 20 mL of a saturated aqueous solution of NaHCO₃, dried over Na₂SO₄, filtered and evaporated to dryness. The crude was purified by flash chromatography on silica gel to afford the title compound as a brown oil (420 mg, 68%). ¹H-NMR (CDCl₃): δ 1.70-2.71 (10H, m), 2.75-3.15 (3H, m), 4.80-4.85 (1H, d), 7.25-7.27 (1H, m), 7.48-7.50 (1H, m); TLC (DCM/MeOH 10:1) R_{f}=0.3.

### Intermediate 188: 2-[(8aR)-hexahydropyrrolo[1,2-a]pyrazin-2(1H)-yl]-2-(1-methyl-1H-imidazol-5-yl)acetamide-diastereomeric mixture

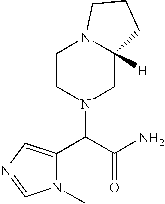

To a solution of 400 mg of (8aR)-hexahydropyrrolo[1,2-a]pyrazin-2(1H)-yl(1-methyl-1H-imidazol-5-yl)acetonitrile-diastereomeric mixture (intermediate 187, 1.63 mmole) in 10 mL of n-hexane, cooled at 0° C., were added 10 mL of H₂SO₄ (200 mmole). The mixture was stirred overnight at RT. The mixture was cooled to 0° C. and ice was added. Then, the acid was neutralized with NH₃H₂O 28% and the mixture was freeze-dried. The residue was extracted with DCM/MeOH (10/1) and the organic was evaporated under reduced pressure. The crude was purified by preparative HPLC (see HPLC condition A) to give the title compound as a brown solid (90 mg, 21%). ¹H-NMR (CD₃OD): δ 1.25-1.47 (1H, m), 1.70-2.00 (3H, m), 2.10-2.20 (3H, m), 2.20-2.55 (2H, m), 2.60-2.80 (1H, m), 2.80-3.08 (3H, m), 3.73 (3H, s), 4.18-4.25 (1H, d), 6.98 (1H, s), 7.60 (1H, s); MS: m/z (ES): 264 [M+H]⁺, 549 [2M+Na]⁺.

### Compound 124: N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide-mixture of diastereoisomer

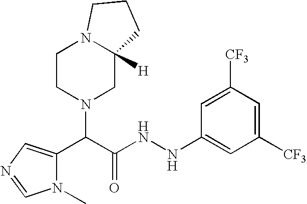

In a 10 mL round-bottomed flask, 90 mg of 2-[(8aR)-hexahydropyrrolo[1,2-a]pyrazin-2(1H)-yl]-2-(1-methyl-1H-imidazol-5-yl)acetamide-diastereomeric mixture (intermediate 188, 0.342 mmole) were dissolved in 3 mL of DCM. Then, 163 μL of BOC₂O (0.701 mmole, Aldrich) were added followed by 4.2 mg of DMAP (0.034 mmole, Aldrich). The mixture was stirred at RT for 4 h. Then 8.4 mg of DMAP (0.068 mmole, Aldrich) and 100 mg of [3,5-bis(trifluoromethyl)phenyl]hydrazine (0.410 mmole, Aldrich) were added. The solution volume was reduced to the third and the mixture was left under stirring over the weekend. Solvents were removed under reduced pressure and the residue was purified via biotage (4 g silica gel column, starting from DCM to 30% MeOH in DCM), repurified via MDAP (method B) and then by SCX cartridge (from DCM to MeOH and then 0.5M NH₃ in MeOH) to give the title compound (50 mg, 30%). ¹H-NMR (400 MHz, CDCl₃): δ 1.45 (1H, m), 1.71-1.89 (4H, m), 2.14-2.36 (2H, m), 2.55-2.71 (1H, m), 2.87 (1H, d), 2.99-3.09 (2H, m), 3.19 (1H, d), 3.72 (3H, s), 4.24 (1H, m), 6.96 (1H, d), 7.12 (2H, s), 7.33 (1H, s), 7.46 (1H, m), 9.43 (1H, br s); MS: m/z (ES): 491.18 [M+H]⁺.

### Compound 125: N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide-diastereoisomer 2

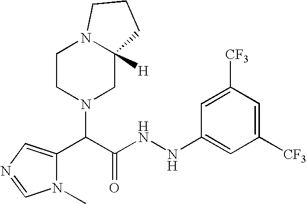

50 mg of N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide-mixture of diastereoisomer (compound 124) were separated by chiral preparative HPLC to give the title compound (diastereoisomer 2, 15 mg, 30%). Chiral HPLC [Column: Chiralcel OD-H; Mobile phase: n-Hexane/Ethanol 80/20% v/v, Flow rate: 0.8 ml/min, DAD: 210-340 nm, CD: 245 nm]: R=6.86 min. (100% ee)].

### Intermediate 189: Ethyl (8aS)-hexahydropyrrolo[1,2-a]pyrazin-2(1H)-yl(3-pyridinyl)acetate-mixture of diastereoisomers

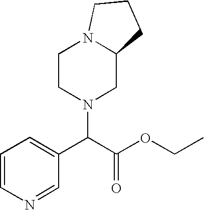

65 mg of ethyl(±)-hydroxy(3-pyridinyl)acetate (intermediate 125, 0.36 mmole) were dissolved in 2 mL of DCM to give a colorless solution. 60 μL of TEA (0.43 mmole, Aldrich) were added and the solution was cooled to 0° C. 30 μL of Mesyl-Cl (0.40 mmole, Fluka) were added and the pale yellow solution stirred at 0° C. for 30 min. Further TEA (0.43 mmole, 60 μL) and Mesyl-Cl (0.18 mmole, 18 μL) were added and stirring was continued for additional 30 min. Then, a solution of 100 mg of (8aS)-octahydropyrrolo[1,2-a]pyrazine (intermediate 114, 0.79 mmole) dissolved in DCM/DMF/TEA was added. The resulted mixture was stirred at RT for 48 h. Solvents were removed under reduced pressure and the crude was passed through a SCX cartridge eluting with DCM, MeOH and a 0.5M solution of NH₃ in MeOH. Solvents were removed under reduced pressure and the residue was purified by flash chromatography eluting with a gradient of DCM/MeOH to give the title compound as a yellow oil (30 mg, 29%). ¹H-NMR (400 MHz, CDCl₃): δ 1.20 (3H, t), 1.30-3.20 (13H, m), 4.20 (5H, m), 7.30 (1H, d), 7.80 (1H, d), 8.60 (1H, d), 8.70 (1H, s).

### Intermediate 190: Potassium (8aS)-hexahydropyrrolo[1,2-a]pyrazin-2(1H)-yl(3-pyridinyl)acetate-mixture of diastereoisomers

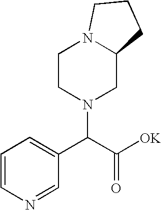

To a stirred solution of 53.7 mg of ethyl(±)-(8aS)-hexahydropyrrolo[1,2-a]pyrazin-2(1H)-yl(3-pyridinyl)acetate (intermediate 189, 0.186 mmole) in 1.2 mL of MeOH were added 11 mg of potassium hydroxide (0.2 mmole, Fluka) previously dissolved in 0.13 mL of water. The solution was stirred at RT for 24 h. The solution was evaporated under reduced pressure to give the title compound as a brown foam (60 mg, quantitative). ¹H-NMR (400 MHz, CDCl₃): δ 1.01-3.50 (13H, m), 4.10 (1H, br s), 7.20 (1H, d), 7.70 (1H, d), 8.30 (1H, d), 8.45 (1H, d); UPLC-MS [Acquity™ UPLC BEH C18, 50×21 mm, 1.7 μm, gradient: A: H₂O+0.1% HCOOH/B: MeCN+0.075% HCOOH: 1% to 6% B in 0.2 min., 6% to 60% B in 1.05 min, 60% to 100% B in 0.5 min., 100% B for 0.2 min, flow rate: 1 mL/min]: Rₜ=0.17 min.

### Intermediate 191: N′-[3,5-bis(trifluoromethyl)phenyl]-2-[(8aS)-hexahydropyrrolo[1,2-a]pyrazin-2(1H)-yl]-2-(3-pyridinyl)acetohydrazide-mixture of diastereoisomers

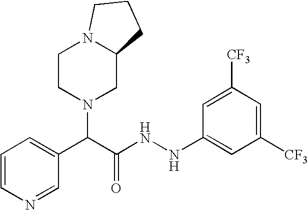

55.5 mg of potassium (8aS)-hexahydropyrrolo[1,2-a]pyrazin-2(1H)-yl(3-pyridinyl)acetate (intermediate 190, 0.19 mmole) were dissolved in 0.8 mL of DMF. To this solution, were added successively 48 mg of 3,5-bis(trifluoromethyl)phenylhydrazine (0.2 mmole, Lancaster) and 248 mg of py-BOP (0.37 mmole, Fluka). The reaction mixture turned red and then colourless and was stirred at RT for 1 h. Further 3,5-bis(trifluoromethyl)phenylhydrazine (24 mg, 0.1 mmole) and py-BOP (0.19 mmole, 124 mg) were added and stirring continued for additional 3 h. Then the reaction mixture was diluted with AcOEt and then washed twice with 50 mL of NaOH 1N, twice with 50 mL of a saturated solution of NaHCO₃, twice with 50 mL of brine, dried over Na₂SO₄, filtered and evaporated to dryness. The crude was then purified by Isco Companion on a silica gel column eluting from DCM 100% to DCM/MeOH 85:15 to give the title compound as a mixture 60:40 of two diastereoisomers (36.4 mg, 40%). ¹H-NMR (400 MHz, CDCl₃): 1.20-3.30 (13H, m), 4.20 (1H, d), 6.50 (1H, s), 7.10 (2H, s), 7.30 (1H, d), 7.70 (1H, dd), 8.60 (2H, dd), 8.90 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: Rₜ=1.51 min.

### Compound 126: N′-[3,5-bis(trifluoromethyl)phenyl]-2-[(8aS)-hexahydropyrrolo[1,2-a]pyrazin-2(1H)-yl]-2-(3-pyridinyl)acetohydrazide-diastereoisomer 1

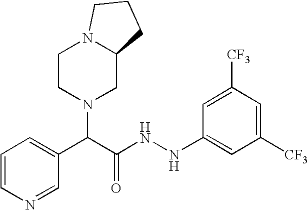

36.4 mg of N′-[3,5-bis(trifluoromethyl)phenyl]-2-[(8aS)-hexahydropyrrolo[1,2-a]pyrazin-2(1H)-yl]-2-(3-pyridinyl)acetohydrazide (intermediate 191, 0.07 mmole) were purified by chiral HPLC [Chiralpak AD-H, Flow rate: 14 mL/min, UV detection: 249 nm, modifier: n-Hex/IPA 92/8% v/v]. Solvents were removed under reduced pressure to give the title compound as a white solid (7.5 mg, 35%). ¹H-NMR (400 MHz, CDCl₃): δ 1.50 (1H, m), 1.80 (4H, m), 2.20 (4H, m), 2.30 (1H, t), 2.70 (1H, d), 3.10 (2H, m), 3.20 (1H, d), 4.20 (1H, s), 6.30 (1H, s), 7.20 (2H, s), 7.40 (2H, m), 7.70 (1H, d), 8.60 (2H, dd), 8.80 (1H, br s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.54 min; Chiral HPLC [Chiralpak AD-H, 25×0.46 cm, Flow rate: 1 mL/min, CD: 249 nm, modifier: n-Hex/IPA 92/8] Rₜ=8.96 min. (de>99.5%).

### Compound 127: N′-[3,5-bis(trifluoromethyl)phenyl]-2-[(8aS)-hexahydropyrrolo[1,2-a]pyrazin-2(1H)-yl]-2-(3-pyridinyl)acetohydrazide-diastereoisomer 2

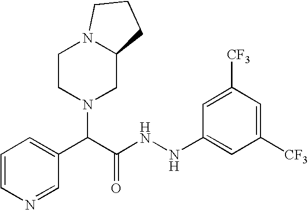

36.4 mg of W-[3,5-bis(trifluoromethyl)phenyl]-2-[(8aS)-hexahydropyrrolo[1,2-a]pyrazin-2(1H)-yl]-2-(3-pyridinyl)acetohydrazide (intermediate 191, 0.07 mmole) were purified by chiral HPLC [Chiralpak AD-H, Flow rate: 14 mL/min, UV detection: 249 nm, modifier: n-Hex/IPA 92/8% v/v]. Solvents were removed under reduced pressure to give the title compound as a white solid (5 mg, 34%). ¹H-NMR (400 MHz, CDCl₃): δ 1.30 (1H, m), 1.80 (4H, m), 2.20 (2H, m), 2.40 (1H, t), 2.60 (1H, t), 2.90 (1H, d), 3.10 (3H, m), 4.30 (1H, s), 6.40 (1H, s), 7.20 (2H, s), 7.40 (2H, m), 7.70 (1H, d), 8.60 (2H, dd), 8.80 (1H, br s); LC-MS [Supelcosil ABZ+Plus, 33×4.6 mm, 3 μm, gradient: A: H₂O+0.1% HCOOH/B: MeCN: 0% to 95% B in 3 min., 95% B for 1 min., 95% to 0% B in 0.1 min., flow rate: 2 mL/min]: R=1.54 min; Chiral HPLC [Chiralpak AD-H, 25×0.46 cm, Flow rate: 1 mL/min, CD: 249 nm, modifier: n-Hex/IPA 92/8] Rₜ=10.08 min. (de>99.5%).

### Intermediate 192: (±)-1,3-Benzodioxol-5-yl(4-methyl-1-piperazinyl)acetic acid

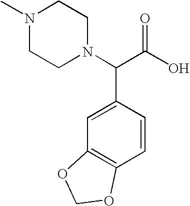

13.4 mL of N-Methylpiperazine (Aldrich, 120 mmole) were added to a solution of 11.1 g of glyoxylic acid monohydrate (Aldrich, 120 mmole) in 60 mL of EtOH at 25° C. and the mixture was stirred for 10 minutes, then it was treated with 20 g of 1,3-benzodioxol-5-ylboronic acid (Aldrich, 120 mmole). The reaction was stirred at 25° C. for 3 weeks, and then was filtered (EtOH/DCM wash). The resulting solid was suspended in Et₂O and treated with an excess of pinacol (Aldrich) and stirred at 25° C. for 1 week. The solid was collected (Et₂O wash) and dried in a vacuum oven at 55° C. overnight to afford the acid intermediate as a brown solid (20.8 g, 59%): ¹H-NMR (400 MHz, DMSO-d₆): δ 2.18 (3H, s), 2.37-2.49 (8H, br m), 3.85 (1H, s), 5.99 (2H, m), 6.85 (2H, m), 6.94 (1H, s); LC-MS (ES+): m/z 279 [M+H]⁺.

### Intermediate 193: (±)-methyl 3-(1,3-benzodioxol-5-yl)-3-(4-methyl-1-piperazinyl)-2-oxopropanoate

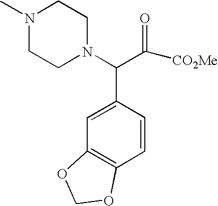

2.1 g of (±)-1,3-Benzodioxol-5-yl(4-methyl-1-piperazinyl)acetic acid (intermediate 192, 7.55 mmole) was suspended in 21 mL of dry DMF and 2.6 mL of DIPEA (15.1 mmole, Aldrich) and 2.91 g of TBTU (9.06 mmole, Fluka) were added. The mixture was stirred at 25° C. for 30 min, then 1.2 mL of MeOH (30.2 mmole, J. T. Baker) was added. The mixture was stirred at 25° C. overnight. The reaction mixture was quenched with NaHCO₃ sat. sol. and AcOEt was added. Phases were separated. Organic phase, dried (Na₂SO₄), was evaporated and the residue chromatographed over silica (230-400 Mesh) eluting with DCM/MeOH 95/5 to give title compound as yellow oil (1.515 g, 68.7%): ¹H-NMR (400 MHz, DMSO-d₆): δ 2.11 (3H, s), 2.18-2.43 (8H, br m), 3.58 (3H, s), 3.92 (1H, s), 6.00 (2H, d), 6.79-6.89 (2H, m), 6.92 (1H, d).

### Intermediate 194: (±)-methyl 3-(1,3-benzodioxol-5-yl)-3-(4-methyl-1-piperazinyl)-2-oxobutanoate

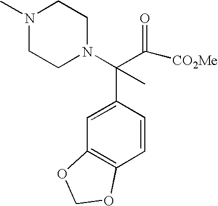

To a 9.96 mL of NaHMDS (0.6M in toluene, 5.97 mmole) diluted in 20 mL of dry THF and cooled to −50° C., 1.34 g of methyl 3-(1,3-benzodioxol-5-yl)-3-(4-methyl-1-piperazinyl)-2-oxopropanoate (intermediate 193, 4.59 mmole) dissolved in 20 mL of dry THF was added dropwise in 10 min. The mixture (yellow, clear) was stirred at −50° C. for 10 min. then 0.372 mL of MeI (Aldrich, 5.97 mmole) was added. Temperature was allowed to rise to 0° C. in 20 min. (colour turned to orange and then to yellow again). Reaction mixture was quenched with 50 mL of NaHCO₃ sat. sol. and extracted with 2×50 mL of AcOEt. Combined organic layers, dried (Na₂SO₄), were evaporated and the residue chromatographed over silica (230-400 Mesh) eluting with DCM/MeOH 95/5 to give 0.956 g of a mixture of title compound and starting material (3/1 by NMR). This mixture was separated by prep. HPLC: Column: Gemini C18, 100×21 mm, 5 micron; Mobile phase:A: NH₄HCO₃ sol. 10 mM, pH10; B: CH₃CN; Gradient: 10% (B) for 1 min, 10% (B)→55% (B) in 9 min, 55% (B)→100% (B) in 2 min, 100% (B) for 4 min; Flow rate: 17 ml/min; UV wavelength range: 210-350 nm; Mass range: 100-900 amu; lonization:ES+, to give title compound (0.480 g, 34%): ¹H-NMR (400 MHz, DMSO-d₆): δ 1.47 (3H, s), 2.15 (3H, s), 2.26-2.43 (8H, br m), 3.59 (3H, s), 5.99 (2H, m), 6.80-6.89 (2H, m), 7.03 (1H, m).

### Intermediate 195: (±)-3-(1,3-benzodioxol-5-yl)-3-(4-methyl-1-piperazinyl)-2-oxobutanoic acid

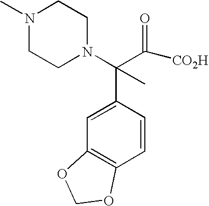

0.1 g of (±)-methyl 3-(1,3-benzodioxol-5-yl)-3-(4-methyl-1-piperazinyl)-2-oxobutanoate (intermediate 194, 0.326 mmole) was dissolved in 4 mL of MeOH and 0.068 g of LiOH (Aldrich, 1.633 mmole) dissolved in 1.2 mL of H₂O was added. The reaction mixture was refluxed for 62 h. The mixture was cooled to room temperature; HCl 1M aqueous was added dropwise (8 drops) and the mixture concentrated. The residue was taken up with 5 mL of H₂O and HCl 1M aqueous was added dropwise till pH=5.5-6.0 (slight torbidity). The solvent was evaporated to give a white solid. The solid was taken up with 5 mL H₂O and filtered (paper) to give title product as white solid (0.018 g, 18%): ¹H-NMR (400 MHz, DMSO-d₆): δ 1.47 (3H, s), 2.20 (3H, s), 2.26-2.43 (8H, br m), 5.99 (2H, m), 6.85 (1H, m), 6.95 (1H, m), 7.1 (1H, m). Mother liquors of filtration were concentrated, HCl 1M aqueous was added till pH=4 (complete dissolution), and purified on OASIS HLB Cartridge eluting with H₂O/MeOH 100/0 to 0/100 to give further title compound as white solid (0.034 g, 34%): ¹H-NMR (400 MHz, DMSO-d₆): δδ 1.47 (3H, s), 2.20 (3H, s), 2.26-2.43 (8H, br m), 5.99 (2H, m), 6.85 (1H, m), 6.95 (1H, m), 7.1 (1H, m).

### Intermediate 196: (±)-2-(1,3-benzodioxol-5-yl)-N′-[3,5-bis(trifluoromethyl)phenyl]-2-(4-methyl-1-piperazinyl)propanohydrazide

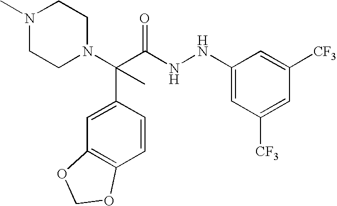

To a suspension of 50 mg of Intermediate 195 (0.171 mmole) in 4 mL of anhydrous DMF, 0.059 mL of DIPEA (0.342 mmole) and 0.066 g of TBTU (0.205 mmole) were added at 25° C. The reaction mixture was stirred at RT for 10 minutes (dissolution) and 0.046 g of (3,5-dichlorophenyl)hydrazine (0.188 mmole, Aldrich) were added. The reaction mixture was left over week-end at RT. The reaction mixture was quenched with 10 mL of NaHCO₃ sat. sol. and 2 mL of water and extracted with AcOEt. Organics, dried (Na₂SO₄), were evaporated and the residue was purified by flash chromatography on silica (230-400 Mesh) eluting with DCM/MeOH 9/1 to give title compound as glassy solid (46 mg, 52%). ¹H-NMR (DMSO-d₆): δ 1.44 (3H, s), 2.18 (3H, s), 2.21-2.71 (8H, m), 5.97 (1H, m), 6.04 (1H, m), 6.81 (2H, m), 6.86 (1H, d), 7.01 (1H, d), 7.18 (1H, m), 7.43 (1H, m), 8.51 (1H, m), 10.01 (1H, m).

### Compound 128: 2-(1,3-benzodioxol-5-yl)-N′-[3,5-bis(trifluoromethyl)phenyl]-2-(4-methyl-1-piperazinyl)propanohydrazide-enantiomer 1

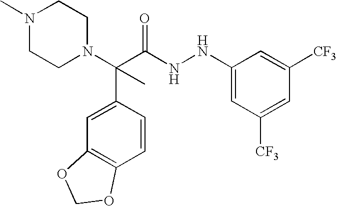

The two enantiomers of intermediate 196 (racemate 46 mg, 0.888 mmole) were separated by semipreparative chiral HPLC [Chiralpak AD-H. Mobile phase: n-Hex/2-propanol 85/15% v/v. Flow rate: 14 mL/min. UV detection: 245 nm. Solvents were removed under reduced pressure to give the title compound as a solid (20 mg, 43%); Chiral HPLC [Chiralpak AD-H, 250×4.6 mm. Flow rate: 1.0 mL/min. UV detection: 200-400 nm. Mobile phase: n-Hex/2-propanol 85/15% v/v]. Rₜ=6.9 min (100% ee).

### Compound 129: 2-(1,3-benzodioxol-5-yl)-N′-[3,5-bis(trifluoromethyl)phenyl]-2-(4-methyl-1-piperazinyl)propanohydrazide-enantiomer 2

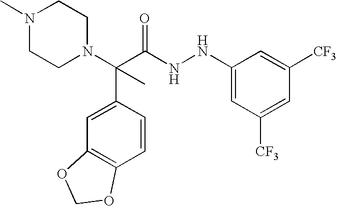

The enantiomers of intermediate 196 (racemate, 46 mg, 0.888 mmole) were separated by semipreparative chiral HPLC [Chiralpak AD-H. Mobile phase: n-Hex/2-propanol 85/15% v/v. Flow rate: 14 mL/min. UV detection: 245 nm. Solvents were removed under reduced pressure to give the title compound as a solid (20 mg, 43%); Chiral HPLC [Chiralpak AD-H, 250×4.6 mm. Flow rate: 1.0 mL/min. UV detection: 200-400 nm. Mobile phase: n-Hex/2-propanol 85/15% v/v]. Rₜ=9.8 min (100% ee).

### Intermediate 197: ethyl(±)-2-(4-methyl-1-piperazinyl)-2-(2-pyridinyl)propanoate

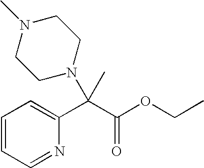

1 g of ethyl(±)-(4-methyl-1-piperazinyl)(2-pyridinyl)acetate (intermediate 95, 3.80 mmole) was dissolved in 16.52 mL of dry THF. The solution was cooled to −78° C. and 4.94 mL of LiHMDS were added dropwise. The solution was stirred at −78° C. for 20 min. Then, 248 μL of MeI were added dropwise and the mixture was allowed to reach RT slowly. The solution was stirred at RT for 12 h. 3 mL of water were added and the solution was evaporated to dryness under reduced pressure. The resulting residue was then purified by flash chromatography on silica gel eluting with 750 mL of AcOEt/TEA 3% to give the title compound as a brown oil (94 mg, 9%). ¹H-NMR (400 MHz, CDCl₃): δ 1.21 (3H, t), 1.63 (3H, s), 2.27 (3H, s), 2.47-2.71 (8H, m), 4.14-4.22 (2H, m), 7.12-7.14 (1H, m), 7.63-7.64 (2H, m), 8.52 (1H, dt); UPLC-MS [BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]: Rₜ=0.62 min (100%) m/z (ES): 278.1 [M+H]⁺, 139.5 [(M+H)/2]⁺.

### Intermediate 198: (±)-N′-[3,5-bis(trifluoromethyl)phenyl]-2-(4-methyl-1-piperazinyl)-2-(2-pyridinyl)propanohydrazide

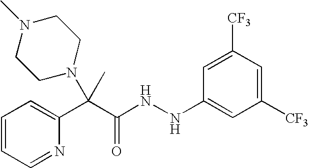

76 mg of ethyl(±)-2-(4-methyl-1-piperazinyl)-2-(2-pyridinyl)propanoate (intermediate 197, 0.27 mmole) were dissolved in 1.62 mL of MeOH. Then, 16.1 mg of KOH (0.29 mmole) were added followed by 185 μL of water. The mixture was then stirred at RT for 6 days and then heated at 70° C. for 13.5 days. The solution was cooled to RT and solvents were removed under reduced pressure. The resulting crude compound was dried under high vacuum for 1 h to give 66 mg (0.23 mmole) of the resulting salt as an orange oil which was dissolved in 822 μL of DMF. To this solution were added 59 mg of [3,5-bis(trifluoromethyl)phenyl]hydrazine (0.24 mmole) followed by 107 mg of BOP (0.24 mmole). The mixture was stirred at RT overnight. Solvents were removed under reduced pressure to give 230 mg of a yellow wax. This residue was dissolved in MeOH and passed through a 2 g SCX cartridge. The cartridge was washed with 20 mL of DCM, 20 mL of MeOH and the product was released with 20 mL of NH₃ 2M in MeOH. Solvents were removed under reduced pressure and the crude compound was purified by MDAP. Solvents were removed under reduced pressure to give the title compound as a colourless oil (17.8 mg, 16%). ¹H-NMR (400 MHz, CD₃OD): δ 1.77 (3H, s), 2.77-2.87 (7H, m), 3.22 (4H, m), 7.15 (2H, s), 7.21 (1H, s), 7.39 (1H, dd), 7.79 (1H, d), 7.89 (1H, td), 8.65 (1H, d); UPLC-MS [BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]: Rₜ=1.13 min (100%) m/z (ES): 476.05 [M+H]⁺, 375.99 [M-piperazine]⁺, 238.63 [(M+H)/2]⁺.

### Compound 130: N′-[3,5-bis(trifluoromethyl)phenyl]-2-(4-methyl-1-piperazinyl)-2-(2-pyridinyl)propanohydrazide-enantiomer 1

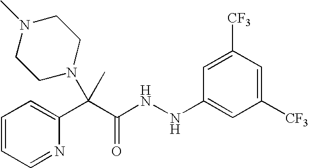

17.8 mg of (±)-N′-[3,5-bis(trifluoromethyl)phenyl]-2-(4-methyl-1-piperazinyl)-2-(2-pyridinyl)propanohydrazide (intermediate 198) were purified by semipreparative chiral SFC [CHIRALCEL OD-H, 25×2.1 cm, modifier: 12% of Ethanol+0.1% isopropylamine, Temp: 36° C., Pressure: 165 bar, flow rate: 22 mL/min]. Solvents were removed under reduced pressure to give the title compound as a colourless oil (6.3 mg, 35%). ¹H-NMR (400 MHz, CD₃OD): δ 1.73 (3H, s), 2.33 (3H, s), 2.51-2.89 (8H, m), 7.10 (2H, s), 7.21 (1H, s), 7.30-7.46 (1H, m), 7.84-7.96 (2H, m), 8.64 (1H, d); LC-MS [Supelcosil ABZ+Plus, 33×4.6 mm, 3 μm, gradient: A: H₂O+0.1% HCOOH/B: CH₃CN: 0% to 95% B in 3 min, 95% for 1 min, 95% to 0% B in 0.1 min, flow rate: 2 mL/min]: Rₜ=1.74 min (100%) m/z (ES): 476.1 [M+H]⁺, 376.0 [M-piperazine]⁺; Chiral SFC [CHIRALCEL OD-H, 25×0.46 cm, modifier: 12% of Ethanol+0.1% of isopropylamine, Temp: 35° C., Pressure: 100 bar, flow rate: 2.0 mL/min]: Rₜ=3.71 min (100% ee).

### Compound 131: N′-[3,5-bis(trifluoromethyl)phenyl]-2-(4-methyl-1-piperazinyl)-2-(2-pyridinyl)propanohydrazide-enantiomer 2

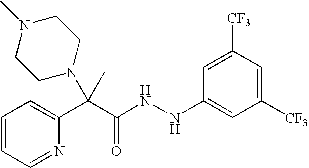

17.8 mg of (±)-N′-[3,5-bis(trifluoromethyl)phenyl]-2-(4-methyl-1-piperazinyl)-2-(2-pyridinyl)propanohydrazide (intermediate 198) were purified by semipreparative chiral SFC [CHIRALCEL OD-H, 25×2.1 cm, modifier: 12% of Ethanol+0.1% isopropylamine, Temp: 36° C., Pressure: 165 bar, flow rate: 22 mL/min]. Solvents were removed under reduced pressure to give the title compound as a colourless oil (6.0 mg, 34%). ¹H-NMR (400 MHz, CD₃OD): δ 1.73 (3H, s), 2.33 (3H, s), 2.47-2.83 (8H, m), 7.10 (2H, s), 7.21 (1H, s), 7.37-7.39 (1H, m), 7.87-7.89 (2H, m), 8.63-8.64 (1H, m); LC-MS [Supelcosil ABZ+Plus, 33×4.6 mm, 3 μm, gradient: A: H₂O+0.1% HCOOH/B: CH₃CN: 0% to 95% B in 3 min, 95% for 1 min, 95% to 0% B in 0.1 min, flow rate: 2 mL/min]: Rₜ=1.74 min (100%) m/z (ES): 476.0 [M+H]⁺, 376.0 [M-piperazine]⁺; Chiral SFC [CHIRALCEL OD-H, 25×0.46 cm, modifier: 12% of Ethanol+0.1% of isopropylamine, Temp: 35° C., Pressure: 100 bar, flow rate: 2.0 mL/min]: Rₜ=5.85 min (100% ee).

### Compound 132: N′-[3,5-bis(trifluoromethyl)phenyl]-2-(2,6-dibromo-3-pyridinyl)-2-[(8aR)-hexahydropyrrolo[1,2-a]pyrazin-2(1H)-yl]acetohydrazide-diastereoisomer 1;

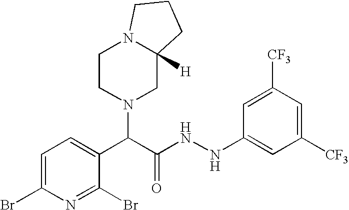

Analogously to what described for compound 122, the title compound was prepared starting from 2,6-dibromo-3-pyridinecarbaldehyde. ¹H-NMR (400 MHz, DMSO-d₆): δ 1.20-1.36 (1H, m), 1.56-1.77 (3H, m), 1.92-2.25 (5H, m), 2.53 (1H, d), 2.79-3.09 (3H, m), 4.41 (1H, s), 7.15 (2H, s), 7.33 (1H, s), 7.78 (1H, d), 8.01 (1H, d), 8.88 (1H, s), 10.68 (1H, s); UPLC-MS [BEH C18 column (50×21 mm, 1.7 μm particle size), column temperature 40° C. (mobile phase: A-water+0.1% HCOOH/B-MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]: Rₜ=0.64 min (100%) m/z (ES): 643.96 [M-1]⁺, 645.96 [M+H]⁺, 647.94 [M+H+2]⁺; Chiral HPLC [Chiralpak AD-H, 25×0.46 cm, mobile phase: n-hexane/2-propanol 90/10, flow rate: 1.0 mL/min]: Rₜ=5.48 min (100% d.e.).

### Biological Assays

The ability of a compound of formula (I), or a pharmaceutically acceptable salt or solvate thereof to modulate the growth hormone secretagogue receptor may be determined using the following assay:

### Cloning of the Ghrelin Receptor GHS-R

Human GHS-R was cloned from human hypothalamus cDNA and TOPO Ta cloned into pCR2.1. The sequence was confirmed. The full-length gene was transferred into pCDN for expression analysis. The sequence was confirmed again and the plasmid was electroporated into CHO cells. The clones were screened by FLIPR.

Generation of the GHS-R Bacmam Virus and Viral Titre Determination Virus Generation

The open reading frame of GHS-R was transferred from pCDN into pFastBacmam vector. This vector was used to generate recombinant baculoviruses in which the insect cell-specific polyhedrin promoter has been replaced with a mammalian cell-active promoter, in this case CMV. This was then used with the Bac to Bac expression system (Invitrogen). Briefly the vector was transformed into DH10 bac E. coli and the bacmid isolated from the transformed cells. The bacmid was then transfected into Sf9 insect cells grown in ExCell 420 (JRH) medium in 6-well dishes for the production of recombinant baculovirus particules.

The supernatant from these cells was harvested containing the recombinant GHS-R bacmam virus. This P0 viral stock was then used to infect 200 mls of 1×10⁻⁶ cells/ml Sf9 cells in ExCell 420 medium to further amplify the virus and provide a P1 stock. This P1 viral stock was then used to amplify a P2 viral stock of 10×1 litre erlemeyer shake flasks again harvesting the supernatant from the cells. This was then used to transduce mammalian cells for assay

### Viral Titre Determination

Viral titres were determined at all stages of the virus scale up with a plaque elisa method using a gp64 envelope protein monoclonal antibody.

SF9 cells were plated out into a 96 well plate and a dilution range of virus was added to the cells for 1 h. The virus was removed and a 1% methylcellulose and media mix was added to the cells and incubated for 48 h. The cells were then fixed in a formaldehyde and acetone mix for 8 min. The cells were then washed with a phosphate buffered saline solution (PBS) and normal goat serum added for 25 min. This was then removed and a gp64 monoclonal antibody added for 25 min. The wells were then washed with PBS and a goat anti-mouse/HRP conjugated antibody added for 25 min. The wells were again washed with PBS and True Blue peroxidase substrate solution (Kirke.g.aard & Perry Laboratories) was added and the wells were incubated for 60 min.

Individual wells were counted for blue foci taking into account the dilution factor, the plaque forming units/ml of the virus was determined.

### Generation of U2OS Cells Transiently Expressing the Ghrelin Receptor GHS-R

24 h prior to assay U2OS cells at confluence 100% were harvested and spun down. The supernatant was removed and the cells resuspended in media (DMEM+10% FBS+1% L-Glutamine). A cell count was performed using the Cedex instrumentation, and the concentration of cells was adjusted using media to give 200K cells per mL (10K cells/50 μL).

Human GHSR BACMAM virus was added to the cell suspension at an appropriate % volume (calculated for individual batches of BACMAM virus as viral titres vary). The transduced cell suspension was dispensed into FLIPR 384-well clear bottom plates, 50 μL per well. Cell plates were incubated at 37° C. overnight.

### Compound Preparation

### Method A

Master compound plates were prepared in 100% DMSO. 3 mM was the top concentration (giving 10 μM final concentration) and they were serially diluted 1 in 4. 1 μL from the master plate was transferred to a daughter plate, to which was added 50 μL of compound dilution (Tyrodes {Elga water+145 mM NaCl+5 mM KCl+20 mM HEPES+10 mM glucose+1 mM MgCl₂+1.5 mM CaCl₂}. For the agonist assay, the compound buffer also contained 0.1% BSA. This plate was used for the assay. Ghrelin was always prepared in buffer containing 0.1% BSA.

### Method B

Master compound plates were prepared in 100% DMSO. 3 mM was the top concentration (giving 10 μM final concentration) and they were serially diluted 1 in 4. 1 μL from the master plate was transferred to a daughter plate, to which was added 50 μL of compound dilution (HBSS {Elga water+137 mM NaCl+5 mM KCl+0.41 mMa KH₂PO₄(anhyd)+20 mM HEPES+5 mM glucose+0.81 mM MgSO₄(anhyd)+1.3 mM CaCl₂+4.16 mM NaHCO₃}. For the agonist assay, the compound buffer also contained 0.1% BSA. This plate was used for the assay. Ghrelin was always prepared in buffer containing 0.1% BSA.

### Method C

Master compound plates were prepared in 100% DMSO. 0.6 mM was the top concentration (giving 3 μM final concentration) and they were serially diluted 1 in 4. 1 μL from the master plate was transferred to a daughter plate, to which was added 50 μL of compound dilution (NaCl (145 mM) KCl (5 mM) CaCl₂ (2 mM) MgCl₂ (1 mM) HEPES (20 mM) D-(+)-Glucose (1 g/l), pluronic acid (0.05%), pH 7.3). This plate was used for the assay. Ghrelin was always prepared in compound dilution buffer containing 0.1% BSA.

### GHSR Agonist BACMAM FLIPR Assay Protocol

### Method X

Media was aspirated from cell plates using a cell washer (leaving 10 μL of media). Cells were immediately loaded with loading buffer (Tyrodes buffer-(Elga water+145 mM NaCl+5 mM KCl+20 mM HEPES+10 mM glucose+1 mM MgCl₂)+1.5 mM CaCl₂+0.714 mg/mL Probenecid (predissolved in 1 M NaOH)+0.25 mM brilliant black+2 μM Fluo 4 dye, and incubated at 37.5° C., 5% CO for 90 min. The plates were placed in a FLuorimetric Imaging Plate Reader (FLIPR, Molecular Devices) where 10 μL of compound, prepared according to method A above, was added to the cells and fluorescence measurements were taken. Maximum changes in fluorescence were plotted as a percentage of the maximum response elicited by 300 nM hGhrelin and curves fitted using a 4-parameter logistic equation to generate pEC50 values. Intrinsic activity of the compounds was calculated by using the maximum asymptote of it's concentration response curve relative to the maximum asymptote of the hGhrelin concentration response curve.

### Method Y

Media was aspirated from cell plates using a cell washer (leaving 10 μL of media). Cells were immediately loaded with loading buffer (HBSS (Elga water+137 mM NaCl+5 mM KCl+0.41 mM KH₂PO₄(anhyd)+20 mM HEPES+5 mM glucose+0.81 mM MgSO₄(anhyd))+1.3 mM CaCl₂+4.16 mM NaHCO₃+0.714 mg/mL Probenecid (predissolved in 1 M NaOH)+0.25 mM brilliant black+2 μM Fluo 4 dye, and incubated at 37.5° C., 5% CO for 90 min. The plates were placed in a FLuorimetric Imaging Plate Reader (FLIPR, Molecular Devices) where 10 μL of compound, prepared according to method B above, was added to the cells and fluorescence measurements were taken. Maximum changes in fluorescence were plotted as a percentage of the maximum response elicited by 300 nM hGhrelin and curves fitted using a 4-parameter logistic equation to generate pEC₅₀ values. Intrinsic activity of the compounds was calculated by using the maximum asymptote of it's concentration response curve relative to the maximum asymptote of the hGhrelin concentration response curve.

### Method Z

Media was washed from cell plates, using a cell washer (leaving 20 μL of washing buffer (NaCl (145 mM)+KCl (5 mM)+CaCl₂ (2 mM)+MgCl₂ (1 mM)+HEPES (20 mM)+D-(+)-Glucose (1 g/l)+Probenecid (2.5 mM), pH 7.3). Cells were immediately loaded with loading buffer (washing buffer+pluronic acid (0.02%)+Fluo 4 dye (2 μM), and incubated at 37° C., for 60 min. After a second washing step (same as for the pre-loading, but leaving 30 μL of washing buffer), the plates were placed in a FLuorimetric Imaging Plate Reader (FLIPR, Molecular Devices) where 10 μL of compound, prepared according to method C above, was added to the cells and fluorescence measurements were taken. Maximum changes in fluorescence were plotted as a percentage of the maximum response elicited by 100 nM hGhrelin and curves fitted using a 4-parameter logistic equation to generate pEC₅₀ values. Percentage activation of the compounds was calculated by using the maximum asymptote of it's concentration response curve relative to the maximum response of the hGhrelin at 100 nM concentration.

### GHSR Antagonist BACMAM FLIPR Assay Protocol

10 μL of compound was added to cell plates, prepared according to either Method A or B above, using an FX robot and the plate was then incubated (37.5° C., 5% CO₂) for a further 30 min before being assayed on a FLIPR, where 10 μL of an EC₈₀ concentration of hGhrelin was added to the cells and fluorescence measurements were taken. Maximum changes in fluorescence were plotted as a percentage of the maximum inhibition elicited by 1 μM (2R)—N-[(3,5-dichlorophenyl)methyl]-2-(4-methyl-1-piperazinyl)-2-(1-naphthalenyl)ethanamide hydrochloride (see below for synthesis) and curves fitted using a 4-parameter logistic equation to generate pIC₅₀ values.

GHSR Antagonist BACMAM FLIPR Assay protocol—FLIPR

Media was washed from cell plates, using a cell washer (leaving 20 μL of washing buffer (NaCl (145 mM)+KCl (5 mM)+CaCl₂ (2 mM)+MgCl₂ (1 mM)+HEPES (20 mM)+D-(+)-Glucose (1 g/L)+Probenecid (2.5 mM), pH 7.3). Cells were immediately loaded with loading buffer (washing buffer+pluronic acid (0.02%)+Fluo 4 dye (2 μM), and incubated at 37° C., for 60 min. After a second washing step (same as for the pre-loading, but leaving 30 μL of washing buffer), 10 μL of compound, prepared according to method C above, was added to cell plates using an FX robot (or similar) and the plate was then incubated at room temperature for a further 30 min before being assayed on a FLIPR, where 10 μL of an EC₈₀ concentration of hGhrelin was added to the cells and fluorescence measurements were taken. Maximum changes in fluorescence were plotted as a percentage of the maximum inhibition, where 100% inhibition was derived from non-stimulated cells in the same plate. Curves were fitted using a 4-parameter logistic equation to generate pIC₅₀ values.

hGHSR Antagonist GTPγS SPA Assay Protocol

### Protocol Steps

Compound plates preparation:
compound stocks are in columns 1 and 13, 32 compounds per plate. By using a Biomek FX (Beckmam Coulter) serially dilute compounds in neat DMSO.
   Compound transfer to assay plate
Transfer 0.5 mL of compound dilution to the assay plate.
Add 0.5 mL DMSO (0% control) into column 6 and 0.5 mL 100 mM compound 76 (100% control) into column 18
   SPA mix preparation (Human GHRS BacMam membranes, PS-WGA beads and GDP)
Dilute frozen membranes stock and PS-WGA beads in GTPγS assay buffer (20 mM Hepes/NaOH pH 7.4 plus 10 mM MgCl₂ and 100 mM NaCl) containing 0.05% pluronic F-127 and 0.05% BSA at 50 ug/mL and 2 mg/mL, respectively (2× mix suspension, corresponding to 25 ug/mL membrane and 1 mg/mL beads Final Assay Concentration).
Add GDP to have 8 uM (2× solution, corresponding to 4 uM FAC)
Precouple SPA mixture under gentle shaking for 30 minutes before adding to the assay plate.
   Human Ghrelin EC80 solution preparation
Dilute ghrelin stock in GTPγS assay buffer containing 0.05% pluronic F-127 and 0.05% BSA to a concentration at 2×EC80 Final Assay Concentration. EC80 is a nominal amount required to produced 80% of the maximal response and is calculated as 4-fold EC50.
Add [35S]GTPγS stock solution to have 1.2 nM (2× solution, corresponding to 0.6 nM FAC).
   Assay procedure
Add 25 uL [35S]GTPγS/2×EC80 ghrelin solution to all wells using Multidrop Combi (ThermoLabsystem).
Add 25 uL SPA mixture (membrane-beads-GDP) to all wells using Multidrop Combi (ThermoLabsystem).
Seal plate and spin at 800 rpm for 2 min.
Incubate plate at room temperature for 1 h in the dark (reaction is stable for several hours: tested up to 5 hours).
Put plate in ViewLux imager (Perkin Elmer) and read plate for 5 minutes.

The [35S]GTPγS assay measures the level of G protein activation following agonist occupation of a G protein-coupled receptor (GPCR), by determining the binding of the non-hydrolyzable analog [35S]GTPγS to Ga subunits. In the assay [35S]GTPγS replaces endogenous GTP and binds to the Ga subunit following activation of the receptor to form a Ga-[35S]GTPγS species. Since the g-thiophosphatebond is resistant to hydrolysis by the GTPase of Ga, G protein is prevented from reforming as a heterotrimer and thus [35S]GTPγS labeled Ga subunits accumulate and can be measured by counting the amount of [35S]-label incorporated. Thus, the assay measures a functional consequence of receptor occupancy at one of the earliest receptor-mediated events.

During the assay incubation, carbohydrate residues present on the cell membranes bind to the WGA (wheat germin agglutinin) on the SPA beads. This coupling mechanism immobilizes human GHSR in close proximity to the scintillant within the bead. In presence of antagonist compounds that bind to the receptor a lower signal would be measured.

Maximum changes in luminescence is plotted and curves fitted using a 4-parameter logistic equation to generate pIC50 values.

fpKi values are calculated from the IC₅₀ values by using the Cheng and Prusoff equation.

(2R)—N-[(3,5-dichlorophenyl)methyl]-2-(4-methyl-1-piperazinyl)-2-(1-naphthalenyl)ethanamide hydrochloride may be prepared as follows:

### Intermediate A: (±)-(4-methyl-1-piperazinyl)(1-naphthalenyl)acetic acid

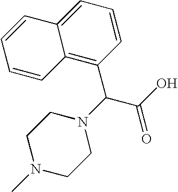

452 μL of N-methylpiperazine (4.07 mmole, Aldrich) were added to a solution of 700 mg of 1-napthaleneboronic acid (4.07 mmole, Alfa Aesar) and 375 mg of glyoxylic acid monohydrate (4.07 mmole, Aldrich) in 7 mL of MeCN. After addition of N-methylpiperazine, the mixture was refluxed for 24 hours. The solution became dark with formation of a beige precipitate. The reaction was cooled to room temperature and poured into AcOEt (14 mL). The precipitate was filtered and dried in vacuo. Then it was triturated with Et₂O for 1 hour to give the title compound as a beige solid (1.11 g, 96%). ¹H-NMR (400 MHz, d⁶DMSO) δ 2.09-2.21 (3H, s), 2.23-2.71 (7H, m), 2.93-3.00 (1H, m), 4.66-4.74 (2H, m), 7.45-7.59 (3H, m), 7.65 (1H, d), 7.90 (2H, dd), 8.54 (1H, d); LC-MS [Supelcosil ABZ+Plus, 33×4.6 mm, 3 μm, gradient: A: H₂O+0.1% HCOOH/B: MeCN: 0% to 95% B in 3 min., 95% B in 1 min., 95% to 0% B in 0.1 min., run time 4.5 min., flow rate: 2 ml/min]. Rₜ=0.261 min. (m/z=285 [M+H]⁺).

### Intermediate B: (±)-N-[(3,5-dichlorophenyl)methyl]-2-(4-methyl-1-piperazinyl)-2-(1-naphthalenyl)ethanamide

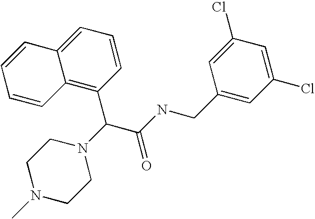

To a suspension of 1.11 g of (±)-(4-methyl-1-piperazinyl)(1-naphthalenyl)acetic acid (intermediate A, 3.9 mmole) in DMF (10 mL), were added 1.36 mL of DIPEA (7.82 mmole, Aldrich) and 1.5 g of TBTU (4.69 mmole, Fluka). After 10 minutes were added 0.625 mL of 3,5-dichlorobenzylamine (4.69 mmole, Maybridge). The solution became clear and dark and it was stirred at room temperature for two days. Then it was diluted with AcOEt and washed with a saturated solution of NaHCO₃ (20 mL). The phases were separated and the organic layer was extracted with AcOEt (3×30 mL). The combined organic layers were washed again with water and ice (2×50 mL), dried over Na₂SO₄ and concentrated to dryness to give the crude compound that was purified by flash chromatography on 25 g silica gel cartridge, using a gradient of DCM/MeOH 10/0 to 8/2 as an eluent. Solvents were removed under reduced pressure to give the title compound as a beige foam (782 mg, 46%). ¹H-NMR (400 MHz, CDCl₃) δ 2.25-2.37 (3H, m), 2.38-2.78 (8H, m), 4.36 (1H, dd), 4.49 (1H, dd), 4.76-4.82 (1H, m), 6.96-7.01 (2H, m), 7.20-7.25 (1H, m), 7.29-7.39 (1H, m), 7.43-7.63 (4H, m), 7.86 (2H, dd), 8.37 (1H, d).

### Intermediate C: N-[(3,5-dichlorophenyl)methyl]-2-(4-methyl-1-piperazinyl)-2-(1-naphthalenyl)ethanamide di-O,O′-p-toluoyl-L-tartaric salt-enantiomer 1

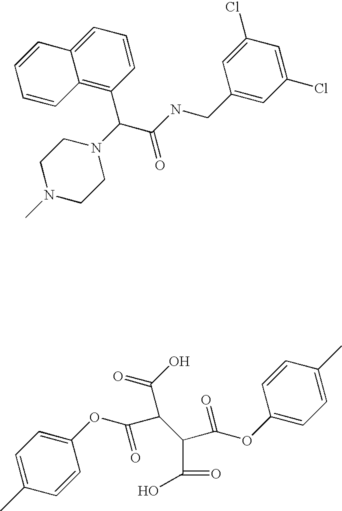

A solution of 685 mg of (−)-di-O,O′-p-toluoyl-L-tartaric acid (1.77 mmole, Fluka) in 4 mL of acetone was added dropwise to a solution of intermediate B ((±)-N-[(3,5-dichlorophenyl)methyl]-2-(4-methyl-1-piperazinyl)-2-(1-naphthalenyl)ethanamide, 782 mg, 1.77 mmole) in 7 mL of acetone. The reaction was stirred for 3 hours to complete precipitation of the salt. The white precipitate was filtered and dried under vacuum to give 522 mg of a white powder. Then it was suspended in 5 mL of acetone, heated at reflux for 2 hours and then cooled to room temperature overnight. The precipitate was filtered and dried to give the title compound as a white solid (354 mg, 48%). ¹H-NMR (400 MHz, d⁶DMSO) δ 2.01-2.14 (3H, m), 2.26-2.40 (6H, m), 2.58-2.74 (2H, m), 2.74-3.05 (2H, m), 3.06-3.62 (4H, m), 4.16-4.38 (2H, m), 4.81-4.85 (1H, m), 5.66-5.66 (2H, m), 7.09 (2H, d), 7.30 (4H, d), 7.38-7.42 (1H, m), 7.46-7.59 (3H, m), 7.68 (1H, d), 7.82 (4H, d), 7.87-7.98 (2H, m), 8.53 (1H, d), 8.77-8.86 (1H, m); Chiral HPLC [Chiralcel OD, 25×4.6 cm. Flow rate: 10.8 ml/min. UV detection: 225 nm. Mobil phase: nHexane/ethanol 70/30]: Rₜ=5.625 min (98.28% ee).

### Compound A: (2R)—N-[(3,5-dichlorophenyl)methyl]-2-(4-methyl-1-piperazinyl)-2-(1-naphthalenyl)ethanamide

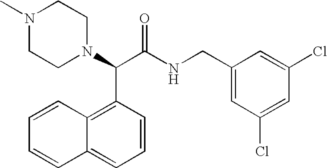

354 mg of intermediate C ((N-[(3,5-dichlorophenyl)methyl]-2-(4-methyl-1-piperazinyl)-2-(1-naphthalenyl)ethanamide di-O,O′-p-toluoyl-L-tartaric salt-enantiomer 1, 0.428 mmole) were partitioned between DCM (7 mL) and a saturated solution of K₂CO₃ (7 mL). The organic layer was washed with a 10% solution of K₂CO₃, dried over Na₂SO₄ and concentrated to dryness to give the title compound as a white solid (190 mg, yield quantitative). ¹H-NMR (400 MHz, CDCl₃) δ 2.27-2.34 (3H, m), 2.37-2.72 (8H, m), 4.34-4.54 (2H, m), 4.79-4.82 (1H, m), 7.00 (2H, d), 7.21-7.26 (1H, m), 7.33-7.43 (1H, m), 7.44-7.67 (4H, m), 7.81-7.94 (2H, m), 8.37 (1H, d); Chiral HPLC [Chiralcel OD, 25×4.6 cm. Flow rate: 10.8 ml/min. UV detection: 225 nm. Mobil phase: nHexane/ethanol 70/30]: Rₜ=5.642 min (98.28% ee).

Absolute stereochemistry determined using comparative vibrational circular dichroism (VCD), employing a closely related analog of known absolute stereochemistry (determined via ab-initio VCD analysis) as the comparator.

### Compound B: (2R)—N-[(3,5-dichlorophenyl)methyl]-2-(4-methyl-1-piperazinyl)-2-(1-naphthalenyl)ethanamide hydrochloride

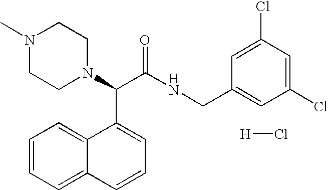

20 mg of compound A ((2R)—N-[(3,5-dichlorophenyl)methyl]-2-(4-methyl-1-piperazinyl)-2-(1-naphthalenyl)ethanamide, 0.045 mmole) were dissolved in 450 μL of dry DCM in a dry flask under nitrogen. Then 45 μL of HCl 1M solution in Et₂O (0.045 mmole, Aldrich) were added. The solution was then stirred for 30 minutes. The solvents were removed under reduced pressure and the resulting crude solid was triturated with Et₂O. The solid was filtered and dried to give the title compound as a sand colored solid (21.3 mg, quantitative). ¹H-NMR (CDCl₃, 400 MHz): δ 2.60-3.04 (4H, m), 2.76-28 (3H, m), 3.06-3.41 (4H, m), 4.18-4.30 (1H, m), 4.30-4.43 (1H, m), 4.83 (1H, br s), 6.80 (3H, d), 7.11-7.17 (1H, m), 7.44-7.61 (3H, m), 7.65-7.75 (1H, m), 7.84-7.94 (2H, m), 8.50 (1H, d), 12.53 (1H, br s); m/z (ES⁺): 442.3 [M+H]⁺.

Compounds 1 to 97, 128 to 131 were tested in the GHSR Antagonist BACMAM FLIPR Assay and found to give pIC₅₀ values of greater than 5.5.

Compounds 98 to 127 and 132 were tested in the GTPγS SPA Antagonist Assay and found to give pIC₅₀ values of greater than 5.5.

Compounds 9, 11 to 18, 20, 22 to 33, 37, 45, 46, 49 to 52, 55, 56, 58 to 67, 73, 78, 80, 83, 85, 126 and 130 were tested in the GHSR Agonist BACMAM FLIPR Assay (either via Method X or Y) and found to give intrinsic activity (IA) values of 0.4.

Compounds 92, 94, 96, were tested in the GHSR Agonist BACMAM FLIPR Assay—(via Method Z) and found to give percentage activation values of 40%.

Compounds 19, 21, 34, 48, 77 and 79 were tested in the GHSR Agonist BACMAM FLIPR Assay (either via Method X or Y) and found to give intrinsic activity (IA) values of less than 0.4

Compounds 81, 82, 84, 86, 87, 89, 90, 93, 95, 97 and 127 were tested in the GHSR Agonist BACMAM FLIPR Assay—Flipr (via Method Z) and found to give percentage activation values of less than 40%.

1. A compound of formula (I) or a pharmaceutically acceptable salt or solvate thereof: 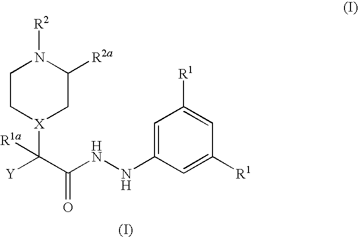
wherein:
each R¹ is independently selected from the group consisting of Cl, Br, CH₃ and CF₃;
X is carbon or nitrogen;
R^{1a} is H or a straight C₁₋₃ alkyl group;
R^{2a} is H or a methyl group;
R² is selected from the group consisting of C₁₋₃alkyl, H and —(CH₂)ₙ—, wherein n is 3 or 4 and the terminal carbon of the chain is bonded to the carbon atom adjacent to the nitrogen bearing the R² group, such that a fused 6,5 or 6,6-bicyclic ring is formed;
Y is selected from the group consisting of: phenyl which may be unsubstituted or substituted by one or more substituents independently selected from the group consisting of C₁₋₃alkyl, C₁₋₃alkoxy, halogen, C₁₋₃alkyl substituted by 1 to 7 fluoro atoms and C₁₋₃alkoxy substituted by 1 to 7 fluoro atoms; pyridyl which may be unsubstituted or substituted by one or more substituents independently selected from the group consisting of C₁₋₃alkyl, OCH₃, CF₃, CN, and halogen; naphthyl which may be unsubstituted or substituted by one or more substituents independently selected from the group consisting of F and OCH₃; pyrimidinyl; imidazo[1,2-a]pyridine-6-yl; benzothiophen-2-yl; benzothiophen-5-yl; benzofuran-2-yl; dibenzo[b,d]furan-3-yl; dibenzo[b,d]thiophen-2-yl; dibenzo[b,d]thiophen-4-yl; 1,3-benzodioxol-5-yl; 2,3-dihydro-1,4-benzodioxin-5-yl; 2,3-dihydro-1,4-benzodioxin-6-yl; 2,3-dihydro-1-benzofuran-4-yl; 2,2-difluoro-1,3-benzodiox-4-yl; pyridazinyl; imidazolyl; oxazolyl; pyrazolyl; thiazolyl; and triazolyl;
with the proviso that when Y is 2,3-dihydro-1,4-benzodioxin-6-yl, R¹ is not Cl.

2. A compound according to claim 1 wherein each R^{1a} and R^{2a} are H.

3. A compound according to claim 1 wherein each R¹ is independently CF₃ or Cl.

4. A compound according to claim 1 wherein R² is hydrogen, methyl, or —(CH₂)₃— wherein the terminal carbon atom of this chain is bonded to the carbon atom adjacent to the nitrogen bearing the R² group such that a fused 6,5-bicyclic ring is formed.

5. A compound according to claim 1 wherein Y is selected from the group consisting of: phenyl which may be unsubstituted or substituted by one or more substituents independently selected from the group consisting of CH₃, OCH₃, OCF₃, F and Cl; pyridyl which may be unsubstituted or substituted by one or more substituents independently selected from the group consisting of CH₃ and Cl; naphthyl which may be unsubstituted or substituted by one or more substituents independently selected from the group consisting of F and OCH₃; pyrimidinyl; imidazo[1,2-a]pyridine-6-yl; benzothiophen-2-yl; benzothiophen-5-yl; benzofuran-2-yl; dibenzo[b,d]furan-3-yl; dibenzo[b,d]thiophen-2-yl; dibenzo[b,d]thiophen-4-yl; 1,3-benzodioxol-5-yl; 2,3-dihydro-1,4-benzodioxin-5-yl; 2,3-dihydro-1,4-benzodioxin-6-yl; 2,3-dihydro-1-benzofuran-4-yl; and 2,2-difluoro-1,3-benzodiox-4-yl.

6. A compound according to claim 1 wherein Y is selected from the group consisting of: 1,3-benzodioxol-5-yl, 2,3-dihydro-1,4-benzodioxin-6-yl, 2-chlorophenyl, 2-pyridyl, 3-pyridyl, 2-fluorophenyl, 2-methoxy-6-fluorophenyl, 3-methylphenyl, 2-methylpyridin-5-yl, 2-methylpyridin-3-yl, naphth-1-yl and 2,6-dimethylpyridin-3-yl.

7. A compound according to claim 1 wherein each R¹ is independently CF₃ or Cl, X is nitrogen, R² is hydrogen or methyl, and Y is selected from the group consisting of: 1,3-benzodioxol-5-yl, 2,3-dihydro-1,4-benzodioxin-6-yl, 2-chlorophenyl, 2-pyridyl, 3-pyridyl, 2-fluorophenyl, 2-methoxy-6-fluorophenyl, 3-methylphenyl, 2-methylpyridin-5-yl, and 2-methylpyridin-3-yl.

8. A compound according to claim 1 wherein each R¹ is independently CF₃ or Cl, X is nitrogen, R² is —(CH₂)₃— wherein the terminal carbon atom of this chain is bonded to the carbon atom adjacent to the nitrogen bearing the R² group such that a fused 6,5-bicyclic ring is formed, and Y is selected from the group consisting of 2,6-dimethylpyridin-3-yl, 2-chlorophenyl, pyridin-3-yl and naphth-1-yl.

9. A compound according to claim 1 of formula (IA) or a pharmaceutically acceptable salt or solvate thereof: 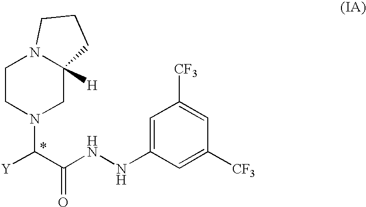
wherein:
Y is selected from the group consisting of pyridyl which may be unsubstituted or substituted once or twice by C₁₋₃alkyl; and phenyl substituted once by halogen.

10. A compound of claim 1 selected from the group consisting of:
formic acid-(±)-2-dibenzo[b,d]thien-4-yl-N′-(3,5-dichlorophenyl)-2-(4-methyl-1-piperazinyl)acetohydrazide, mixture of enantiomers;
formic acid-(±)-2-dibenzo[b,d]furan-4-yl-N′-(3,5-dichlorophenyl)-2-(4-methyl-1-piperazinyl)acetohydrazide, mixture of enantiomers;
formic acid-(±)-N′-(3,5-dichlorophenyl)-2-(4-methyl-1-piperazinyl)-2-(2-naphthalenyl)-acetohydrazide, mixture of enantiomers;
formic acid-(±)-2-(1-benzofuran-2-yl)-N′-(3,5-dichlorophenyl)-2-(4-methyl-1-piperazinyl)acetohydrazide, mixture of enantiomers;
(±)-N′-(3,5-dichlorophenyl)-2-(4-methyl-1-piperazinyl)-2-{4-[(trifluoromethyl)-oxy]phenyl}acetohydrazide, mixture of enantiomers;
formic acid-(±)-2-(1-benzothien-2-yl)-N′-(3,5-dichlorophenyl)-2-(4-methyl-1-piperazinyl)acetohydrazide, mixture of enantiomers;
formic acid-(±)-2-(1-benzothien-5-yl)-N′-(3,5-dichlorophenyl)-2-(4-methyl-1-piperazinyl)acetohydrazide, mixture of enantiomers;
formic acid-(±)-2-dibenzo[b,d]thien-2-yl-N′-(3,5-dichlorophenyl)-2-(4-methyl-1-piperazinyl)acetohydrazide, mixture of enantiomers;
N′-[3,5-bis(trifluoromethyl)phenyl]-2-(2-chlorophenyl)-2-(4-methyl-1-piperazinyl)-acetohydrazide, enantiomer 1;
N′[3,5-bis(trifluoromethyl)phenyl]-2-(2-chlorophenyl)-2-(4-methyl-1-piperazinyl)-acetohydrazide, enantiomer 2;
(±)-N′[3,5-bis(trifluoromethyl)phenyl]-2-(4-methyl-1-piperazinyl)-2-{2-[(trifluoromethyl)-oxy]phenyl}acetohydrazide, mixture of enantiomers;
N′-[3,5-bis(trifluoromethyl)phenyl]-2-(3-methylphenyl)-2-(4-methyl-1-piperazinyl)-acetohydrazide, enantiomer 1;
N′-[3,5-bis(trifluoromethyl)phenyl]-2-(3-methylphenyl)-2-(4-methyl-1-piperazinyl)acetohydrazide, enantiomer 2;
N′-[3,5-bis(trifluoromethyl)phenyl]-2-(2-methylphenyl)-2-(4-methyl-1-piperazinyl)-acetohydrazide, enantiomer 1;
N′-[3,5-bis(trifluoromethyl)phenyl]-2-(2-methylphenyl)-2-(4-methyl-1-piperazinyl)-acetohydrazide, enantiomer 2;
N′-[3,5-bis(trifluoromethyl)phenyl]-2-(2-fluorophenyl)-2-(4-methyl-1-piperazinyl)-acetohydrazide, enantiomer 1;
N′-[3,5-bis(trifluoromethyl)phenyl]-2-(2-fluorophenyl)-2-(4-methyl-1-piperazinyl)-acetohydrazide, enantiomer 2;
N′-(3,5-dichlorophenyl)-2-(2,3-dihydro-1-benzofuran-5-yl)-2-(4-methyl-1-piperazinyl)-acetohydrazide, enantiomer 1;
N′-(3,5-dichlorophenyl)-2-(2,3-dihydro-1-benzofuran-5-yl)-2-(4-methyl-1-piperazinyl)-acetohydrazide, enantiomer 2;
N′-[3,5-bis(trifluoromethyl)phenyl]-2-(2,3-dihydro-1-benzofuran-5-yl)-2-(4-methyl-1-piperazinyl)acetohydrazide, enantiomer 1;
N′-[3,5-bis(trifluoromethyl)phenyl]-2-(2,3-dihydro-1-benzofuran-5-yl)-2-(4-methyl-1-piperazinyl)acetohydrazide, enantiomer 2;
N′-[3,5-bis(trifluoromethyl)phenyl]-2-(2,3-difluorophenyl)-2-(4-methyl-1-piperazinyl)-acetohydrazide, enantiomer 1;
N′-[3,5-bis(trifluoromethyl)phenyl]-2-(2,3-difluorophenyl)-2-(4-methyl-1-piperazinyl)-acetohydrazide, enantiomer 2;
N′-[3,5-bis(trifluoromethyl)phenyl]-2-(2,6-difluorophenyl)-2-(4-methyl-1-piperazinyl)-acetohydrazide, enantiomer 1;
N′-[3,5-bis(trifluoromethyl)phenyl]-2-(2,6-difluorophenyl)-2-(4-methyl-1-piperazinyl)-acetohydrazide, enantiomer 2;
N′-[3,5-bis(trifluoromethyl)phenyl]-2-[2-(methyloxy)phenyl]-2-(4-methyl-1-piperazinyl)-acetohydrazide, enantiomer 1;
N′-[3,5-bis(trifluoromethyl)phenyl]-2-[2-(methyloxy)phenyl]-2-(4-methyl-1-piperazinyl)-acetohydrazide, enantiomer 2;
N′-[3,5-bis(trifluoromethyl)phenyl]-2-[2-fluoro-6-(methyloxy)phenyl]-2-(4-methyl-1-piperazinyl)acetohydrazide, enantiomer
N′-[3,5-bis(trifluoromethyl)phenyl]-2-[2-fluoro-6-(methyloxy)phenyl]-2-(4-methyl-1-piperazinyl)acetohydrazide, enantiomer 2;
N′-[3,5-bis(trifluoromethyl)phenyl]-2-(2,3-dihydro-1,4-benzodioxin-5-yl)-2-(4-methyl-1-piperazinyl)acetohydrazide, enantiomer 1;
N′-[3,5-bis(trifluoromethyl)phenyl]-2-(2,3-dihydro-1,4-benzodioxin-5-yl)-2-(4-methyl-1-piperazinyl)acetohydrazide, enantiomer 2;
(±)-N′-(3,5-dichlorophenyl)-2-(4-fluoro-1-naphthalenyl)-2-(4-methyl-1-piperazinyl)-ethanohydrazide, mixture of enantiomers;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 1;
N′-[3,5-bis(trifluoromethyl)phenyl]-2-(2,3-dihydro-1,4-benzodioxin-6-yl)-2-(4-methyl-1-piperazinyl)ethanohydrazide, enantiomer 2;
(±)-N′-[3,5-bis(trifluoromethyl)phenyl]-2-(4-methyl-1-piperazinyl)-2-{3-[(trifluoromethyl)-oxy]phenyl}acetohydrazide, mixture of enantiomers;
(±)-N′-(3,5-dichlorophenyl)-2-[6-(methyloxy)-2-naphthalenyl]-2-(4-methyl-1-piperazinyl)-acetohydrazide, mixture of enantiomers;
(±)-N′-(3,5-Dimethylphenyl)-2-(4-methyl-1-piperazinyl)-2-(1-naphthalenyl)-acetohydrazide, mixture of enantiomers;
(±)-N′-[3,5-Bis(trifluoromethyl)phenyl]-2-(4-methyl-1-piperazinyl)-2-(1-naphthalenyl)-acetohydrazide hydrochloride, mixture of enantiomers;
2-(1,3-Benzodioxol-5-yl)-N′-[3,5-bis(trifluoromethyl)phenyl]-2-(4-methyl-1-piperazinyl)-acetohydrazide hydrochloride, mixture of enantiomers;
(±)-2-(2-chlorophenyl)-N′-(3,5-dichlorophenyl)-2-(4-methyl-1-piperazinyl)acetohydrazide, mixture of enantiomers;
2-(3-chlorophenyl)-N′-(3,5-dichlorophenyl)-2-(4-methyl-1-piperazinyl)acetohydrazide, enantiomer 1;
2-(3-chlorophenyl)-N′-(3,5-dichlorophenyl)-2-(4-methyl-1-piperazinyl)acetohydrazide, enantiomer 2;
formic acid-(±)-N′-[3,5-bis(trifluoromethyl)phenyl]-2-(3-chlorophenyl)-2-(4-methyl-1-piperazinyl)acetohydrazide (1:1), mixture of enantiomers;
(±)-N′-[3,5-bis(trifluoromethyl)phenyl]-2-(2,2-difluoro-1,3-benzodioxol-4-yl)-2-(4-methyl-1-piperazinyl)acetohydrazide, mixture of enantiomers;
2-(1,3-benzodioxol-5-yl)-N′-(3,5-dichlorophenyl)-2-(4-methyl-1-piperazinyl)ethano-hydrazide hydrochloride, enantiomer 2;
(±)-N′-(3,5-dichlorophenyl)-2-(4-methyl-1-piperazinyl)-2-(1-naphthalenyl)acetohydrazide formate, mixture of enantiomers;
N′-[3,5-bis(trifluoromethyl)phenyl]-2-(4-methyl-1-piperazinyl)-2-(3-pyridinyl)acetohydrazide, enantiomer 1;
N′-[3,5-bis(trifluoromethyl)phenyl]-2-(4-methyl-1-piperazinyl)-2-(3-pyridinyl)acetohydrazide, enantiomer 2;
N′-[3,5-bis(trifluoromethyl)phenyl]-2-imidazo[1,2-a]pyridin-6-yl-2-(4-methyl-1-piperazinyl)-acetohydrazide, enantiomer 1;
N′-[3,5-bis(trifluoromethyl)phenyl]-2-imidazo[1,2-a]pyridin-6-yl-2-(4-methyl-1-piperazinyl)-acetohydrazide, enantiomer 2;
N′-[3,5-bis(trifluoromethyl)phenyl]-2-(4-methyl-1-piperazinyl)-2-(2-methyl-3-pyridinyl)-acetohydrazide, enantiomer 2;
N′-[3,5-bis(trifluoromethyl)phenyl]-2-(4-methyl-1-piperazinyl)-2-(4-methyl-1-piperazinyl)-acetohydrazide, enantiomer 1;
N′-[3,5-bis(trifluoromethyl)phenyl]-2-(4-methyl-1-piperazinyl)-2-(4-methyl-1-piperazinyl)-acetohydrazide, enantiomer 2;
N′-[3,5-bis(trifluoromethyl)phenyl]-2-(4-methyl-1-piperazinyl)-2-(6-methyl-3-pyridinyl)-acetohydrazide, enantiomer 2;
N′-[3,5-bis(trifluoromethyl)phenyl]-2-(3-chloro-2-pyridinyl)-2-(4-methyl-1-piperazinyl)-acetohydrazide, enantiomer 1;
N′-[3,5-bis(trifluoromethyl)phenyl]-2-(3-chloro-2-pyridinyl)-2-(4-methyl-1-piperazinyl)-acetohydrazide, enantiomer 2;
N′-[3,5-bis(trifluoromethyl)phenyl]-2-(1-piperazinyl)-2-(2-pyridinyl)acetohydrazide, enantiomer 1;
N′-[3,5-bis(trifluoromethyl)phenyl]-2-(1-piperazinyl)-2-(2-pyridinyl)acetohydrazide, enantiomer 2;
N′-[3,5-bis(trifluoromethyl)phenyl]-2-(4-methyl-1-piperazinyl)-2-(4-methyl-2-pyridinyl)-acetohydrazide, enantiomer 1;
N′-[3,5-bis(trifluoromethyl)phenyl]-2-(4-methyl-1-piperazinyl)-2-(4-methyl-2-pyridinyl)-acetohydrazide, enantiomer 2;
N′-[3,5-bis(trifluoromethyl)phenyl]-2-(4-methyl-1-piperazinyl)-2-(6-methyl-2-pyridinyl)-acetohydrazide, enantiomer 1;
N′-[3,5-bis(trifluoromethyl)phenyl]-2-(4-methyl-1-piperazinyl)-2-(6-methyl-2-pyridinyl)-acetohydrazide, enantiomer 2;
N′-[3,5-bis(trifluoromethyl)phenyl]-2-(4-methyl-1-piperazinyl)-2-(2-pyridinyl)-acetohydrazide, enantiomer 1;
N′-[3,5-bis(trifluoromethyl)phenyl]-2-(4-methyl-1-piperazinyl)-2-(2-pyridinyl)-acetohydrazide, enantiomer 2;
(±)-2-(1,3-benzodioxol-5-yl)-N′-(3,5-dichlorophenyl)-2-(1-methyl-4-piperidinyl)-acetohydrazide, mixture of enantiomers;
2-(1,3-benzodioxol-5-yl)-N′-(3,5-dichlorophenyl)-2-(1-methyl-4-piperidinyl)acetohydrazide, enantiomer 1;
2-(1,3-benzodioxol-5-yl)-N′-(3,5-dichlorophenyl)-2-(1-methyl-4-piperidinyl)acetohydrazide, enantiomer 2;
(±)-N′-(3,5-dichlorophenyl)-2-(1-methyl-4-piperidinyl)-2-(1-naphthalenyl)acetohydrazide, mixture of enantiomers;
N′-(3,5-dichlorophenyl)-2-(1-methyl-4-piperidinyl)-2-(1-naphthalenyl)acetohydrazide, enantiomer 1;
N′-(3,5-dichlorophenyl)-2-(1-methyl-4-piperidinyl)-2-(1-naphthalenyl)acetohydrazide, enantiomer 2;
N′-[3,5-bis(trifluoromethyl)phenyl]-2-(2-chlorophenyl)-2-(1-piperazinyl)acetohydrazide hydrochloride, enantiomer 2;
N′-[3,5-bis(trifluoromethyl)phenyl]-2-(2-chlorophenyl)-2-(4-methyl-1-piperazinyl)acetohydrazide hydrochloride, enantiomer 1;
N′-[3,5-bis(trifluoromethyl)phenyl]-2-(2,6-dimethyl-3-pyridinyl)-2-(4-methyl-1-piperazinyl)-acetohydrazide, enantiomer
N′-[3,5-bis(trifluoromethyl)phenyl]-2-(4-methyl-1-piperazinyl)-2-(5-pyrimidinyl)acetohydrazide, enantiomer 1;
N′-[3,5-bis(trifluoromethyl)phenyl]-2-(4-methyl-1-piperazinyl)-2-(5-pyrimidinyl)acetohydrazide, enantiomer 2;
N′-[3,5-bis(trifluoromethyl)phenyl]-2-(2,6-dimethyl-3-pyridinyl)-2-[(8aR)-hexahydropyrrolo[1,2-a]pyrazin-2(1H)-yl]acetohydrazide, diastereoisomer 1;
N′-[3,5-bis(trifluoromethyl)phenyl]-2-(2,6-dimethyl-3-pyridinyl)-2-[(8aR)-hexahydropyrrolo[1,2-a]pyrazin-2(1H)-yl]acetohydrazide, diastereoisomer 2;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 1;
N′-[3,5-bis(trifluoromethyl)phenyl]-2-[(8aS)-hexahydropyrrolo[1,2-a]pyrazin-2(1H)-yl]-2-(3-pyridinyl)acetohydrazide, diastereoisomer 2;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aS)-hexahydropyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 1;
N′-(3,5-dichlorophenyl)-2-[(8aS)-hexahydropyrrolo[1,2-a]pyrazin-2(1H)-yl]-2-(1-naphthalenyl)acetohydrazide, diastereoisomer 2;
(2S)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide;
(2R)—N′-[3,5-bis(trifluoromethyl)phenyl]-2-[(8aR)-hexahydropyrrolo[1,2-a]pyrazin-2(1H)-yl]-2-(3-pyridinyl)acetohydrazide hydrochloride;
N′-[3,5-bis(trifluoromethyl)phenyl]-2-[(8aR)-hexahydropyrrolo[1,2-a]pyrazin-2(1H)-yl]-2-(1,3-thiazol-2-yl)acetohydrazide, diastereomeric mixture;
N′-[3,5-bis(trifluoromethyl)phenyl]-2-(8a-methylhexahydropyrrolo[1,2-a]pyrazin-2(1H)-yl)-2-(3-methylphenyl)acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereomeric mixture;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 1;
N′-[3,5-bis(trifluoromethyl)phenyl]-2-(2-ethylphenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[2-(ethyloxy)phenyl]-2-[(8aR)-hexahydropyrrolo[1,2-a]pyrazin-2(1H)-yl]acetohydrazide, mixture of diastereoisomers;
N′-[3,5-bis(trifluoromethyl)phenyl]-2-(2-chloro-3-pyridinyl)-2-[(8aR)-hexahydropyrrolo[1,2-a]pyrazin-2(1H)-yl]acetohydrazide, diastereomeric mixture;
N′-[3,5-bis(trifluoromethyl)phenyl]-2-(4-fluoro-2-methylphenyl)-2-[(8aR)-hexahydropyrrolo[1,2-a]pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-methyl-2-pyridinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(6-methyl-2-pyridinyl)propanohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-methyl-4-(methyloxy)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(3-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[6-(trifluoromethyl)-3-pyridinyl]acetohydrazide, diastereomeric mixture;
N′-[3,5-bis(trifluoromethyl)phenyl]-2-[(8aR)-hexahydropyrrolo[1,2-a]pyrazin-2(1H)-yl]-2-{2-[(trifluoromethyl)oxy]phenyl}acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-(methyloxy)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(2-methylphenyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide, diastereomeric mixture;
N′-[3,5-bis(trifluoromethyl)phenyl]-2-[(8aR)-hexahydropyrrolo[1,2-a]pyrazin-2(1H)-yl]-2-[2-(1-methylethyl)phenyl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, mixture of diastereoisomers;
N′-[3,5-bis(trifluoromethyl)phenyl]-2-[(8aR)-hexahydropyrrolo[1,2-a]pyrazin-2(1H)-yl]-2-(1H-pyrazol-3-yl)acetohydrazide, diastereoisomers 2;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereomeric mixture;
N′-[3,5-bis(trifluoromethyl)phenyl]-2-(2-fluorophenyl)-2-[(8aR)-hexahydro pyrrolo[1,2-a]-pyrazin-2(1H)-yl]acetohydrazide, diastereoisomer 2;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, mixture of diastereoisomers;
N′-[3,5-bis(trifluoromethyl)phenyl]-2-(2-chlorophenyl)-2-[(8aR)-hexahydropyrrolo[1,2-a]-pyrazin-2(1H)-yl]acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereomeric mixture;
N′-[3,5-bis(trifluoromethyl)phenyl]-2-[(8aR)-hexahydropyrrolo[1,2-a]pyrazin-2(1H)-yl]-2-(5-pyrimidinyl)acetohydrazide, diastereoisomer 2;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, mixture of diastereoisomer;
N′-[3,5-bis(trifluoromethyl)phenyl]-2-[(8aR)-hexahydropyrrolo[1,2-a]pyrazin-2(1H)-yl]-2-(1-methyl-1H-imidazol-5-yl)acetohydrazide, diastereoisomer 2;
N′-[3,5-bis(trifluoromethyl)phenyl]-2-(2,6-dibromo-3-pyridinyl)-2-[(8aR)-hexahydropyrrolo[1,2-a]pyrazin-2(1H)-yl]acetohydrazide diastereoisomer 1;
N′-(3,5-dichlorophenyl)-2-(2,3-dihydro-1-benzofuran-5-yl)-2-(4-methylpiperazin-1-yl)acetohydrazide enantiomer 1;
N′-(3,5-dichlorophenyl)-2-(2,3-dihydro-1-benzofuran-5-yl)-2-(4-methylpiperazin-1-yl)acetohydrazide enantiomer 2;
N′-[3,5-bis(trifluoromethyl)phenyl]-2-(2,3-dihydro-1-benzofuran-5-yl)-2-(4-methyl-1-piperazinyl)acetohydrazide, enantiomer 1;
N′-[3,5-bis(trifluoromethyl)phenyl]-2-(2,3-dihydro-1-benzofuran-5-yl)-2-(4-methyl-1-piperazinyl)acetohydrazide, enantiomer 2;
N′-[3,5-bis(trifluoromethyl)phenyl]-2-(2-chlorophenyl)-2-(1-piperazinyl)acetohydrazide-enantiomer 1;
N′-[3,5-bis(trifluoromethyl)phenyl]-2-(2-chlorophenyl)-2-(1-piperazinyl)acetohydrazide-enantiomer 2;
(±)-2-(1,3-Benzodioxol-5-yl)-N′-[3,5-bis(trifluoromethyl)phenyl]-2-(4-methyl-1-piperazinyl)acetohydrazide hydrochloride;
2-(1,3-benzodioxol-5-yl)-N′-(3,5-dichlorophenyl)-2-(4-methyl-1-piperazinyl)-acetohydrazide hydrochloride, enantiomer 2;
N′-[3,5-bis(trifluoromethyl)phenyl]-2-(4-methyl-1-piperazinyl)-2-(6-methyl-3-pyridinyl)-acetohydrazide, enantiomer 1;
(±)-N′-(3,5-dichlorophenyl)-2-(4-methylpiperazin-1-yl)-2-(1-naphthyl)aceto-hydrazide diformate;
2-(4-methylpiperazin-1-yl)-N′-[3,5-bis(trifluoromethyl)phenyl]-2-pyridin-3-ylacetohydrazide, enantiomer 1;
2-(4-methyl piperazin-1-yl)-N′-[3,5-bis(trifluoromethyl)phenyl]-2-pyridin-3-ylacetohydrazide, enantiomer 2;
N′-[3,5-bis(trifluoromethyl)phenyl]-2-(4-methylpiperazin-1-yl)-2-(2-methylpyridin-3-yl)acetohydrazide, enantiomer 1;
N′-[3,5-bis(trifluoromethyl)phenyl]-2-(4-methylpiperazin-1-yl)-2-(2-methylpyridin-3-yl)acetohydrazide, enantiomer 2;
N′-[3,5-bis(trifluoromethyl)phenyl]-2-(4-methyl-1-piperazinyl)-2-(3-methyl-2-pyridinyl)acetohydrazide, enantiomer 1;
N′-[3,5-bis(trifluoromethyl)phenyl]-2-(4-methyl-1-piperazinyl)-2-(3-methyl-2-pyridinyl)acetohydrazide, enantiomer 2;
N′-[3,5-bis(trifluoromethyl)phenyl]-2-(4-methyl-1-piperazinyl)-2-(2-methyl-3-pyridinyl)-acetohydrazide, enantiomer 1;
2-(1,3-benzodioxol-5-yl)-N′-[3,5-bis(trifluoromethyl)phenyl]-2-(4-methyl-1-piperazinyl)propanohydrazide, enantiomer 1;
2-(1,3-benzodioxol-5-yl)-N′-[3,5-bis(trifluoromethyl)phenyl]-2-(4-methyl-1-piperazinyl)propanohydrazide, enantiomer 2;
N′-[3,5-bis(trifluoromethyl)phenyl]-2-(4-methyl-1-piperazinyl)-2-(2-pyridinyl)propanohydrazide, enantiomer 1;
N′-[3,5-bis(trifluoromethyl)phenyl]-2-(4-methyl-1-piperazinyl)-2-(2-pyridinyl)propanohydrazide, enantiomer 2;
and pharmaceutically acceptable salts and solvates thereof, wherein “diastereoisomer 1” and “diastereoisomer 2” indicates a single diastereoisomer in homochiral form whose absolute configuration at one stereocentre was not determined;
and pharmaceutically acceptable salts and solvates thereof, wherein “enantiomer 1” or “enantiomer 2” indicates a single enantiomer of unknown absolute stereochemistry.

11. A compound of claim 1 which is:
(2R)—N′-[3,5-bis(trifluoromethyl)phenyl]-2-[(8aR)-hexahydropyrrolo[1,2-a]pyrazin-2(1H)-yl]-2-(3-pyridinyl)ethanohydrazide and pharmaceutically acceptable salts and solvates thereof.

12. A method of treatment of a mammal suffering from a disorder mediated by the ghrelin receptor, which comprises administering to said subject an effective amount of a compound of claim 1 or a pharmaceutically acceptable salt or solvate thereof.

13-14. (canceled)

15. A pharmaceutical composition which comprises a compound of claim 1 or a pharmaceutically acceptable salt or solvate thereof in admixture with one or more pharmaceutically acceptable carriers, diluents or excipients.

16. (canceled)
